(19) United States
(12) Patent Application Publication
(10) Pub. No.: US 2024/0334820 A1
(43) Pub. Date: Oct. 3, 2024

(54) LIGHT-EMITTING DEVICE INCLUDING HETEROCYCLIC COMPOUND, ELECTRONIC APPARATUS INCLUDING THE LIGHT-EMITTING DEVICE, AND THE HETEROCYCLIC COMPOUND

(71) Applicants: Samsung Display Co., Ltd., Yongin-si (KR); Solus Advanced Materials Co., Ltd., Iksan-si (KR)

(72) Inventors: Hyewon Choi, Yongin-si (KR); Youngmo Kim, Yongin-si (KR); Taehyung Kim, Yongin-si (KR); Jaeyi Sim, Yongin-si (KR); Seunggyu Yang, Yongin-si (KR); Jaehoon Lee, Yongin-si (KR); Yeongrong Park, Yongin-si (KR); Hyun Shin, Yongin-si (KR); Ilsoo Oh, Yongin-si (KR); Daewoong Lee, Yongin-si (KR); Hyeonjeong Im, Yongin-si (KR); Illhun Cho, Yongin-si (KR)

(21) Appl. No.: 18/582,243

(22) Filed: Feb. 20, 2024

(30) Foreign Application Priority Data
Mar. 7, 2023 (KR) 10-2023-0030201
Feb. 8, 2024 (KR) 10-2024-0019826

(51) Int. Cl. H10K 85/60; C07D 251/24; C07D 401/14; C07D 405/14; C09K 11/06; H10K 50/16

(52) U.S. Cl. CPC H10K 85/654; C07D 251/24; C07D 401/14; C07D 405/14; C09K 11/06; H10K 85/615; H10K 85/626; H10K 85/6574; C09K 2211/1018; H10K 50/16; H10K 50/166

(57) ABSTRACT

A light-emitting device includes a first electrode, a second electrode facing the first electrode, an interlayer between the first electrode and the second electrode and including an emission layer, and a heterocyclic compound represented by Formula 1. In addition, an electronic apparatus including the light-emitting device and the heterocyclic compound represented by Formula 1 are also provided.

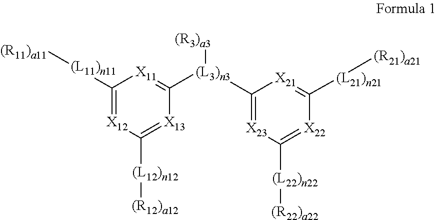

### CROSS-REFERENCE TO RELATED APPLICATION

This application claims priority to and the benefit of Korean Patent Application No. 10-2023-0030201, filed on Mar. 7, 2023, and Korean Patent Application No. 10-2024-0019826, filed on Feb. 8, 2024, in the Korean Intellectual Property Office, the content of each of which is incorporated by reference herein in its entirety.

### BACKGROUND

### 1. Field

One or more embodiments of the present disclosure relate to a light-emitting device including a heterocyclic compound, an electronic apparatus including the light-emitting device, and the heterocyclic compound.

### 2. Description of the Related Art

Among light-emitting devices, self-emissive devices (e.g., organic light-emitting devices) have relatively wide viewing angles, high contrast ratios, short response times, and excellent or suitable characteristics in terms of luminance, driving voltage, and response speed.

In a light-emitting device, a first electrode is located on a substrate, and a hole transport region, an emission layer, an electron transport region, and a second electrode are sequentially arranged on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition and decay from an excited state to a ground state to thereby generate light.

### SUMMARY

One or more aspects of embodiments of the present disclosure are directed toward a light-emitting device including a heterocyclic compound, an electronic apparatus including the light-emitting device, and the heterocyclic compound.

Additional aspects will be set forth in part in the description which follows and, in part, will be apparent from the description, or may be learned by practice of the presented embodiments of the present disclosure.

According to one or more embodiments of the present disclosure, a light-emitting device includes:
a first electrode,
a second electrode facing the first electrode, and
an interlayer between the first electrode and the second electrode and including an emission layer, and
a heterocyclic compound represented by Formula 1:

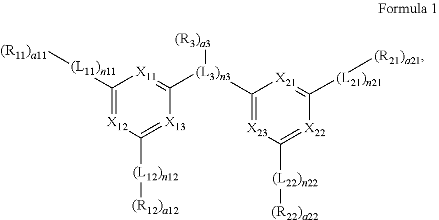

wherein, in Formula 1,
X₁₁ may be C(Y₁₁) or N, X₁₂ may be C(Y₁₂) or N, X₁₃ may be C(Y₁₃) or N, X₂₁ may be C(Y₂₁) or N, X₂₂ may be C(Y₂₂) or N, and X₂₃ may be C(Y₂₃) or N,
At least one selected from among X₁₁ to X₁₃ may be N, and at least one selected from among X₂₁ to X₂₃ may be N,
L₁₁, L₁₂, L₂₁, and L₂₂ may each independently be a single bond, a C₃-C₆₀ carbocyclic group unsubstituted or substituted with at least one R₁₀ₐ, a C₁-C₆₀ heterocyclic group unsubstituted or substituted with at least one R₁₀ₐ, *—C(R₁ₐ)(R_{1b})—*′, *—C(R₁ₐ)═**, *═C(R₁ₐ)—**, *—C(R₁ₐ)═C(R_{1b})—**, *—C≡C—**, *—N(R₁ₐ)—*′, or *—Si(R₁ₐ)(R_{1b})—**,
L₃ may be a single bond, a C₃-C₆₀ carbocyclic group unsubstituted or substituted with at least one R₁₀ₐ, a C₁-C₆₀ heterocyclic group unsubstituted or substituted with at least one R₁₀ₐ,
R₃ may be absent when L₃ is a single bond,
n11, n12, n21, n22, and n3 may each independently be an integer from 1 to 20,
R₁₁, R₁₂, R₂₁, R₂₂, R₃, R₁ₐ, R_{1b}, Y₁₁ to Y₁₃, and Y₂₁ to Y₂₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a11, a12, a21, a22, and a3 may each independently be an integer from 1 to 20,
at least one selected from among R₁₁, R₁₂, R₂₁, R₂₂ and R₃ may be a bridged C₅-C₁₂ polycyclic group unsubstituted or substituted with at least one R₁₀ₐ, wherein R₁₁, R₁₂, R₂₁, R₂₂, and R₃ may each not be an adamantyl group,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁) (Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments of the present disclosure, an electronic apparatus includes the light-emitting device.

According to one or more embodiments of the present disclosure, electronic equipment includes the light-emitting device.

According to one or more embodiments of the present disclosure, provided is a heterocyclic compound represented by Formula 1.

### BRIEF DESCRIPTION OF THE DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic view of a structure of a light-emitting device according to one or more embodiments of the present disclosure;

FIG. 2 is a schematic view of a structure of an electronic apparatus according to one or more embodiments of the present disclosure;

FIG. 3 is a schematic view of a structure of an electronic apparatus according to one or more embodiments of the present disclosure; and

FIGS. 4, 5, 6A, 6B, and 6C are each a schematic view of a structure of electronic equipment according to one or more embodiments of the present disclosure.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As utilized herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

According to one or more embodiments of the present disclosure, a light-emitting device may include:
a first electrode;
a second electrode facing the first electrode;
an interlayer between the first electrode and the second electrode and including an emission layer; and
a heterocyclic compound represented by Formula 1:

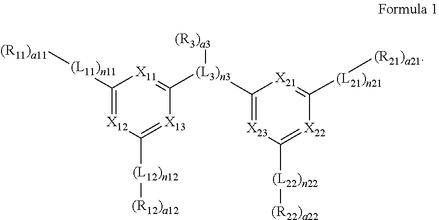

Details on Formula 1 may be the same as described herein.

In one or more embodiments,
the first electrode of the light-emitting device may be an anode,
the second electrode of the light-emitting device may be a cathode,
the interlayer of the light-emitting device may further include a hole transport region between the first electrode and the emission layer and an electron transport region between the emission layer and the second electrode,
the hole transport region may include a hole injection layer, a hole transport layer, an emission auxiliary layer, an electron blocking layer, or any combination thereof, and
the electron transport region may include a hole blocking layer, an electron transport layer, an electron injection layer, or any combination thereof.

In one or more embodiments, the emission layer in the interlayer of the light-emitting device may include a dopant and a host. For example, in some embodiments, the dopant may include a transition metal and ligand(s) in the number of m, and m may be an integer from 1 to 6. The ligand(s) in the number of m may be identical to or different from each other, at least one of the ligand(s) in the number of m may be bonded to the transition metal via a carbon-transition metal bond, and the carbon-transition metal bond may be a coordinate bond. For example, in some embodiments, at least one of the ligand(s) in the number of m may be a carbene ligand (e.g., in Ir(pmp)₃ and/or the like). The transition metal may be, for example, iridium, platinum, osmium, palladium, rhodium, gold, or the like. More details on the emission layer and the dopant may be the same as described herein.

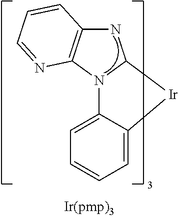

In one or more embodiments, the electron transport region (e.g., a buffer layer, a hole blocking layer, an electron control layer, or an electron transport layer in the electron transport region) may include a metal-free compound including at least one π electron-deficient nitrogen-containing C₁-C₆₀ heterocyclic group.

In one or more embodiments, the electron transport region (e.g., an electron transport layer in the electron transport region) may further include a metal-containing material. The metal-containing material may include an alkali metal complex, an alkaline earth metal complex, or any combination thereof.

In one or more embodiments, the electron transport region may include an electron injection layer to facilitate the injection of electrons from the second electrode. The electron injection layer may be in direct contact with the second electrode. The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

In one or more embodiments, the electron transport region of the light-emitting device may further include an electron transport auxiliary layer. For example, in some embodiments, the electron transport auxiliary layer may be in direct contact with the emission layer.

More details on the electron transport region may be the same as described in the present disclosure.

In one or more embodiments, the heterocyclic compound may be included between the first electrode and the second electrode of the light-emitting device. Therefore, the heterocyclic compound may be included in the interlayer of the light-emitting device.

In one or more embodiments, the heterocyclic compound may be included in the electron transport region.

In one or more embodiments, the heterocyclic compound may be included in the electron transport layer.

In one or more embodiments, the heterocyclic compound may be included in the electron transport auxiliary layer.

In one or more embodiments, the heterocyclic compound may be included in the emission layer.

In one or more embodiments, the emission layer may be to emit blue light. The blue light may have a maximum emission wavelength in a range of, for example, about 430 nm to about 480 nm.

In one or more embodiments, the light emitted from the emission layer may have a maximum emission wavelength in a range of about 400 nm to about 500 nm, about 410 nm to about 490 nm, about 420 nm to about 480 nm, about 430 nm to about 475 nm, about 440 nm to about 475 nm, about 450 nm to about 475 nm, about 430 nm to about 470 nm, about 440 nm to about 470 nm, about 450 nm to about 470 nm, about 430 nm to about 465 nm, about 440 nm to about 465 nm, about 450 nm to about 465 nm, about 430 nm to about 460 nm, about 440 nm to about 460 nm, or about 450 nm to about 460 nm.

In one or more embodiments, the light-emitting device may have a driving voltage of less than or equal to 4.0 eV. For example, in some embodiments, the light-emitting device may have a driving voltage in a range of about 2.0 eV to about 4.0 eV, about 2.0 eV to about 3.8 eV, about 2.0 eV to about 3.6 eV, about 2.0 eV to about 3.5 eV, about 2.5 eV to about 4.0 eV, about 2.5 eV to about 3.8 eV, about 2.5 eV to about 3.6 eV, about 2.5 eV to about 3.5 eV, about 3.0 eV to about 4.0 eV, about 3.0 eV to about 3.8 eV, about 3.0 eV to about 3.6 eV, about 3.0 eV to about 3.5 eV, about 3.1 eV to about 4.0 eV, about 3.1 eV to about 3.8 eV, about 3.1 eV to about 3.6 eV, or about 3.1 eV to about 3.5 eV.

In one or more embodiments, the light-emitting device may have current efficiency of greater than or equal to 7.5 cd/A. For example, in some embodiments, the light-emitting device may have current efficiency in a range of about 7.5 cd/A to about 9.0 cd/A, about 7.5 cd/A to about 8.8 cd/A, about 7.5 cd/A to about 8.6 cd/A, about 7.5 cd/A to about 8.5 cd/A, about 7.6 cd/A to about 9.0 cd/A, about 7.6 cd/A to about 8.8 cd/A, about 7.6 cd/A to about 8.6 cd/A, about 7.6 cd/A to about 8.5 cd/A, about 7.7 cd/A to about 9.0 cd/A, about 7.7 cd/A to about 8.8 cd/A, about 7.7 cd/A to about 8.6 cd/A, about 7.7 cd/A to about 8.5 cd/A, about 7.8 cd/A to about 9.0 cd/A, about 7.8 cd/A to about 8.8 cd/A, about 7.8 cd/A to about 8.6 cd/A, or about 7.8 cd/A to about 8.5 cd/A.

In one or more embodiments, the light-emitting device may further include a capping layer arranged outside (e.g., on) the first electrode and/or outside (e.g., on) the second electrode.

In one or more embodiments, the light-emitting device may further include at least one of a first capping layer arranged outside (e.g., on) the first electrode or a second capping layer arranged outside (e.g., on) the second electrode, and the heterocyclic compound represented by Formula 1 may be included in at least one of the first capping layer or the second capping layer. More details on the first capping layer and/or second capping layer may be the same as described herein.

In one or more embodiments, the light-emitting device may further include:
a first capping layer arranged outside (e.g., on) the first electrode and including the heterocyclic compound represented by Formula 1;
a second capping layer arranged outside (e.g., on) the second electrode and including the heterocyclic compound represented by Formula 1; or
the first capping layer and the second capping layer.

The expression “(an interlayer and/or a capping layer) include(s) a heterocyclic compound represented by Formula 1” as utilized herein may include an embodiment in which “(an interlayer and/or a capping layer) include(s) identical heterocyclic compounds represented by Formula 1” and an embodiments in which “(an interlayer and/or a capping layer) include(s) two or more different heterocyclic compounds represented by Formula 1.”

For example, in some embodiments, the interlayer and/or the capping layer may include, as the heterocyclic compound, Compound 1 only. In those embodiments, Compound 1 may be included in the electron transport region of the light-emitting device. In one or more embodiments, the interlayer may include, as the heterocyclic compound, Compounds 1 and 2. In those embodiments, Compounds 1 and 2 may exist (e.g., be present) in an identical layer (for example, Compounds 1 and 2 may all exist (e.g., be present) in an electron transport region), or different layers (for example, Compound 1 may exist (e.g., be present) in an electron transport region and Compound 2 may exist in an emission layer).

The term “interlayer” as utilized herein refers to a single layer and/or all of a plurality of layers between the first electrode and the second electrode of the light-emitting device.

According to one or more embodiments of the present disclosure, an electronic apparatus may include the light-emitting device. In one or more embodiments, the electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode of the thin-film transistor. In one or more embodiments, the electronic apparatus may further include a color filter, a color conversion layer, a touch screen layer, a polarizing layer, or any combination thereof. More details on the electronic apparatus may be the same as described herein.

According to one or more embodiments of the present disclosure, electronic equipment may include the light-emitting device.

For example, in one or more embodiments, the electronic equipment may be at least one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, or a signboard.

According to one or more embodiments of the present disclosure, provided is a heterocyclic compound represented by Formula 1:

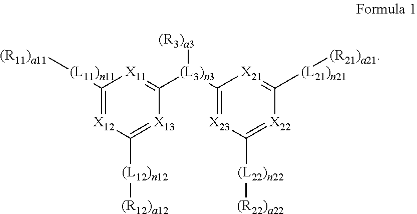

In Formula 1, X₁₁ may be C(Y₁₁) or N, X₁₂ may be C(Y₁₂) or N, X₁₃ may be C(Y₁₃) or N, X₂₁ may be C(Y₂₁) or N, X₂₂ may be C(Y₂₂) or N, and X₂₃ may be C(Y₂₃) or N.

In one or more embodiments, at least one selected from among X₁₁ to X₁₃ may be N, and at least one selected from among X₂₁ to X₂₃ may be N.

In one or more embodiments, X₁₁ to X₁₃ and X₂₁ to X₂₃ may all be N.

In one or more embodiments, Y₁₁ to Y₁₃ and Y₂₁ to Y₂₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In one or more embodiments, R₁₀ₐ and Q₁ to Q₃ may each be the same as described herein.

In one or more embodiments, Y₁₁ to Y₁₃ and Y₂₁ to Y₂₃ may each independently be:
hydrogen, deuterium, —F, —Cl, —Br, —I, a hydroxyl group, a cyano group, a nitro group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzosilolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or
—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).
Q₁ to Q₃ and Q₃₁ to Q₃₃ may each be the same as described herein.

In Formula 1, L₁₁, L₁₂, L₂₁, and L₂₂ may each independently be a single bond, a C₅-C₆₀ carbocyclic group unsubstituted or substituted with at least one R₁₀ₐ, a C₁-C₆₀ heterocyclic group unsubstituted or substituted with at least one R₁₀ₐ, *—C(R₁ₐ)(R_{1b})—*′, *—C(R₁ₐ)═**, *═C(R₁ₐ)—**, *—C(R₁ₐ)═C(R_{1b})—**, *—C≡C—**, *—N(R₁ₐ)—**, or *—Si(R₁ₐ)(R_{1b})—**.

In Formula 1, L₃ may be a single bond, a C₃-C₆₀ carbocyclic group unsubstituted or substituted with at least one R₁₀ₐ, a C₁-C₆₀ heterocyclic group unsubstituted or substituted with at least one R₁₀ₐ, and R₃ may be absent when L₃ is a single bond.

In Formula 1, n11, n12, n21, n22, and n3 indicate the number of L₁₁(s), the number of L₁₂(s), the number of L₂₁(s), the number of L₂₂(s), and the number of L₃(s), respectively, and may each independently be an integer from 1 to 20. When n11 is 2 or more, two or more of L₁₁(s) may be identical to or different from each other, when n12 is 2 or more, two or more of L₁₂(s) may be identical to or different from each other, when n21 is 2 or more, two or more of L₂₁(s) may be identical to or different from each other, when n22 is 2 or more, two or more of L₂₂(s) may be identical to or different from each other, and when n3 is 2 or more, two or more of L₃(s) may be identical to or different from each other.

In one or more embodiments, L₁₁, L₁₂, L₂₁, L₂₂, and L₃ may each independently be:
a single bond; or
a benzene group, a pentalene group, an indene group, a naphthalene group, an azulene group, a heptalene group, an indacene group, an acenaphthylene group, a fluorene group, a spiro-bifluorene group, a benzofluorene group, a phenalene group, a phenanthrene group, an anthracene group, a fluoranthene group, a triphenylene group, a pyrene group, a chrysene group, a naphthacene group, a picene group, a perylene group, a pentaphenylene group, a hexacene group, a pyrrole group, an imidazole group, a pyrazole group, a pyridine group, a pyrazine group, a pyrimidine group, a pyridazine group, an isoindole group, an indole group, an indazole group, a purine group, a quinoline group, an isoquinoline group, a benzoquinoline group, a phthalazine group, a naphthyridine group, a quinoxaline group, a quinazoline group, a cinnoline group, a carbazole group, a dibenzosilole group, a phenanthridine group, an acridine group, a phenanthroline group, a phenazine group, a benzoxazole group, a benzimidazole group, a furan group, a benzofuran group, a thiophene group, a benzothiophene group, a thiazole group, an isothiazole group, a benzothiazole group, an isoxazole group, an oxazole group, a triazole group, a tetrazole group, an oxadiazole group, a triazine group, a benzoxazole group, a dibenzofuran group, a dibenzothiophene group, a benzocarbazole group, or a dibenzocarbazole group, each unsubstituted or substituted with at least one of deuterium, —F, —Cl, —Br, —I, a hydroxyl group, a cyano group, a nitro group, a C₁-C₂₀ alkyl group, a C₁-C₂₀ alkoxy group, a C₆-C₂₀ aryl group, a C₁-C₂₀ heteroaryl group, —C(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), or a combination thereof.

Q₃₁ to Q₃₃ may each be the same as described herein.

In one or more embodiments, L₁₁, L₁₂, L₂₁, L₂₂, and L₃ may each independently be:
a single bond, a benzene group, a naphthalene group, an azulene group, a heptalene group, a phenalene group, a phenanthrene group, an anthracene group, a pyrrole group, a furan group, a thiophene group, a cyclopentadiene group, a silole group, a pyridine group, a pyrimidine group, a pyrazine group, a triazine group, a carbazole group, a dibenzofuran group, a dibenzothiophene group, a fluorene group, or a dibenzosilole group.

In one or more embodiments, L₁₁, L₁₂, L₂₁, and L₂₂ may each independently be selected from groups represented by Formulae 1-1 to 1-37:

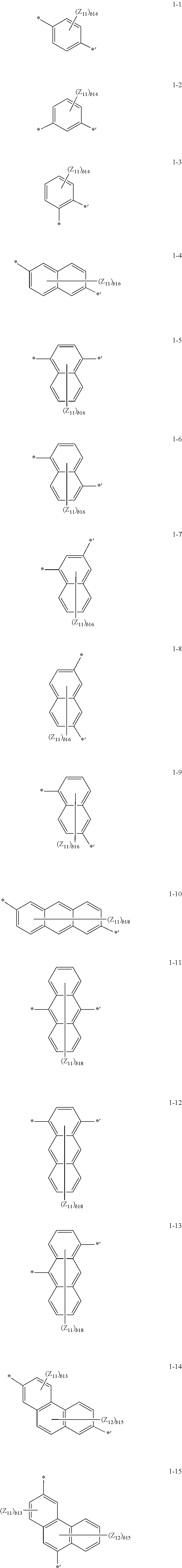

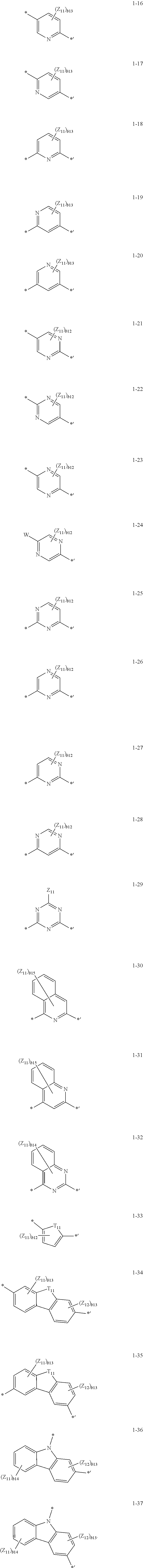

In Formulae 1-1 to 1-37,
T₁₁ may be O, S, C(Z₁₃)(Z₁₄), N(Z₁₅), or Si(Z₁₆)(Z₁₇),
Z₁₁ to Z₁₇ may each independently be selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, —Si(Q₃₁)(Q₃₂)(Q₃₃), —N(Q₃₁)(Q₃₂), and —B(Q₃₁)(Q₃₂),
b12 may be 1 or 2,
b13 may be an integer from 1 to 3,
b14 may be an integer from 1 to 4,
b15 may be an integer from 1 to 5,
b16 may be an integer from 1 to 6,
b18 may be an integer from 1 to 8,
Q₃₁ to Q₃₃ may each be the same as described herein, and
* and *′ each indicate a binding site to a neighboring atom.

In an embodiment, L₃ may be a benzene group, a naphthalene group, a phenanthrene group, or an anthracene group, and n3 may be 1 or 2.

In one or more embodiments, L₃ may be selected from groups represented by Formulae 2-1 to 2-22:

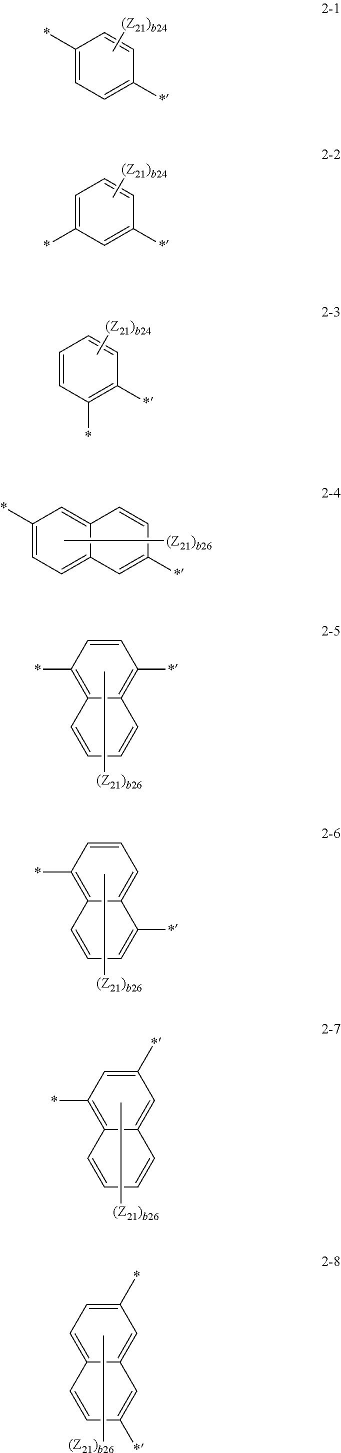

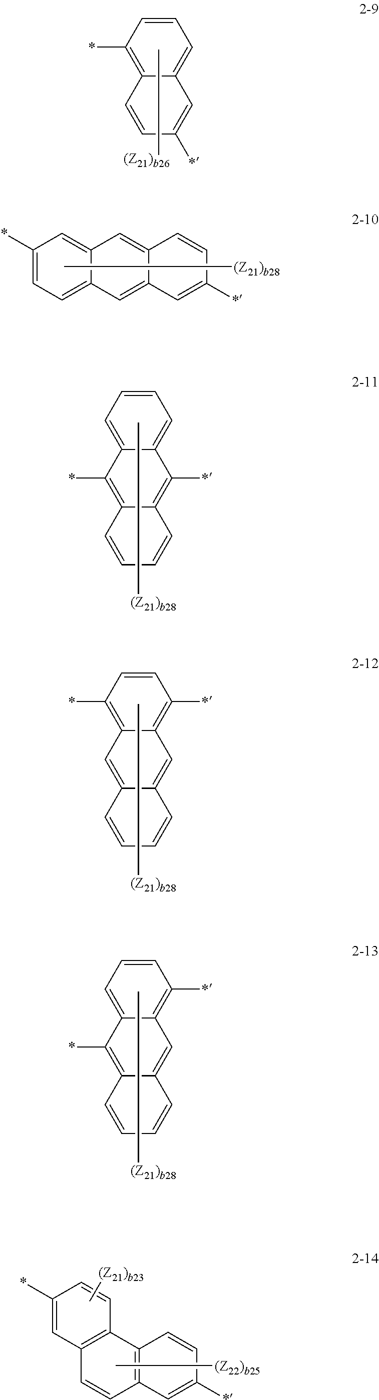

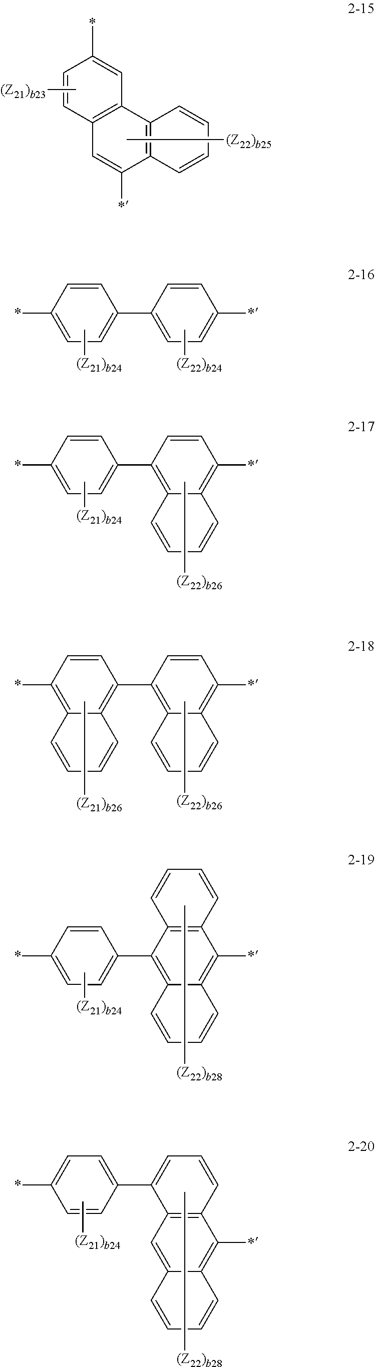

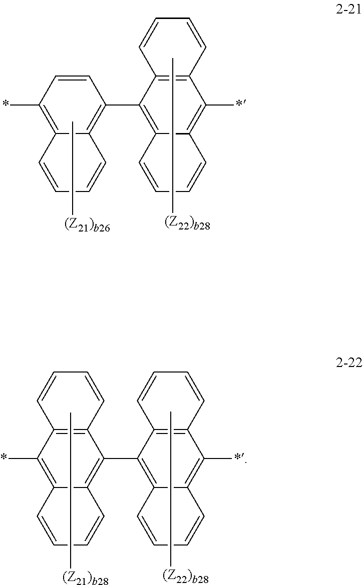

In Formulae 2-1 to 2-22,
Z₂₁ and Z₂₂ may each independently be selected from *″, hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, —Si(Q₃₁)(Q₃₂)(Q₃₃), —N(Q₃₁)(Q₃₂), and —B(Q₃₁)(Q₃₂),
b23 may be an integer from 1 to 3,
b24 may be an integer from 1 to 4,
b25 may be an integer from 1 to 5,
b26 may be an integer from 1 to 6,
b28 may be an integer from 1 to 8,
Q₃₁ to Q₃₃ may each be the same as described herein, and
*, *′, and *″ each indicate a binding site to a neighboring atom.

For example, *″ may indicate a binding site to R₃ in Formula 1.

In one or more embodiments, Ra and R_{1b} may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

R₁₀ₐ and Q₁ to Q₃ may each be the same as described herein.

In one or more embodiments, R₁ₐ and R_{1b} may each independently be:
hydrogen, deuterium, —F, —Cl, —Br, —I, a hydroxyl group, a cyano group, a nitro group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzosilolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or
—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).
Q₁ to Q₃ and Q₃₁ to Q₃₃ may each be the same as described herein.

In Formula 1, R₁₁, R₁₂, R₂₁, R₂₂, and R₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

R₁₀ₐ and Q₁ to Q₃ may each be the same as described herein.

In Formula 1, a11, a12, a21, a22, and a3 indicate the number of R₁₁(s), the number of R₁₂(s), the number of R₂₁(s), the number of R₂₂(s), and the number of R₃(s), respectively, and may each independently be an integer from 1 to 20. When a11 is 2 or more, two or more of R₁₁(s) may be identical to or different from each other, when a12 is 2 or more, two or more of R₁₂(s) may be identical to or different from each other, when a21 is 2 or more, two or more of R₂₁(s) may be identical to or different from each other, when a22 is 2 or more, two or more of R₂₂(s) may be identical to or different from each other, and when a3 is 2 or more, two or more of R₃(s) may be identical to or different from each other.

In one or more embodiments, at least one selected from among R₁₁, R₁₂, R₂₁, R₂₂, and R₃ may be a bridged C₅-C₁₂ polycyclic group unsubstituted or substituted with at least one R₁₀ₐ.

In one or more embodiments, R₁₁, R₁₂, R₂₁, R₂₂, and R₃ may each not be an adamantyl group.

In one or more embodiments, L₁₁ is not a single bond when R₁₁ is a bridged C₅-C₁₂ polycyclic group unsubstituted or substituted with at least one R₁₀ₐ,
L₁₂ is not a single bond when R₁₂ is a bridged C₅-C₁₂ polycyclic group unsubstituted or substituted with at least one R₁₀ₐ,
L₂₁ is not a single bond when R₂₁ is a bridged C₅-C₁₂ polycyclic group unsubstituted or substituted with at least one R₁₀ₐ,
L₂₂ is not a single bond when R₂₂ is a bridged C₅-C₁₂ polycyclic group unsubstituted or substituted with at least one R₁₀ₐ, and
L₃ is not a single bond when R₃ is a bridged C₅-C₁₂ polycyclic group unsubstituted or substituted with at least one R₁₀ₐ.

In one or more embodiments, R₁₁, R₁₂, R₂₁, R₂₂, and R₃ may each independently be:
hydrogen, deuterium, —F, —Cl, —Br, —I, a hydroxyl group, a cyano group, a nitro group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, a bicyclo[1.1.1]pentanyl group, a bicyclo[2.1.1]hexanyl group, a bicyclo[2.2.1]heptanyl group, a bicyclo[2.2.2]octanyl group, a bicyclo[3.2.1]octanyl group, a bicyclo[3.2.2]nonanyl group, a bicyclo[3.3.1]nonanyl group, a tricyclo[3.1.1.0(3,6)]heptanyl group, a tricyclo[4.1.1.0(3,7)]octanyl group, a bicyclo[1.1.1]cyclopentenyl group, a bicyclo[2.1.1]cyclohexenyl group, a bicyclo[2.2.1]cycloheptenyl group, a bicyclo[2.2.2]octenyl group, a bicyclo[3.2.1]octenyl group, a bicyclo[3.2.2]nonenyl group, a bicyclo[3.3.1]nonenyl group, a tricyclo[3.1.1.0(3,6)]cycloheptenyl group, or a tricyclo[4.1.1.0(3,7)]octenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzosilolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or
—C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

Q₁ to Q₃ and Q₃₁ to Q₃₃ may each be the same as described herein.

In one or more embodiments, R₁₁, R₁₂, R₂₁, R₂₂, and R₃ may each independently be:
a cyclopentyl group, a cyclohexyl group, a cycloheptyl group, a cyclooctyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a pyrrolyl group, an imidazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a triazolyl group, a triazinyl group, a benzocarbazolyl group, a dibenzocarbazolyl group, an azacarbazolyl group, an azadibenzofuranyl group, a bicyclo[1.1.1]pentanyl group, a bicyclo[2.1.1]hexanyl group, a bicyclo[2.2.1]heptanyl group, a bicyclo[2.2.2]octanyl group, a bicyclo[3.2.1]octanyl group, a bicyclo[3.2.2]nonanyl group, a bicyclo[3.3.1]nonanyl group, a tricyclo[3.1.1.0(3,6)]heptanyl group, a tricyclo[4.1.1.0(3,7)]octanyl group, a bicyclo[1.1.1]cyclopentenyl group, a bicyclo[2.1.1]cyclohexenyl group, a bicyclo[2.2.1]cycloheptenyl group, a bicyclo[2.2.2]octenyl group, a bicyclo[3.2.1]octenyl group, a bicyclo[3.2.2]nonenyl group, a bicyclo[3.3.1]nonenyl group, a tricyclo[3.1.1.0(3,6)]cycloheptenyl group, or a tricyclo[4.1.1.0(3,7)]octenyl group, each unsubstituted or substituted with a deuterium, a cyano group, a C₁-C₂₀ alkyl group, a cyclopentyl group, a cyclohexyl group, a cycloheptyl group, a cyclooctyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a benzimidazolyl group, a triazolyl group, a triazinyl group, a benzocarbazolyl group, a dibenzocarbazolyl group, —N(Q₃₁)(Q₃₂), or any combination thereof,
Q₃₁ and Q₃₂ may each independently be: hydrogen; deuterium; a cyano group; a C₁-C₆₀ alkyl group; a C₂-C₆₀ alkenyl group; a C₂-C₆₀ alkynyl group; or a C₃-C₆₀ carbocyclic group or a C₁-C₆₀ heterocyclic group, each unsubstituted or substituted with deuterium, a cyano group, a C₁-C₆₀ alkyl group, a phenyl group, a biphenyl group, or any combination thereof.

In one or more embodiments, at least one selected from R₁₁, R₁₂, R₂₁, R₂₂, and R₃ may each independently be:

a bicyclo[1.1.1]pentanyl group, a bicyclo[2.1.1]hexanyl group, a bicyclo[2.2.1]heptanyl group, a bicyclo[2.2.2]octanyl group, a bicyclo[3.2.1]octanyl group, a bicyclo[3.2.2]nonanyl group, a bicyclo[3.3.1]nonanyl group, a tricyclo[3.1.1.0(3,6)]heptanyl group, a tricyclo[4.1.1.0(3,7)]octanyl group, a bicyclo[1.1.1]cyclopentenyl group, a bicyclo[2.1.1]cyclohexenyl group, a bicyclo[2.2.1]cycloheptenyl group, a bicyclo[2.2.2]octenyl group, a bicyclo[3.2.1]octenyl group, a bicyclo[3.2.2]nonenyl group, a bicyclo[3.3.1]nonenyl group, a tricyclo[3.1.1.0(3,6)]cycloheptenyl group, or a tricyclo[4.1.1.0(3,7)]octenyl group.

In one or more embodiments, at least one selected from R₁₁, R₁₂, R₂₁, R₂₂, and R₃ may be selected from groups represented by Formulae 3-1 to 3-24:

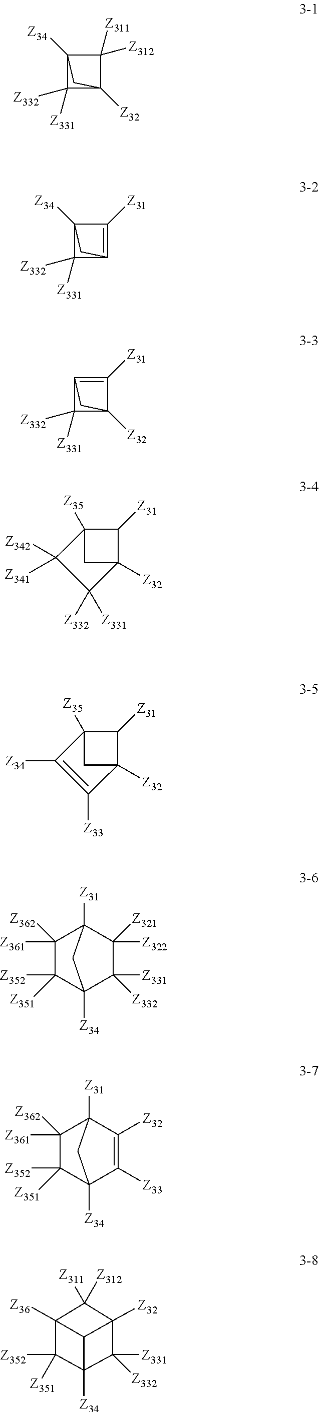

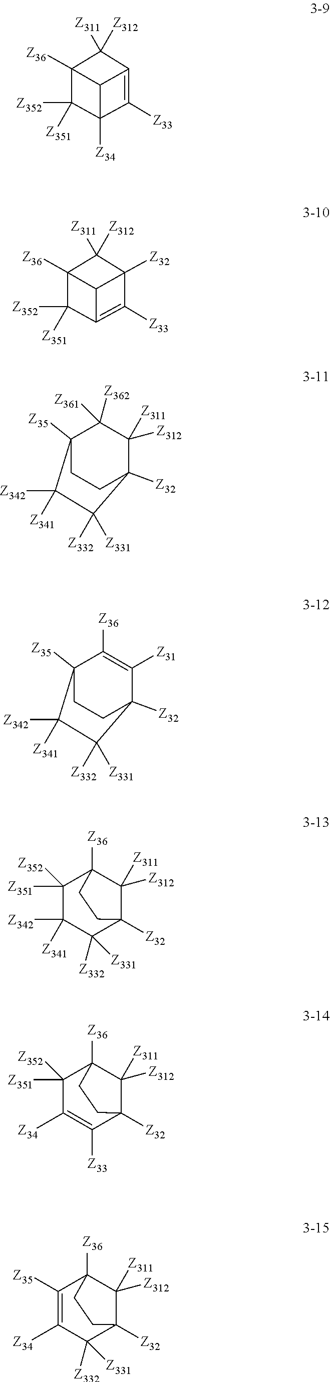

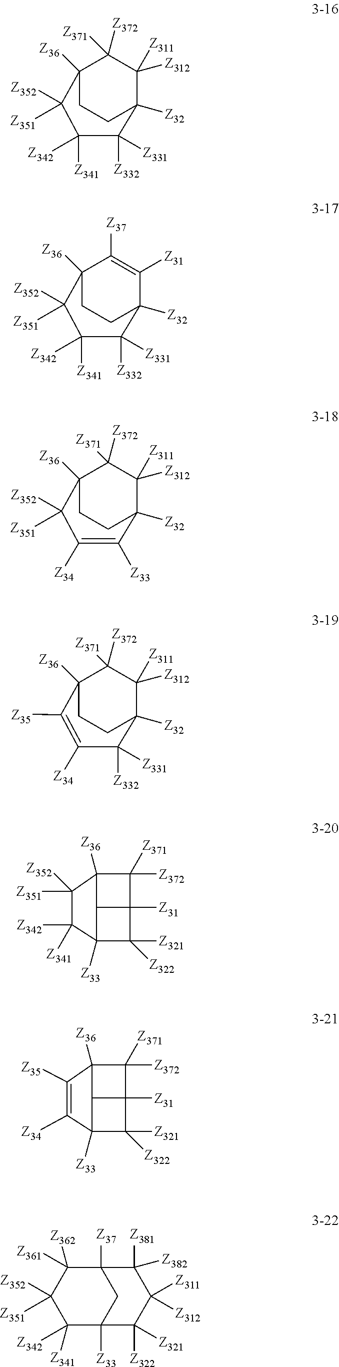

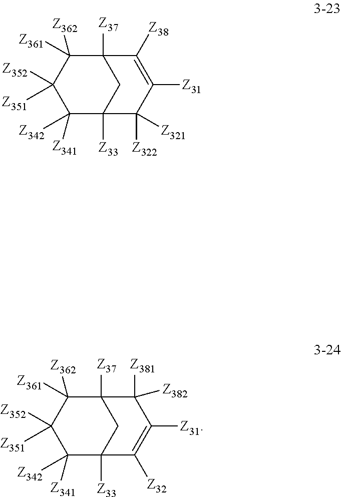

In Formulae 3-1 to 3-24,
Z₃₁ to Z₃₈, Z₃₁₁, Z₃₁₂, Z₃₂₁, Z₃₂₂, Z₃₃₁, Z₃₃₂, Z₃₄₁, Z₃₄₂, Z₃₅₁, Z₃₅₂, Z₃₆₁, Z₃₆₂, Z₃₇₁, Z₃₇₂, Z₃₈₁, and Z₃₈₂ may each independently be selected from:
hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, —Si(Q₃₁)(Q₃₂)(Q₃₃), —N(Q₃₁)(Q₃₂), or —B(Q₃₁)(Q₃₂); and
*,
wherein Q₃₁ to Q₃₃ may each be the same as described herein, and
* indicates a binding site to a neighboring atom.

Unless defined otherwise, for example, R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁) (Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Unless defined otherwise, in the present disclosure, Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₈-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

For example, in some embodiments of the present disclosure, R₁₀ₐ may each independently be:
hydrogen, deuterium, —F, —Cl, —Br, —I, a hydroxyl group, a cyano group, a nitro group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —O(Q₃₁), —S(Q₃₁), —Si(Q₃₁) (Q₃₂)(Q₃₃), —N(Q₃₁)(Q₃₂), —B(Q₃₁)(Q₃₂), —P(Q₃₁) (Q₃₂), —C(═O)(Q₃₁), —S(═O)₂(Q₃₁), —P(═O)(Q₃₁)(Q₃₂), or any combination thereof; or
—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
wherein Q₁ to Q₃ and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with deuterium, a C₁-C₁₀ alkyl group, a phenyl group, a biphenyl group, a pyridinyl group, a pyrimidinyl group, a pyridazinyl group, a pyrazinyl group, a triazinyl group, or any combination thereof.

For example, in one or more embodiments, in Formula 1, i) R₁₁, R₁₂, R₂₁, R₂₂, R₃, and ii) R₁₀ₐ may each independently be hydrogen, deuterium, —F, a cyano group, a nitro group, —CH₃, —CD₃, —CD₂H, —CDH₂, —CF₃, —CF₂H, —CFH₂, a group represented by one selected from Formulae 9-1 to 9-19, a group represented by one selected from Formulae 10-1 to 10-246, —C(Q₁)(Q₂)(Q₃), —Si(Q₁)(Q₂)(Q₃), —P(═O)(Q₁)(Q₂) (wherein Q₁ to Q₃ may each be the same as described herein), a bicyclo[1.1.1]pentanyl group, a bicyclo[2.1.1]hexanyl group, a bicyclo[2.2.1]heptanyl group, a bicyclo[2.2.2]octanyl group, a bicyclo[3.2.1]octanyl group, a bicyclo[3.2.2]nonanyl group, a bicyclo[3.3.1]nonanyl group, a tricyclo[3.1.1.0(3,6)]heptanyl group, a tricyclo[4.1.1.0(3,7)]octanyl group, a bicyclo[1.1.1]cyclopentenyl group, a bicyclo[2.1.1]cyclohexenyl group, a bicyclo[2.2.1]cycloheptenyl group, a bicyclo[2.2.2]octenyl group, a bicyclo[3.2.1]octenyl group, a bicyclo[3.2.2]nonenyl group, a bicyclo[3.3.1]nonenyl group, a tricyclo[3.1.1.0(3,6)]cycloheptenyl group, or a tricyclo[4.1.1.0(3,7)]octenyl group:

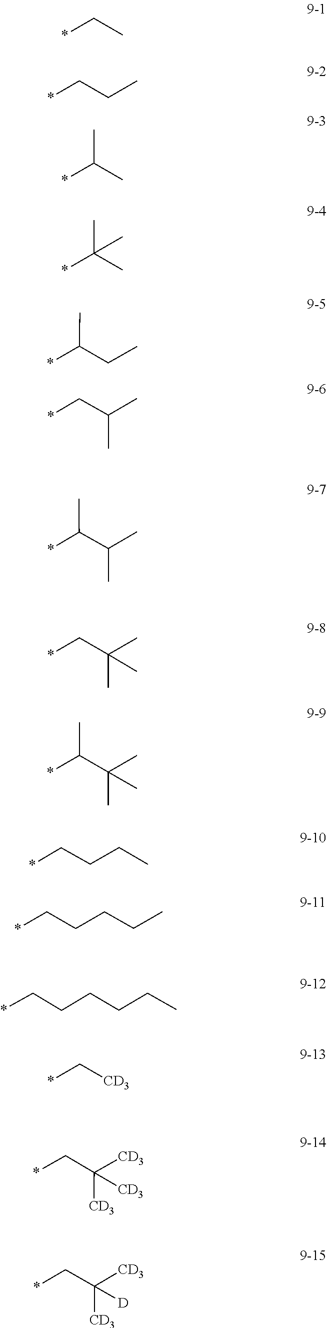

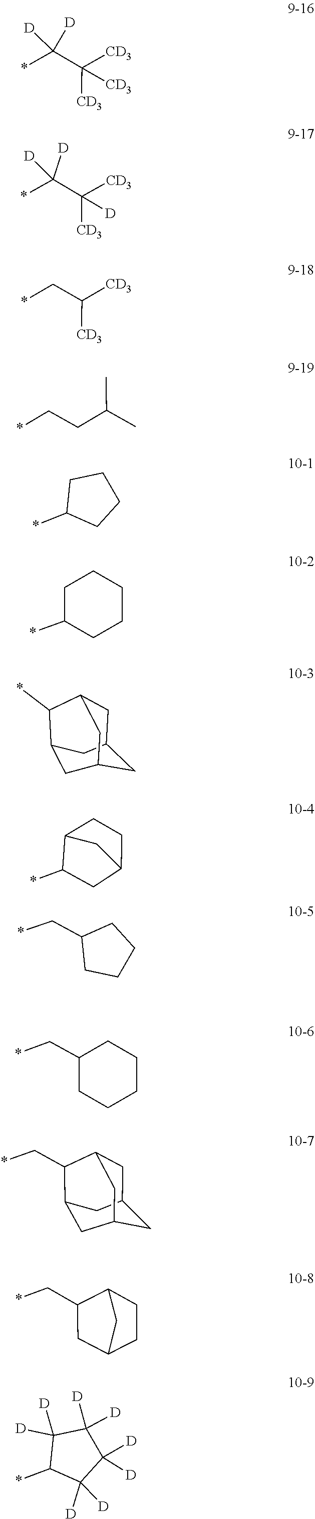

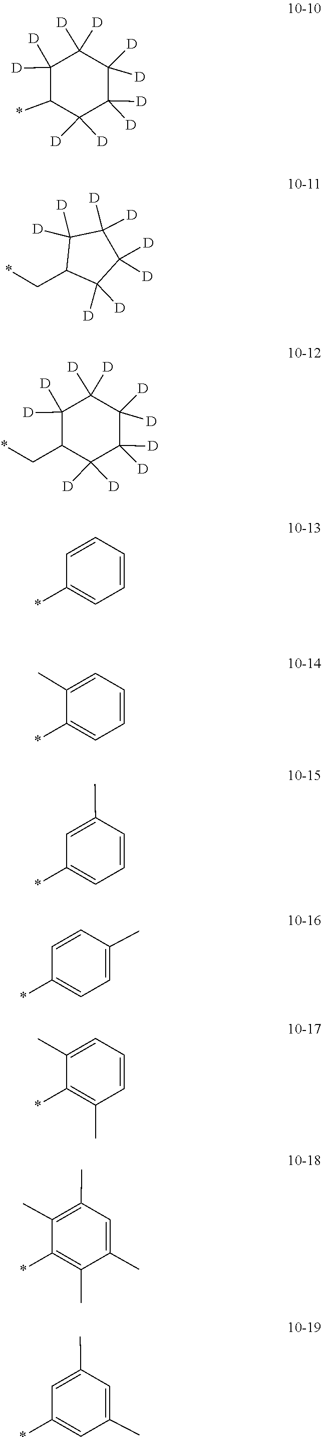

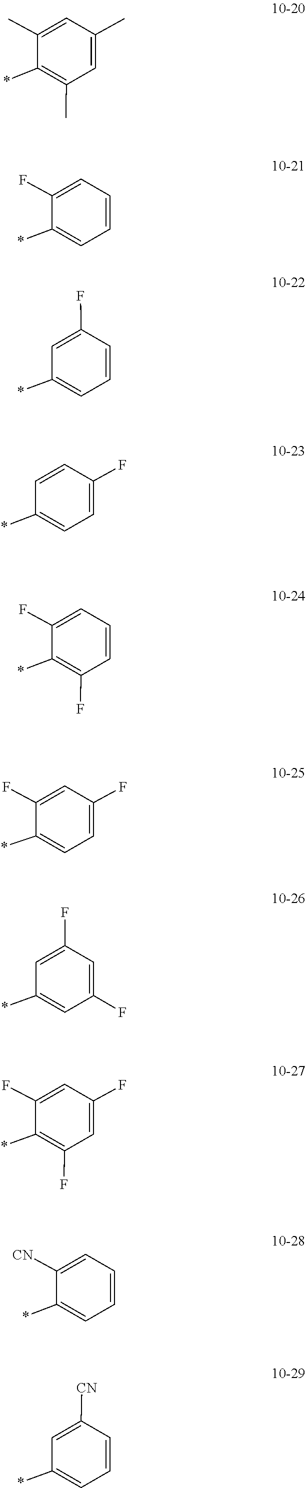

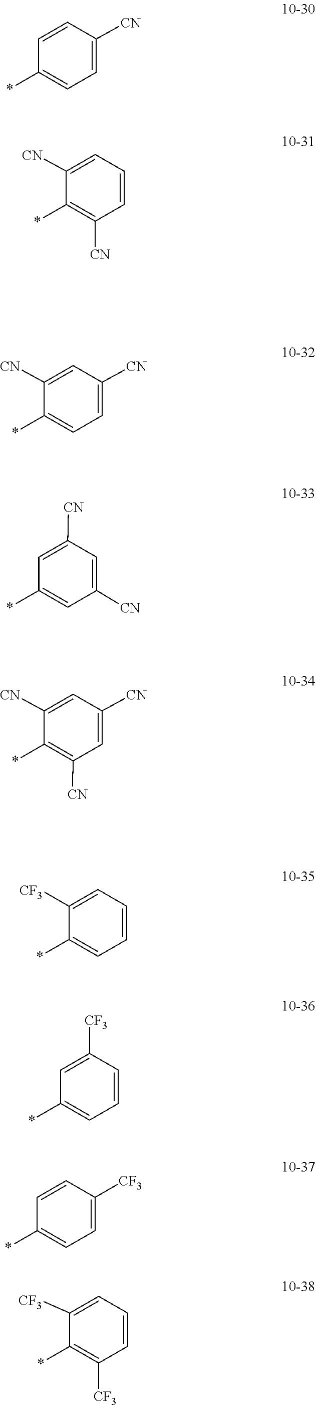

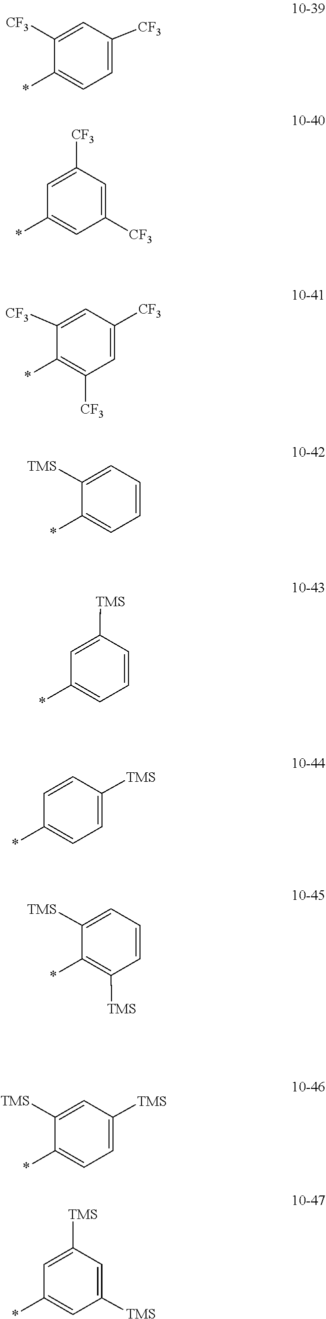

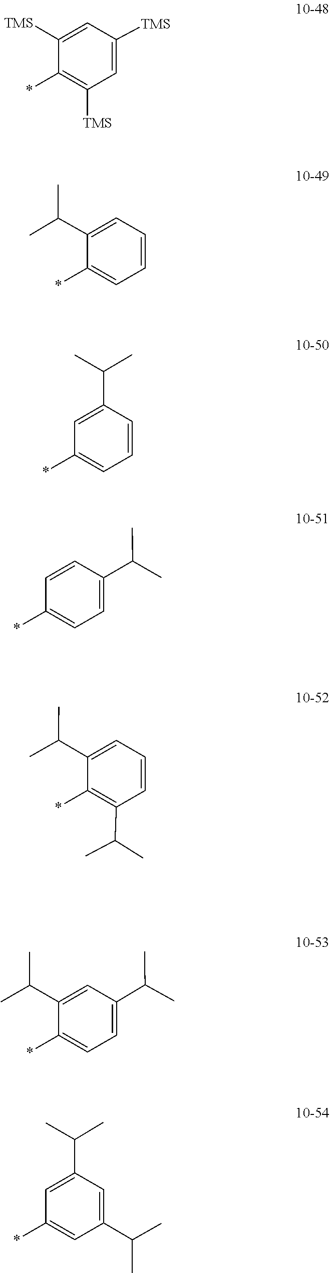

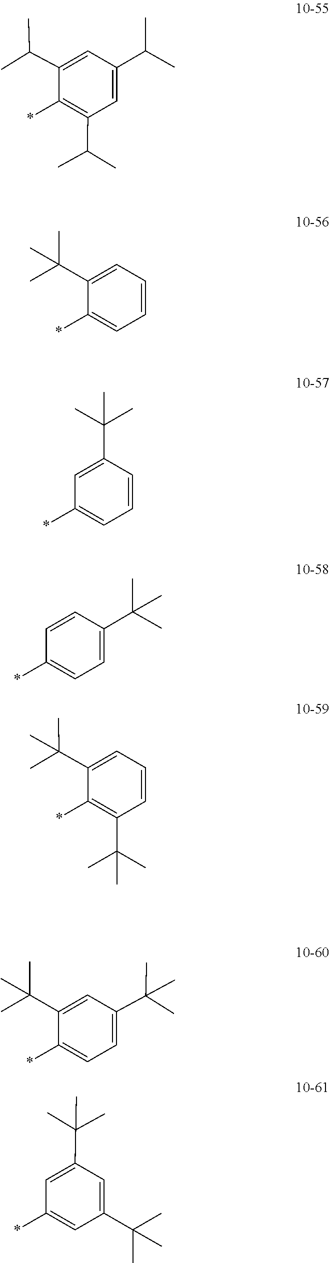

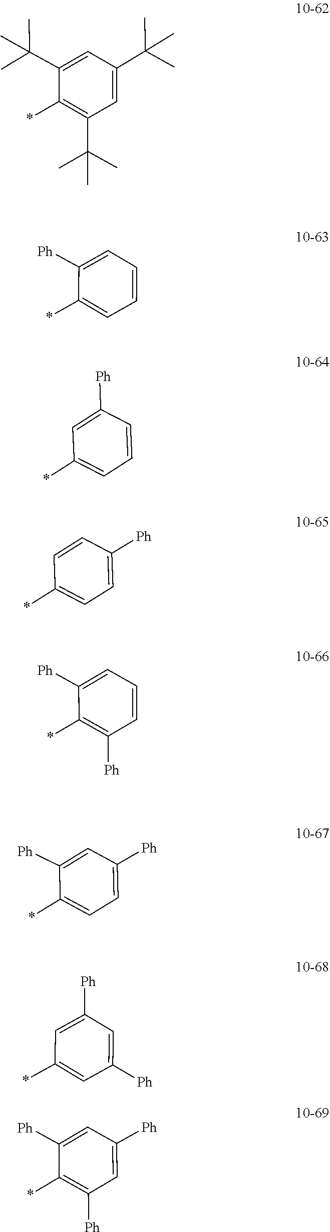

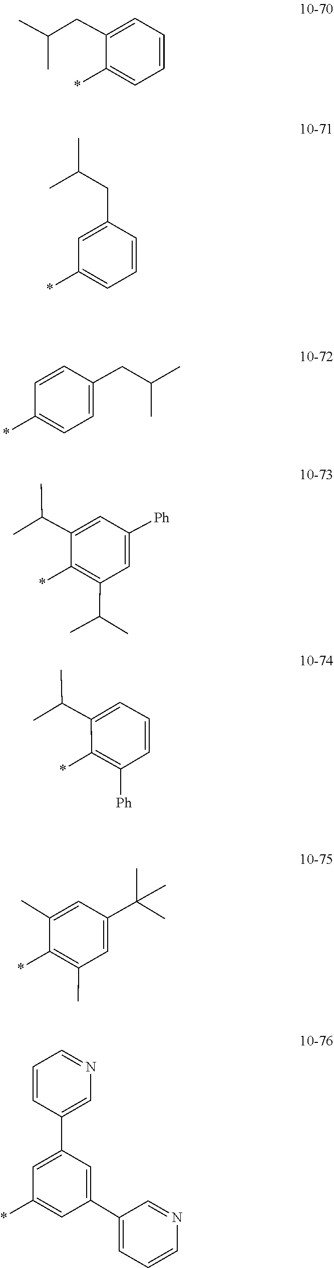

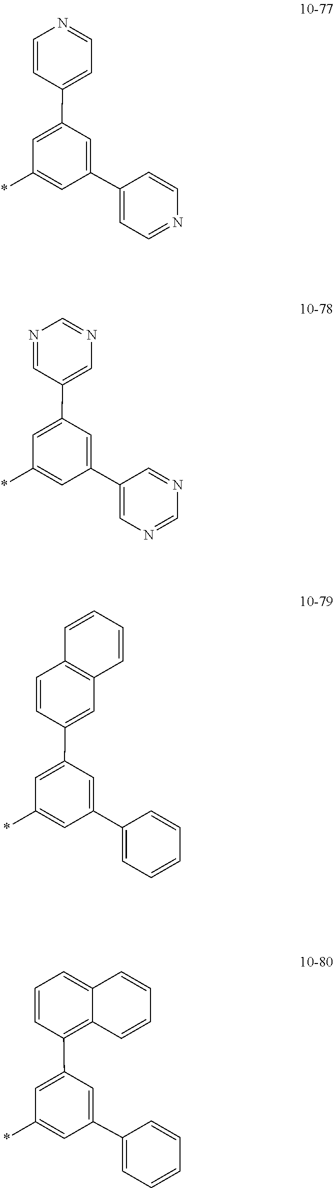 [SMILES: *c1cc(-c2ccncc2)cc(-c2ccncc2)c1 ; *c1cc(-c2cncnc2)cc(-c2cncnc2)c1 ; *c1cc(-c2ccccc2)cc(-c2ccc3ccccc3c2)c1 ; *c1cc(-c2ccccc2)cc(-c2cccc3ccccc23)c1]

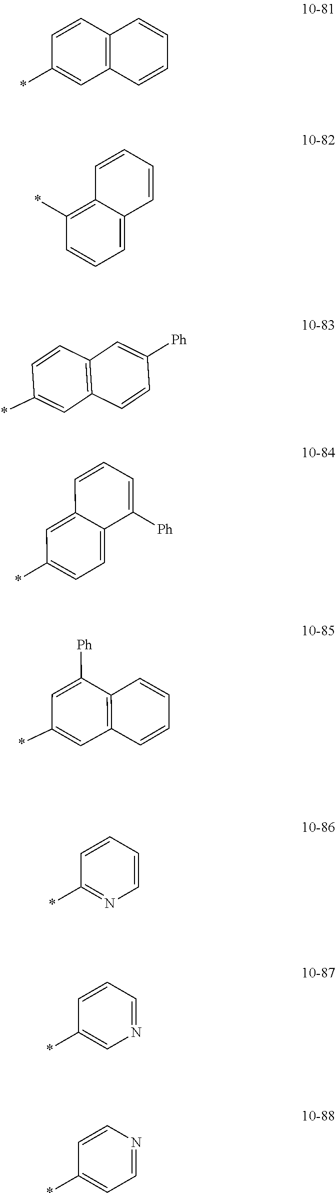

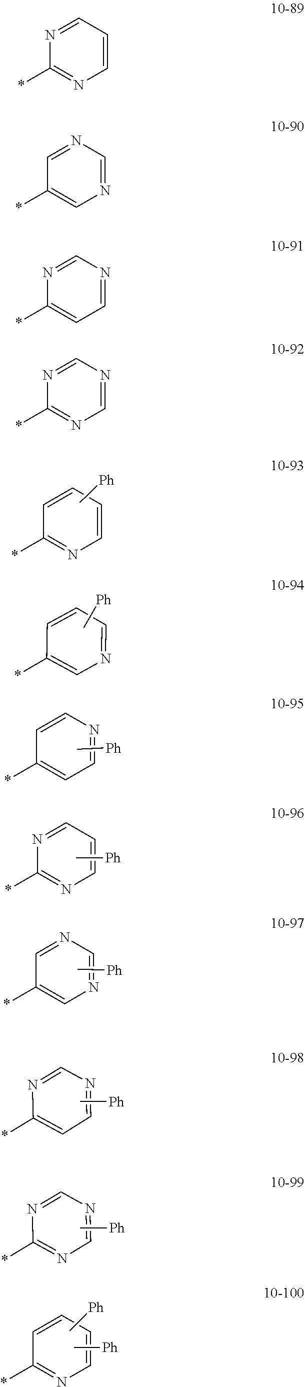

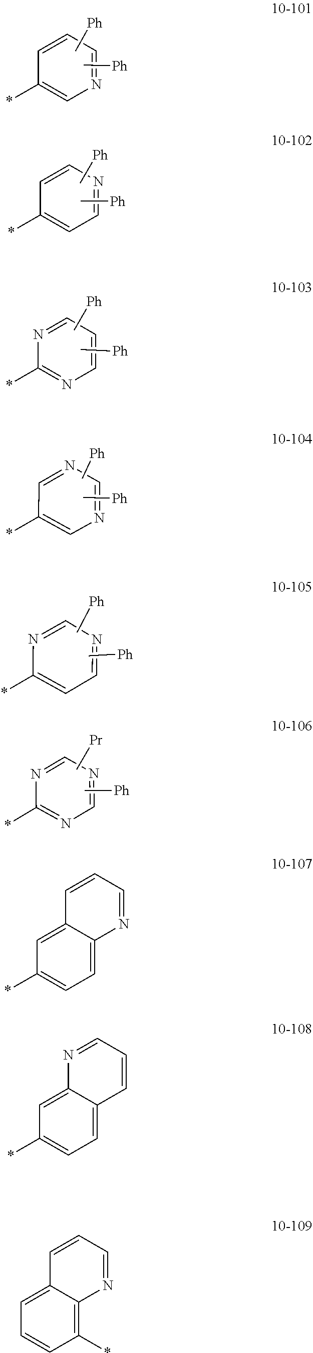

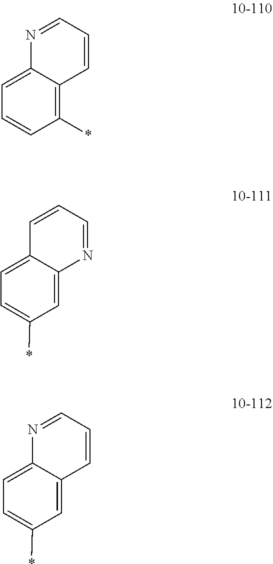 [SMILES: *c1cccc2ncccc12 ; *c1ccc2cccnc2c1 ; *c1ccc2ncccc2c1]

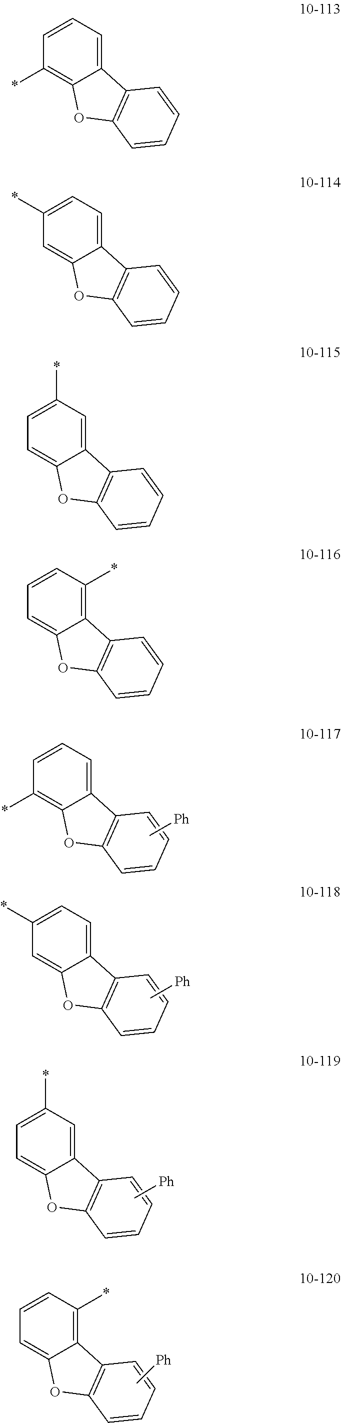

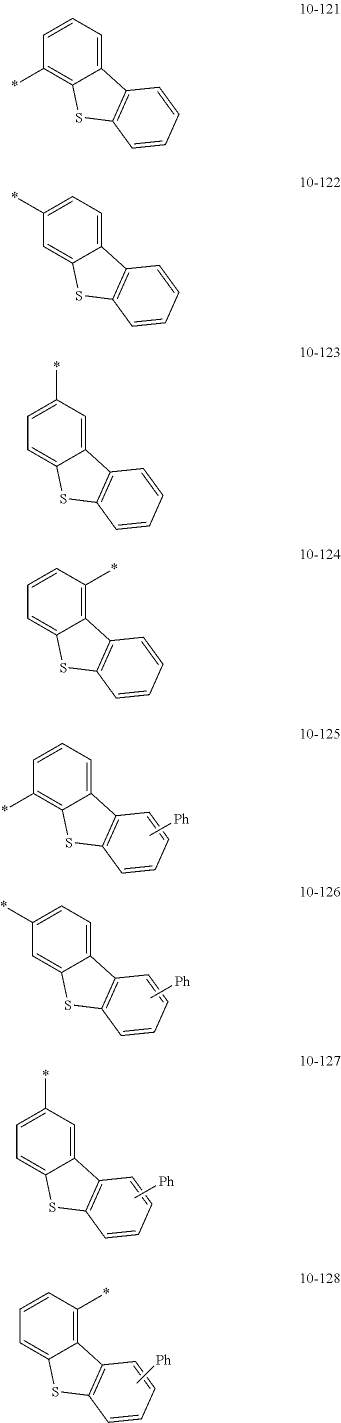

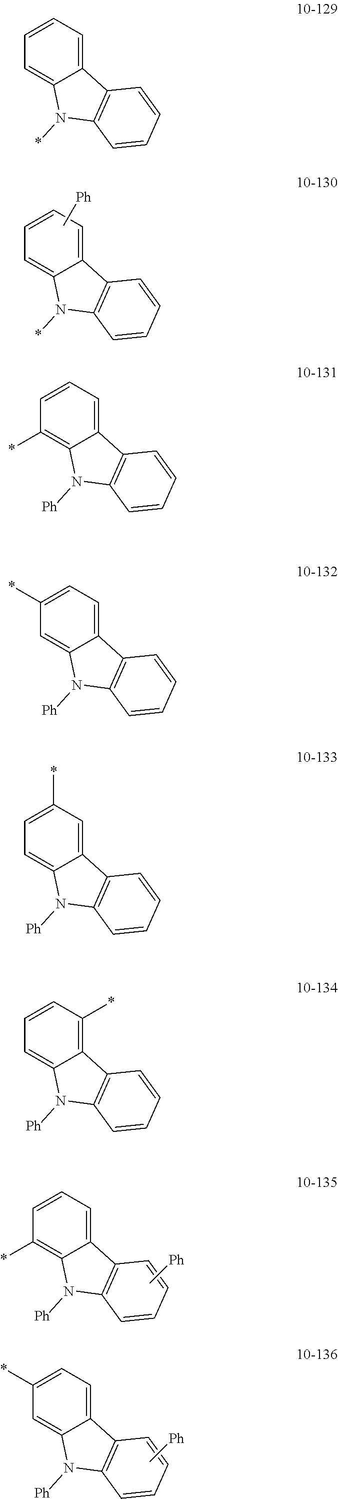

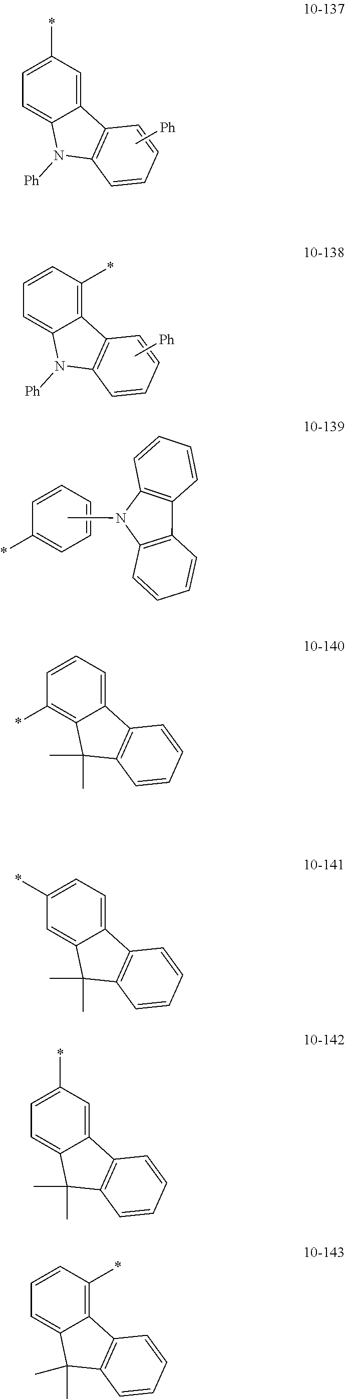

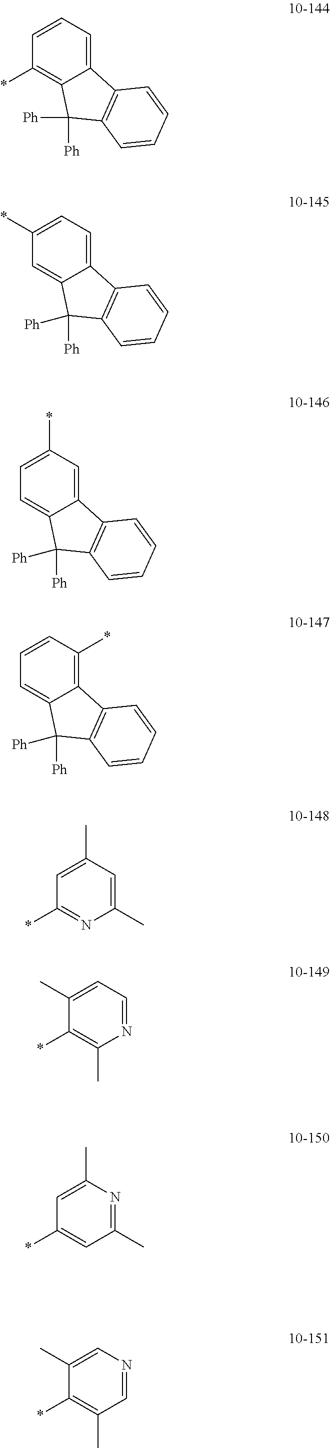

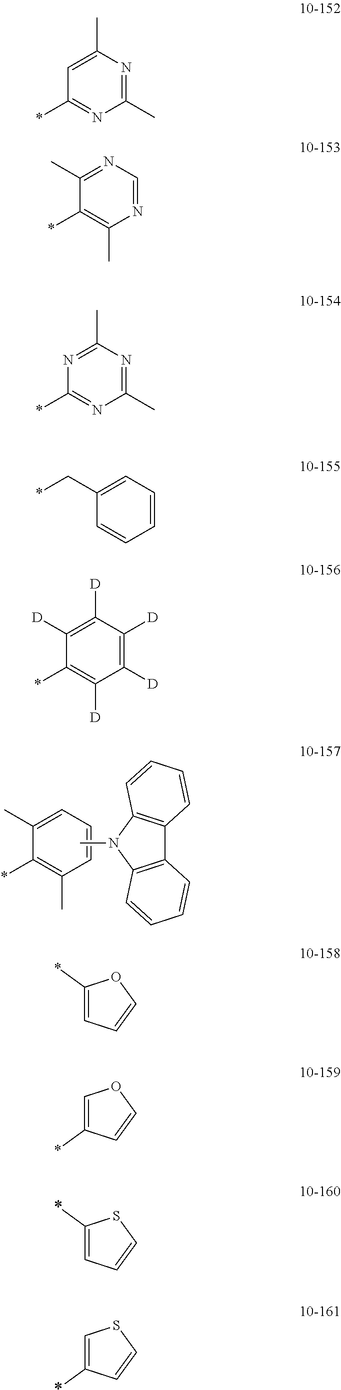

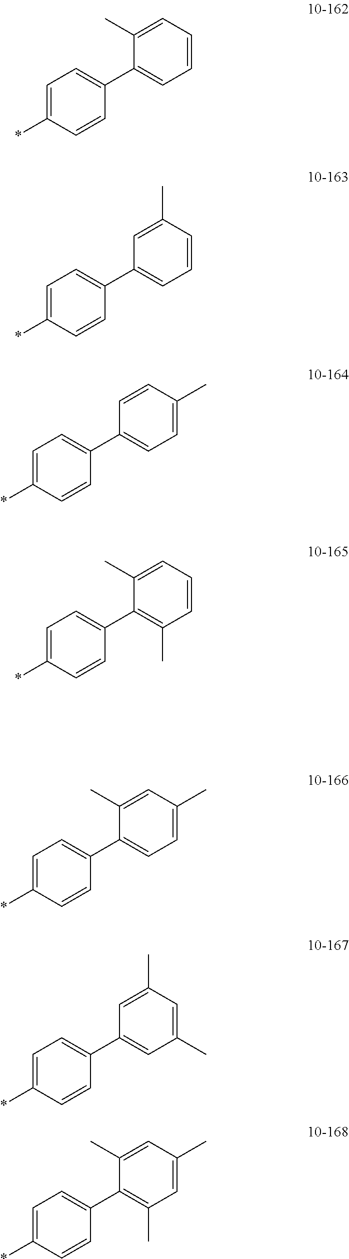

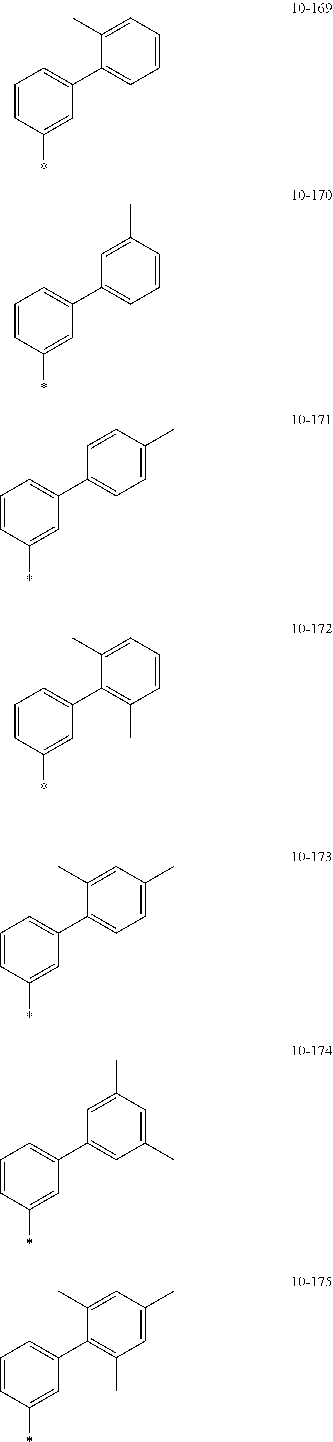

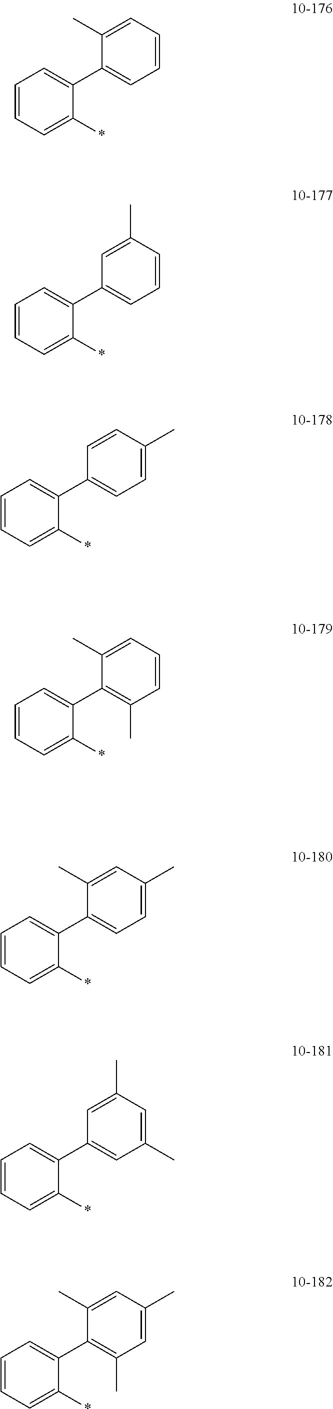

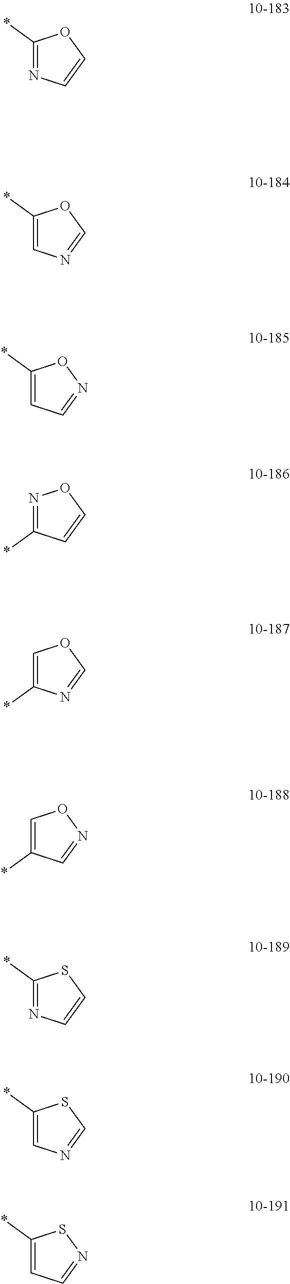

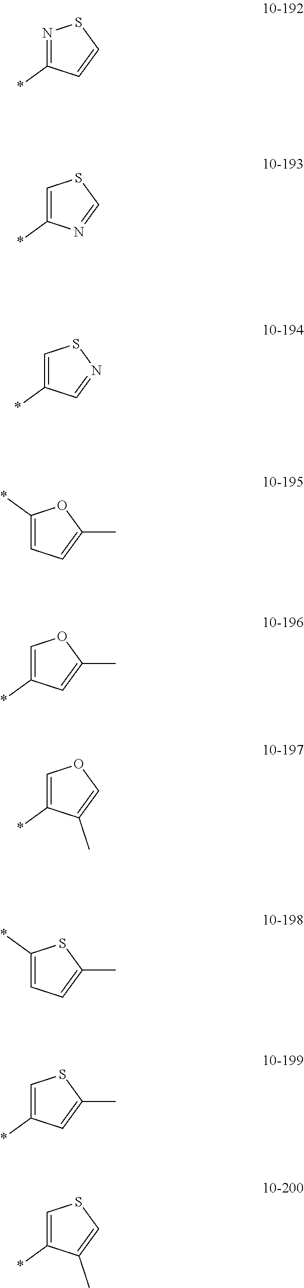

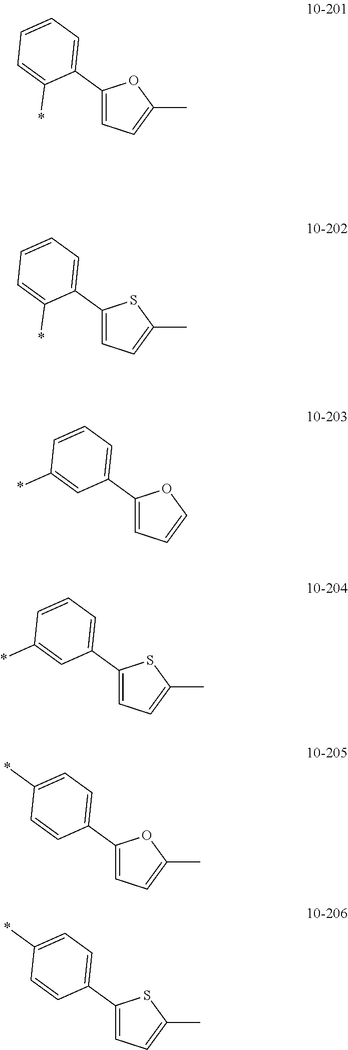 [SMILES: *c1ccccc1-c1ccc(C)o1 ; *c1ccccc1-c1ccc(C)s1 ; *c1cccc(-c2ccco2)c1 ; *c1cccc(-c2ccc(C)s2)c1 ; *c1ccc(-c2ccc(C)o2)cc1 ; *c1ccc(-c2ccc(C)s2)cc1]

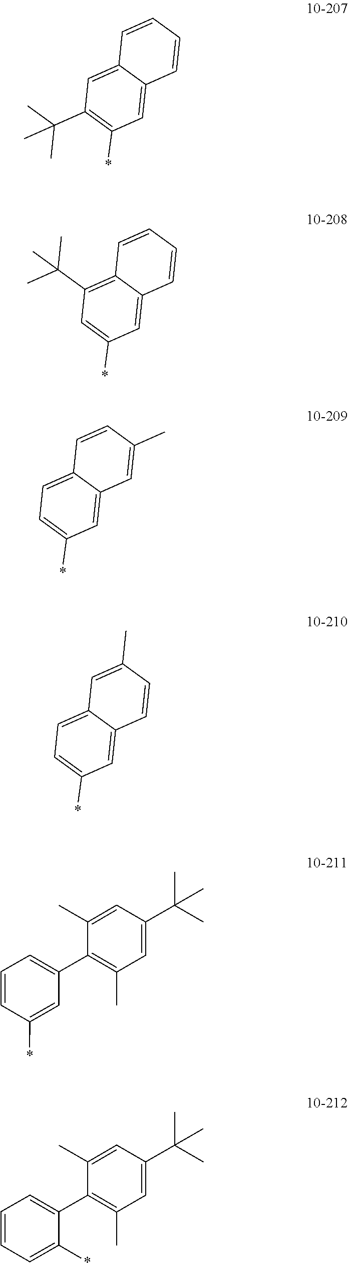 [SMILES: *c1cc2ccccc2cc1C(C)(C)C ; *c1cc(C(C)(C)C)c2ccccc2c1 ; *c1ccc2ccc(C)cc2c1 ; *c1ccc2cc(C)ccc2c1 ; *c1cccc(-c2c(C)cc(C(C)(C)C)cc2C)c1 ; *c1ccccc1-c1c(C)cc(C(C)(C)C)cc1C]

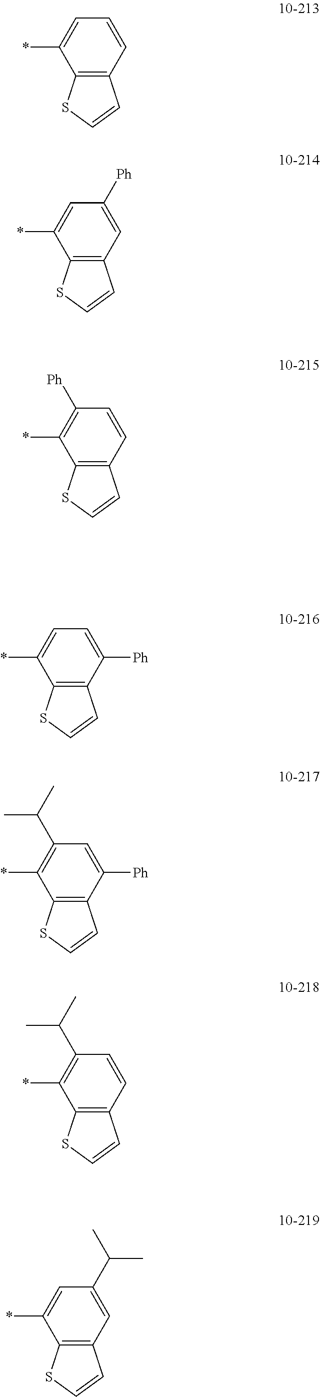

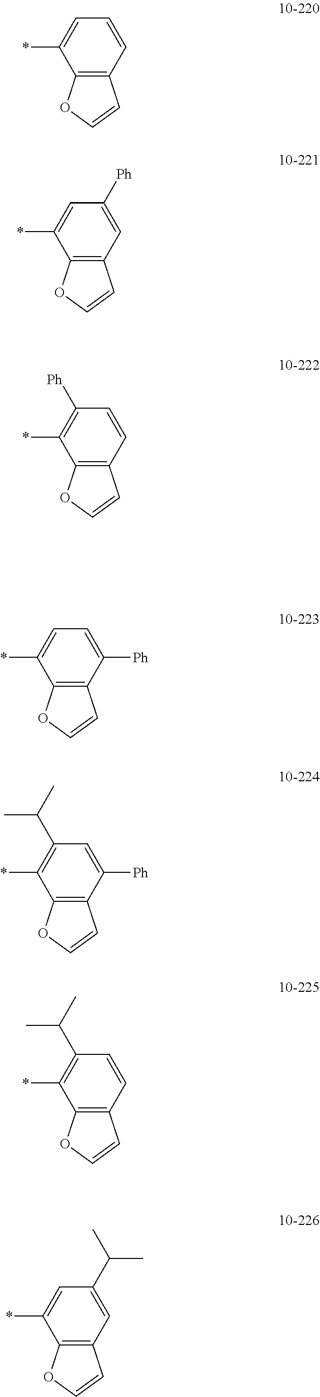

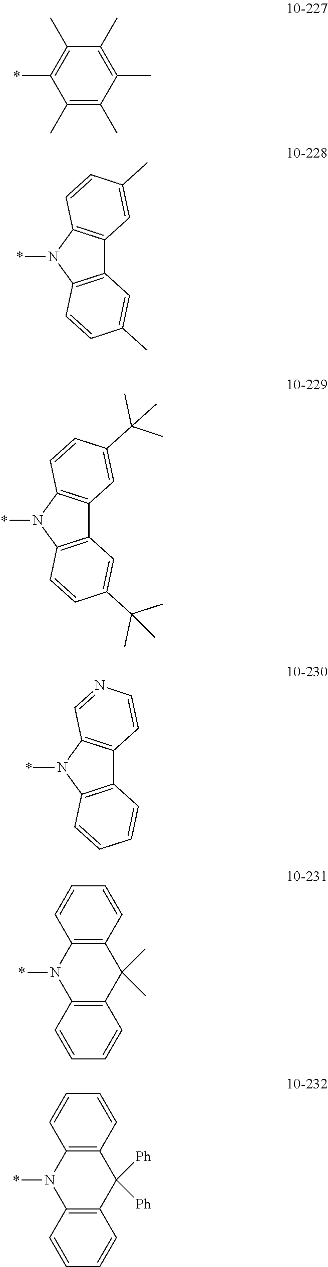

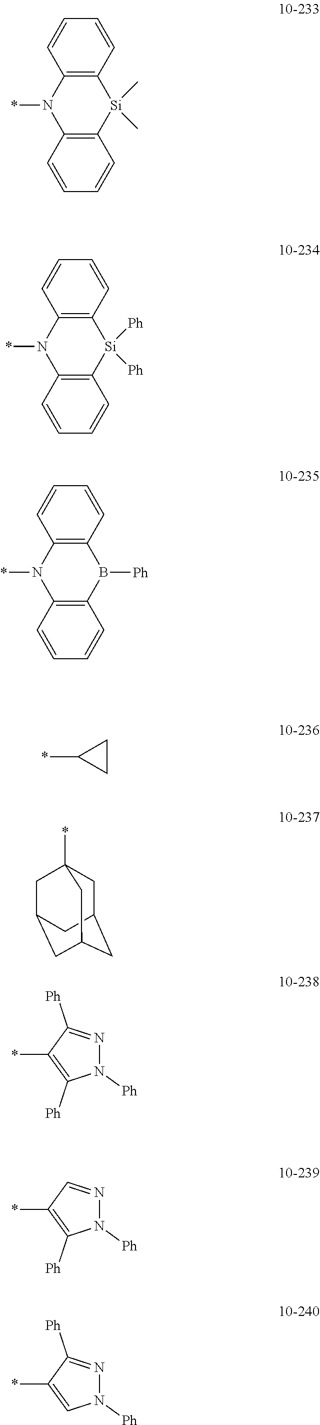

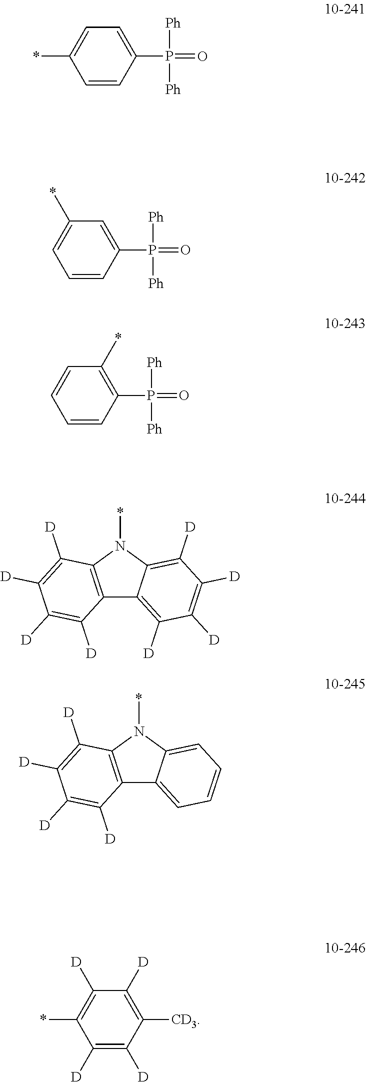

In Formulae 9-1 to 9-19 and 10-1 to 10-246, * indicates a binding site to a neighboring atom, “Ph” represents a phenyl group, “D” represents deuterium, and “TMS” represents a trimethylsilyl group.

In one or more embodiments, the heterocyclic compound represented by Formula 1 may be one selected from Compounds 1 to 396:

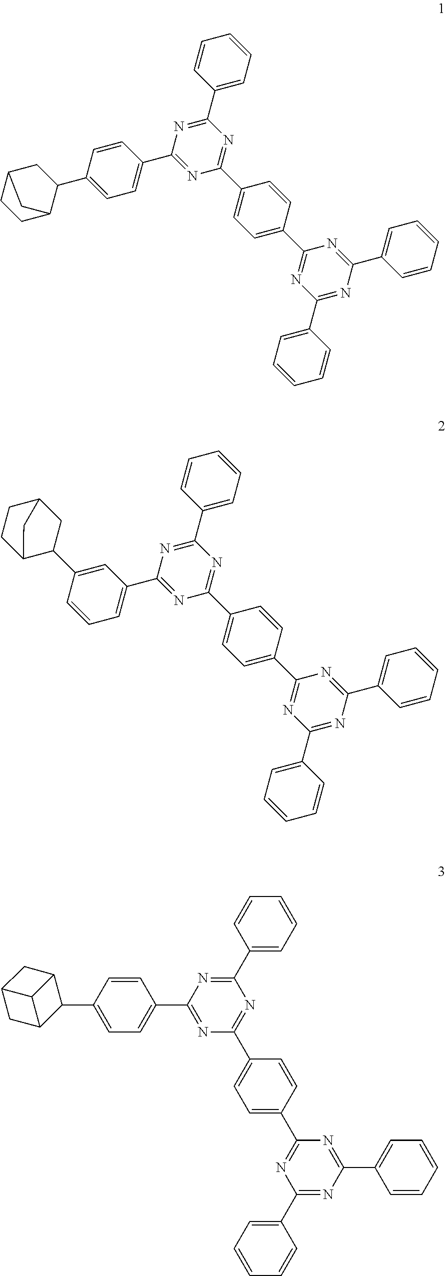 [SMILES: c1ccc(-c2nc(-c3ccccc3)nc(-c3ccc(-c4nc(-c5ccccc5)nc(-c5ccc(C6CC7CCC6C7)cc5)n4)cc3)n2)cc1 ; c1ccc(-c2nc(-c3ccccc3)nc(-c3ccc(-c4nc(-c5ccccc5)nc(-c5cccc(C6CC7CCC6C7)c5)n4)cc3)n2)cc1 ; c1ccc(-c2nc(-c3ccccc3)nc(-c3ccc(-c4nc(-c5ccccc5)nc(-c5ccc(C6C7CC8CC6C87)cc5)n4)cc3)n2)cc1]

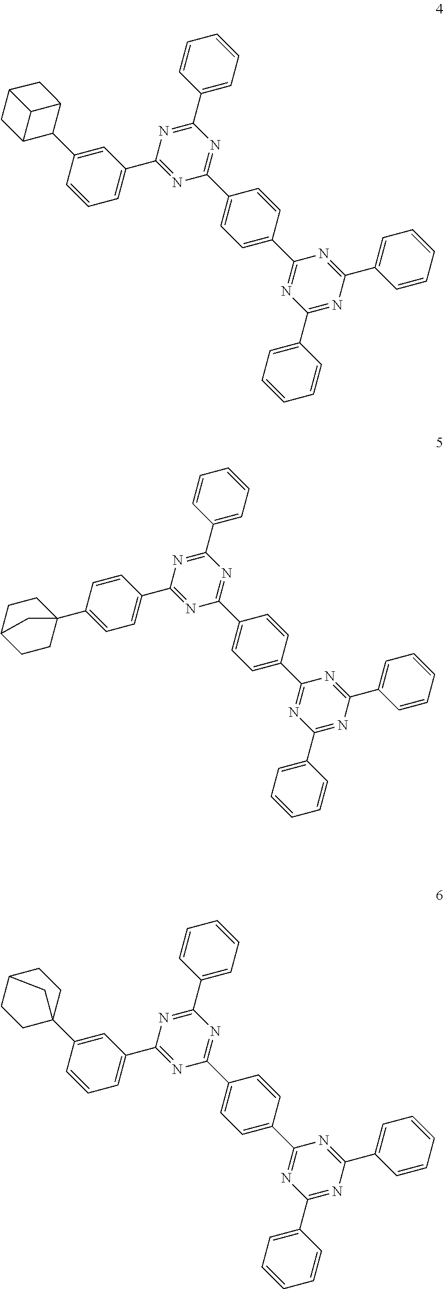 [SMILES: c1ccc(-c2nc(-c3ccccc3)nc(-c3ccc(-c4nc(-c5ccccc5)nc(-c5cccc(C6C7CC8CC6C87)c5)n4)cc3)n2)cc1 ; c1ccc(-c2nc(-c3ccccc3)nc(-c3ccc(-c4nc(-c5ccccc5)nc(-c5ccc(C67CCC(CC6)C7)cc5)n4)cc3)n2)cc1 ; c1ccc(-c2nc(-c3ccccc3)nc(-c3ccc(-c4nc(-c5ccccc5)nc(-c5cccc(C67CCC(CC6)C7)c5)n4)cc3)n2)cc1]

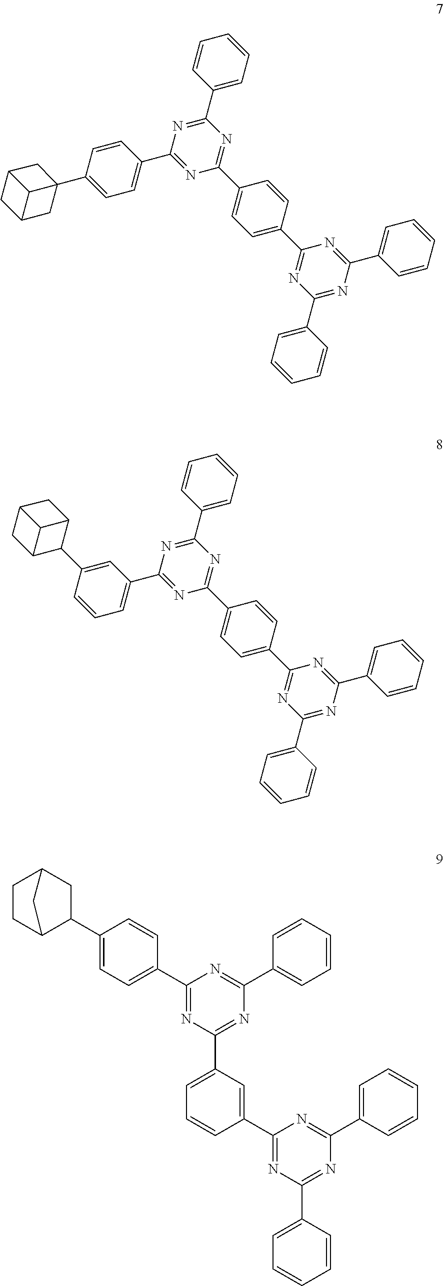 [SMILES: c1ccc(-c2nc(-c3ccccc3)nc(-c3ccc(-c4nc(-c5ccccc5)nc(-c5ccc(C67CC8CC(C6)C87)cc5)n4)cc3)n2)cc1 ; c1ccc(-c2nc(-c3ccccc3)nc(-c3ccc(-c4nc(-c5ccccc5)nc(-c5cccc(C6C7CC8CC6C87)c5)n4)cc3)n2)cc1 ; c1ccc(-c2nc(-c3ccccc3)nc(-c3cccc(-c4nc(-c5ccccc5)nc(-c5ccc(C6CC7CCC6C7)cc5)n4)c3)n2)cc1]

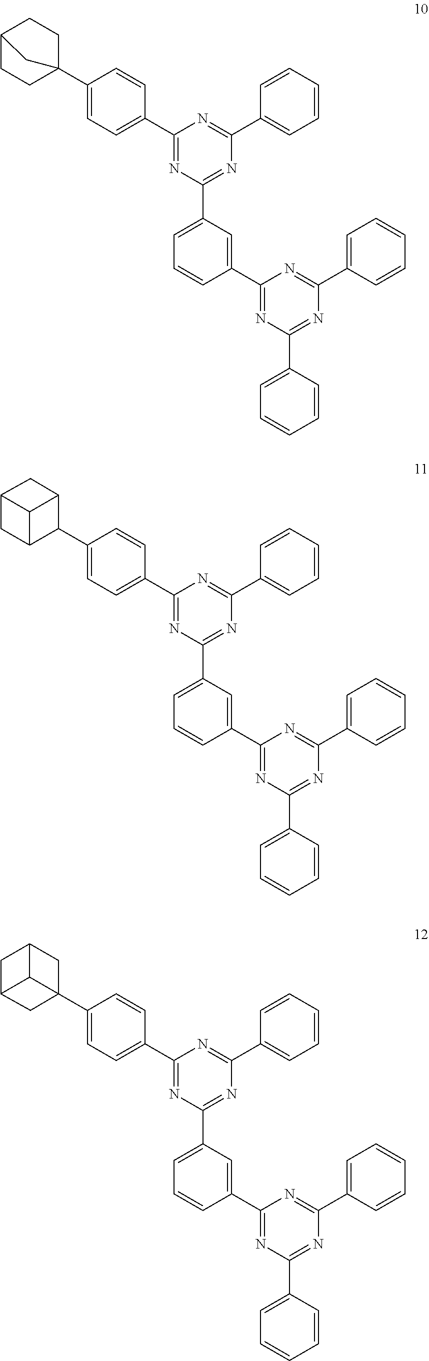 [SMILES: c1ccc(-c2nc(-c3ccccc3)nc(-c3cccc(-c4nc(-c5ccccc5)nc(-c5ccc(C67CCC(CC6)C7)cc5)n4)c3)n2)cc1 ; c1ccc(-c2nc(-c3ccccc3)nc(-c3cccc(-c4nc(-c5ccccc5)nc(-c5ccc(C6C7CC8CC6C87)cc5)n4)c3)n2)cc1 ; c1ccc(-c2nc(-c3ccccc3)nc(-c3cccc(-c4nc(-c5ccccc5)nc(-c5ccc(C67CC8CC(C6)C87)cc5)n4)c3)n2)cc1]

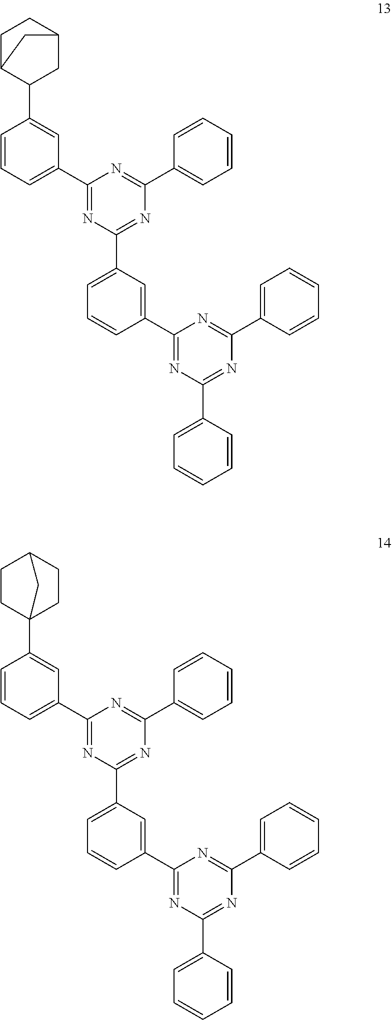 [SMILES: c1ccc(-c2nc(-c3ccccc3)nc(-c3cccc(-c4nc(-c5ccccc5)nc(-c5cccc(C6CC7CCC6C7)c5)n4)c3)n2)cc1 ; c1ccc(-c2nc(-c3ccccc3)nc(-c3cccc(-c4nc(-c5ccccc5)nc(-c5cccc(C67CCC(CC6)C7)c5)n4)c3)n2)cc1]

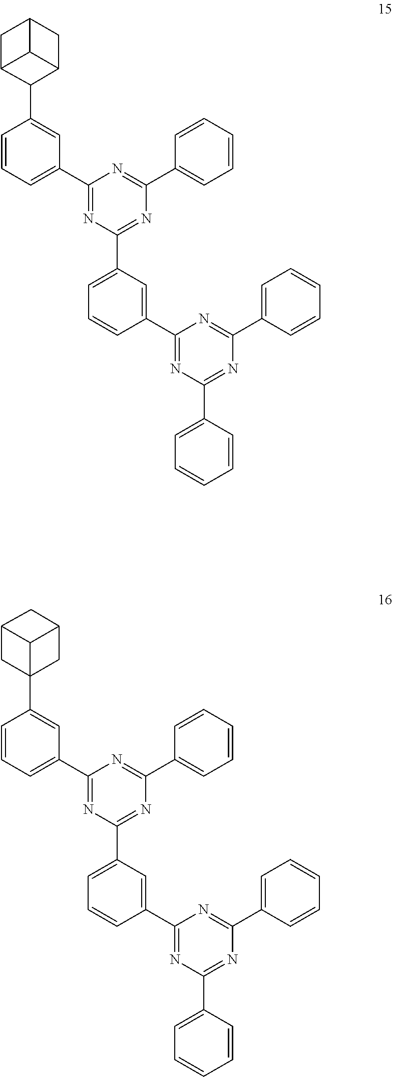 [SMILES: c1ccc(-c2nc(-c3ccccc3)nc(-c3cccc(-c4nc(-c5ccccc5)nc(-c5cccc(C6C7CC8CC6C87)c5)n4)c3)n2)cc1 ; c1ccc(-c2nc(-c3ccccc3)nc(-c3cccc(-c4nc(-c5ccccc5)nc(-c5cccc(C67CC8CC(C6)C87)c5)n4)c3)n2)cc1]

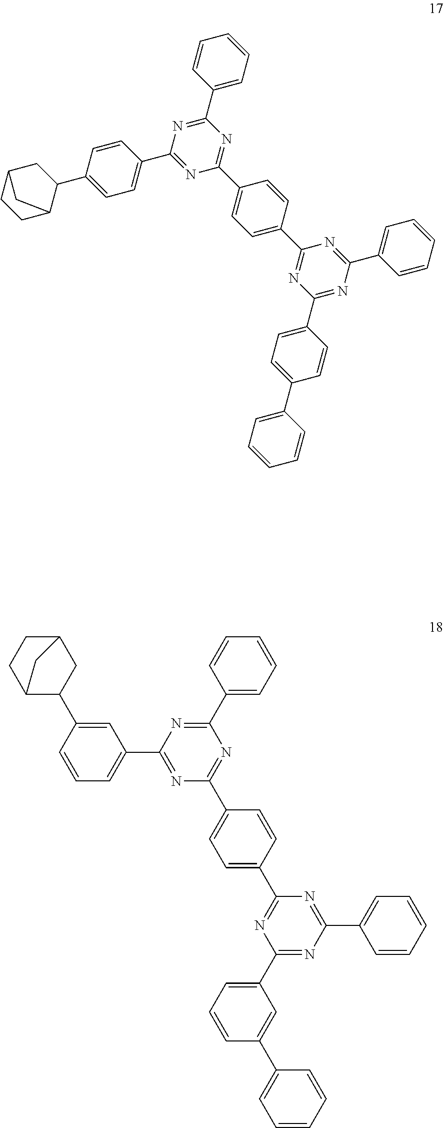 [SMILES: c1ccc(-c2ccc(-c3nc(-c4ccccc4)nc(-c4ccc(-c5nc(-c6ccccc6)nc(-c6ccc(C7CC8CCC7C8)cc6)n5)cc4)n3)cc2)cc1 ; c1ccc(-c2cccc(-c3nc(-c4ccccc4)nc(-c4ccc(-c5nc(-c6ccccc6)nc(-c6cccc(C7CC8CCC7C8)c6)n5)cc4)n3)c2)cc1]

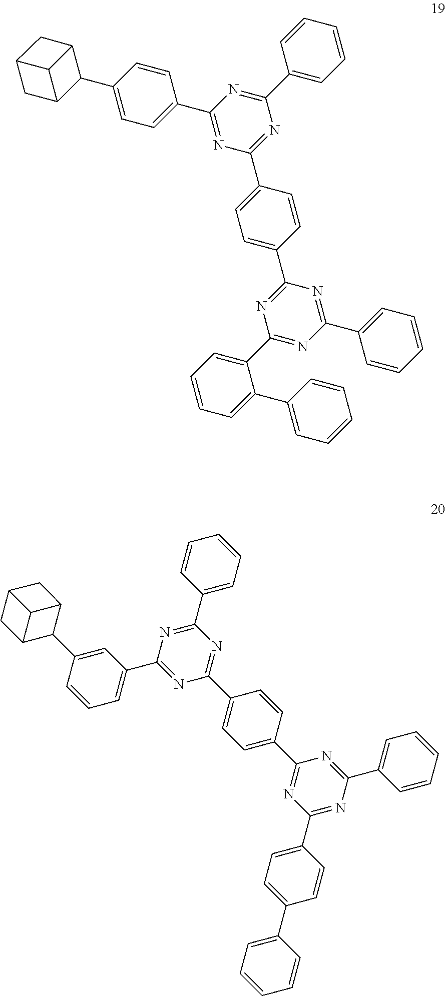 [SMILES: c1ccc(-c2nc(-c3ccc(-c4nc(-c5ccccc5)nc(-c5ccccc5-c5ccccc5)n4)cc3)nc(-c3ccc(C4C5CC6CC4C65)cc3)n2)cc1 ; c1ccc(-c2ccc(-c3nc(-c4ccccc4)nc(-c4ccc(-c5nc(-c6ccccc6)nc(-c6cccc(C7C8CC9CC7C98)c6)n5)cc4)n3)cc2)cc1]

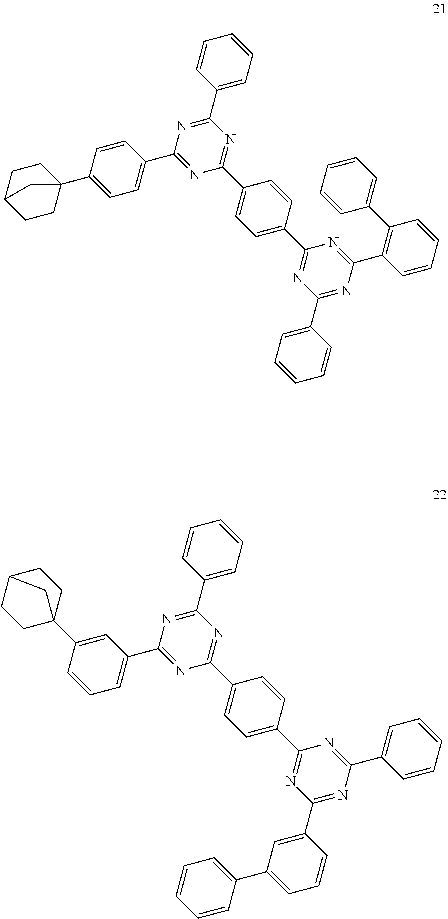 [SMILES: c1ccc(-c2nc(-c3ccc(-c4nc(-c5ccccc5)nc(-c5ccccc5-c5ccccc5)n4)cc3)nc(-c3ccc(C45CCC(CC4)C5)cc3)n2)cc1 ; c1ccc(-c2cccc(-c3nc(-c4ccccc4)nc(-c4ccc(-c5nc(-c6ccccc6)nc(-c6cccc(C78CCC(CC7)C8)c6)n5)cc4)n3)c2)cc1]

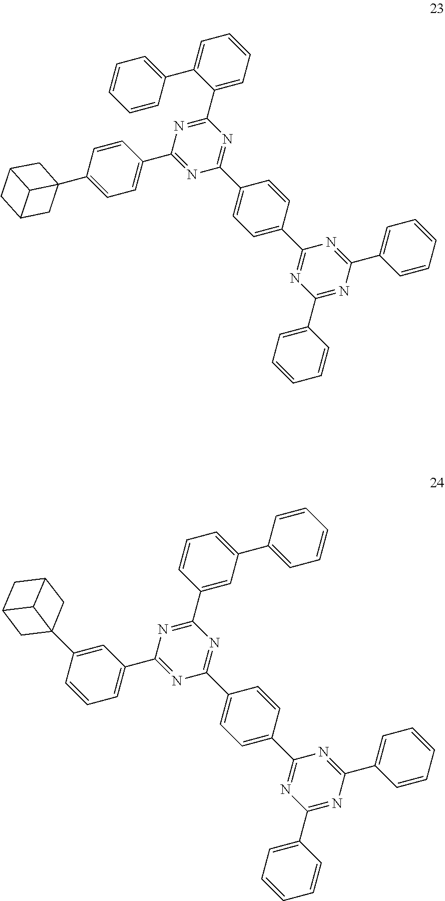 [SMILES: c1ccc(-c2nc(-c3ccccc3)nc(-c3ccc(-c4nc(-c5ccc(C67CC8CC(C6)C87)cc5)nc(-c5ccccc5-c5ccccc5)n4)cc3)n2)cc1 ; c1ccc(-c2cccc(-c3nc(-c4ccc(-c5nc(-c6ccccc6)nc(-c6ccccc6)n5)cc4)nc(-c4cccc(C56CC7CC(C5)C76)c4)n3)c2)cc1]

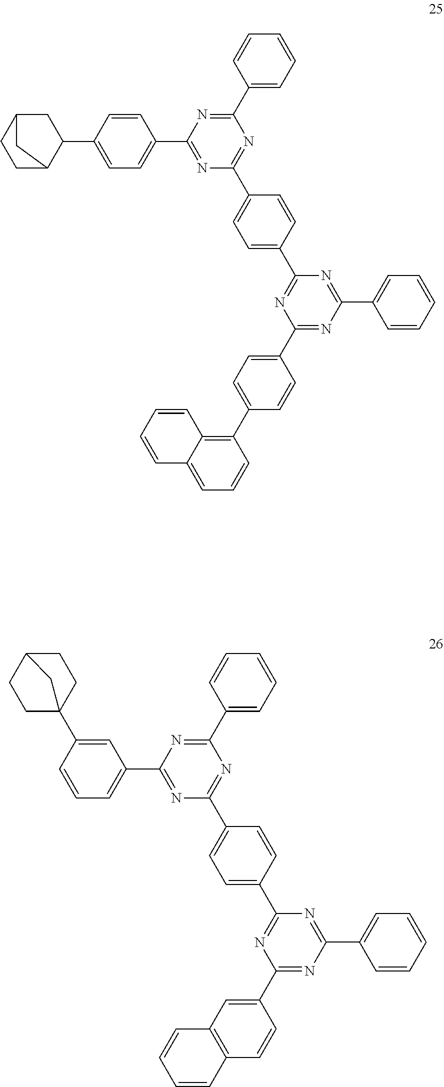 [SMILES: c1ccc(-c2nc(-c3ccc(-c4nc(-c5ccccc5)nc(-c5ccc(C6CC7CCC6C7)cc5)n4)cc3)nc(-c3ccc(-c4cccc5ccccc45)cc3)n2)cc1 ; c1ccc(-c2nc(-c3ccc(-c4nc(-c5ccccc5)nc(-c5ccc6ccccc6c5)n4)cc3)nc(-c3cccc(C45CCC(CC4)C5)c3)n2)cc1]

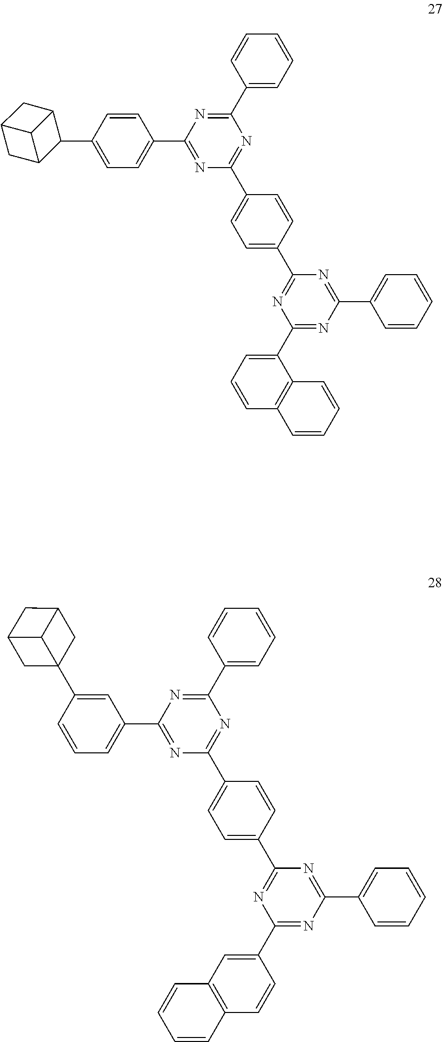 [SMILES: c1ccc(-c2nc(-c3ccc(-c4nc(-c5ccccc5)nc(-c5cccc6ccccc56)n4)cc3)nc(-c3ccc(C4C5CC6CC4C65)cc3)n2)cc1 ; c1ccc(-c2nc(-c3ccc(-c4nc(-c5ccccc5)nc(-c5ccc6ccccc6c5)n4)cc3)nc(-c3cccc(C45CC6CC(C4)C65)c3)n2)cc1]

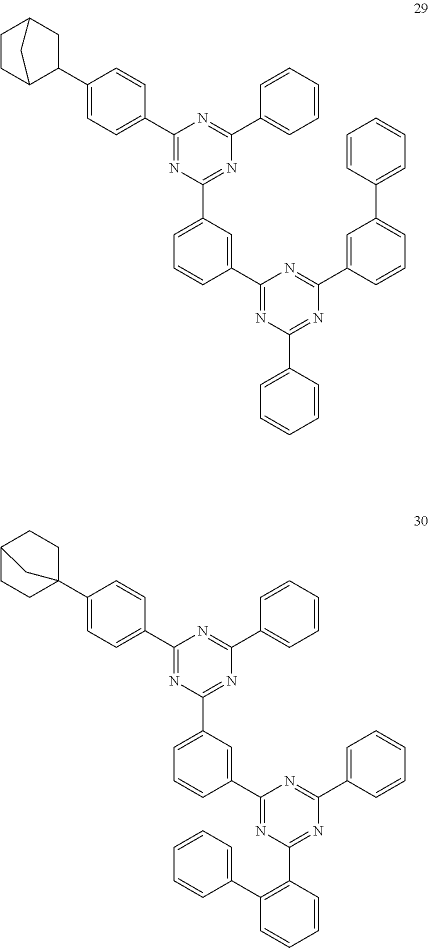 [SMILES: c1ccc(-c2cccc(-c3nc(-c4ccccc4)nc(-c4cccc(-c5nc(-c6ccccc6)nc(-c6ccc(C7CC8CCC7C8)cc6)n5)c4)n3)c2)cc1 ; c1ccc(-c2nc(-c3ccc(C45CCC(CC4)C5)cc3)nc(-c3cccc(-c4nc(-c5ccccc5)nc(-c5ccccc5-c5ccccc5)n4)c3)n2)cc1]

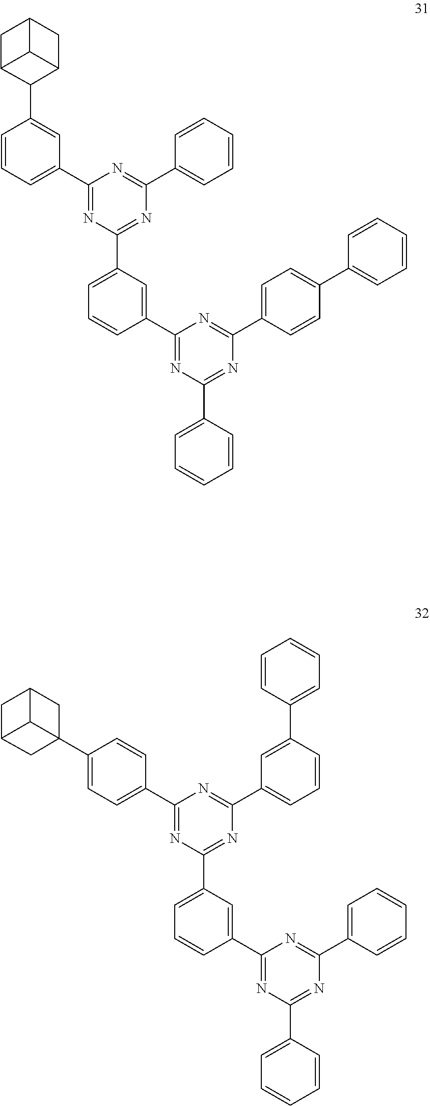 [SMILES: c1ccc(-c2ccc(-c3nc(-c4ccccc4)nc(-c4cccc(-c5nc(-c6ccccc6)nc(-c6cccc(C7C8CC9CC7C98)c6)n5)c4)n3)cc2)cc1 ; c1ccc(-c2cccc(-c3nc(-c4ccc(C56CC7CC(C5)C76)cc4)nc(-c4cccc(-c5nc(-c6ccccc6)nc(-c6ccccc6)n5)c4)n3)c2)cc1]

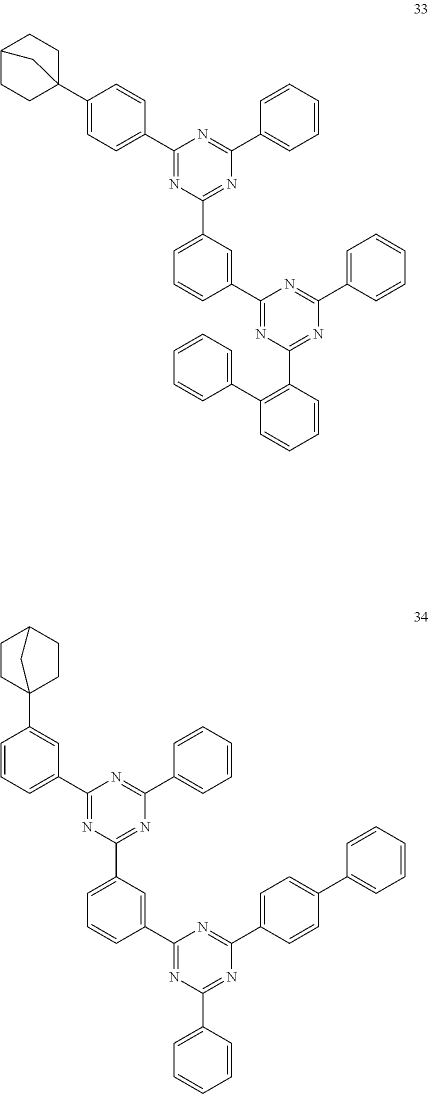 [SMILES: c1ccc(-c2nc(-c3ccc(C45CCC(CC4)C5)cc3)nc(-c3cccc(-c4nc(-c5ccccc5)nc(-c5ccccc5-c5ccccc5)n4)c3)n2)cc1 ; c1ccc(-c2ccc(-c3nc(-c4ccccc4)nc(-c4cccc(-c5nc(-c6ccccc6)nc(-c6cccc(C78CCC(CC7)C8)c6)n5)c4)n3)cc2)cc1]

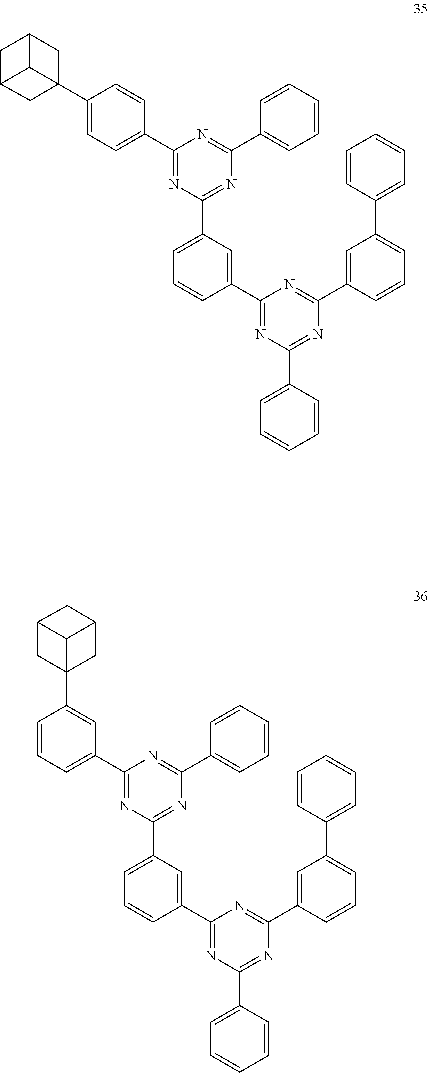 [SMILES: c1ccc(-c2cccc(-c3nc(-c4ccccc4)nc(-c4cccc(-c5nc(-c6ccccc6)nc(-c6ccc(C78CC9CC(C7)C98)cc6)n5)c4)n3)c2)cc1 ; c1ccc(-c2cccc(-c3nc(-c4ccccc4)nc(-c4cccc(-c5nc(-c6ccccc6)nc(-c6cccc(C78CC9CC(C7)C98)c6)n5)c4)n3)c2)cc1]

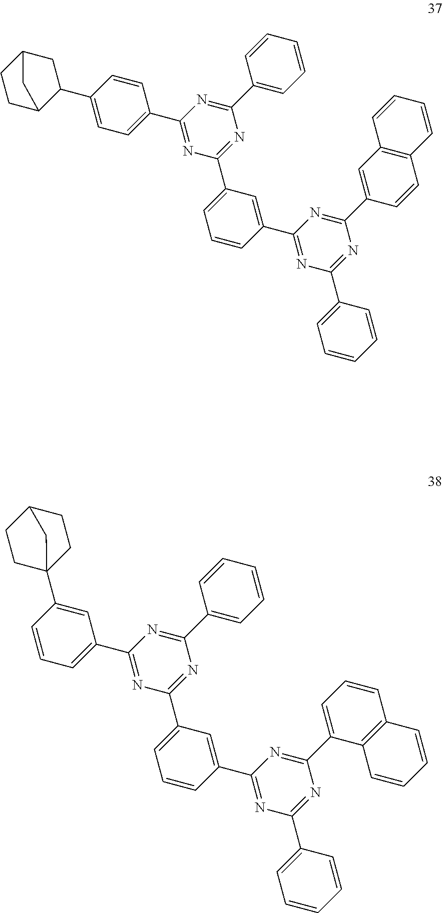 [SMILES: c1ccc(-c2nc(-c3ccc(C4CC5CCC4C5)cc3)nc(-c3cccc(-c4nc(-c5ccccc5)nc(-c5ccc6ccccc6c5)n4)c3)n2)cc1 ; c1ccc(-c2nc(-c3cccc(-c4nc(-c5ccccc5)nc(-c5cccc6ccccc56)n4)c3)nc(-c3cccc(C45CCC(CC4)C5)c3)n2)cc1]

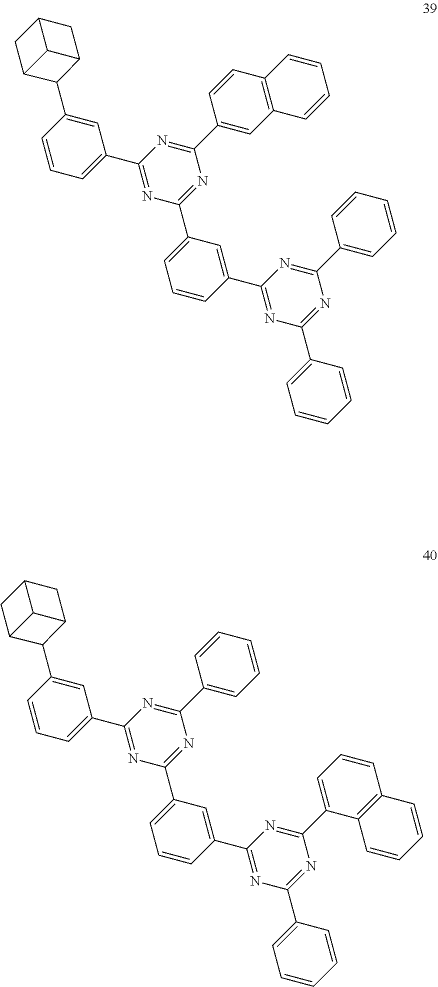 [SMILES: c1ccc(-c2nc(-c3ccccc3)nc(-c3cccc(-c4nc(-c5cccc(C6C7CC8CC6C87)c5)nc(-c5ccc6ccccc6c5)n4)c3)n2)cc1 ; c1ccc(-c2nc(-c3cccc(-c4nc(-c5ccccc5)nc(-c5cccc6ccccc56)n4)c3)nc(-c3cccc(C4C5CC6CC4C65)c3)n2)cc1]

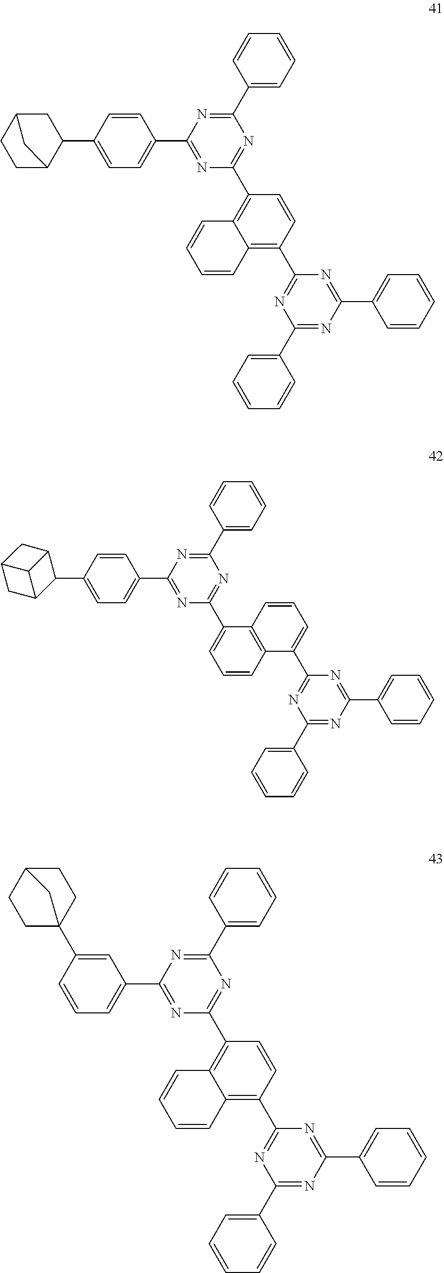 [SMILES: c1ccc(-c2nc(-c3ccccc3)nc(-c3ccc(-c4nc(-c5ccccc5)nc(-c5ccc(C6CC7CCC6C7)cc5)n4)c4ccccc34)n2)cc1 ; c1ccc(-c2nc(-c3ccccc3)nc(-c3cccc4c(-c5nc(-c6ccccc6)nc(-c6ccc(C7C8CC9CC7C98)cc6)n5)cccc34)n2)cc1 ; c1ccc(-c2nc(-c3ccccc3)nc(-c3ccc(-c4nc(-c5ccccc5)nc(-c5cccc(C67CCC(CC6)C7)c5)n4)c4ccccc34)n2)cc1]

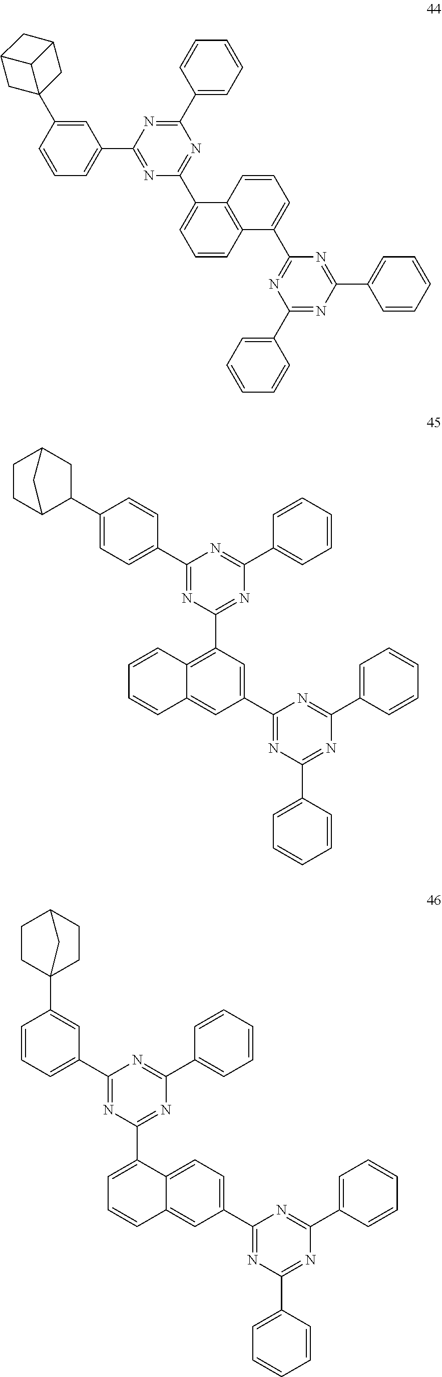 [SMILES: c1ccc(-c2nc(-c3ccccc3)nc(-c3cccc4c(-c5nc(-c6ccccc6)nc(-c6cccc(C78CC9CC(C7)C98)c6)n5)cccc34)n2)cc1 ; c1ccc(-c2nc(-c3ccccc3)nc(-c3cc(-c4nc(-c5ccccc5)nc(-c5ccc(C6CC7CCC6C7)cc5)n4)c4ccccc4c3)n2)cc1 ; c1ccc(-c2nc(-c3ccccc3)nc(-c3ccc4c(-c5nc(-c6ccccc6)nc(-c6cccc(C78CCC(CC7)C8)c6)n5)cccc4c3)n2)cc1]

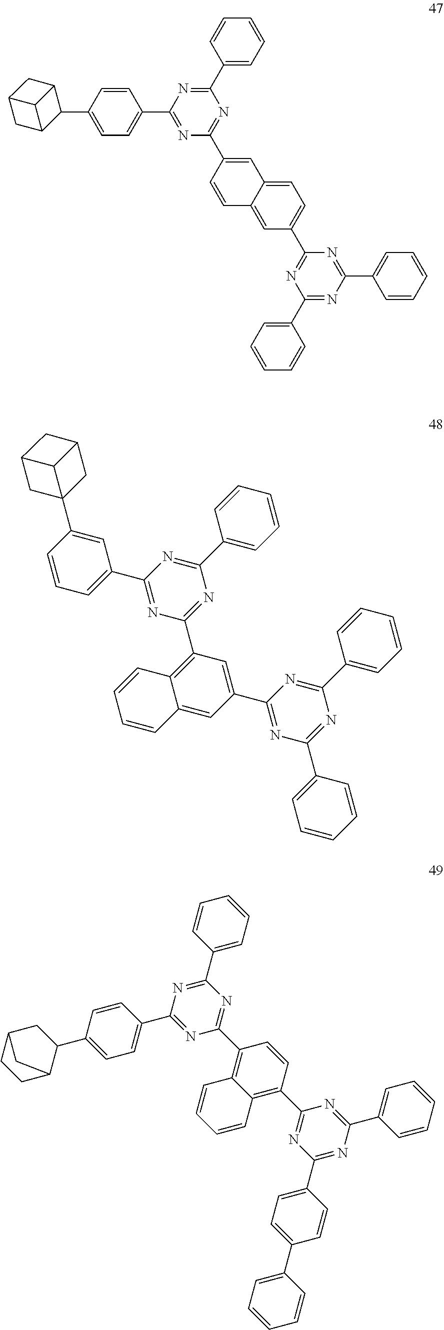 [SMILES: c1ccc(-c2nc(-c3ccccc3)nc(-c3ccc4cc(-c5nc(-c6ccccc6)nc(-c6ccc(C7C8CC9CC7C98)cc6)n5)ccc4c3)n2)cc1 ; c1ccc(-c2nc(-c3ccccc3)nc(-c3cc(-c4nc(-c5ccccc5)nc(-c5cccc(C67CC8CC(C6)C87)c5)n4)c4ccccc4c3)n2)cc1 ; c1ccc(-c2ccc(-c3nc(-c4ccccc4)nc(-c4ccc(-c5nc(-c6ccccc6)nc(-c6ccc(C7CC8CCC7C8)cc6)n5)c5ccccc45)n3)cc2)cc1]

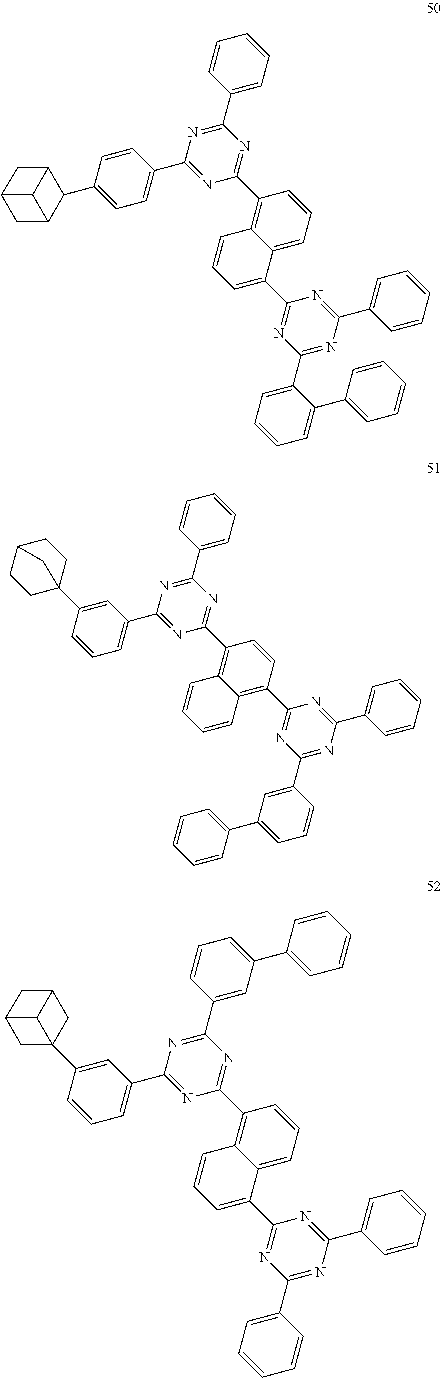 [SMILES: c1ccc(-c2nc(-c3ccc(C4C5CC6CC4C65)cc3)nc(-c3cccc4c(-c5nc(-c6ccccc6)nc(-c6ccccc6-c6ccccc6)n5)cccc34)n2)cc1 ; c1ccc(-c2cccc(-c3nc(-c4ccccc4)nc(-c4ccc(-c5nc(-c6ccccc6)nc(-c6cccc(C78CCC(CC7)C8)c6)n5)c5ccccc45)n3)c2)cc1 ; c1ccc(-c2cccc(-c3nc(-c4cccc(C56CC7CC(C5)C76)c4)nc(-c4cccc5c(-c6nc(-c7ccccc7)nc(-c7ccccc7)n6)cccc45)n3)c2)cc1]

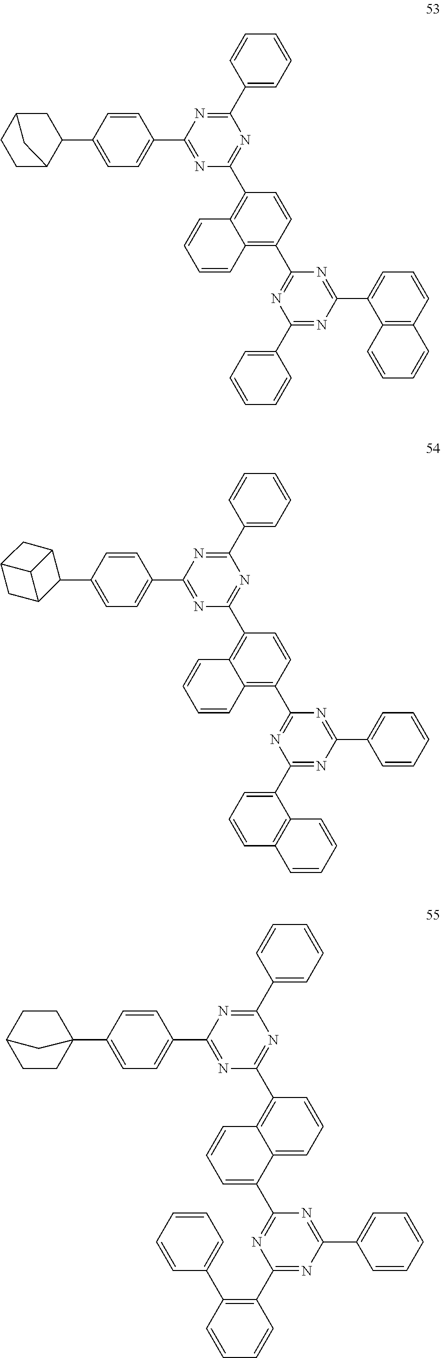 [SMILES: c1ccc(-c2nc(-c3ccc(C4CC5CCC4C5)cc3)nc(-c3ccc(-c4nc(-c5ccccc5)nc(-c5cccc6ccccc56)n4)c4ccccc34)n2)cc1 ; c1ccc(-c2nc(-c3ccc(C4C5CC6CC4C65)cc3)nc(-c3ccc(-c4nc(-c5ccccc5)nc(-c5cccc6ccccc56)n4)c4ccccc34)n2)cc1 ; c1ccc(-c2nc(-c3ccc(C45CCC(CC4)C5)cc3)nc(-c3cccc4c(-c5nc(-c6ccccc6)nc(-c6ccccc6-c6ccccc6)n5)cccc34)n2)cc1]

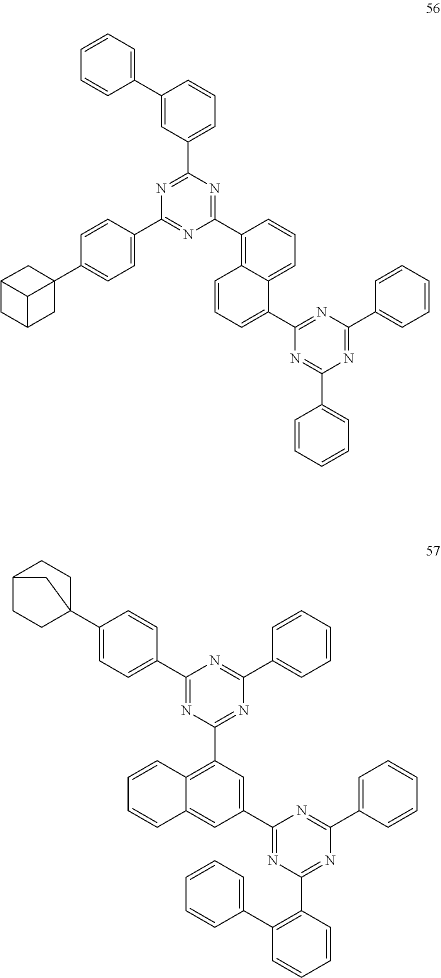 [SMILES: c1ccc(-c2cccc(-c3nc(-c4ccc(C56CC7CC(C5)C76)cc4)nc(-c4cccc5c(-c6nc(-c7ccccc7)nc(-c7ccccc7)n6)cccc45)n3)c2)cc1 ; c1ccc(-c2nc(-c3cc(-c4nc(-c5ccccc5)nc(-c5ccc(C67CCC(CC6)C7)cc5)n4)c4ccccc4c3)nc(-c3ccccc3-c3ccccc3)n2)cc1]

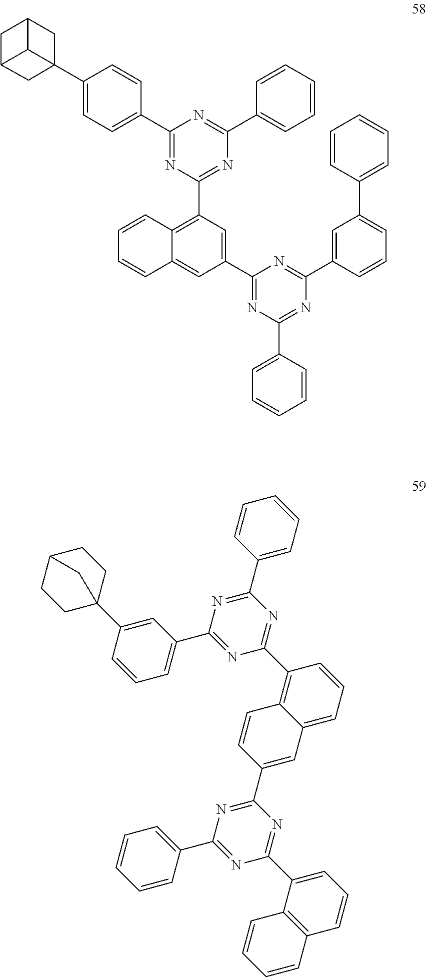 [SMILES: c1ccc(-c2cccc(-c3nc(-c4ccccc4)nc(-c4cc(-c5nc(-c6ccccc6)nc(-c6ccc(C78CC9CC(C7)C98)cc6)n5)c5ccccc5c4)n3)c2)cc1 ; c1ccc(-c2nc(-c3ccc4c(-c5nc(-c6ccccc6)nc(-c6cccc(C78CCC(CC7)C8)c6)n5)cccc4c3)nc(-c3cccc4ccccc34)n2)cc1]

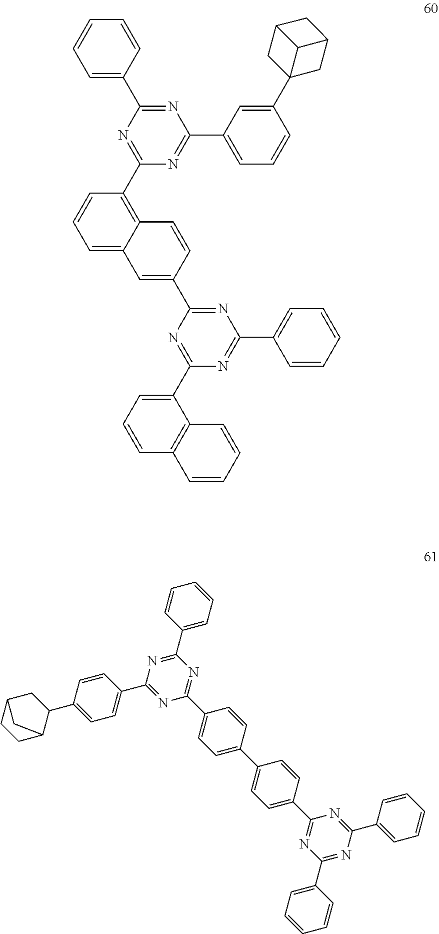 [SMILES: c1ccc(-c2nc(-c3ccc4c(-c5nc(-c6ccccc6)nc(-c6cccc(C78CC9CC(C7)C98)c6)n5)cccc4c3)nc(-c3cccc4ccccc34)n2)cc1 ; c1ccc(-c2nc(-c3ccccc3)nc(-c3ccc(-c4ccc(-c5nc(-c6ccccc6)nc(-c6ccc(C7CC8CCC7C8)cc6)n5)cc4)cc3)n2)cc1]

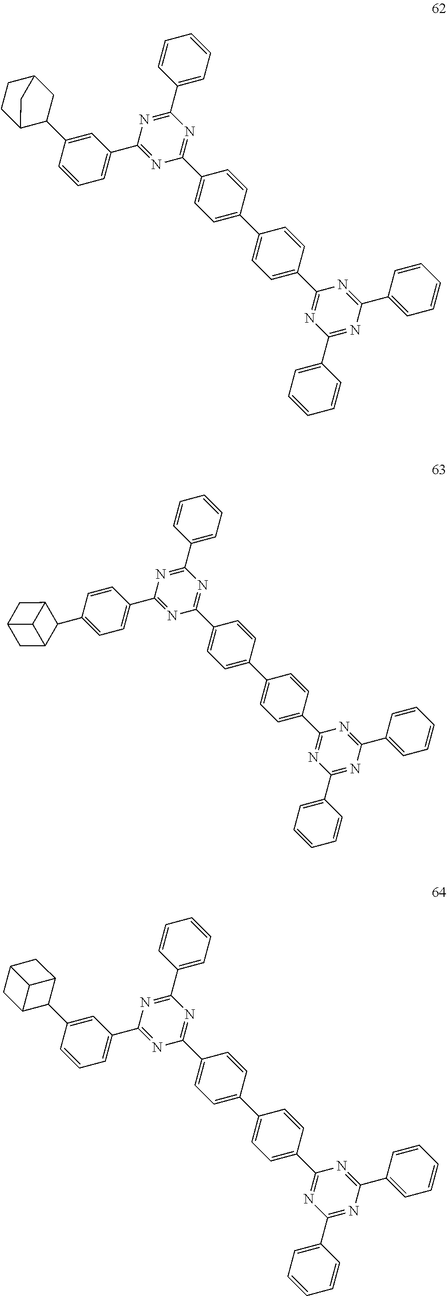 [SMILES: c1ccc(-c2nc(-c3ccccc3)nc(-c3ccc(-c4ccc(-c5nc(-c6ccccc6)nc(-c6cccc(C7CC8CCC7C8)c6)n5)cc4)cc3)n2)cc1 ; c1ccc(-c2nc(-c3ccccc3)nc(-c3ccc(-c4ccc(-c5nc(-c6ccccc6)nc(-c6ccc(C7C8CC9CC7C98)cc6)n5)cc4)cc3)n2)cc1 ; c1ccc(-c2nc(-c3ccccc3)nc(-c3ccc(-c4ccc(-c5nc(-c6ccccc6)nc(-c6cccc(C7C8CC9CC7C98)c6)n5)cc4)cc3)n2)cc1]

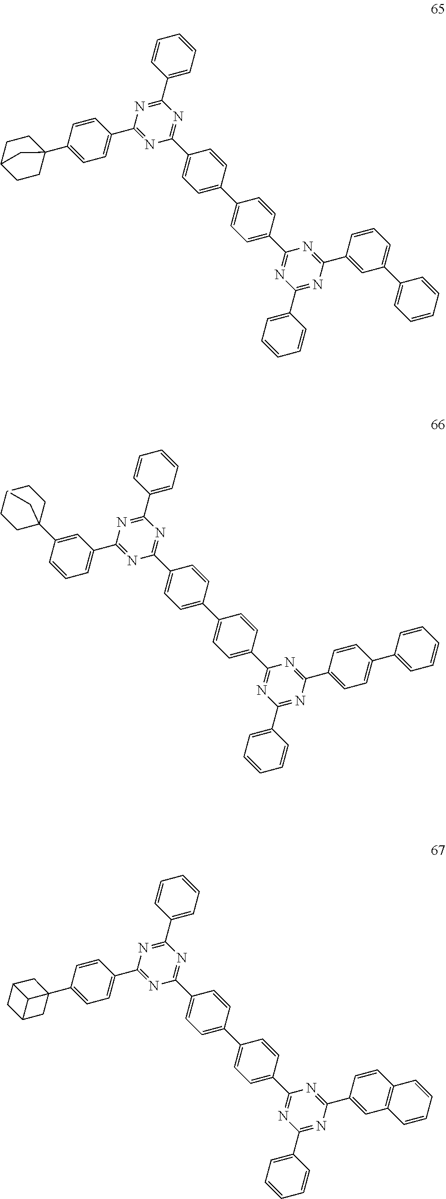

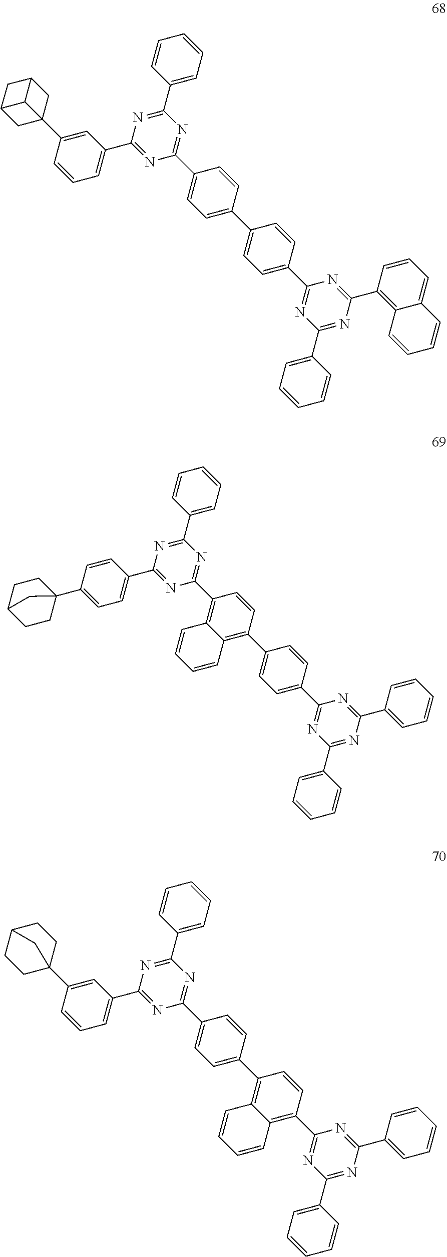 [SMILES: c1ccc(-c2nc(-c3ccc(-c4ccc(-c5nc(-c6ccccc6)nc(-c6cccc7ccccc67)n5)cc4)cc3)nc(-c3cccc(C45CC6CC(C4)C65)c3)n2)cc1 ; c1ccc(-c2nc(-c3ccccc3)nc(-c3ccc(-c4ccc(-c5nc(-c6ccccc6)nc(-c6ccc(C78CCC(CC7)C8)cc6)n5)c5ccccc45)cc3)n2)cc1 ; c1ccc(-c2nc(-c3ccc(-c4ccc(-c5nc(-c6ccccc6)nc(-c6ccccc6)n5)c5ccccc45)cc3)nc(-c3cccc(C45CCC(CC4)C5)c3)n2)cc1]

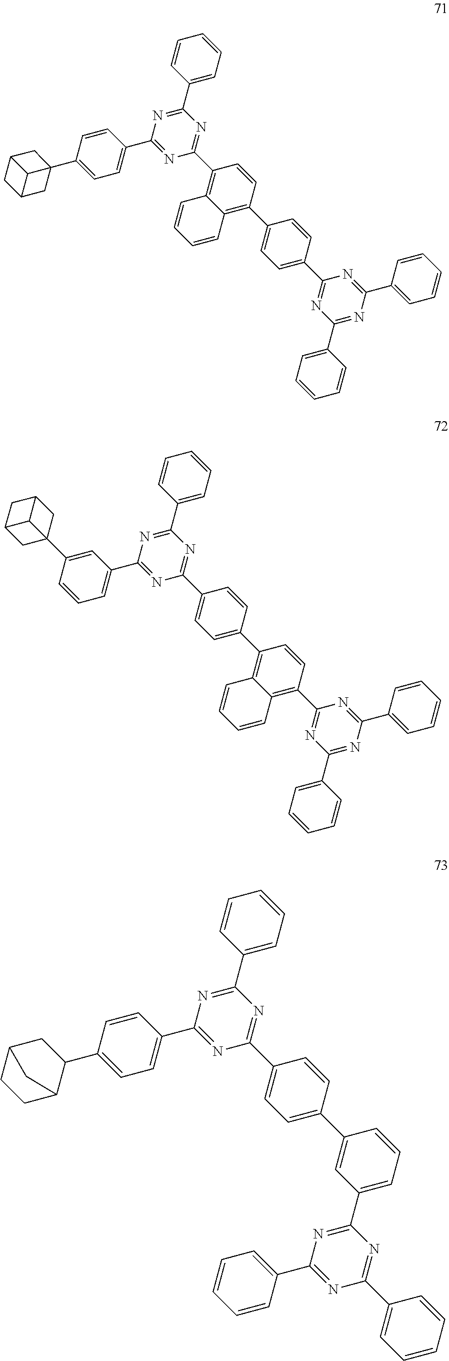 [SMILES: c1ccc(-c2nc(-c3ccccc3)nc(-c3ccc(-c4ccc(-c5nc(-c6ccccc6)nc(-c6ccc(C78CC9CC(C7)C98)cc6)n5)c5ccccc45)cc3)n2)cc1 ; c1ccc(-c2nc(-c3ccc(-c4ccc(-c5nc(-c6ccccc6)nc(-c6ccccc6)n5)c5ccccc45)cc3)nc(-c3cccc(C45CC6CC(C4)C65)c3)n2)cc1 ; c1ccc(-c2nc(-c3ccc(-c4cccc(-c5nc(-c6ccccc6)nc(-c6ccccc6)n5)c4)cc3)nc(-c3ccc(C4CC5CCC4C5)cc3)n2)cc1]

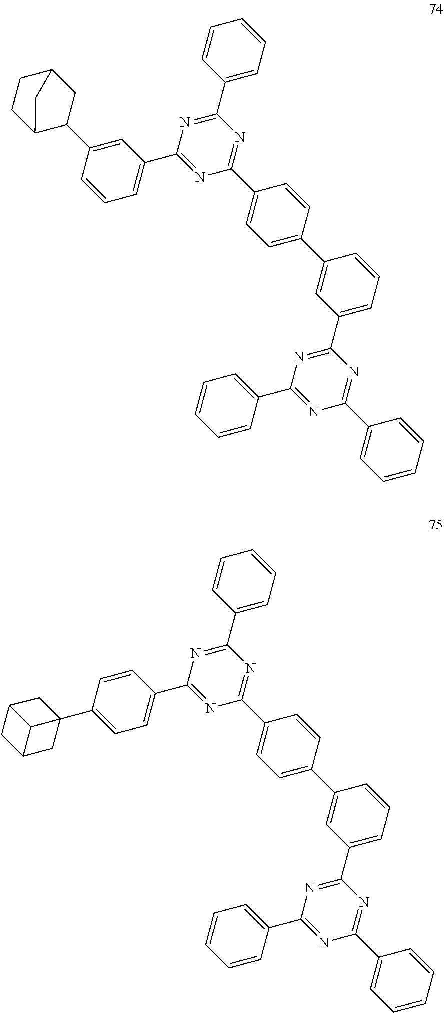 [SMILES: c1ccc(-c2nc(-c3ccccc3)nc(-c3cccc(-c4ccc(-c5nc(-c6ccccc6)nc(-c6cccc(C7CC8CCC7C8)c6)n5)cc4)c3)n2)cc1 ; c1ccc(-c2nc(-c3ccc(-c4cccc(-c5nc(-c6ccccc6)nc(-c6ccccc6)n5)c4)cc3)nc(-c3ccc(C45CC6CC(C4)C65)cc3)n2)cc1]

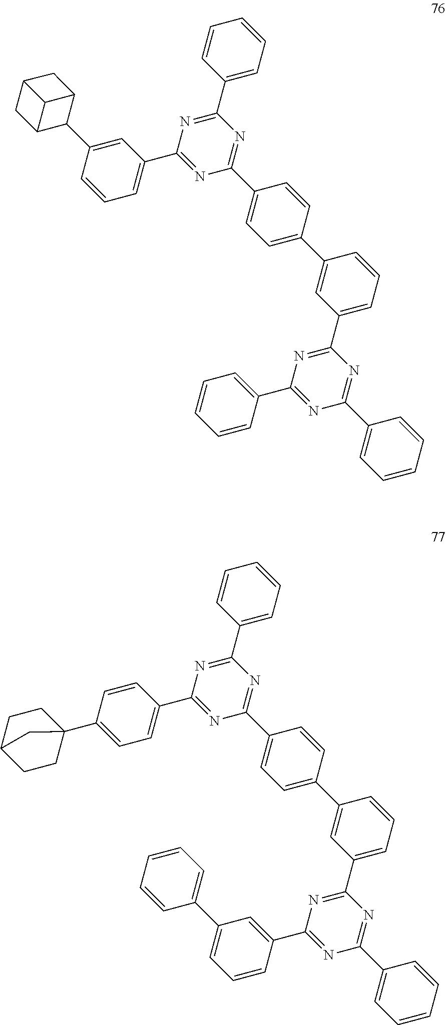 [SMILES: c1ccc(-c2nc(-c3ccccc3)nc(-c3cccc(-c4ccc(-c5nc(-c6ccccc6)nc(-c6cccc(C7C8CC9CC7C98)c6)n5)cc4)c3)n2)cc1 ; c1ccc(-c2cccc(-c3nc(-c4ccccc4)nc(-c4cccc(-c5ccc(-c6nc(-c7ccccc7)nc(-c7ccc(C89CCC(CC8)C9)cc7)n6)cc5)c4)n3)c2)cc1]

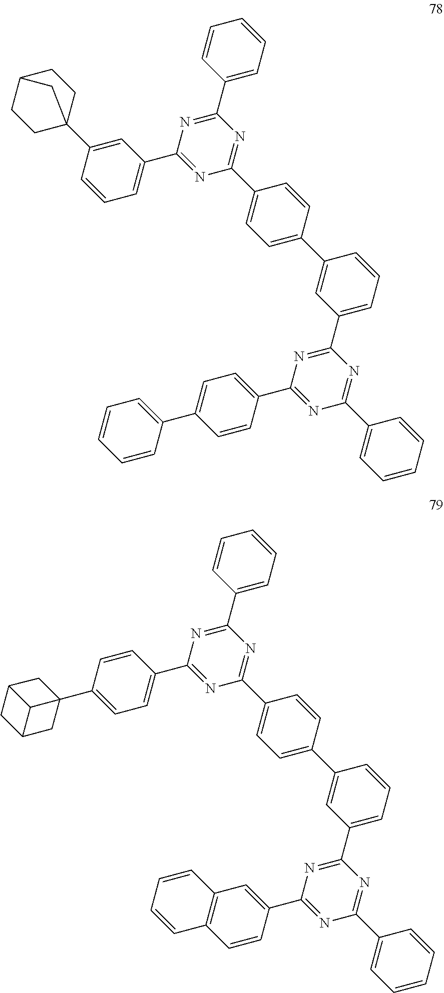 [SMILES: c1ccc(-c2ccc(-c3nc(-c4ccccc4)nc(-c4cccc(-c5ccc(-c6nc(-c7ccccc7)nc(-c7cccc(C89CCC(CC8)C9)c7)n6)cc5)c4)n3)cc2)cc1 ; c1ccc(-c2nc(-c3ccc(-c4cccc(-c5nc(-c6ccccc6)nc(-c6ccc7ccccc7c6)n5)c4)cc3)nc(-c3ccc(C45CC6CC(C4)C65)cc3)n2)cc1]

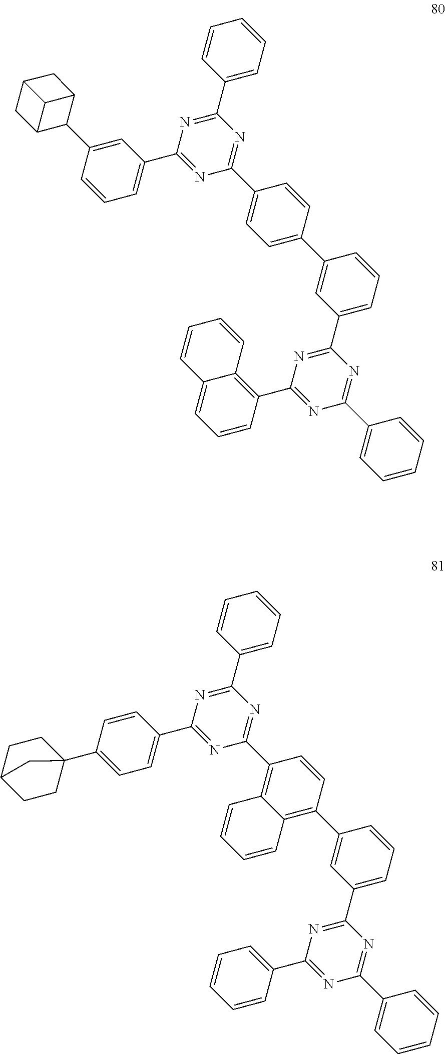 [SMILES: c1ccc(-c2nc(-c3ccc(-c4cccc(-c5nc(-c6ccccc6)nc(-c6cccc7ccccc67)n5)c4)cc3)nc(-c3cccc(C4C5CC6CC4C65)c3)n2)cc1 ; c1ccc(-c2nc(-c3ccccc3)nc(-c3cccc(-c4ccc(-c5nc(-c6ccccc6)nc(-c6ccc(C78CCC(CC7)C8)cc6)n5)c5ccccc45)c3)n2)cc1]

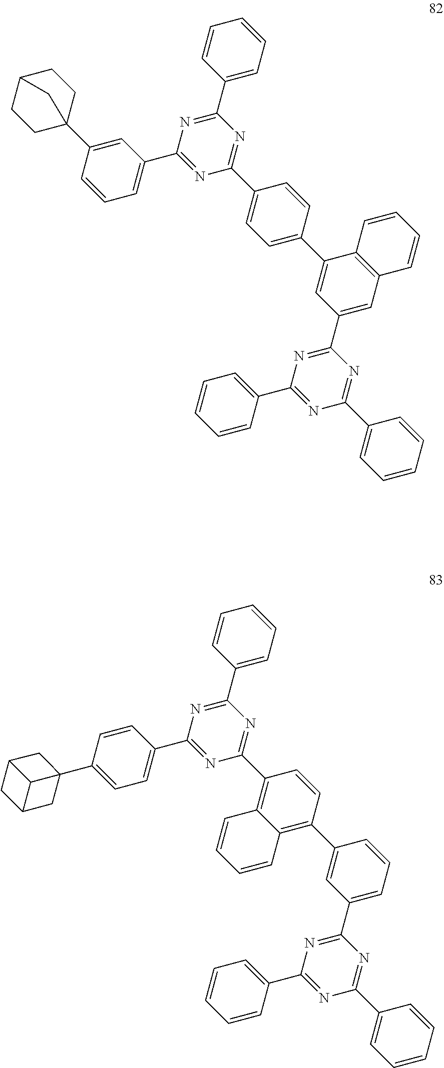 [SMILES: c1ccc(-c2nc(-c3ccc(-c4cc(-c5nc(-c6ccccc6)nc(-c6ccccc6)n5)cc5ccccc45)cc3)nc(-c3cccc(C45CCC(CC4)C5)c3)n2)cc1 ; c1ccc(-c2nc(-c3ccccc3)nc(-c3cccc(-c4ccc(-c5nc(-c6ccccc6)nc(-c6ccc(C78CC9CC(C7)C98)cc6)n5)c5ccccc45)c3)n2)cc1]

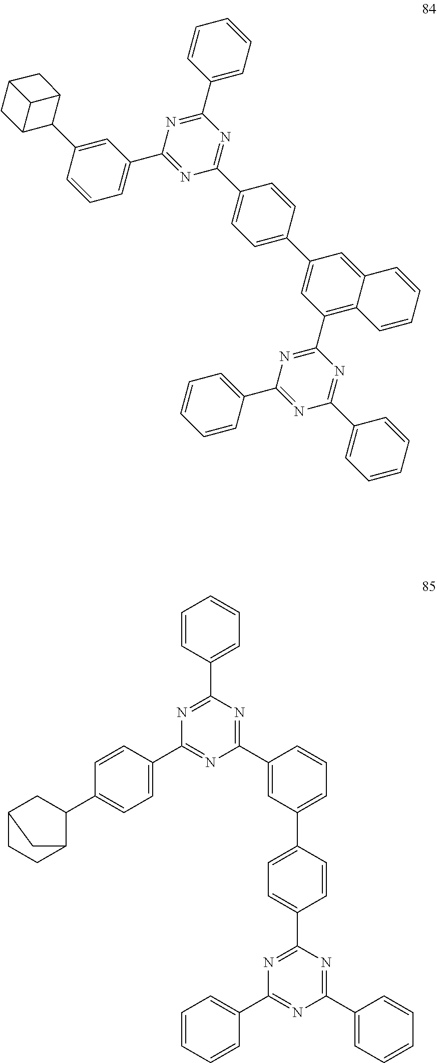 [SMILES: c1ccc(-c2nc(-c3ccc(-c4cc(-c5nc(-c6ccccc6)nc(-c6ccccc6)n5)c5ccccc5c4)cc3)nc(-c3cccc(C4C5CC6CC4C65)c3)n2)cc1 ; c1ccc(-c2nc(-c3ccccc3)nc(-c3ccc(-c4cccc(-c5nc(-c6ccccc6)nc(-c6ccc(C7CC8CCC7C8)cc6)n5)c4)cc3)n2)cc1]

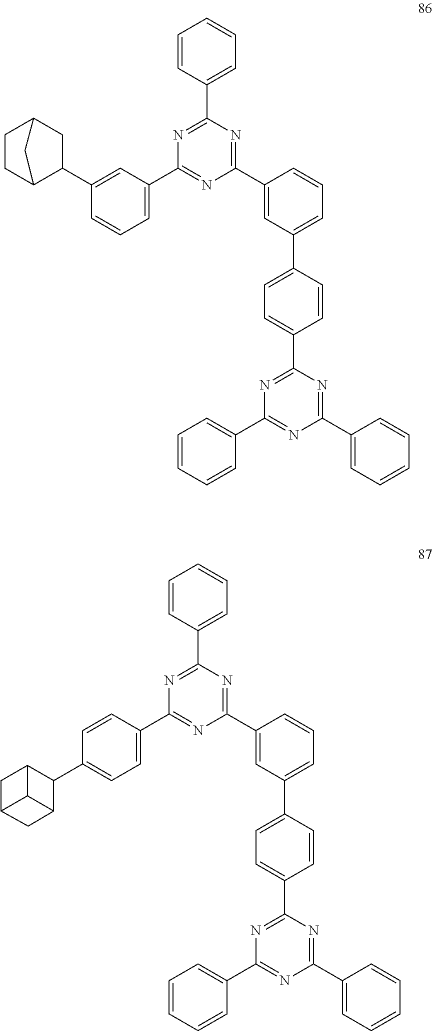 [SMILES: c1ccc(-c2nc(-c3ccccc3)nc(-c3ccc(-c4cccc(-c5nc(-c6ccccc6)nc(-c6cccc(C7CC8CCC7C8)c6)n5)c4)cc3)n2)cc1 ; c1ccc(-c2nc(-c3ccccc3)nc(-c3ccc(-c4cccc(-c5nc(-c6ccccc6)nc(-c6ccc(C7C8CC9CC7C98)cc6)n5)c4)cc3)n2)cc1]

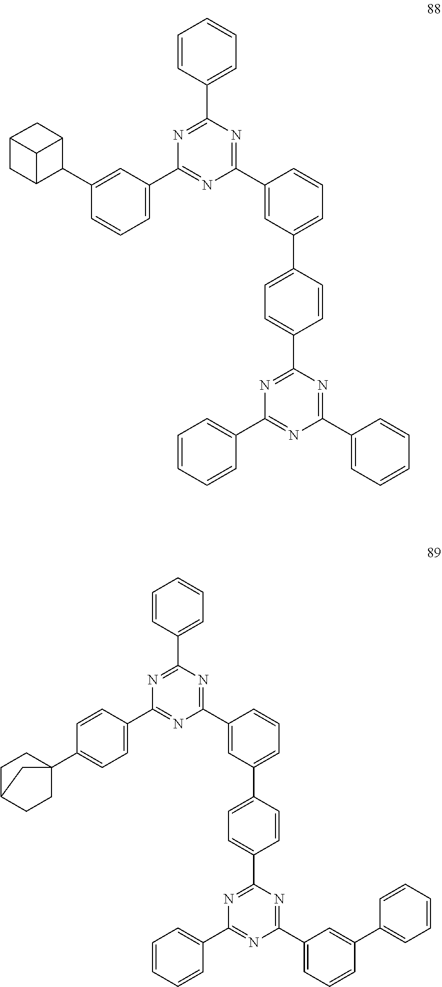 [SMILES: c1ccc(-c2nc(-c3ccccc3)nc(-c3ccc(-c4cccc(-c5nc(-c6ccccc6)nc(-c6cccc(C7C8CC9CC7C98)c6)n5)c4)cc3)n2)cc1 ; c1ccc(-c2cccc(-c3nc(-c4ccccc4)nc(-c4ccc(-c5cccc(-c6nc(-c7ccccc7)nc(-c7ccc(C89CCC(CC8)C9)cc7)n6)c5)cc4)n3)c2)cc1]

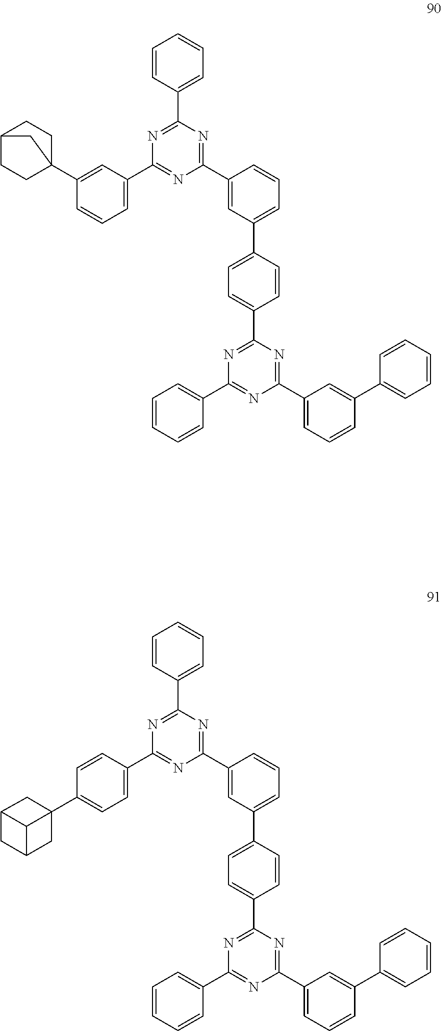 [SMILES: c1ccc(-c2cccc(-c3nc(-c4ccccc4)nc(-c4ccc(-c5cccc(-c6nc(-c7ccccc7)nc(-c7cccc(C89CCC(CC8)C9)c7)n6)c5)cc4)n3)c2)cc1 ; c1ccc(-c2cccc(-c3nc(-c4ccccc4)nc(-c4ccc(-c5cccc(-c6nc(-c7ccccc7)nc(-c7ccc(C89CC%10CC(C8)C%109)cc7)n6)c5)cc4)n3)c2)cc1]

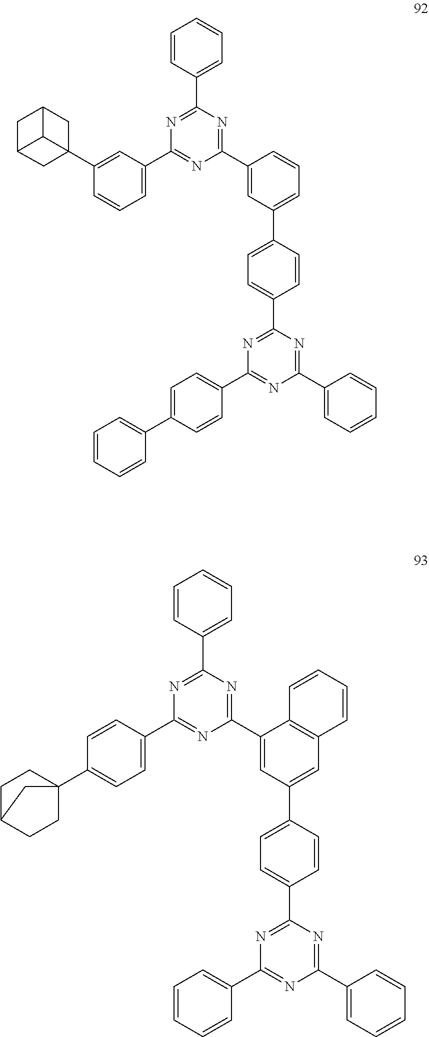 [SMILES: c1ccc(-c2ccc(-c3nc(-c4ccccc4)nc(-c4ccc(-c5cccc(-c6nc(-c7ccccc7)nc(-c7cccc(C89CC%10CC(C8)C%109)c7)n6)c5)cc4)n3)cc2)cc1 ; c1ccc(-c2nc(-c3ccccc3)nc(-c3ccc(-c4cc(-c5nc(-c6ccccc6)nc(-c6ccc(C78CCC(CC7)C8)cc6)n5)c5ccccc5c4)cc3)n2)cc1]

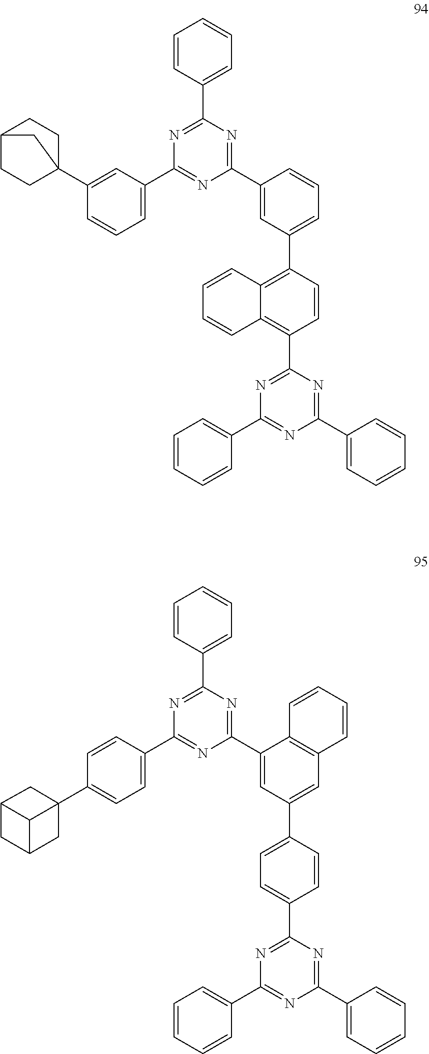 [SMILES: c1ccc(-c2nc(-c3cccc(-c4ccc(-c5nc(-c6ccccc6)nc(-c6ccccc6)n5)c5ccccc45)c3)nc(-c3cccc(C45CCC(CC4)C5)c3)n2)cc1 ; c1ccc(-c2nc(-c3ccccc3)nc(-c3ccc(-c4cc(-c5nc(-c6ccccc6)nc(-c6ccc(C78CC9CC(C7)C98)cc6)n5)c5ccccc5c4)cc3)n2)cc1]

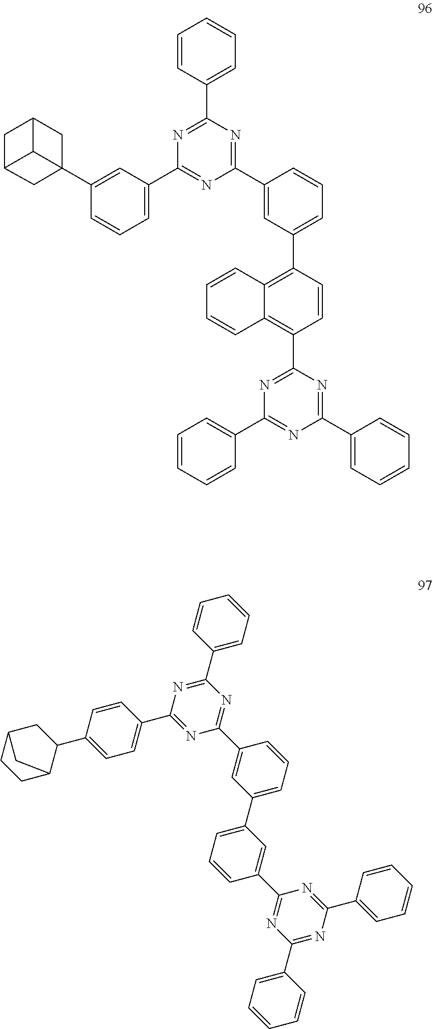 [SMILES: c1ccc(-c2nc(-c3cccc(-c4ccc(-c5nc(-c6ccccc6)nc(-c6ccccc6)n5)c5ccccc45)c3)nc(-c3cccc(C45CC6CC(C4)C65)c3)n2)cc1 ; c1ccc(-c2nc(-c3ccccc3)nc(-c3cccc(-c4cccc(-c5nc(-c6ccccc6)nc(-c6ccc(C7CC8CCC7C8)cc6)n5)c4)c3)n2)cc1]

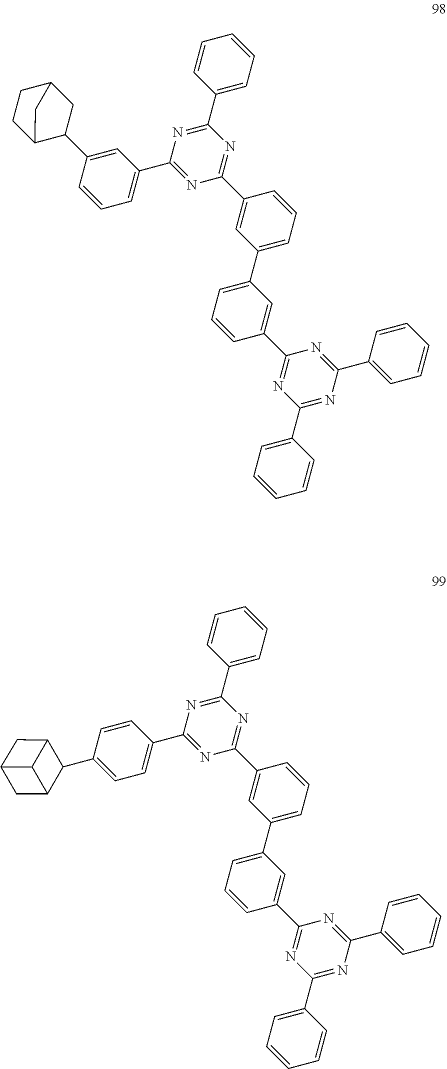 [SMILES: c1ccc(-c2nc(-c3ccccc3)nc(-c3cccc(-c4cccc(-c5nc(-c6ccccc6)nc(-c6cccc(C7CC8CCC7C8)c6)n5)c4)c3)n2)cc1 ; c1ccc(-c2nc(-c3ccccc3)nc(-c3cccc(-c4cccc(-c5nc(-c6ccccc6)nc(-c6ccc(C7C8CC9CC7C98)cc6)n5)c4)c3)n2)cc1]

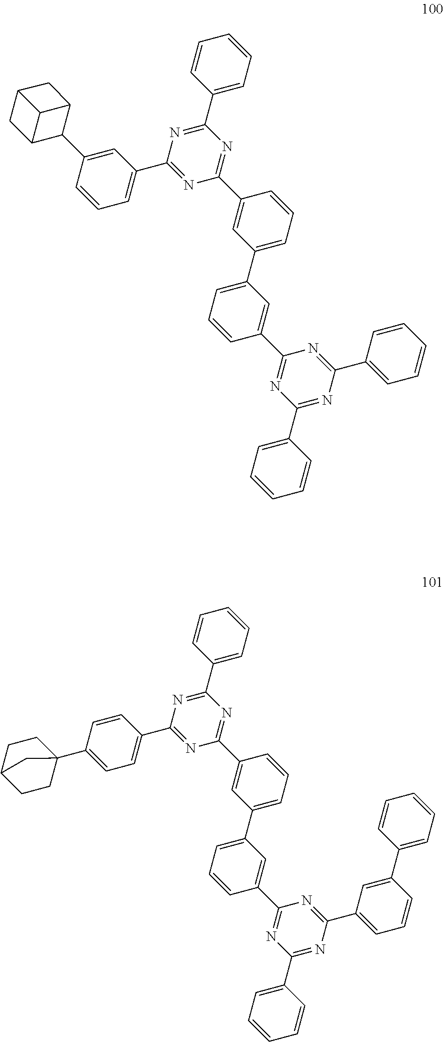 [SMILES: c1ccc(-c2nc(-c3ccccc3)nc(-c3cccc(-c4cccc(-c5nc(-c6ccccc6)nc(-c6cccc(C7C8CC9CC7C98)c6)n5)c4)c3)n2)cc1 ; c1ccc(-c2cccc(-c3nc(-c4ccccc4)nc(-c4cccc(-c5cccc(-c6nc(-c7ccccc7)nc(-c7ccc(C89CCC(CC8)C9)cc7)n6)c5)c4)n3)c2)cc1]

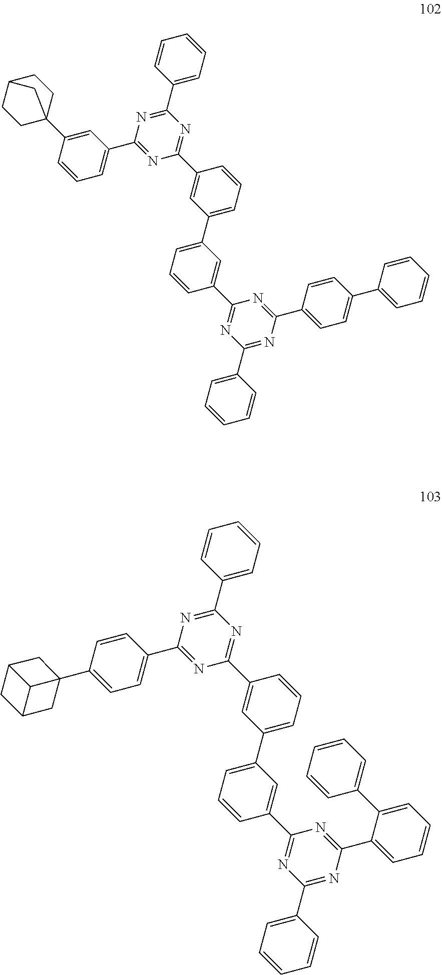 [SMILES: c1ccc(-c2ccc(-c3nc(-c4ccccc4)nc(-c4cccc(-c5cccc(-c6nc(-c7ccccc7)nc(-c7cccc(C89CCC(CC8)C9)c7)n6)c5)c4)n3)cc2)cc1 ; c1ccc(-c2nc(-c3ccc(C45CC6CC(C4)C65)cc3)nc(-c3cccc(-c4cccc(-c5nc(-c6ccccc6)nc(-c6ccccc6-c6ccccc6)n5)c4)c3)n2)cc1]

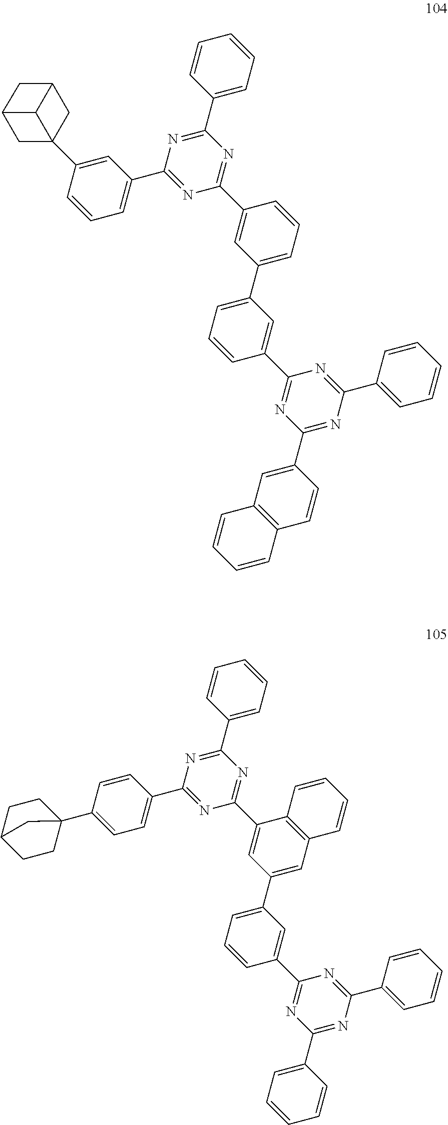 [SMILES: c1ccc(-c2nc(-c3cccc(-c4cccc(-c5nc(-c6ccccc6)nc(-c6ccc7ccccc7c6)n5)c4)c3)nc(-c3cccc(C45CC6CC(C4)C65)c3)n2)cc1 ; c1ccc(-c2nc(-c3ccccc3)nc(-c3cccc(-c4cc(-c5nc(-c6ccccc6)nc(-c6ccc(C78CCC(CC7)C8)cc6)n5)c5ccccc5c4)c3)n2)cc1]

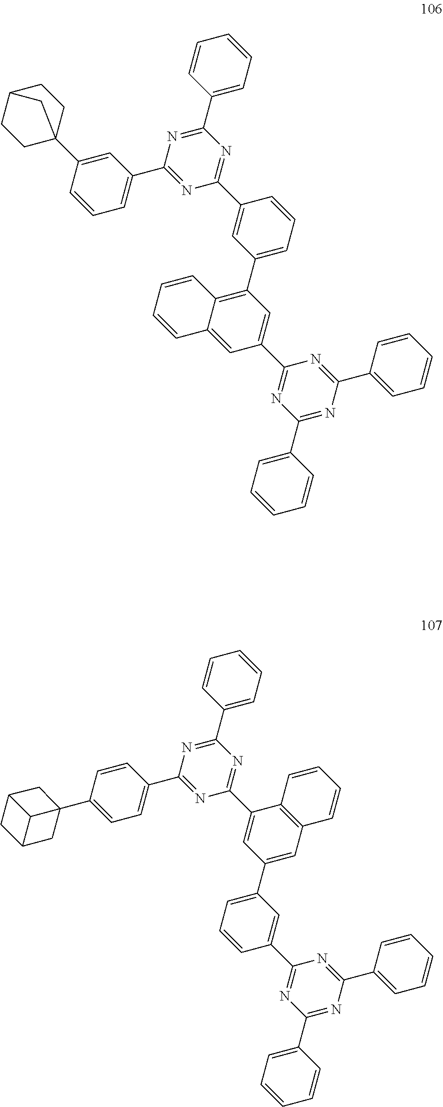 [SMILES: c1ccc(-c2nc(-c3cccc(-c4cc(-c5nc(-c6ccccc6)nc(-c6ccccc6)n5)cc5ccccc45)c3)nc(-c3cccc(C45CCC(CC4)C5)c3)n2)cc1 ; c1ccc(-c2nc(-c3ccccc3)nc(-c3cccc(-c4cc(-c5nc(-c6ccccc6)nc(-c6ccc(C78CC9CC(C7)C98)cc6)n5)c5ccccc5c4)c3)n2)cc1]

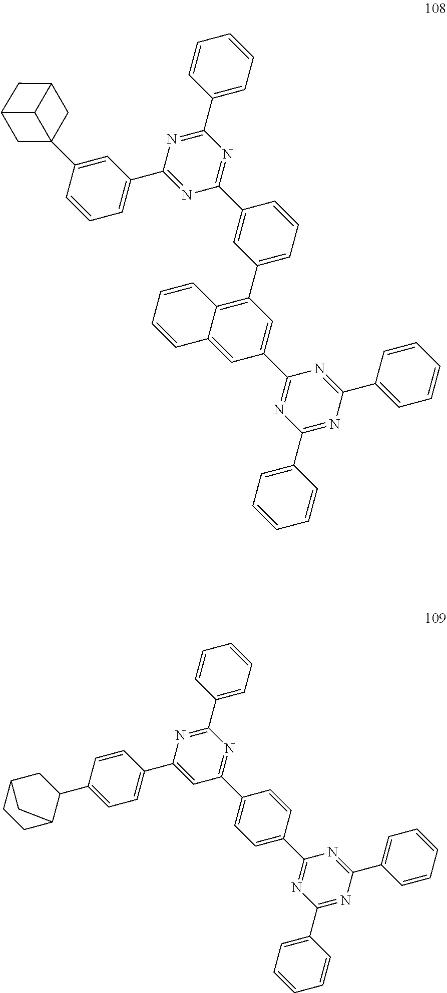 [SMILES: c1ccc(-c2nc(-c3cccc(-c4cc(-c5nc(-c6ccccc6)nc(-c6ccccc6)n5)cc5ccccc45)c3)nc(-c3cccc(C45CC6CC(C4)C65)c3)n2)cc1 ; c1ccc(-c2nc(-c3ccc(-c4nc(-c5ccccc5)nc(-c5ccccc5)n4)cc3)cc(-c3ccc(C4CC5CCC4C5)cc3)n2)cc1]

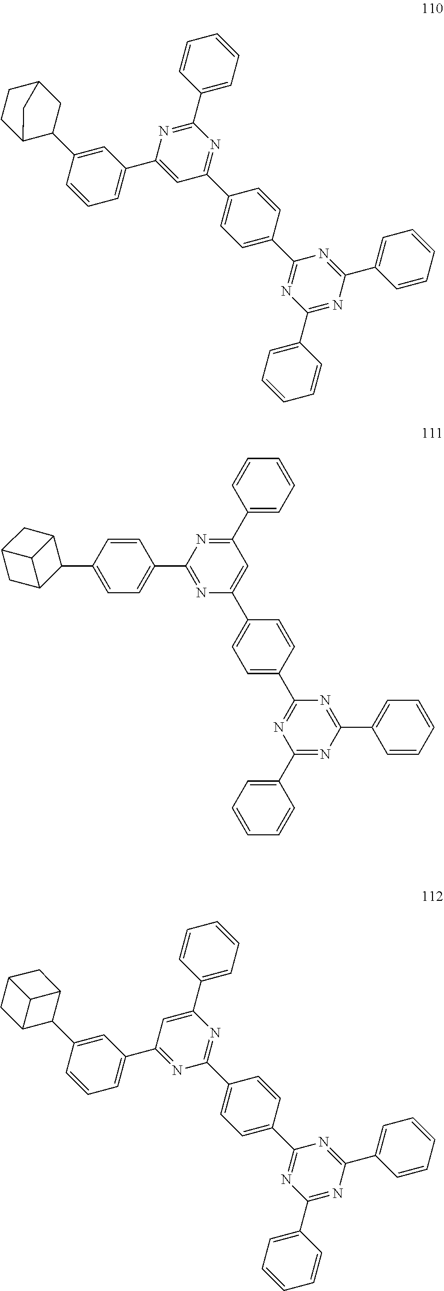 [SMILES: c1ccc(-c2nc(-c3ccc(-c4nc(-c5ccccc5)nc(-c5ccccc5)n4)cc3)cc(-c3cccc(C4CC5CCC4C5)c3)n2)cc1 ; c1ccc(-c2cc(-c3ccc(-c4nc(-c5ccccc5)nc(-c5ccccc5)n4)cc3)nc(-c3ccc(C4C5CC6CC4C65)cc3)n2)cc1 ; c1ccc(-c2cc(-c3cccc(C4C5CC6CC4C65)c3)nc(-c3ccc(-c4nc(-c5ccccc5)nc(-c5ccccc5)n4)cc3)n2)cc1]

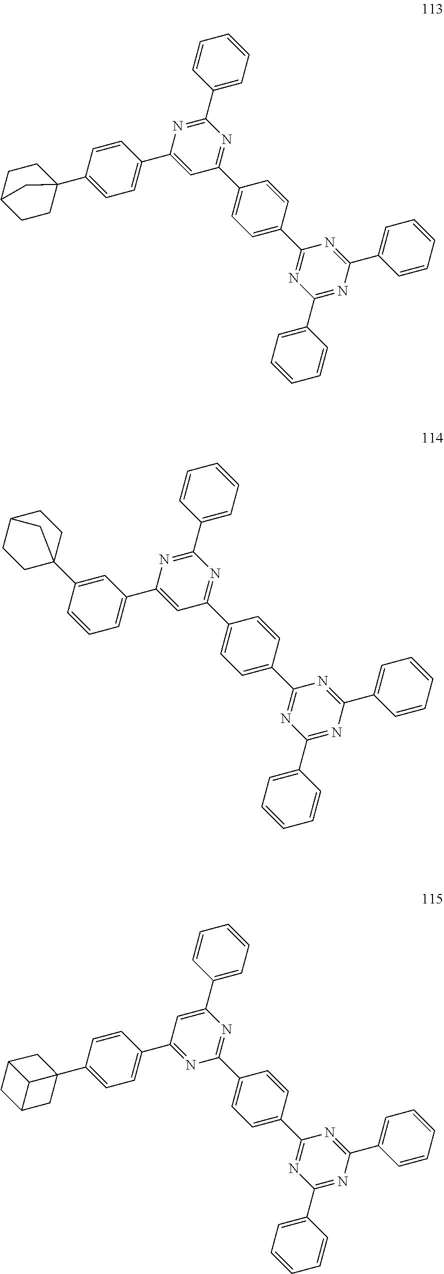 [SMILES: c1ccc(-c2nc(-c3ccc(-c4nc(-c5ccccc5)nc(-c5ccccc5)n4)cc3)cc(-c3ccc(C45CCC(CC4)C5)cc3)n2)cc1 ; c1ccc(-c2nc(-c3ccc(-c4nc(-c5ccccc5)nc(-c5ccccc5)n4)cc3)cc(-c3cccc(C45CCC(CC4)C5)c3)n2)cc1 ; c1ccc(-c2cc(-c3ccc(C45CC6CC(C4)C65)cc3)nc(-c3ccc(-c4nc(-c5ccccc5)nc(-c5ccccc5)n4)cc3)n2)cc1]

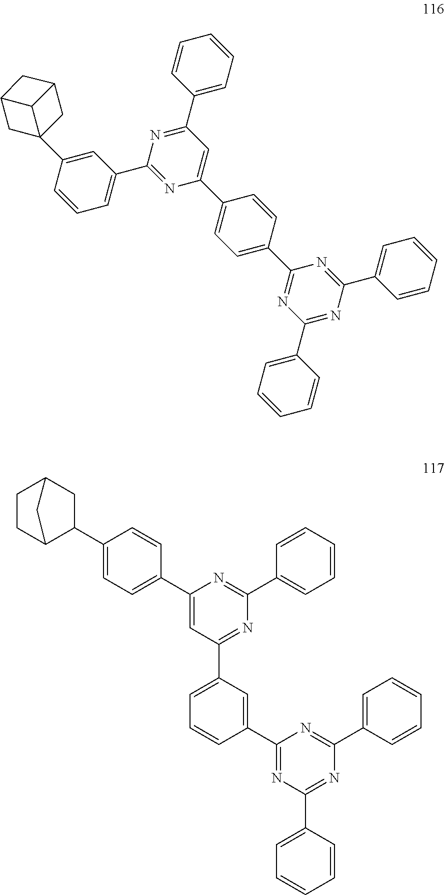 [SMILES: c1ccc(-c2cc(-c3ccc(-c4nc(-c5ccccc5)nc(-c5ccccc5)n4)cc3)nc(-c3cccc(C45CC6CC(C4)C65)c3)n2)cc1 ; c1ccc(-c2nc(-c3ccc(C4CC5CCC4C5)cc3)cc(-c3cccc(-c4nc(-c5ccccc5)nc(-c5ccccc5)n4)c3)n2)cc1]

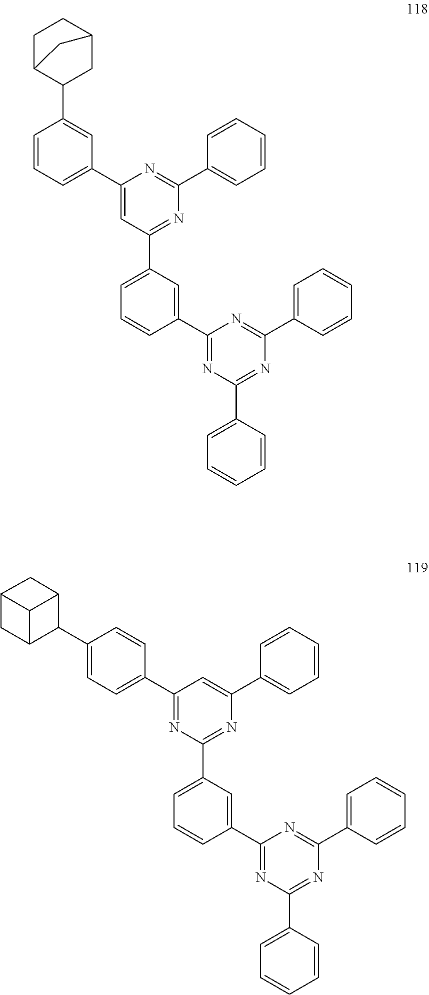 [SMILES: c1ccc(-c2nc(-c3cccc(-c4nc(-c5ccccc5)nc(-c5ccccc5)n4)c3)cc(-c3cccc(C4CC5CCC4C5)c3)n2)cc1 ; c1ccc(-c2cc(-c3ccc(C4C5CC6CC4C65)cc3)nc(-c3cccc(-c4nc(-c5ccccc5)nc(-c5ccccc5)n4)c3)n2)cc1]

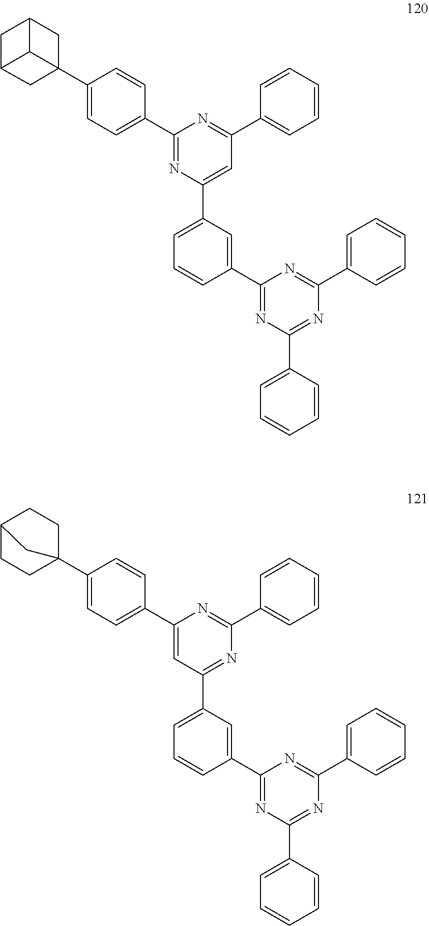 [SMILES: c1ccc(-c2cc(-c3cccc(-c4nc(-c5ccccc5)nc(-c5ccccc5)n4)c3)nc(-c3ccc(C45CC6CC(C4)C65)cc3)n2)cc1 ; c1ccc(-c2nc(-c3ccc(C45CCC(CC4)C5)cc3)cc(-c3cccc(-c4nc(-c5ccccc5)nc(-c5ccccc5)n4)c3)n2)cc1]

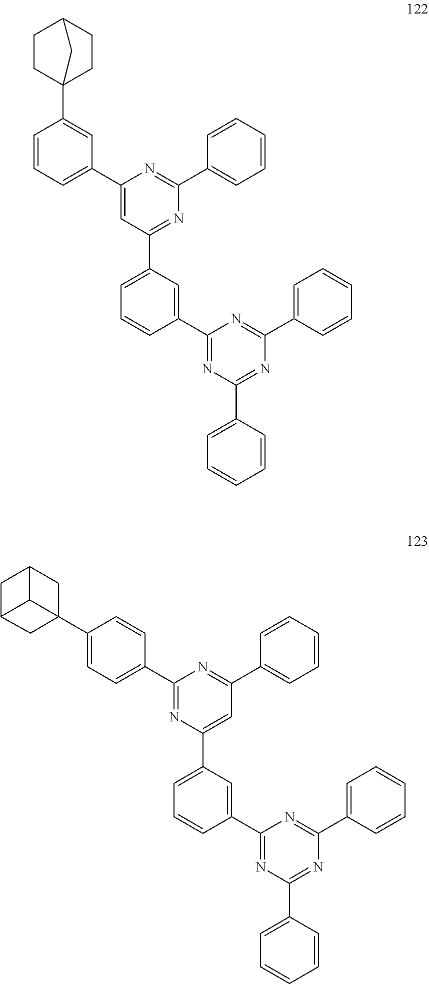 [SMILES: c1ccc(-c2nc(-c3cccc(-c4nc(-c5ccccc5)nc(-c5ccccc5)n4)c3)cc(-c3cccc(C45CCC(CC4)C5)c3)n2)cc1 ; c1ccc(-c2cc(-c3cccc(-c4nc(-c5ccccc5)nc(-c5ccccc5)n4)c3)nc(-c3ccc(C45CC6CC(C4)C65)cc3)n2)cc1]

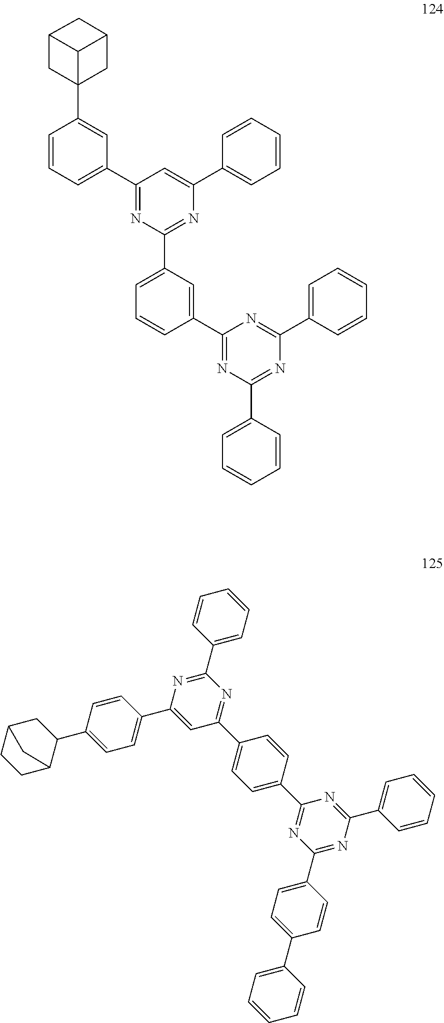 [SMILES: c1ccc(-c2cc(-c3cccc(C45CC6CC(C4)C65)c3)nc(-c3cccc(-c4nc(-c5ccccc5)nc(-c5ccccc5)n4)c3)n2)cc1 ; c1ccc(-c2ccc(-c3nc(-c4ccccc4)nc(-c4ccc(-c5cc(-c6ccc(C7CC8CCC7C8)cc6)nc(-c6ccccc6)n5)cc4)n3)cc2)cc1]

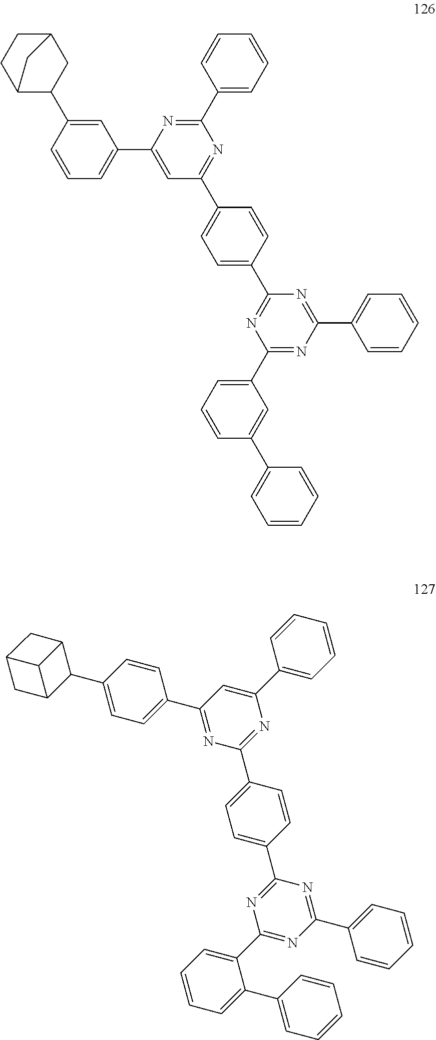 [SMILES: c1ccc(-c2cccc(-c3nc(-c4ccccc4)nc(-c4ccc(-c5cc(-c6cccc(C7CC8CCC7C8)c6)nc(-c6ccccc6)n5)cc4)n3)c2)cc1 ; c1ccc(-c2cc(-c3ccc(C4C5CC6CC4C65)cc3)nc(-c3ccc(-c4nc(-c5ccccc5)nc(-c5ccccc5-c5ccccc5)n4)cc3)n2)cc1]

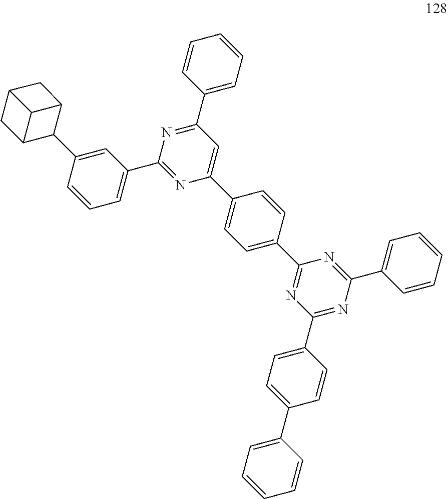 [SMILES: c1ccc(-c2ccc(-c3nc(-c4ccccc4)nc(-c4ccc(-c5cc(-c6ccccc6)nc(-c6cccc(C7C8CC9CC7C98)c6)n5)cc4)n3)cc2)cc1]

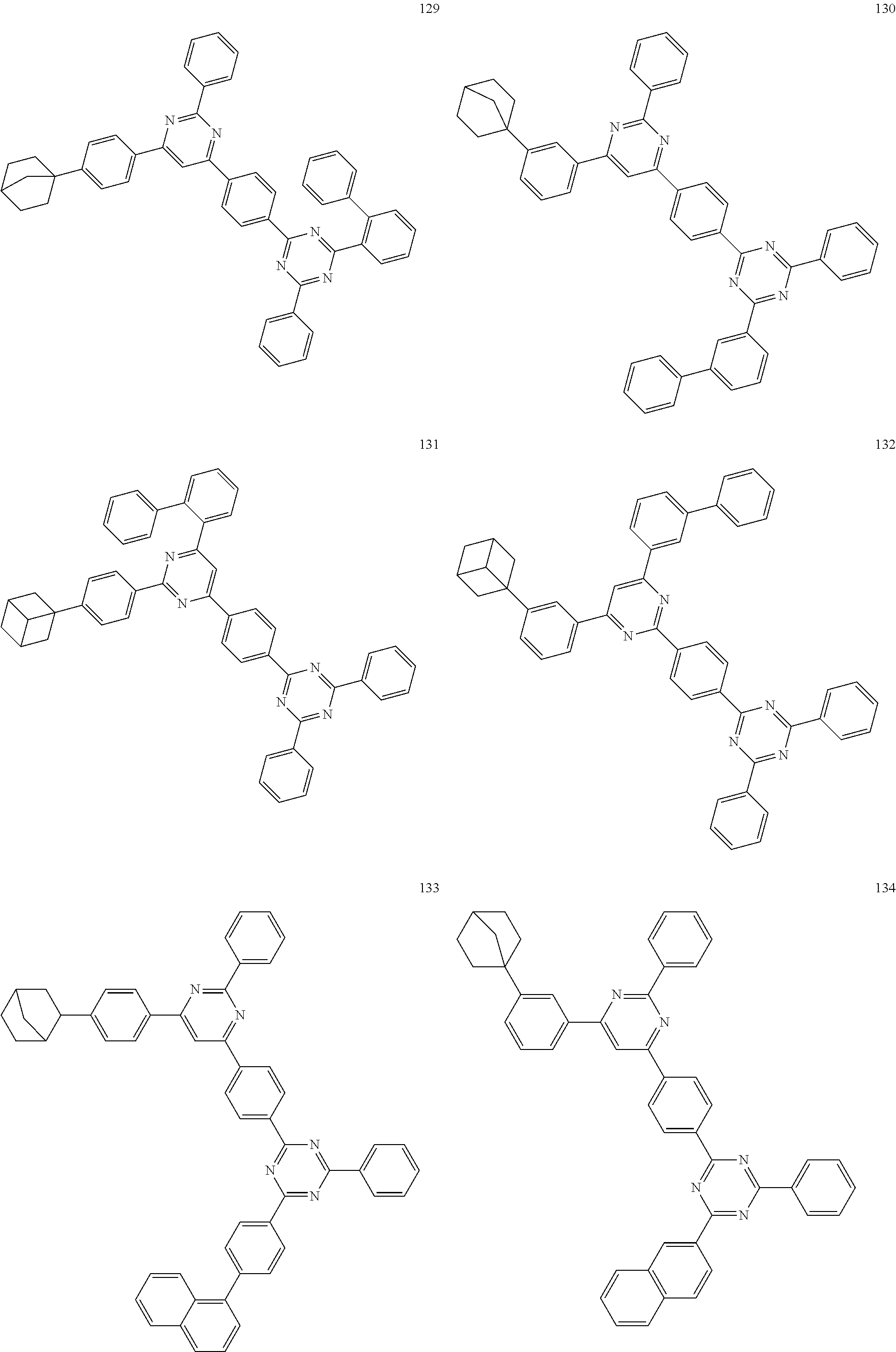

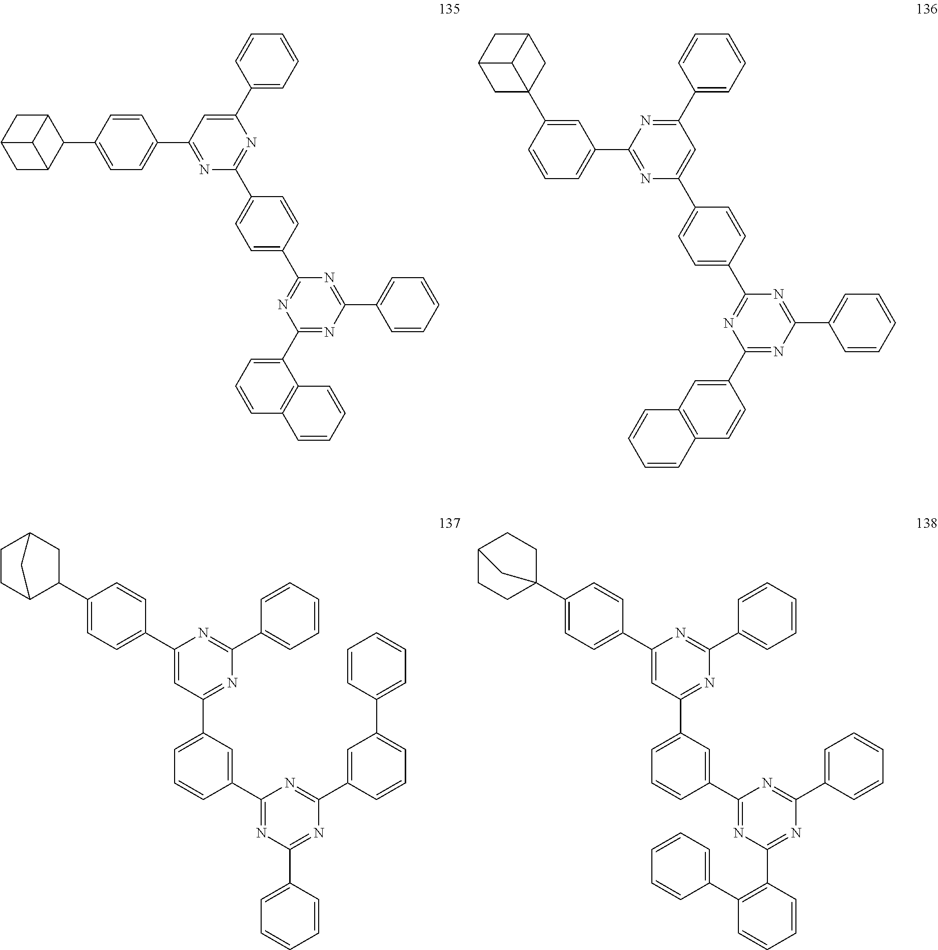 [SMILES: c1ccc(-c2cc(-c3ccc(C4C5CC6CC4C65)cc3)nc(-c3ccc(-c4nc(-c5ccccc5)nc(-c5cccc6ccccc56)n4)cc3)n2)cc1 ; c1ccc(-c2cc(-c3ccc(-c4nc(-c5ccccc5)nc(-c5ccc6ccccc6c5)n4)cc3)nc(-c3cccc(C45CC6CC(C4)C65)c3)n2)cc1 ; c1ccc(-c2cccc(-c3nc(-c4ccccc4)nc(-c4cccc(-c5cc(-c6ccc(C7CC8CCC7C8)cc6)nc(-c6ccccc6)n5)c4)n3)c2)cc1 ; c1ccc(-c2nc(-c3ccc(C45CCC(CC4)C5)cc3)cc(-c3cccc(-c4nc(-c5ccccc5)nc(-c5ccccc5-c5ccccc5)n4)c3)n2)cc1]

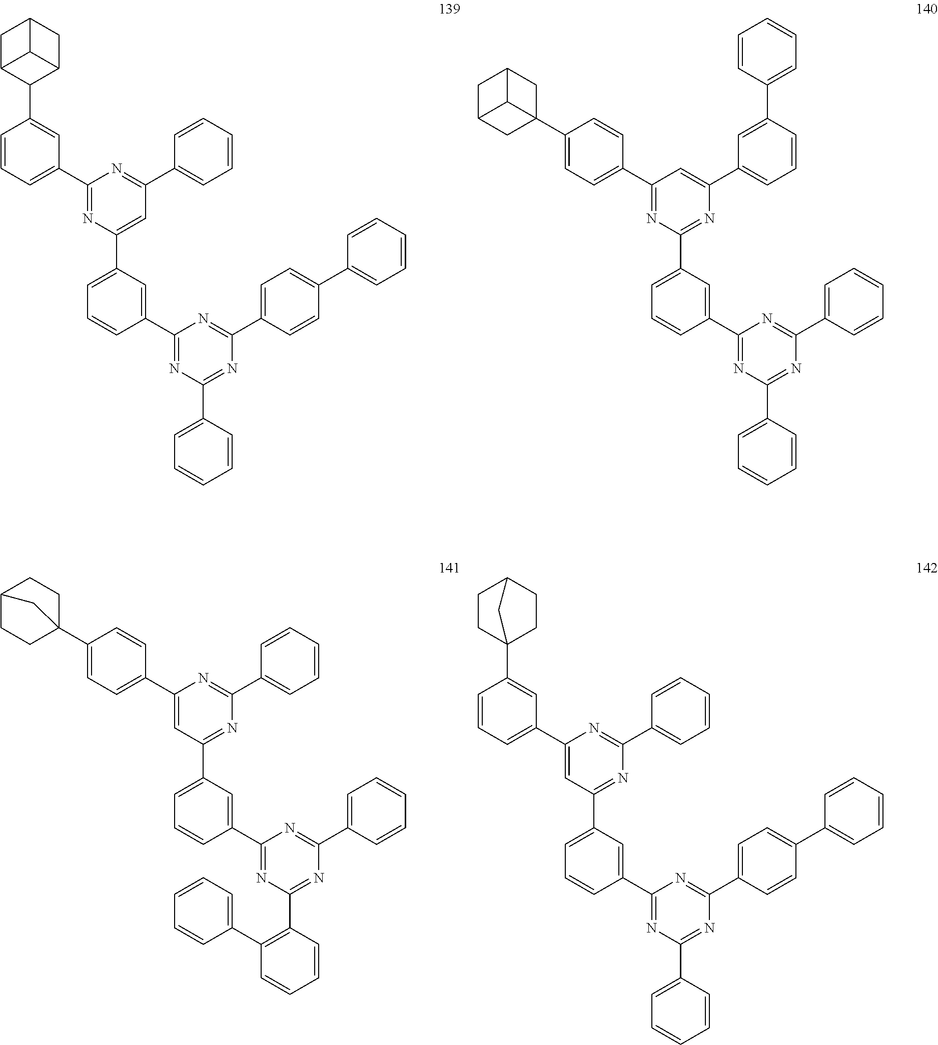 [SMILES: c1ccc(-c2ccc(-c3nc(-c4ccccc4)nc(-c4cccc(-c5cc(-c6ccccc6)nc(-c6cccc(C7C8CC9CC7C98)c6)n5)c4)n3)cc2)cc1 ; c1ccc(-c2cccc(-c3cc(-c4ccc(C56CC7CC(C5)C76)cc4)nc(-c4cccc(-c5nc(-c6ccccc6)nc(-c6ccccc6)n5)c4)n3)c2)cc1 ; c1ccc(-c2nc(-c3ccc(C45CCC(CC4)C5)cc3)cc(-c3cccc(-c4nc(-c5ccccc5)nc(-c5ccccc5-c5ccccc5)n4)c3)n2)cc1 ; c1ccc(-c2ccc(-c3nc(-c4ccccc4)nc(-c4cccc(-c5cc(-c6cccc(C78CCC(CC7)C8)c6)nc(-c6ccccc6)n5)c4)n3)cc2)cc1]

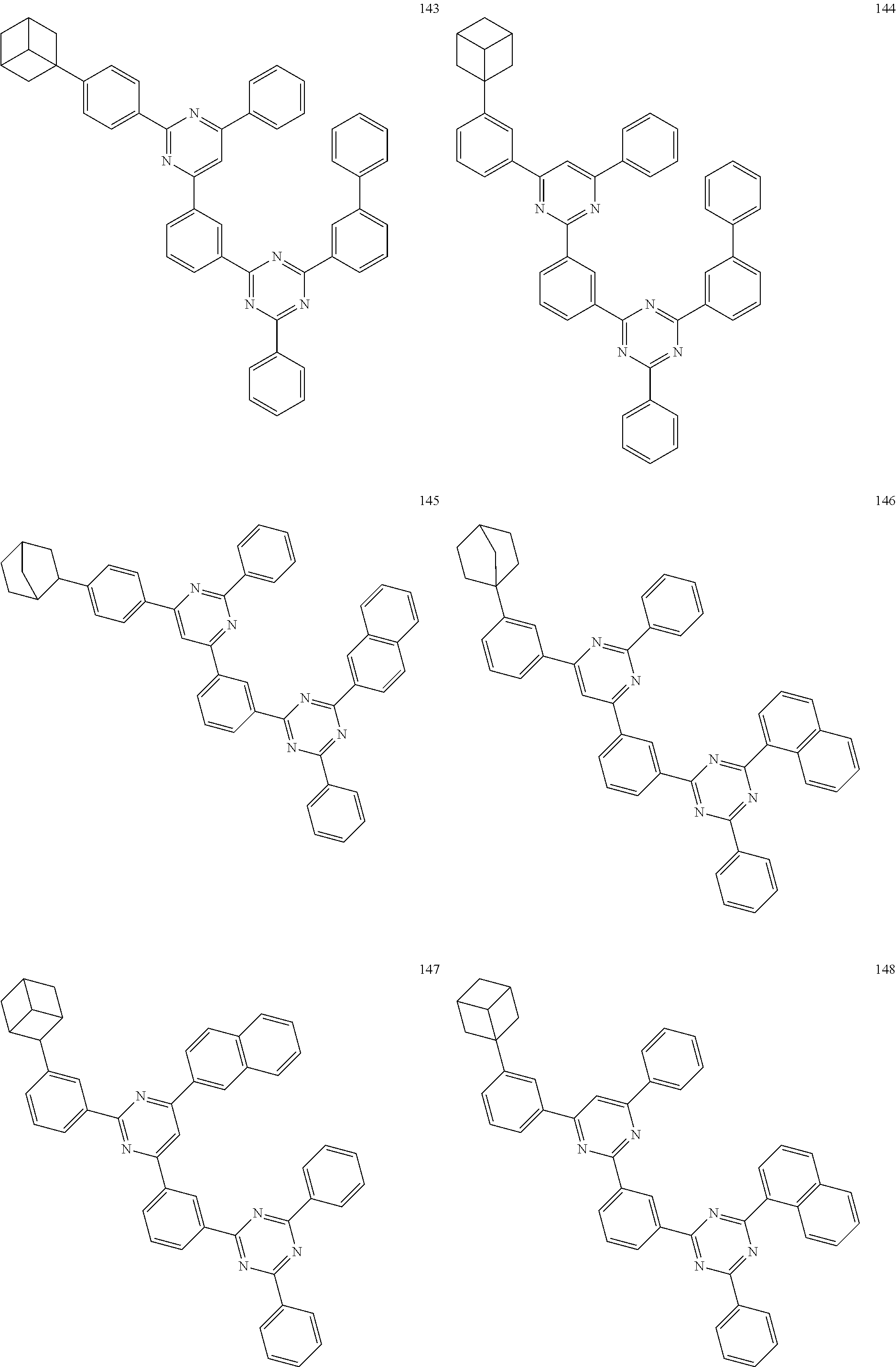

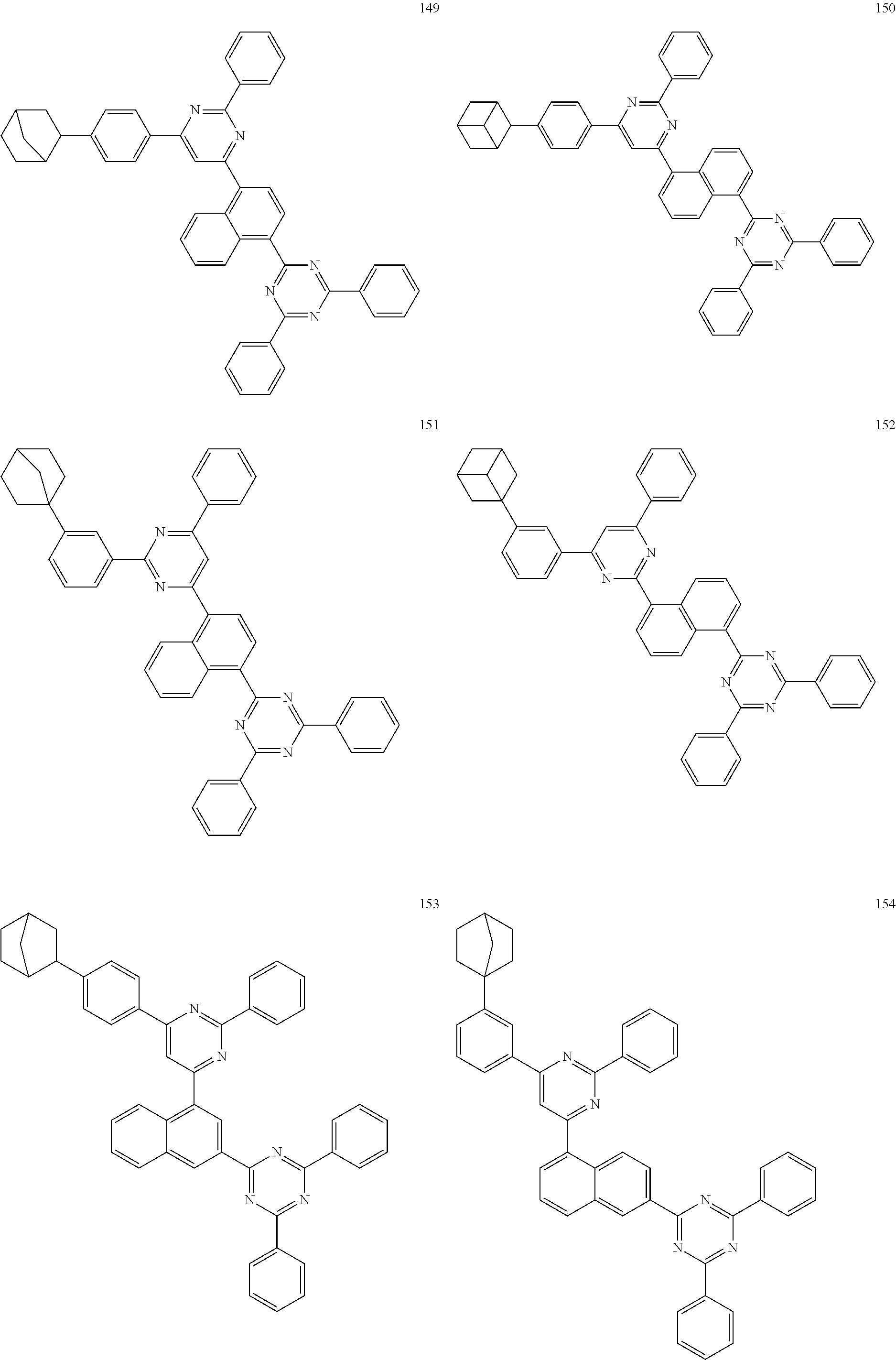

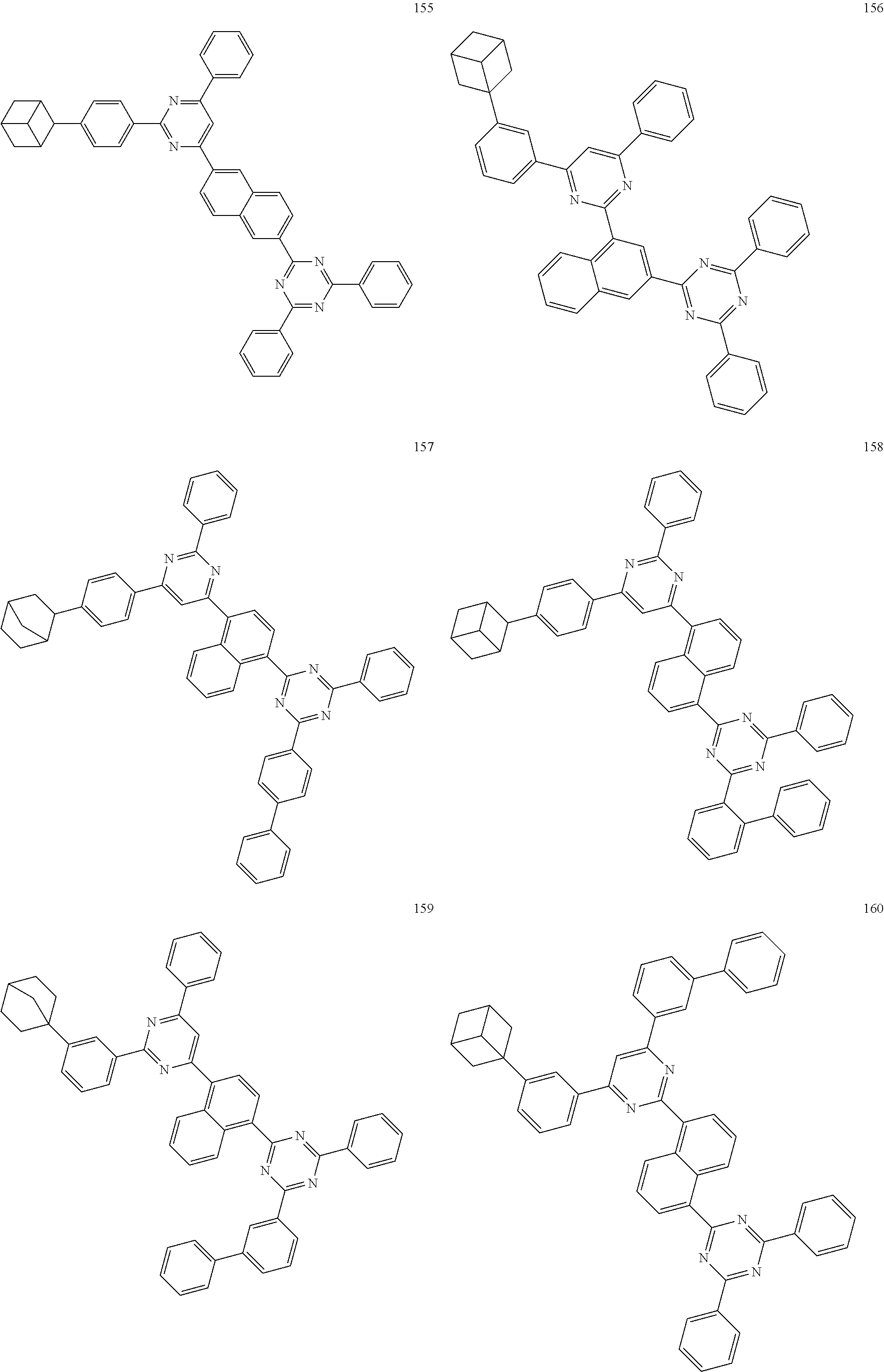

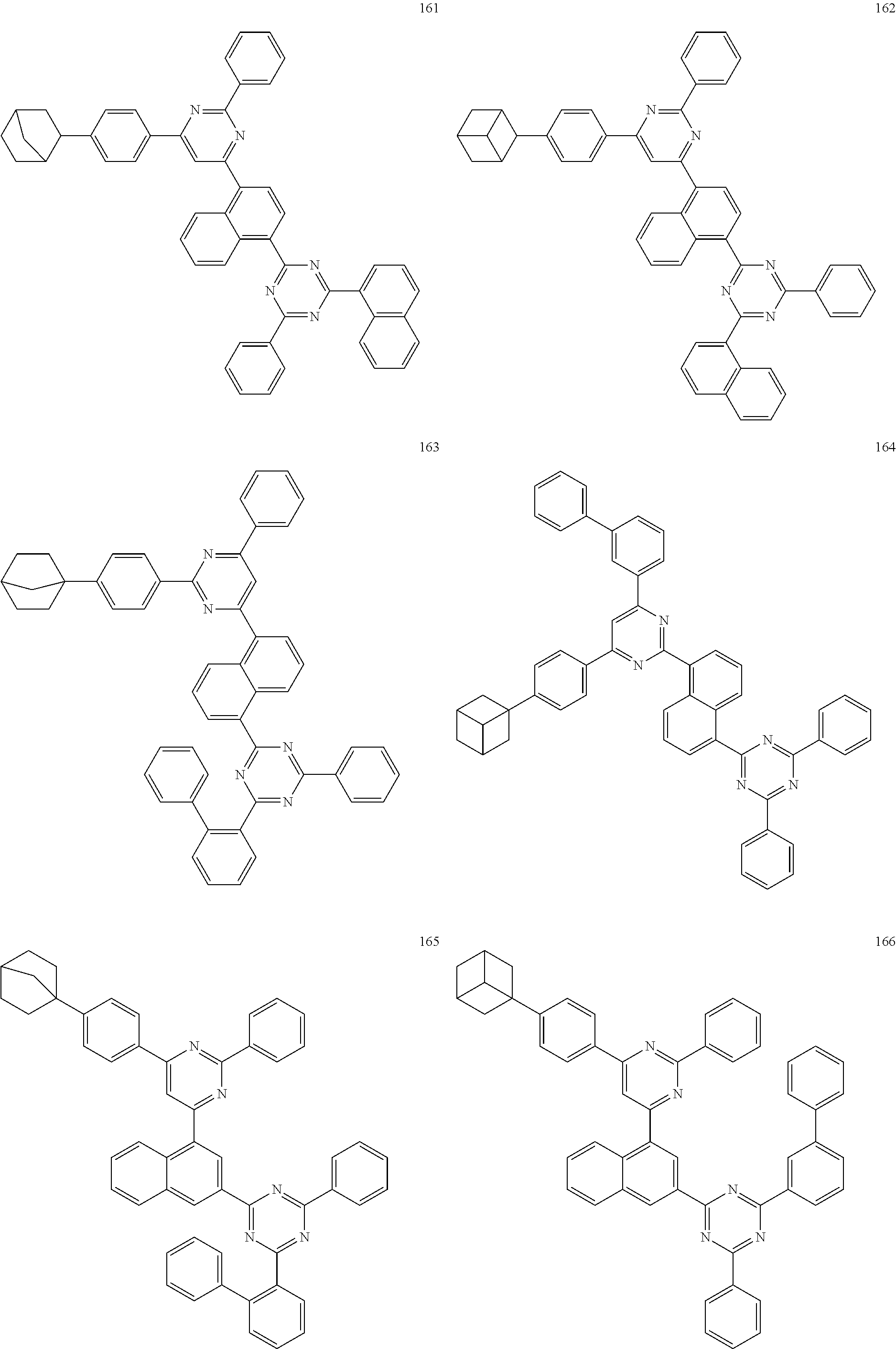

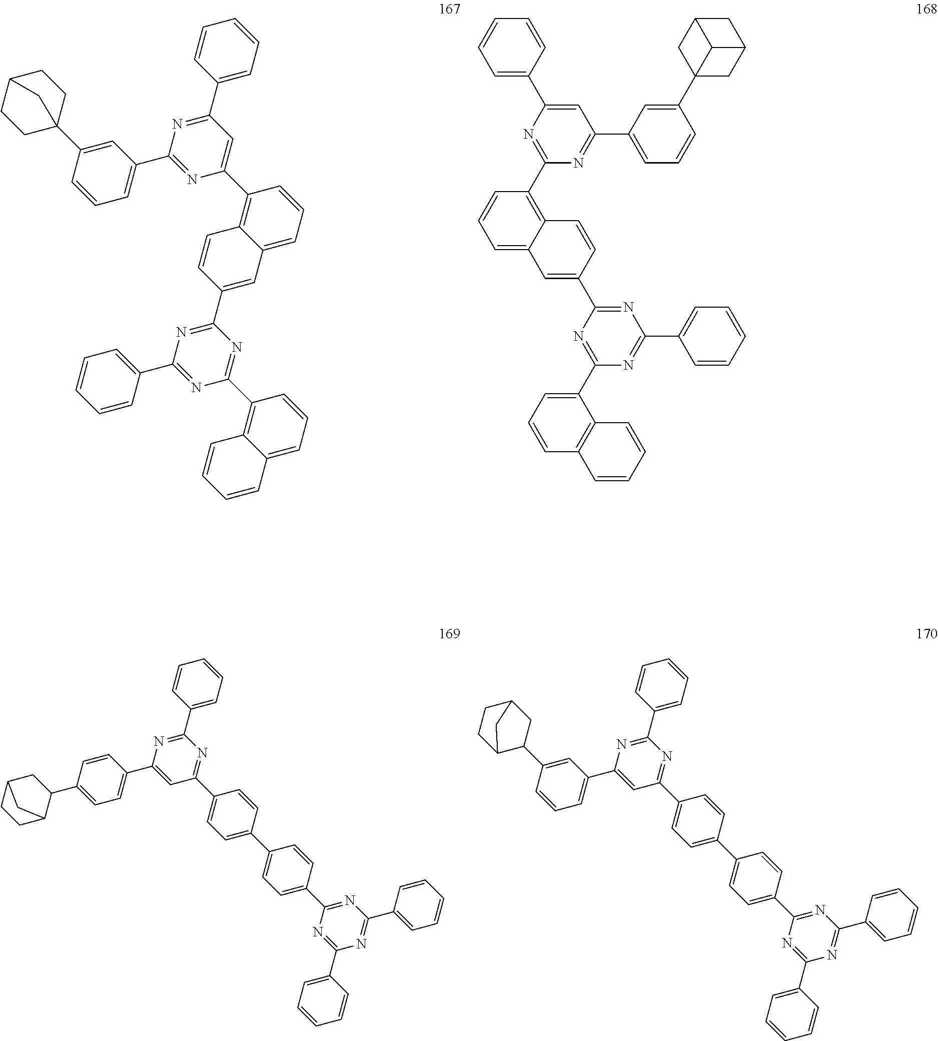 [SMILES: c1ccc(-c2cc(-c3cccc4cc(-c5nc(-c6ccccc6)nc(-c6cccc7ccccc67)n5)ccc34)nc(-c3cccc(C45CCC(CC4)C5)c3)n2)cc1 ; c1ccc(-c2cc(-c3cccc(C45CC6CC(C4)C65)c3)nc(-c3cccc4cc(-c5nc(-c6ccccc6)nc(-c6cccc7ccccc67)n5)ccc34)n2)cc1 ; c1ccc(-c2nc(-c3ccc(-c4ccc(-c5nc(-c6ccccc6)nc(-c6ccccc6)n5)cc4)cc3)cc(-c3ccc(C4CC5CCC4C5)cc3)n2)cc1 ; c1ccc(-c2nc(-c3ccc(-c4ccc(-c5nc(-c6ccccc6)nc(-c6ccccc6)n5)cc4)cc3)cc(-c3cccc(C4CC5CCC4C5)c3)n2)cc1]

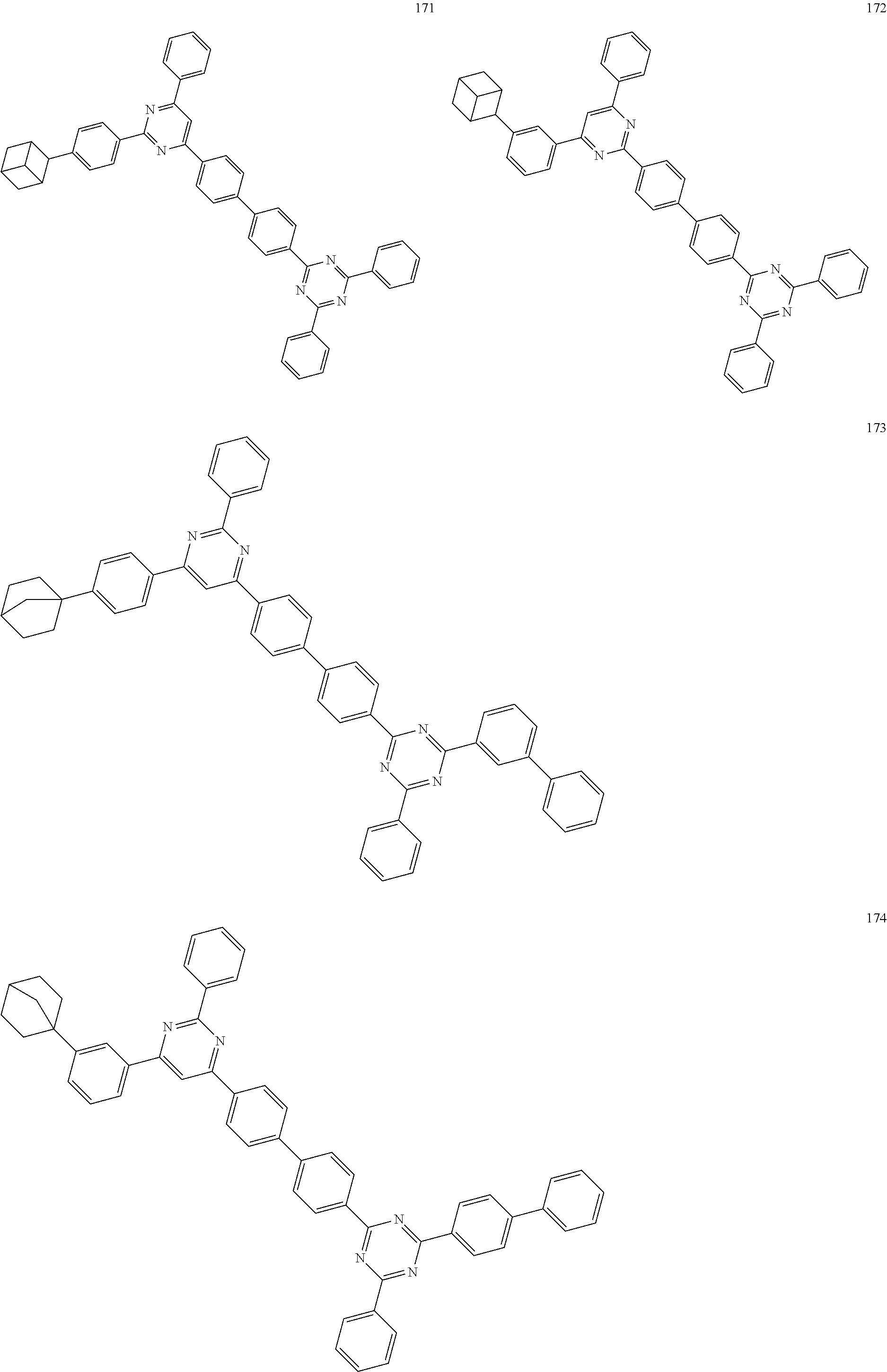 [SMILES: c1ccc(-c2cc(-c3ccc(-c4ccc(-c5nc(-c6ccccc6)nc(-c6ccccc6)n5)cc4)cc3)nc(-c3ccc(C4C5CC6CC4C65)cc3)n2)cc1 ; c1ccc(-c2cc(-c3cccc(C4C5CC6CC4C65)c3)nc(-c3ccc(-c4ccc(-c5nc(-c6ccccc6)nc(-c6ccccc6)n5)cc4)cc3)n2)cc1 ; c1ccc(-c2cccc(-c3nc(-c4ccccc4)nc(-c4ccc(-c5ccc(-c6cc(-c7ccc(C89CCC(CC8)C9)cc7)nc(-c7ccccc7)n6)cc5)cc4)n3)c2)cc1 ; c1ccc(-c2ccc(-c3nc(-c4ccccc4)nc(-c4ccc(-c5ccc(-c6cc(-c7cccc(C89CCC(CC8)C9)c7)nc(-c7ccccc7)n6)cc5)cc4)n3)cc2)cc1]

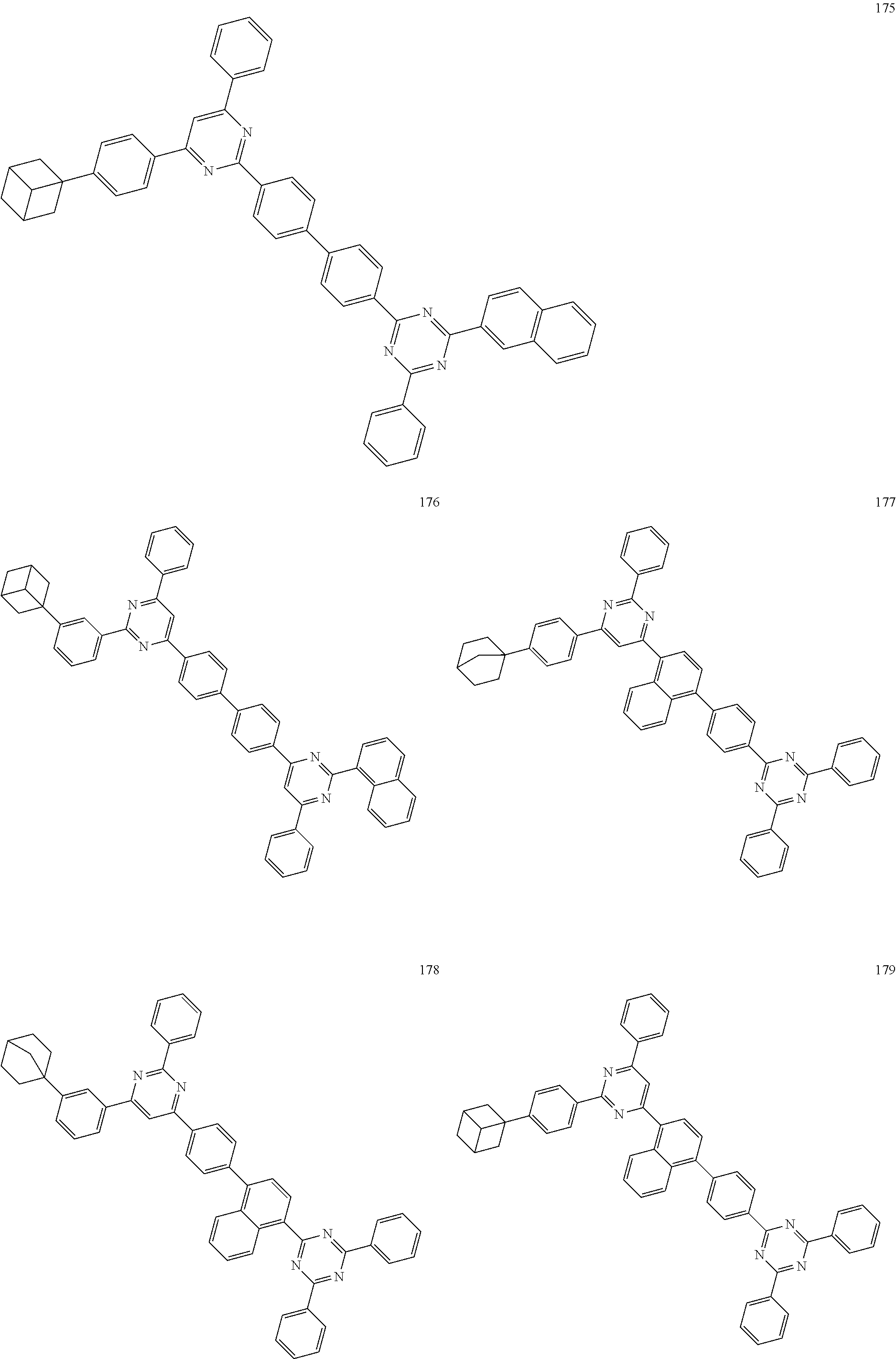

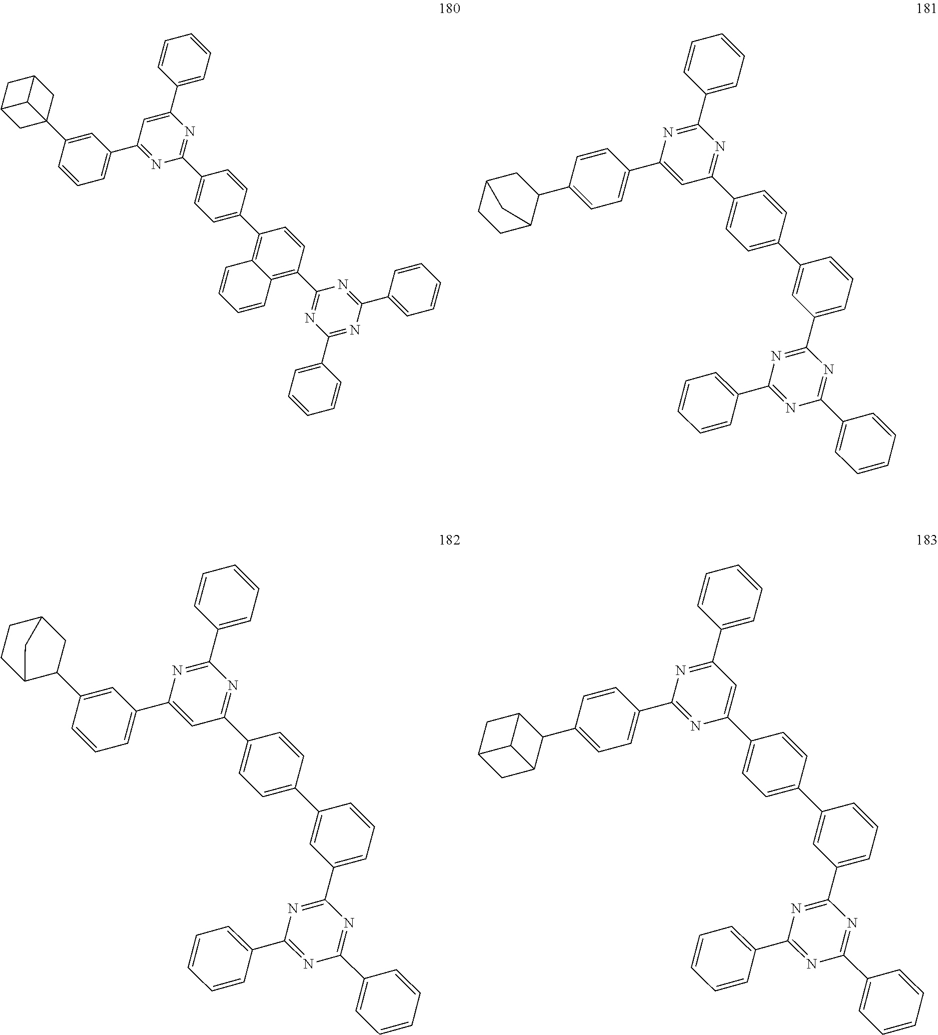 [SMILES: c1ccc(-c2cc(-c3cccc(C45CC6CC(C4)C65)c3)nc(-c3ccc(-c4ccc(-c5nc(-c6ccccc6)nc(-c6ccccc6)n5)c5ccccc45)cc3)n2)cc1 ; c1ccc(-c2nc(-c3ccc(-c4cccc(-c5nc(-c6ccccc6)nc(-c6ccccc6)n5)c4)cc3)cc(-c3ccc(C4CC5CCC4C5)cc3)n2)cc1 ; c1ccc(-c2nc(-c3ccc(-c4cccc(-c5nc(-c6ccccc6)nc(-c6ccccc6)n5)c4)cc3)cc(-c3cccc(C4CC5CCC4C5)c3)n2)cc1 ; c1ccc(-c2cc(-c3ccc(-c4cccc(-c5nc(-c6ccccc6)nc(-c6ccccc6)n5)c4)cc3)nc(-c3ccc(C4C5CC6CC4C65)cc3)n2)cc1]

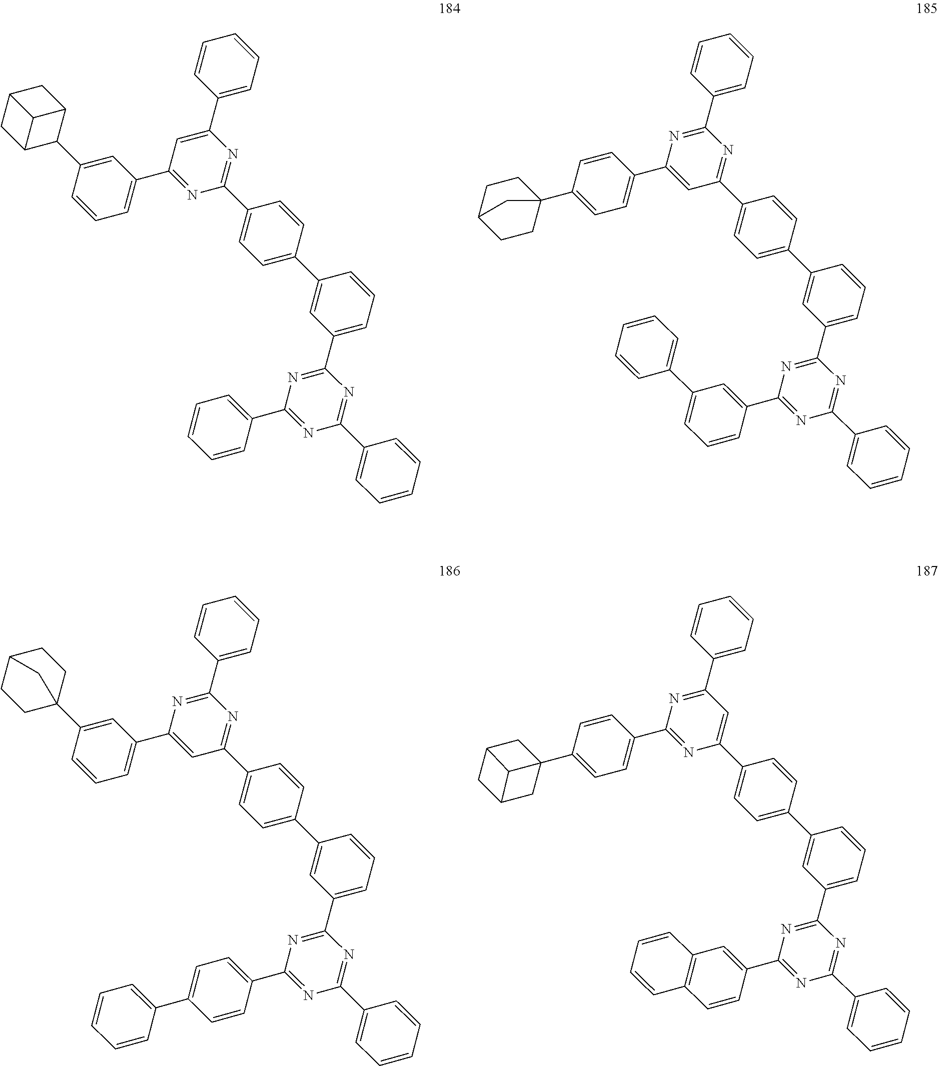 [SMILES: c1ccc(-c2cc(-c3cccc(C4C5CC6CC4C65)c3)nc(-c3ccc(-c4cccc(-c5nc(-c6ccccc6)nc(-c6ccccc6)n5)c4)cc3)n2)cc1 ; c1ccc(-c2cccc(-c3nc(-c4ccccc4)nc(-c4cccc(-c5ccc(-c6cc(-c7ccc(C89CCC(CC8)C9)cc7)nc(-c7ccccc7)n6)cc5)c4)n3)c2)cc1 ; c1ccc(-c2ccc(-c3nc(-c4ccccc4)nc(-c4cccc(-c5ccc(-c6cc(-c7cccc(C89CCC(CC8)C9)c7)nc(-c7ccccc7)n6)cc5)c4)n3)cc2)cc1 ; c1ccc(-c2cc(-c3ccc(-c4cccc(-c5nc(-c6ccccc6)nc(-c6ccc7ccccc7c6)n5)c4)cc3)nc(-c3ccc(C45CC6CC(C4)C65)cc3)n2)cc1]

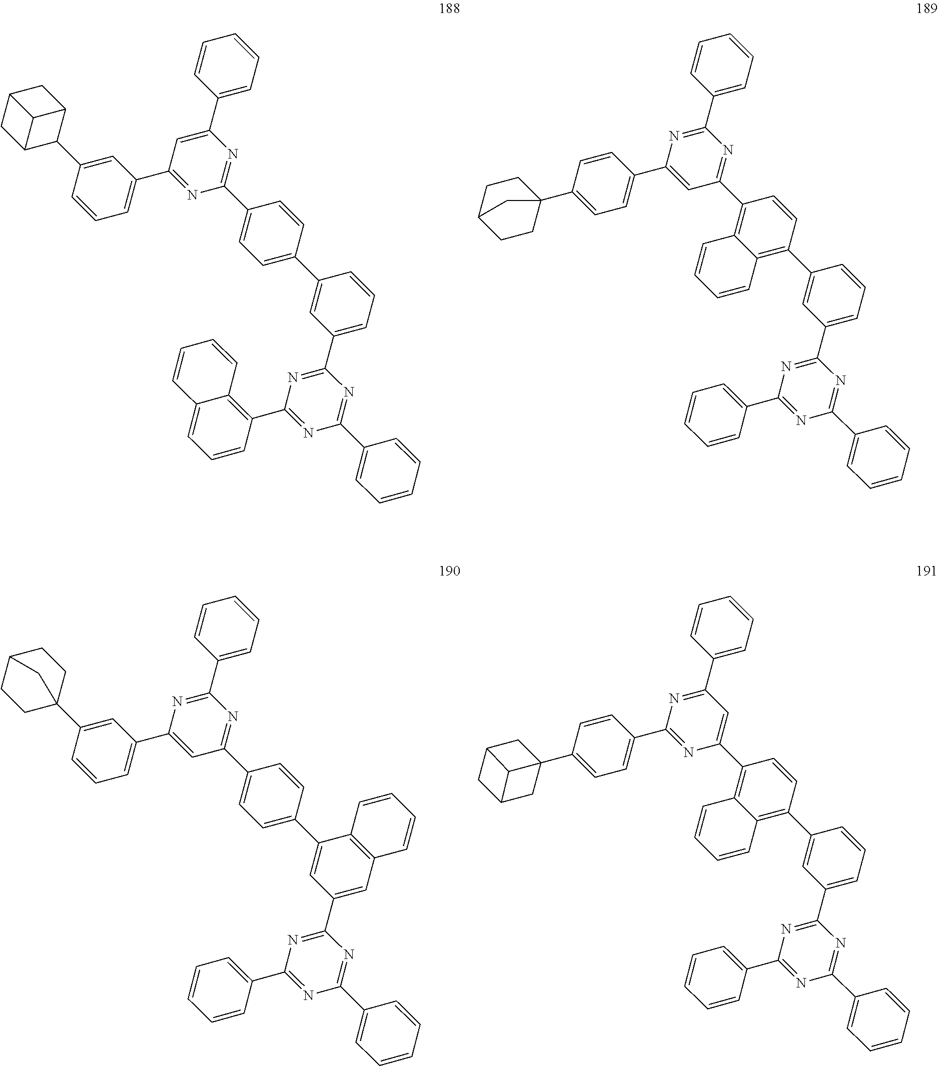 [SMILES: c1ccc(-c2cc(-c3cccc(C4C5CC6CC4C65)c3)nc(-c3ccc(-c4cccc(-c5nc(-c6ccccc6)nc(-c6cccc7ccccc67)n5)c4)cc3)n2)cc1 ; c1ccc(-c2nc(-c3ccc(C45CCC(CC4)C5)cc3)cc(-c3ccc(-c4cccc(-c5nc(-c6ccccc6)nc(-c6ccccc6)n5)c4)c4ccccc34)n2)cc1 ; c1ccc(-c2nc(-c3ccc(-c4cc(-c5nc(-c6ccccc6)nc(-c6ccccc6)n5)cc5ccccc45)cc3)cc(-c3cccc(C45CCC(CC4)C5)c3)n2)cc1 ; c1ccc(-c2cc(-c3ccc(-c4cccc(-c5nc(-c6ccccc6)nc(-c6ccccc6)n5)c4)c4ccccc34)nc(-c3ccc(C45CC6CC(C4)C65)cc3)n2)cc1]

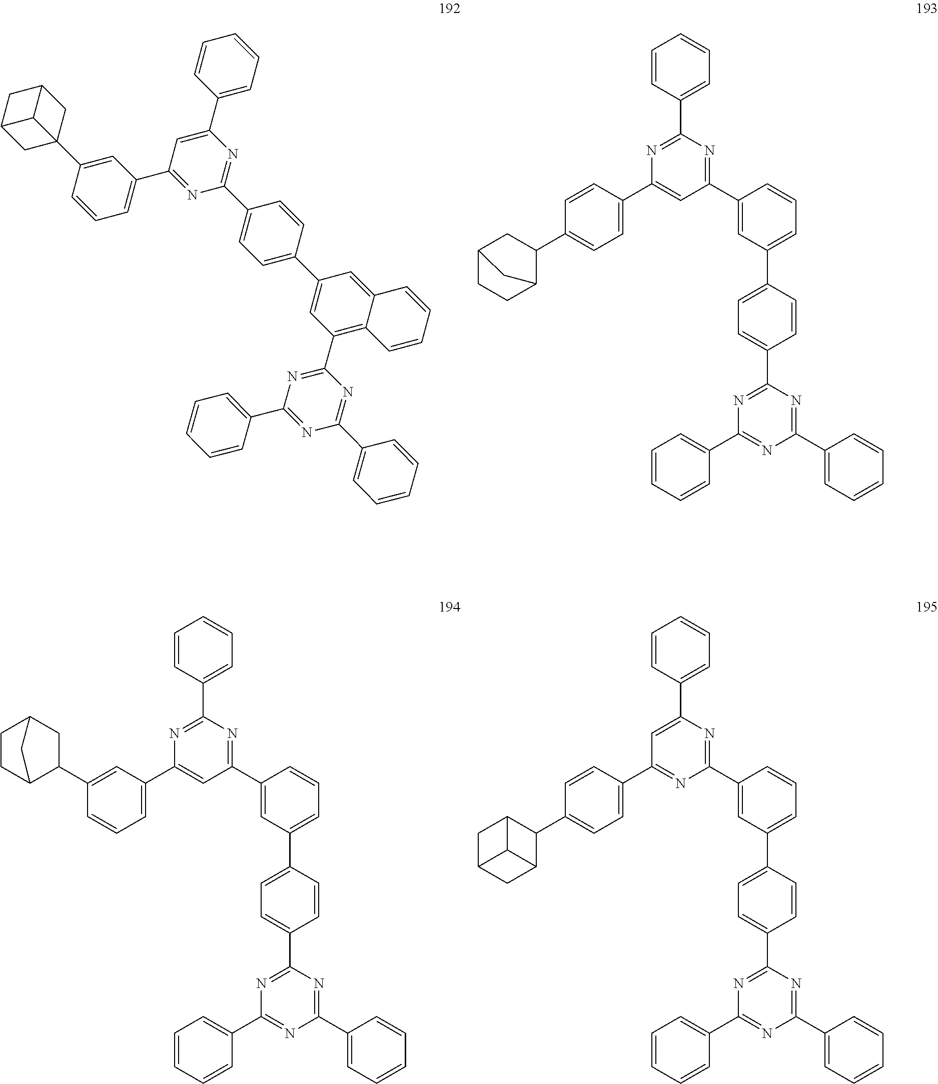 [SMILES: c1ccc(-c2cc(-c3cccc(C45CC6CC(C4)C65)c3)nc(-c3ccc(-c4cc(-c5nc(-c6ccccc6)nc(-c6ccccc6)n5)c5ccccc5c4)cc3)n2)cc1 ; c1ccc(-c2nc(-c3ccc(C4CC5CCC4C5)cc3)cc(-c3cccc(-c4ccc(-c5nc(-c6ccccc6)nc(-c6ccccc6)n5)cc4)c3)n2)cc1 ; c1ccc(-c2nc(-c3cccc(-c4ccc(-c5nc(-c6ccccc6)nc(-c6ccccc6)n5)cc4)c3)cc(-c3cccc(C4CC5CCC4C5)c3)n2)cc1 ; c1ccc(-c2cc(-c3ccc(C4C5CC6CC4C65)cc3)nc(-c3cccc(-c4ccc(-c5nc(-c6ccccc6)nc(-c6ccccc6)n5)cc4)c3)n2)cc1]

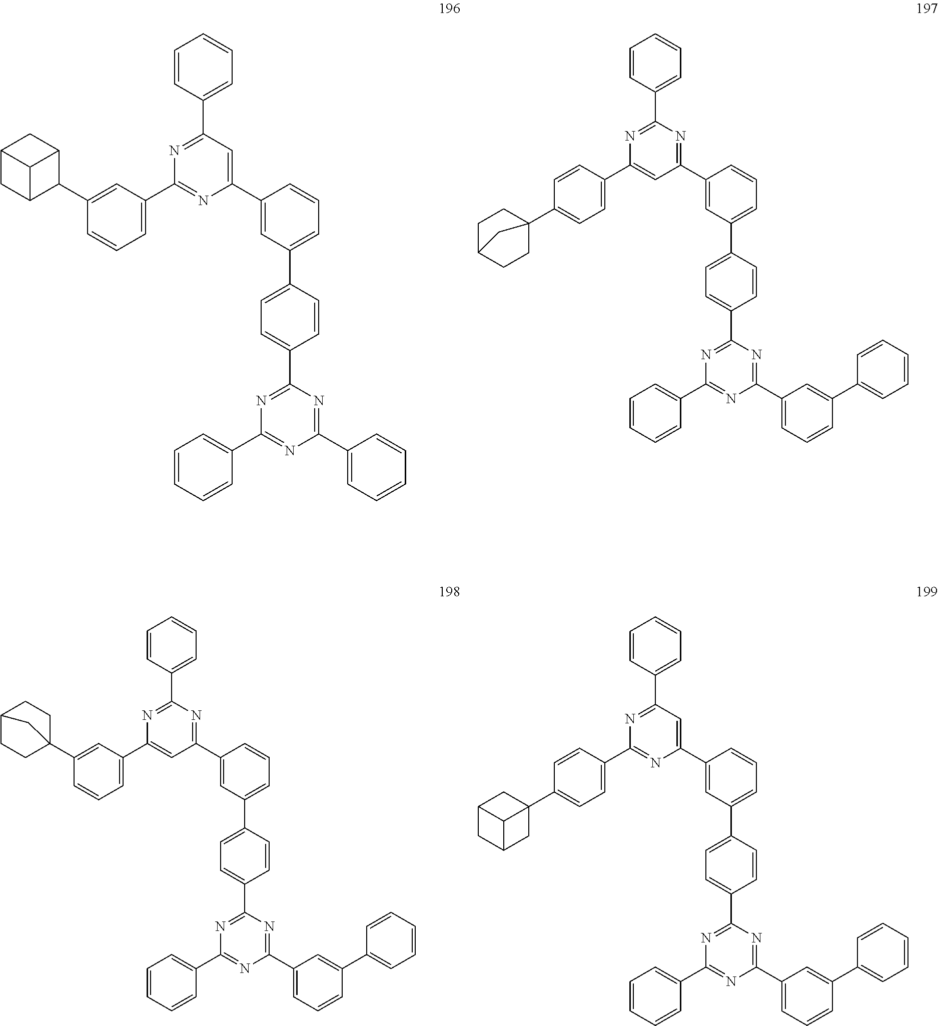 [SMILES: c1ccc(-c2cc(-c3cccc(-c4ccc(-c5nc(-c6ccccc6)nc(-c6ccccc6)n5)cc4)c3)nc(-c3cccc(C4C5CC6CC4C65)c3)n2)cc1 ; c1ccc(-c2cccc(-c3nc(-c4ccccc4)nc(-c4ccc(-c5cccc(-c6cc(-c7ccc(C89CCC(CC8)C9)cc7)nc(-c7ccccc7)n6)c5)cc4)n3)c2)cc1 ; c1ccc(-c2cccc(-c3nc(-c4ccccc4)nc(-c4ccc(-c5cccc(-c6cc(-c7cccc(C89CCC(CC8)C9)c7)nc(-c7ccccc7)n6)c5)cc4)n3)c2)cc1 ; c1ccc(-c2cccc(-c3nc(-c4ccccc4)nc(-c4ccc(-c5cccc(-c6cc(-c7ccccc7)nc(-c7ccc(C89CC%10CC(C8)C%109)cc7)n6)c5)cc4)n3)c2)cc1]

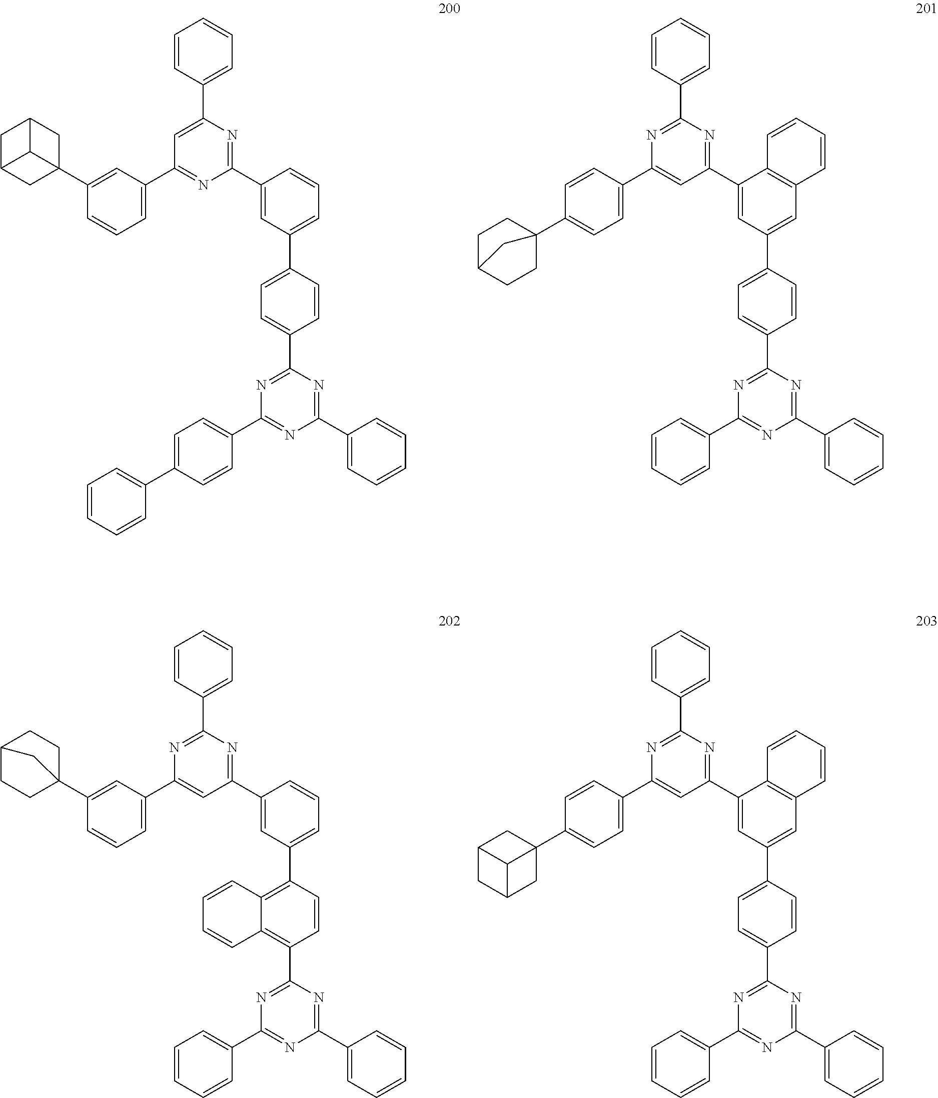 [SMILES: c1ccc(-c2ccc(-c3nc(-c4ccccc4)nc(-c4ccc(-c5cccc(-c6nc(-c7ccccc7)cc(-c7cccc(C89CC%10CC(C8)C%109)c7)n6)c5)cc4)n3)cc2)cc1 ; c1ccc(-c2nc(-c3ccc(C45CCC(CC4)C5)cc3)cc(-c3cc(-c4ccc(-c5nc(-c6ccccc6)nc(-c6ccccc6)n5)cc4)cc4ccccc34)n2)cc1 ; c1ccc(-c2nc(-c3cccc(-c4ccc(-c5nc(-c6ccccc6)nc(-c6ccccc6)n5)c5ccccc45)c3)cc(-c3cccc(C45CCC(CC4)C5)c3)n2)cc1 ; c1ccc(-c2nc(-c3ccc(C45CC6CC(C4)C65)cc3)cc(-c3cc(-c4ccc(-c5nc(-c6ccccc6)nc(-c6ccccc6)n5)cc4)cc4ccccc34)n2)cc1]

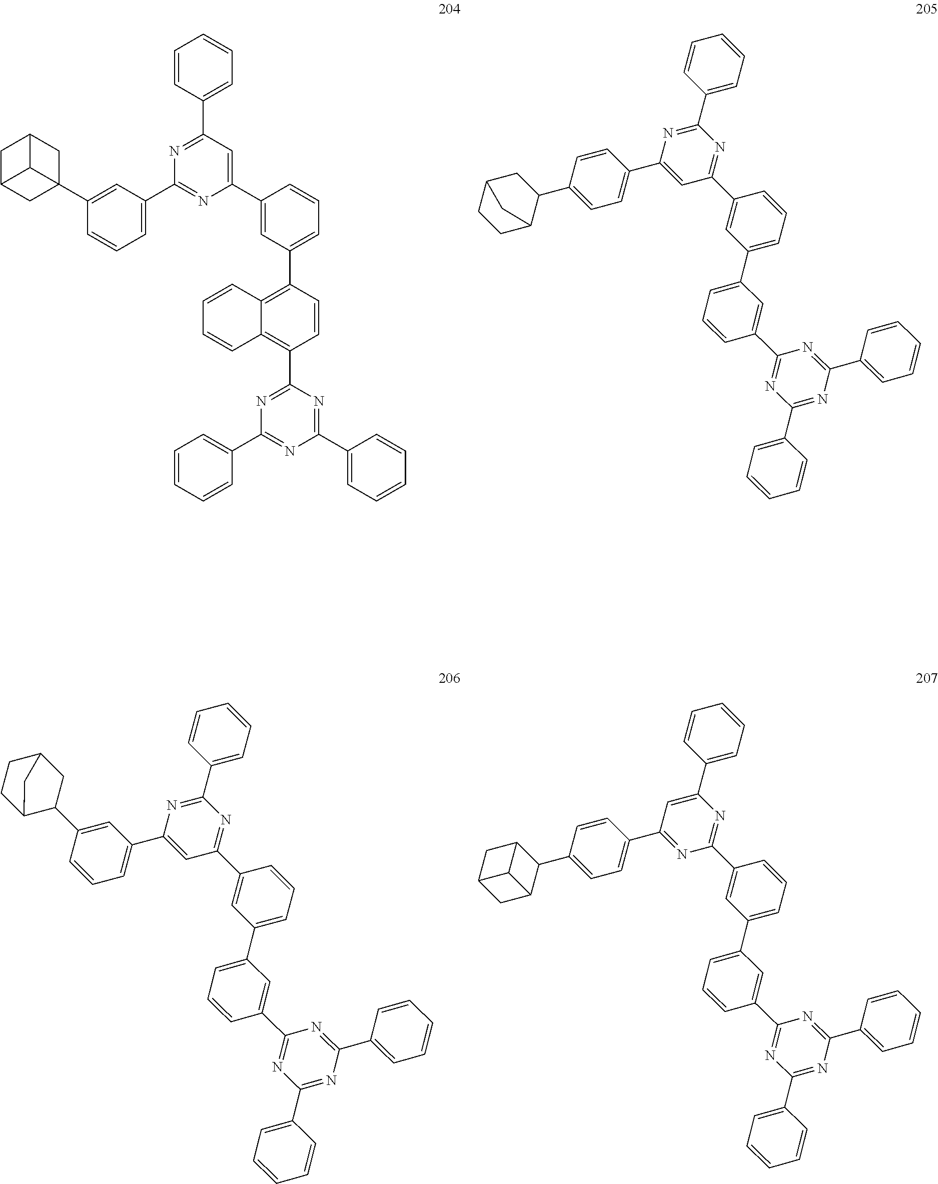 [SMILES: c1ccc(-c2cc(-c3cccc(-c4ccc(-c5nc(-c6ccccc6)nc(-c6ccccc6)n5)c5ccccc45)c3)nc(-c3cccc(C45CC6CC(C4)C65)c3)n2)cc1 ; c1ccc(-c2nc(-c3ccc(C4CC5CCC4C5)cc3)cc(-c3cccc(-c4cccc(-c5nc(-c6ccccc6)nc(-c6ccccc6)n5)c4)c3)n2)cc1 ; c1ccc(-c2nc(-c3cccc(-c4cccc(-c5nc(-c6ccccc6)nc(-c6ccccc6)n5)c4)c3)cc(-c3cccc(C4CC5CCC4C5)c3)n2)cc1 ; c1ccc(-c2cc(-c3ccc(C4C5CC6CC4C65)cc3)nc(-c3cccc(-c4cccc(-c5nc(-c6ccccc6)nc(-c6ccccc6)n5)c4)c3)n2)cc1]

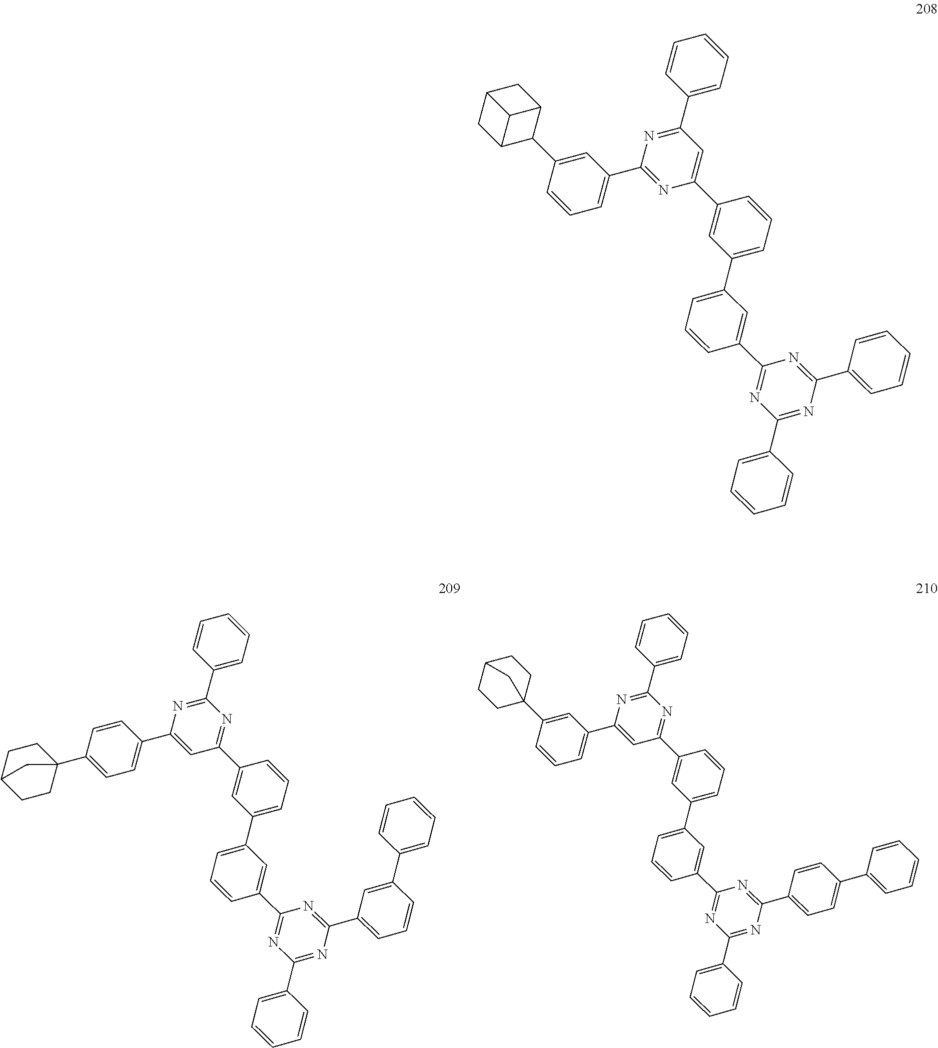 [SMILES: c1ccc(-c2cc(-c3cccc(-c4cccc(-c5nc(-c6ccccc6)nc(-c6ccccc6)n5)c4)c3)nc(-c3cccc(C4C5CC6CC4C65)c3)n2)cc1 ; c1ccc(-c2cccc(-c3nc(-c4ccccc4)nc(-c4cccc(-c5cccc(-c6cc(-c7ccc(C89CCC(CC8)C9)cc7)nc(-c7ccccc7)n6)c5)c4)n3)c2)cc1 ; c1ccc(-c2ccc(-c3nc(-c4ccccc4)nc(-c4cccc(-c5cccc(-c6cc(-c7cccc(C89CCC(CC8)C9)c7)nc(-c7ccccc7)n6)c5)c4)n3)cc2)cc1]

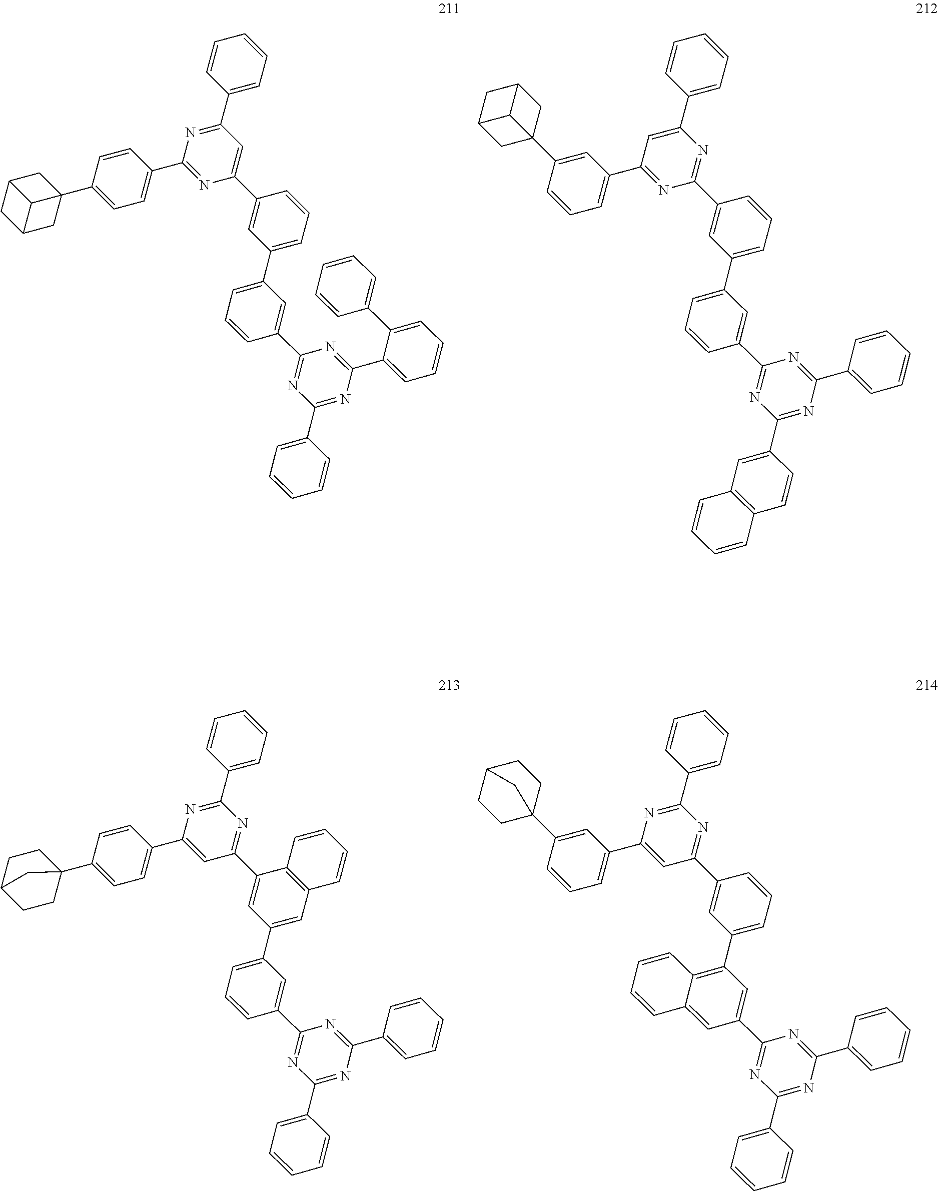 [SMILES: c1ccc(-c2cc(-c3cccc(-c4cccc(-c5nc(-c6ccccc6)nc(-c6ccccc6-c6ccccc6)n5)c4)c3)nc(-c3ccc(C45CC6CC(C4)C65)cc3)n2)cc1 ; c1ccc(-c2cc(-c3cccc(C45CC6CC(C4)C65)c3)nc(-c3cccc(-c4cccc(-c5nc(-c6ccccc6)nc(-c6ccc7ccccc7c6)n5)c4)c3)n2)cc1 ; c1ccc(-c2nc(-c3ccc(C45CCC(CC4)C5)cc3)cc(-c3cc(-c4cccc(-c5nc(-c6ccccc6)nc(-c6ccccc6)n5)c4)cc4ccccc34)n2)cc1 ; c1ccc(-c2nc(-c3cccc(-c4cc(-c5nc(-c6ccccc6)nc(-c6ccccc6)n5)cc5ccccc45)c3)cc(-c3cccc(C45CCC(CC4)C5)c3)n2)cc1]

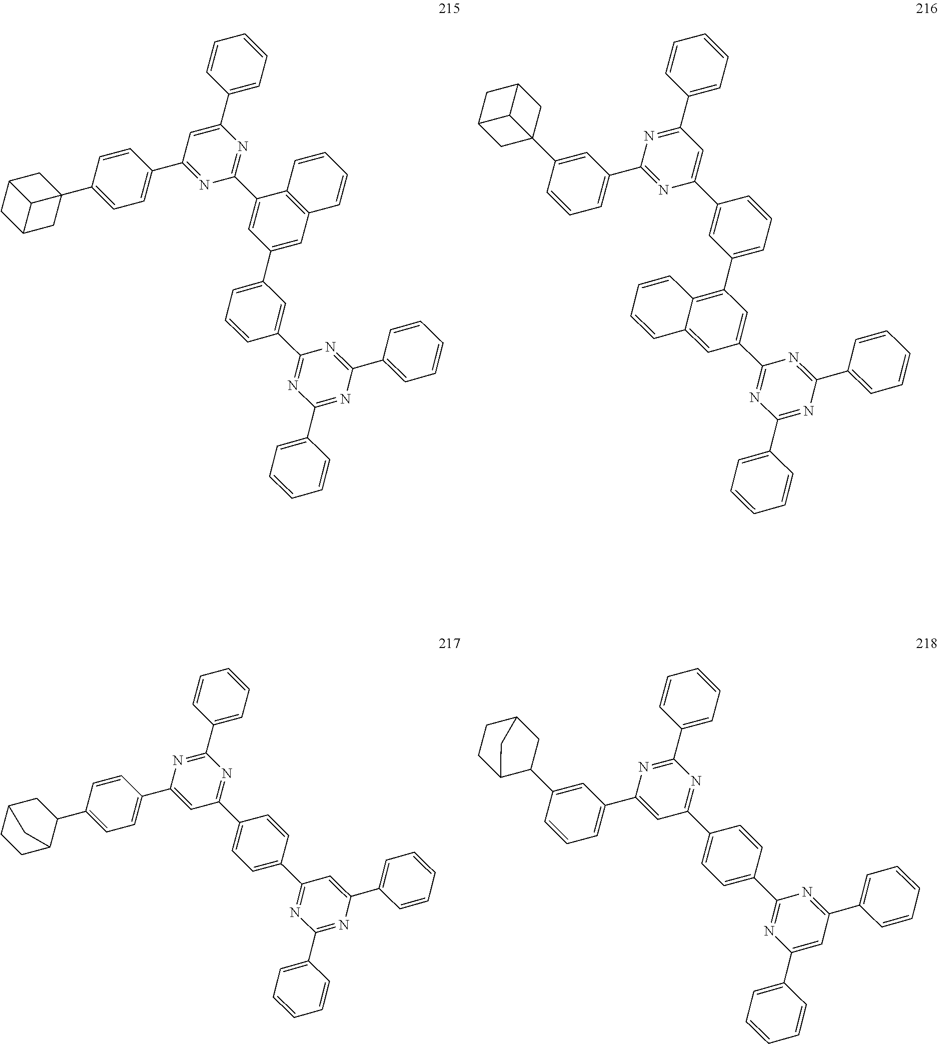 [SMILES: c1ccc(-c2cc(-c3ccc(C45CC6CC(C4)C65)cc3)nc(-c3cc(-c4cccc(-c5nc(-c6ccccc6)nc(-c6ccccc6)n5)c4)cc4ccccc34)n2)cc1 ; c1ccc(-c2cc(-c3cccc(-c4cc(-c5nc(-c6ccccc6)nc(-c6ccccc6)n5)cc5ccccc45)c3)nc(-c3cccc(C45CC6CC(C4)C65)c3)n2)cc1 ; c1ccc(-c2cc(-c3ccc(-c4cc(-c5ccc(C6CC7CCC6C7)cc5)nc(-c5ccccc5)n4)cc3)nc(-c3ccccc3)n2)cc1 ; c1ccc(-c2cc(-c3ccccc3)nc(-c3ccc(-c4cc(-c5cccc(C6CC7CCC6C7)c5)nc(-c5ccccc5)n4)cc3)n2)cc1]

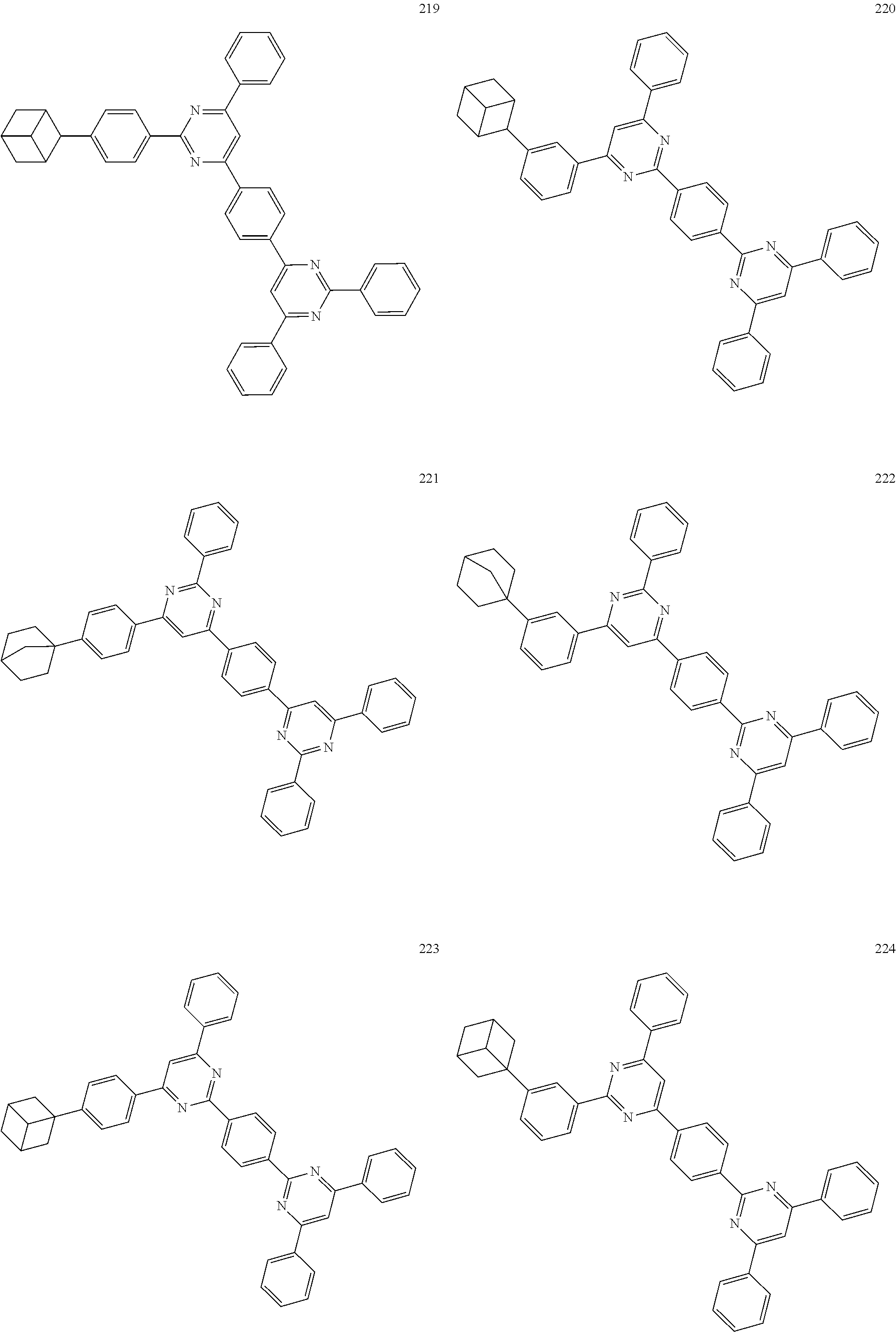

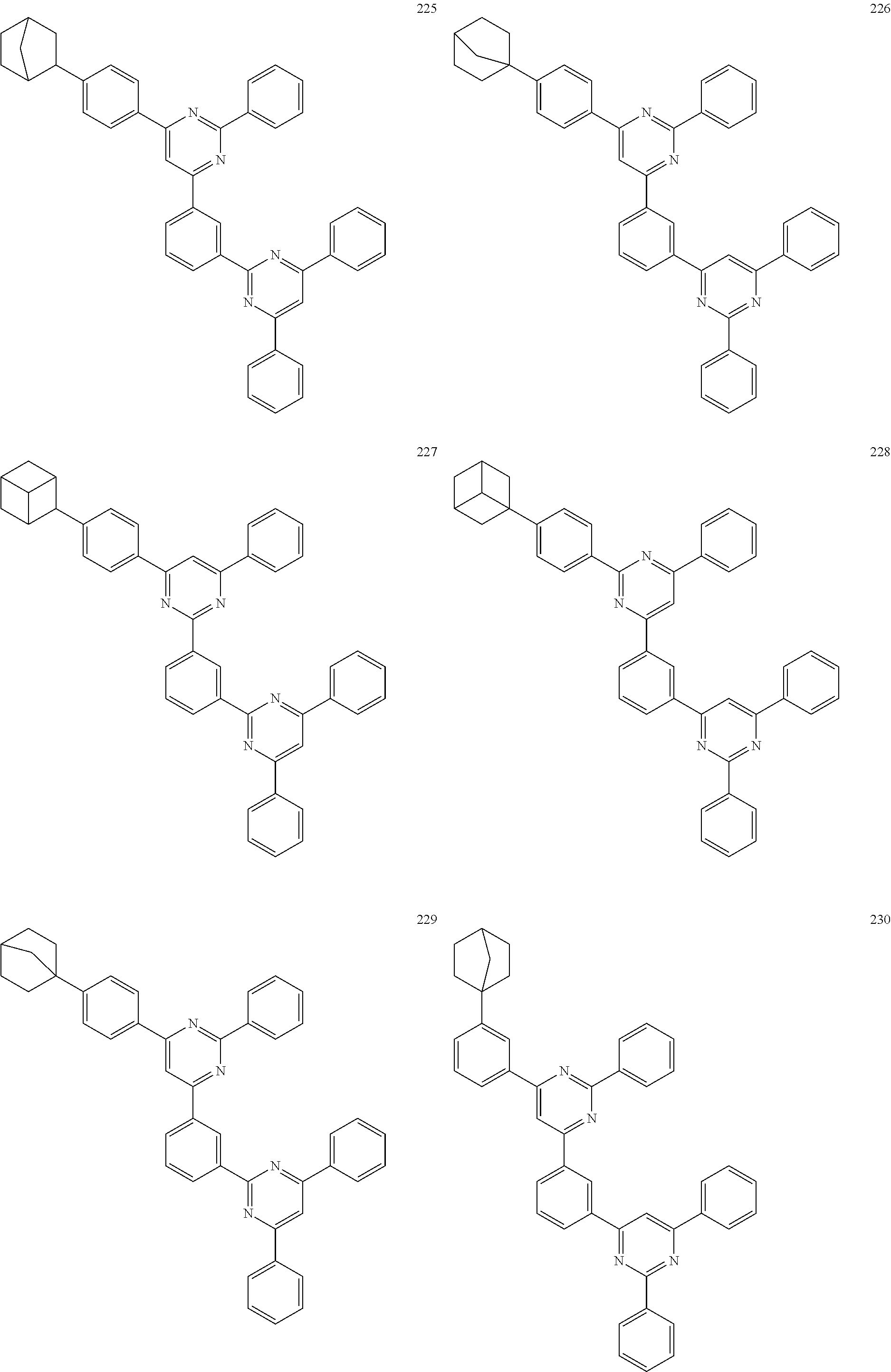

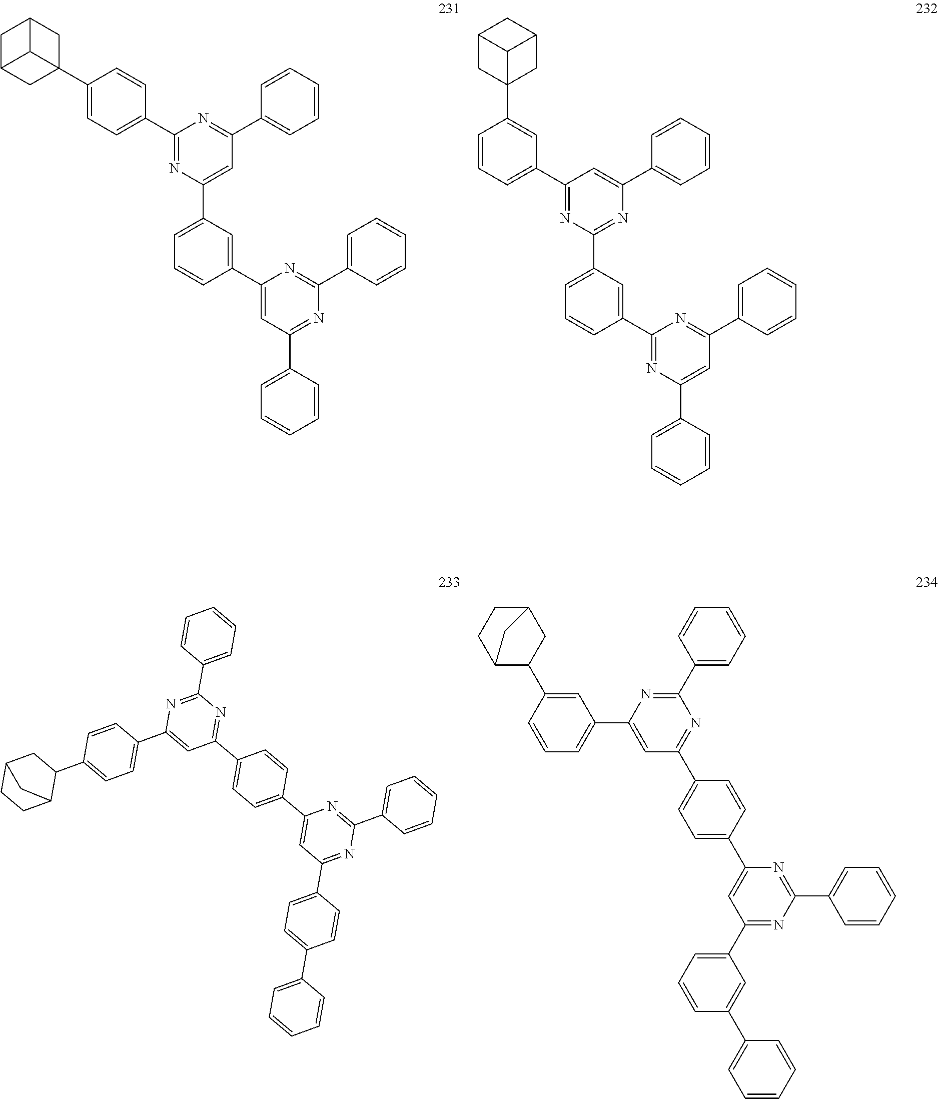 [SMILES: c1ccc(-c2cc(-c3cccc(-c4cc(-c5ccccc5)nc(-c5ccc(C67CC8CC(C6)C87)cc5)n4)c3)nc(-c3ccccc3)n2)cc1 ; c1ccc(-c2cc(-c3ccccc3)nc(-c3cccc(-c4nc(-c5ccccc5)cc(-c5cccc(C67CC8CC(C6)C87)c5)n4)c3)n2)cc1 ; c1ccc(-c2ccc(-c3cc(-c4ccc(-c5cc(-c6ccc(C7CC8CCC7C8)cc6)nc(-c6ccccc6)n5)cc4)nc(-c4ccccc4)n3)cc2)cc1 ; c1ccc(-c2cccc(-c3cc(-c4ccc(-c5cc(-c6cccc(C7CC8CCC7C8)c6)nc(-c6ccccc6)n5)cc4)nc(-c4ccccc4)n3)c2)cc1]

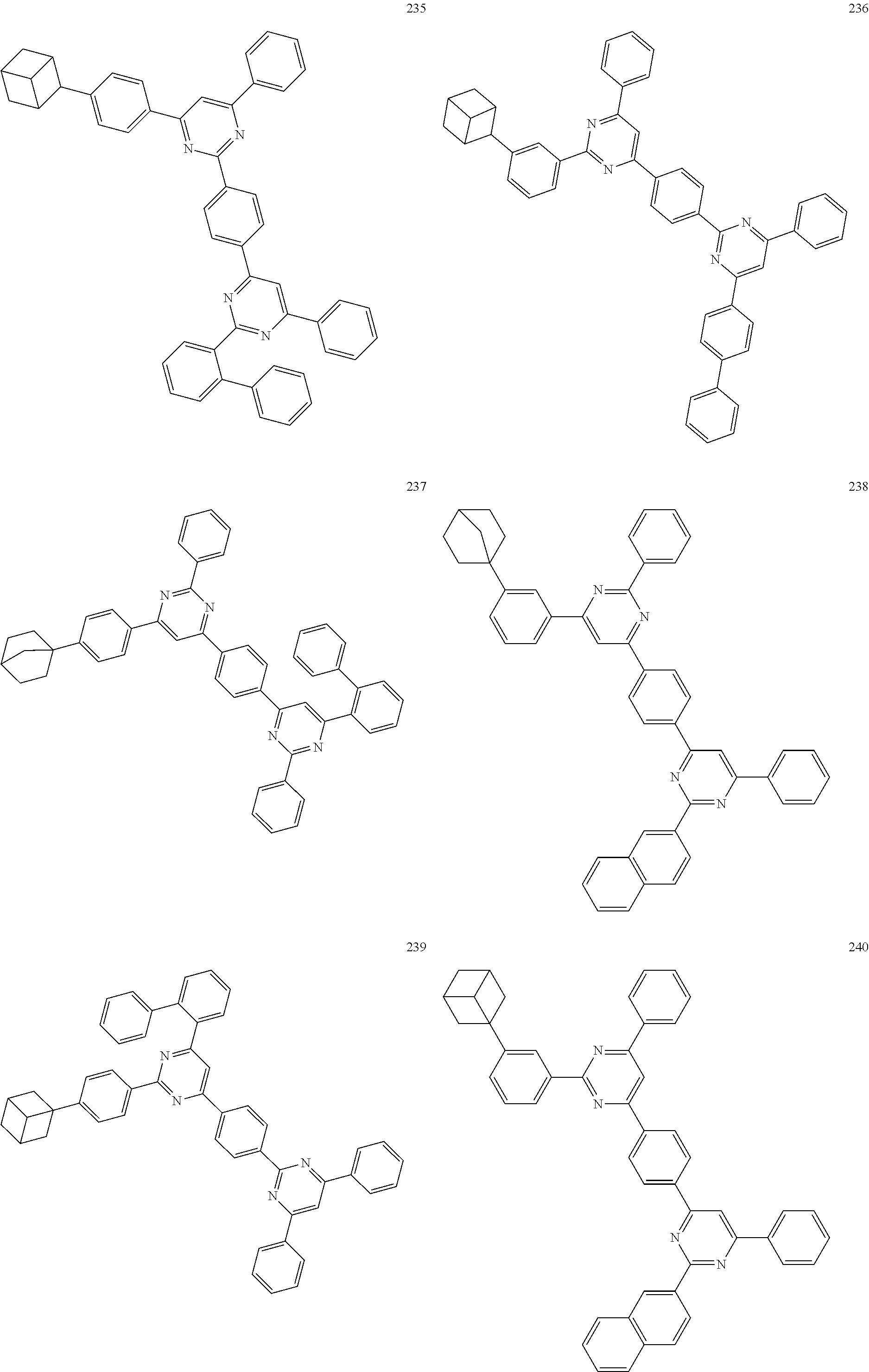

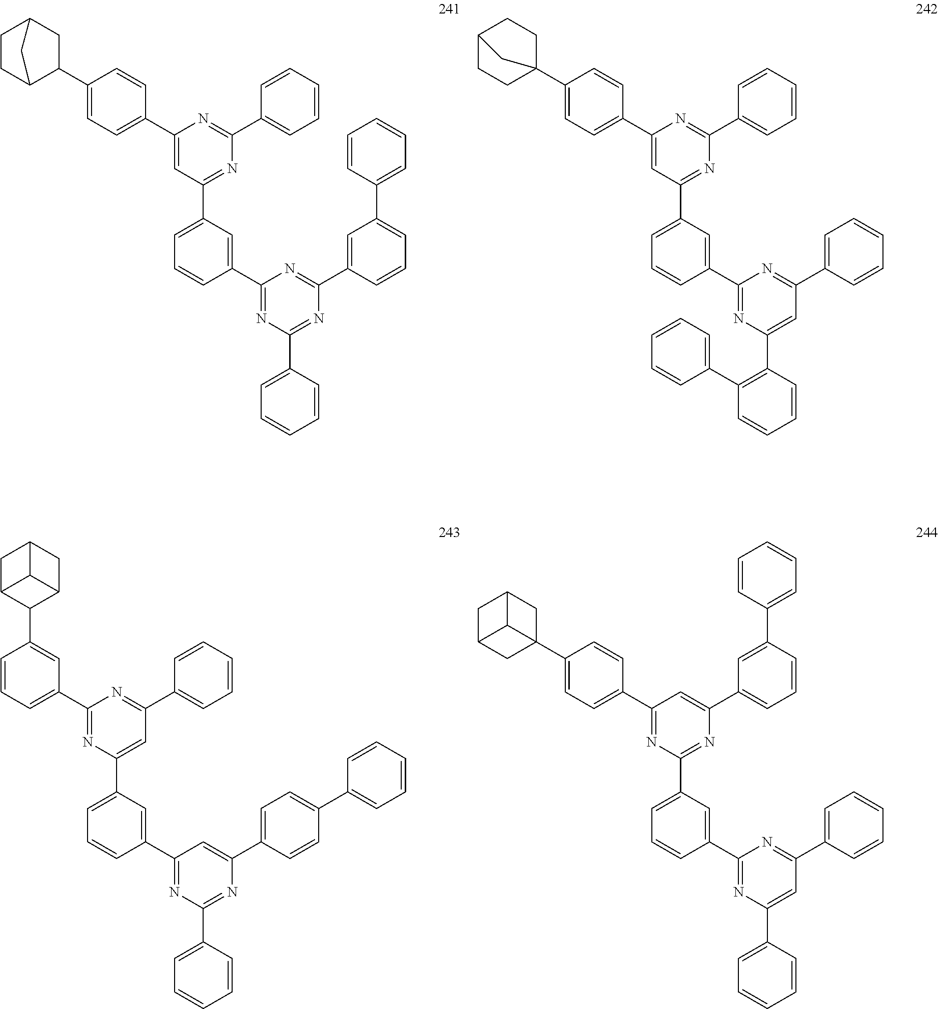 [SMILES: c1ccc(-c2cccc(-c3nc(-c4ccccc4)nc(-c4cccc(-c5cc(-c6ccc(C7CC8CCC7C8)cc6)nc(-c6ccccc6)n5)c4)n3)c2)cc1 ; c1ccc(-c2cc(-c3ccccc3-c3ccccc3)nc(-c3cccc(-c4cc(-c5ccc(C67CCC(CC6)C7)cc5)nc(-c5ccccc5)n4)c3)n2)cc1 ; c1ccc(-c2ccc(-c3cc(-c4cccc(-c5cc(-c6ccccc6)nc(-c6cccc(C7C8CC9CC7C98)c6)n5)c4)nc(-c4ccccc4)n3)cc2)cc1 ; c1ccc(-c2cccc(-c3cc(-c4ccc(C56CC7CC(C5)C76)cc4)nc(-c4cccc(-c5nc(-c6ccccc6)cc(-c6ccccc6)n5)c4)n3)c2)cc1]

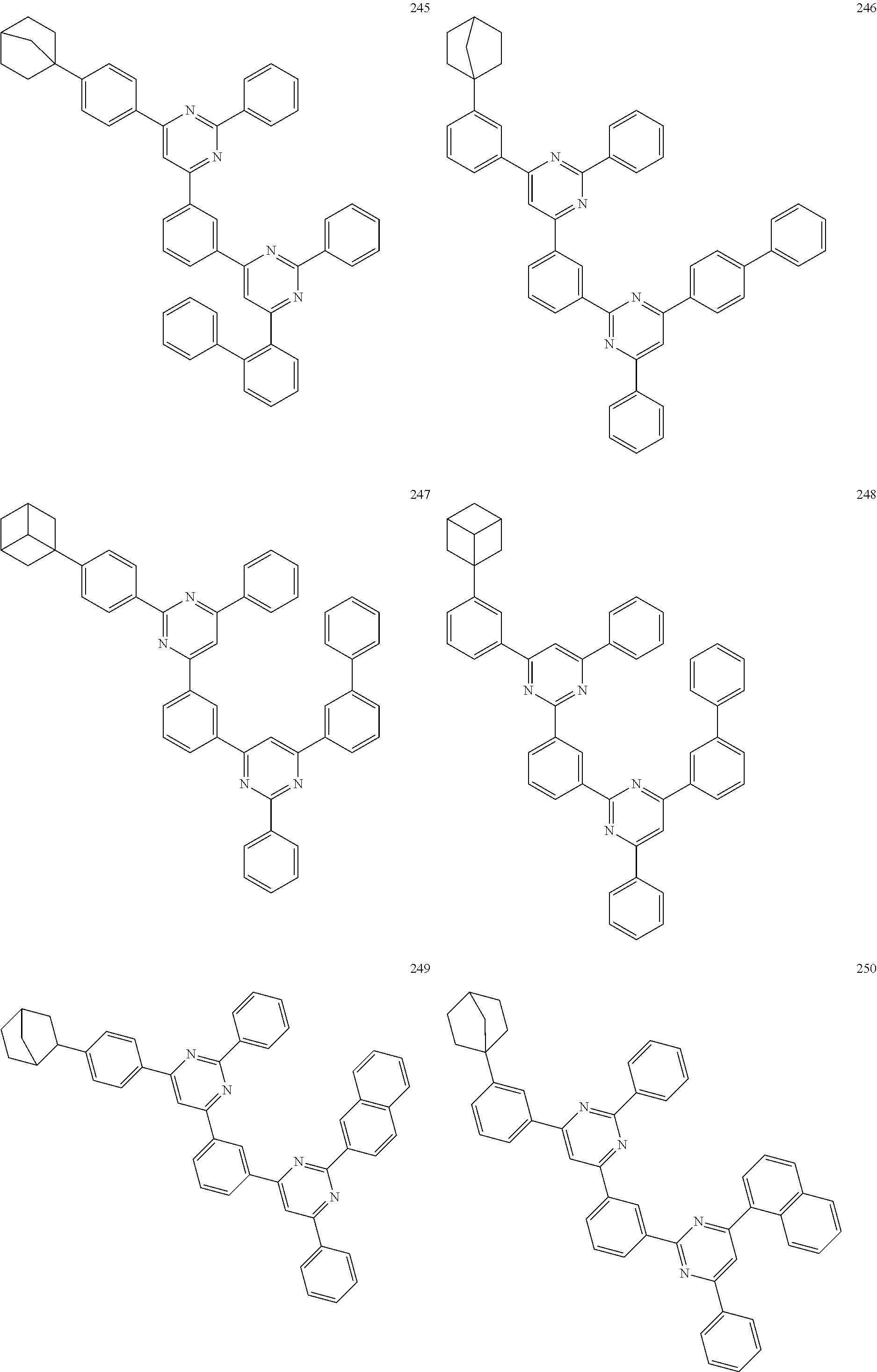

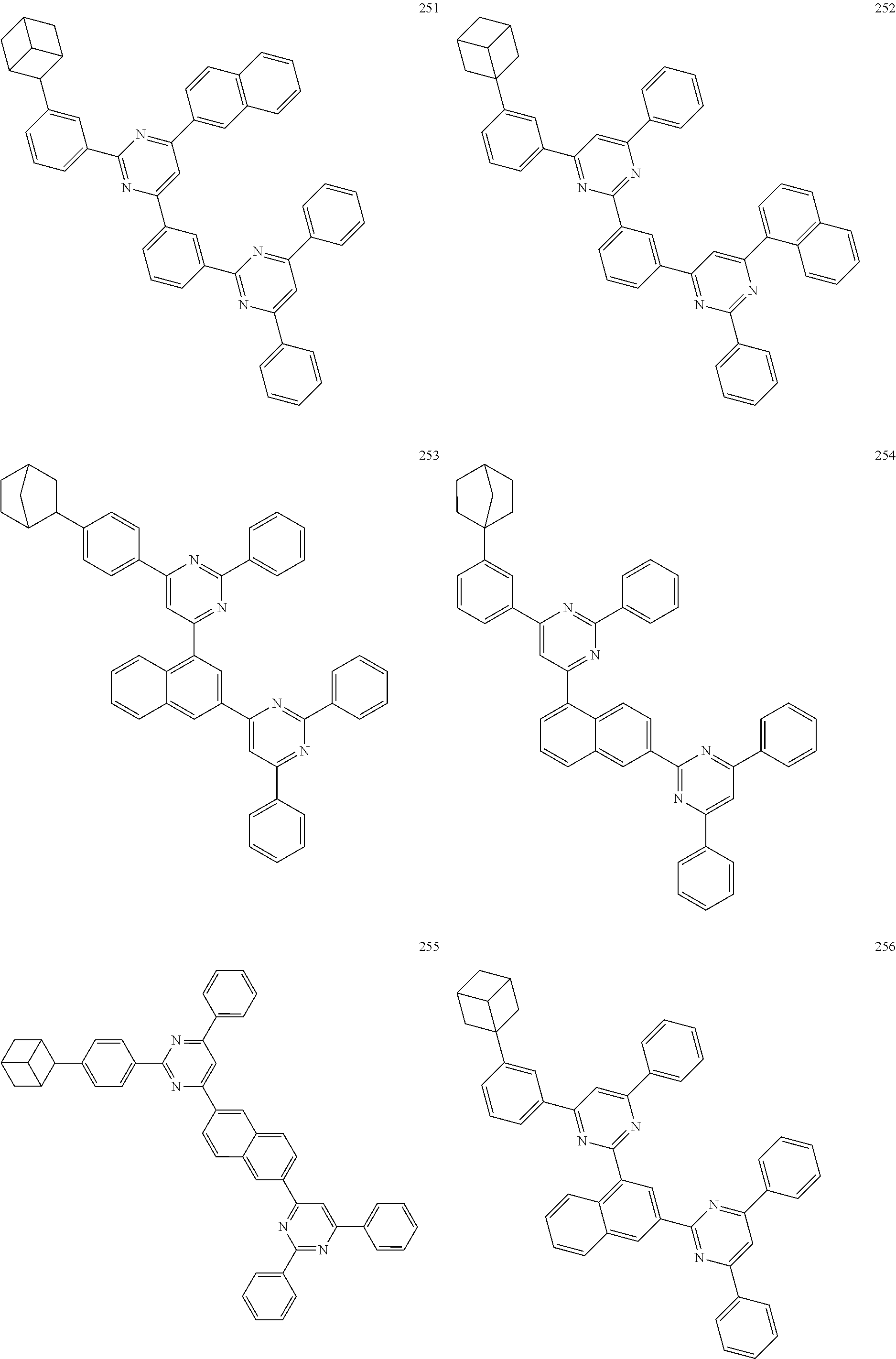

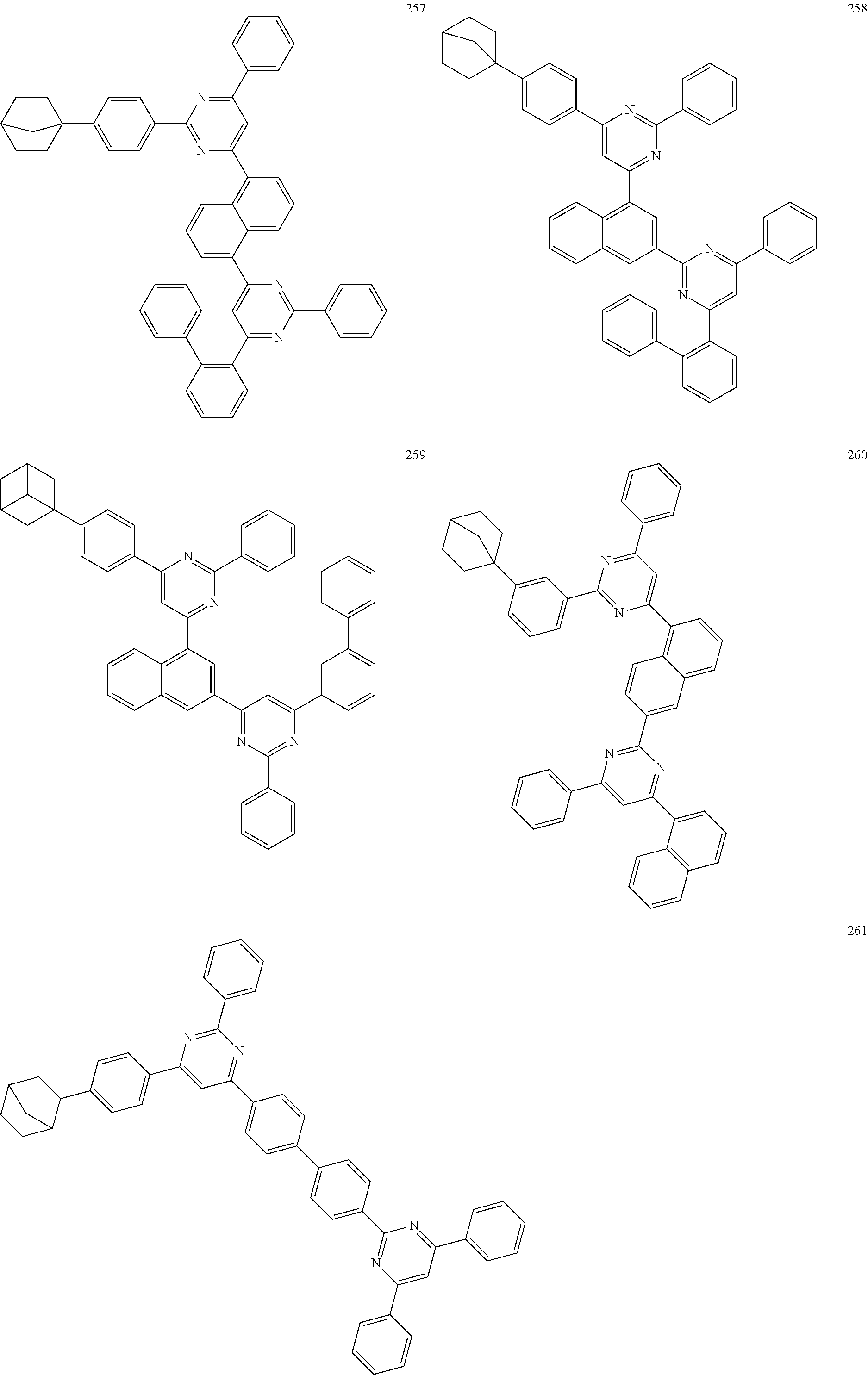

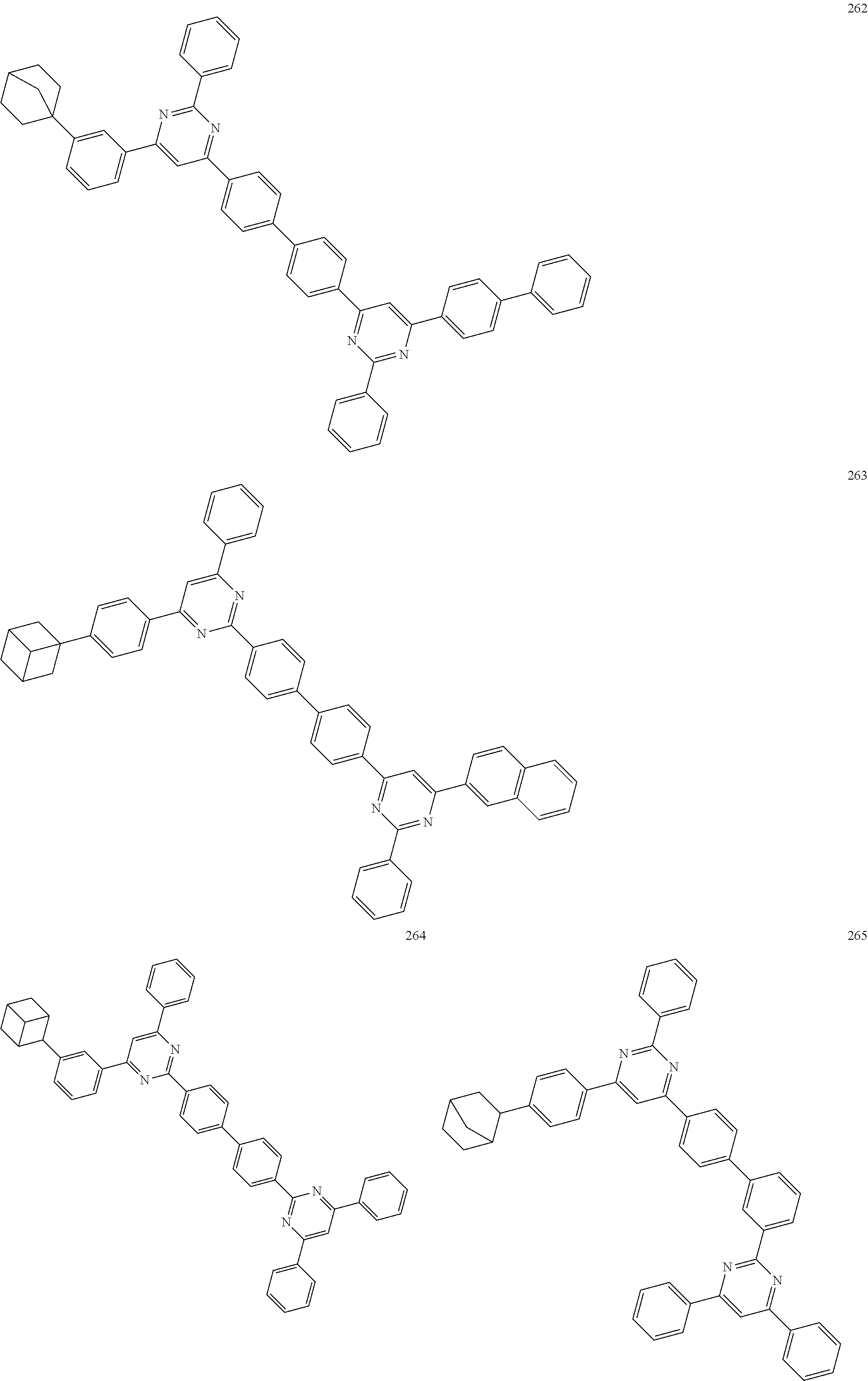 [SMILES: c1ccc(-c2ccc(-c3cc(-c4ccc(-c5ccc(-c6cc(-c7cccc(C89CCC(CC8)C9)c7)nc(-c7ccccc7)n6)cc5)cc4)nc(-c4ccccc4)n3)cc2)cc1 ; c1ccc(-c2cc(-c3ccc(C45CC6CC(C4)C65)cc3)nc(-c3ccc(-c4ccc(-c5cc(-c6ccc7ccccc7c6)nc(-c6ccccc6)n5)cc4)cc3)n2)cc1 ; c1ccc(-c2cc(-c3ccccc3)nc(-c3ccc(-c4ccc(-c5nc(-c6ccccc6)cc(-c6cccc(C7C8CC9CC7C98)c6)n5)cc4)cc3)n2)cc1 ; c1ccc(-c2cc(-c3ccccc3)nc(-c3cccc(-c4ccc(-c5cc(-c6ccc(C7CC8CCC7C8)cc6)nc(-c6ccccc6)n5)cc4)c3)n2)cc1]

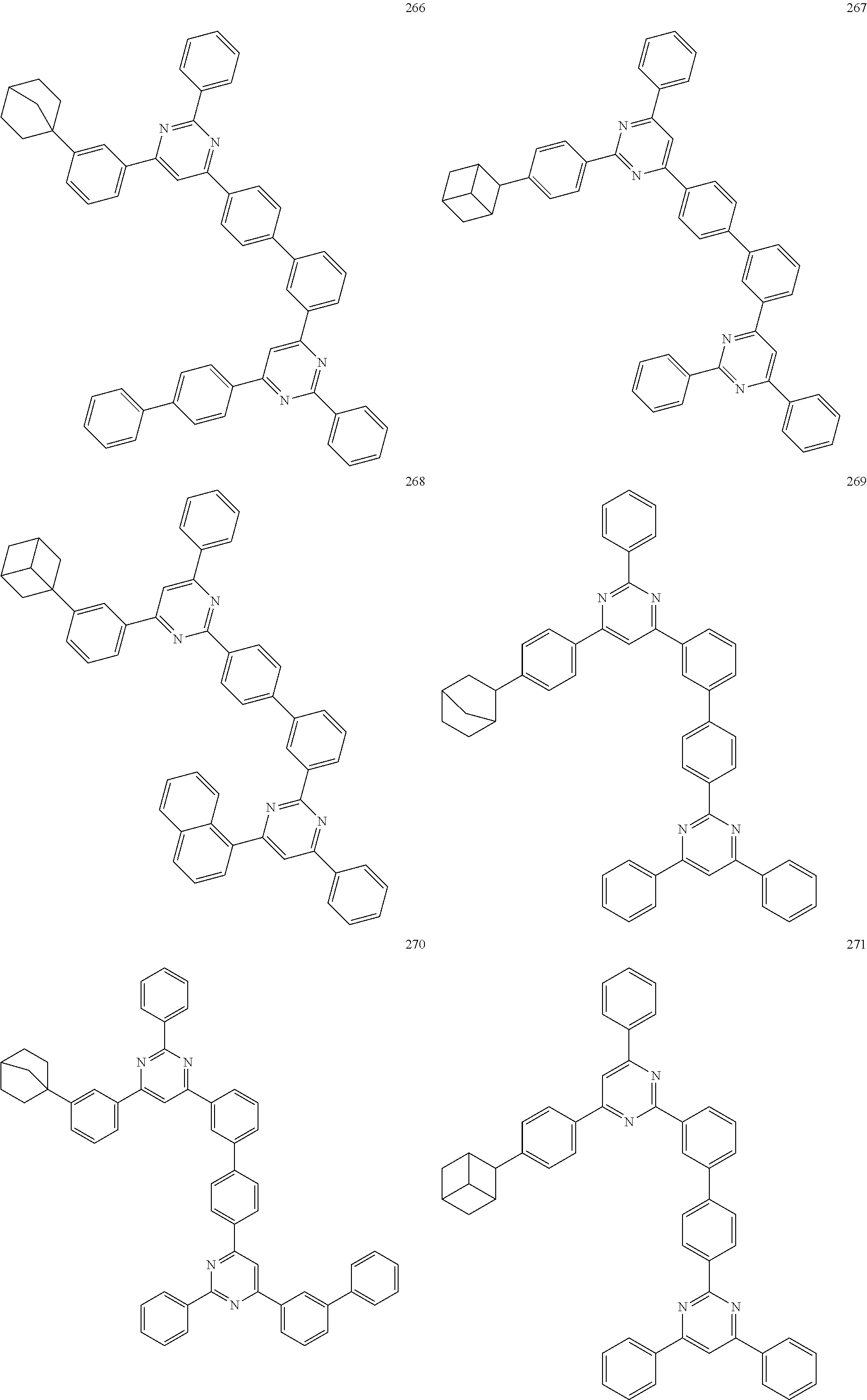

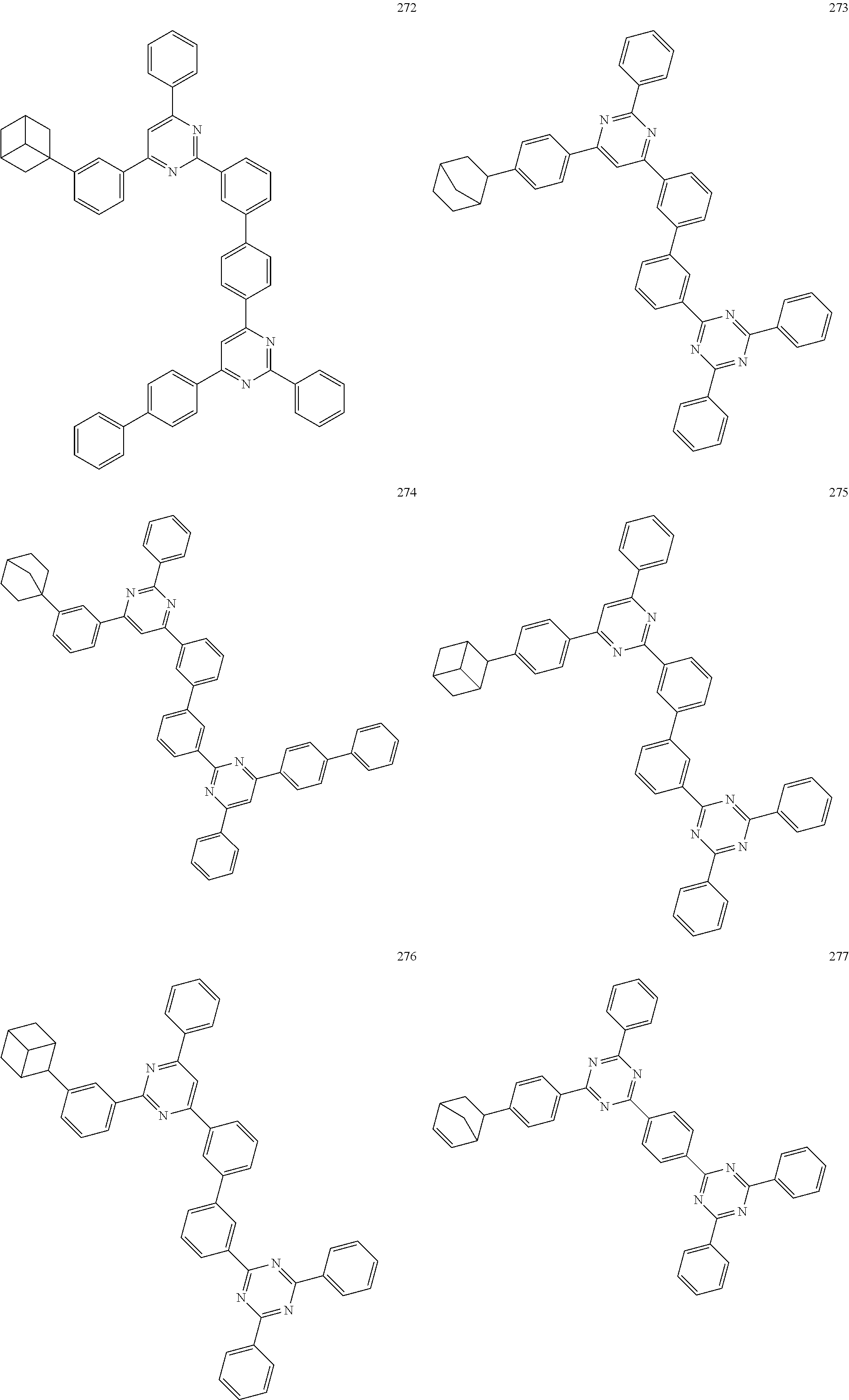

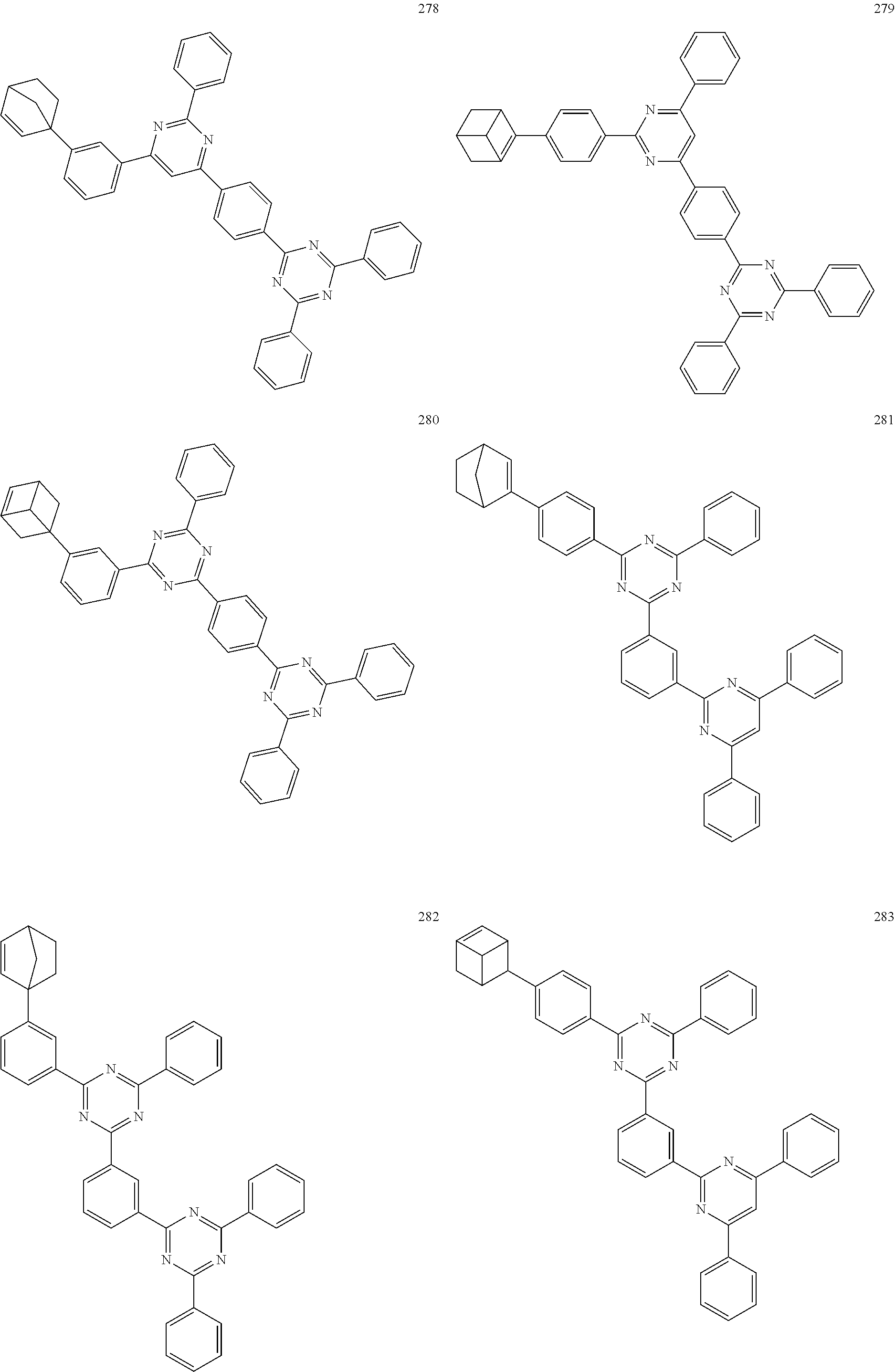

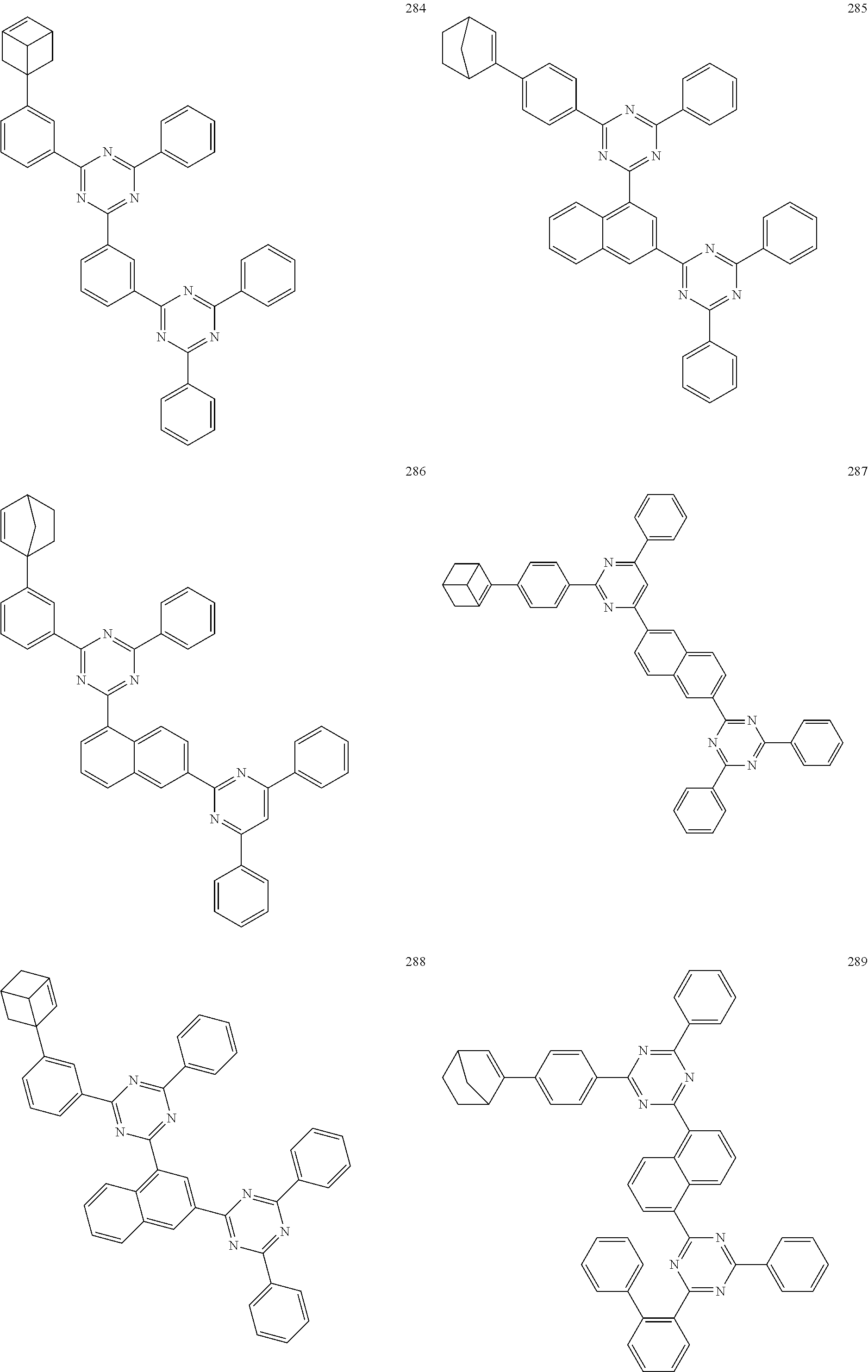

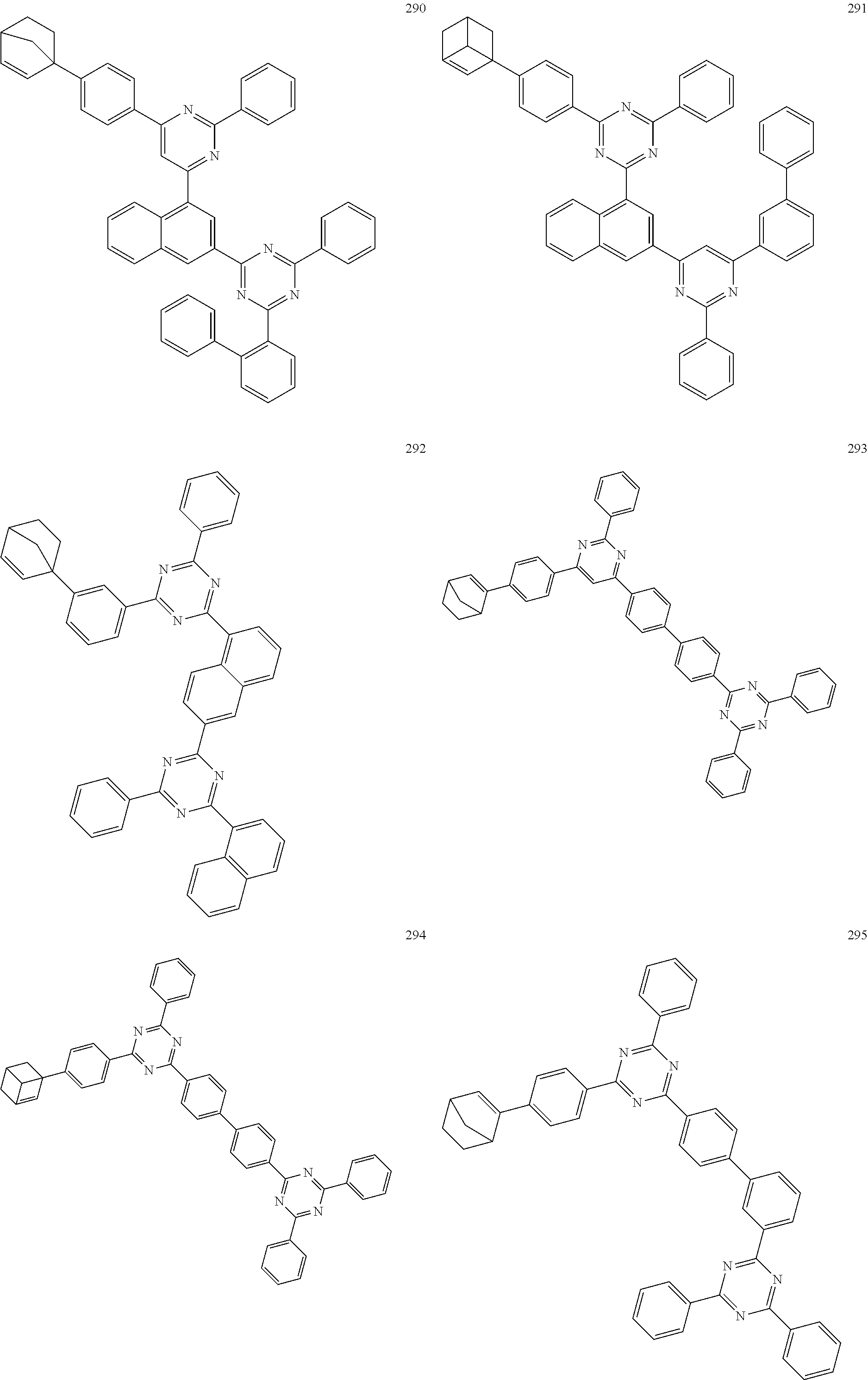

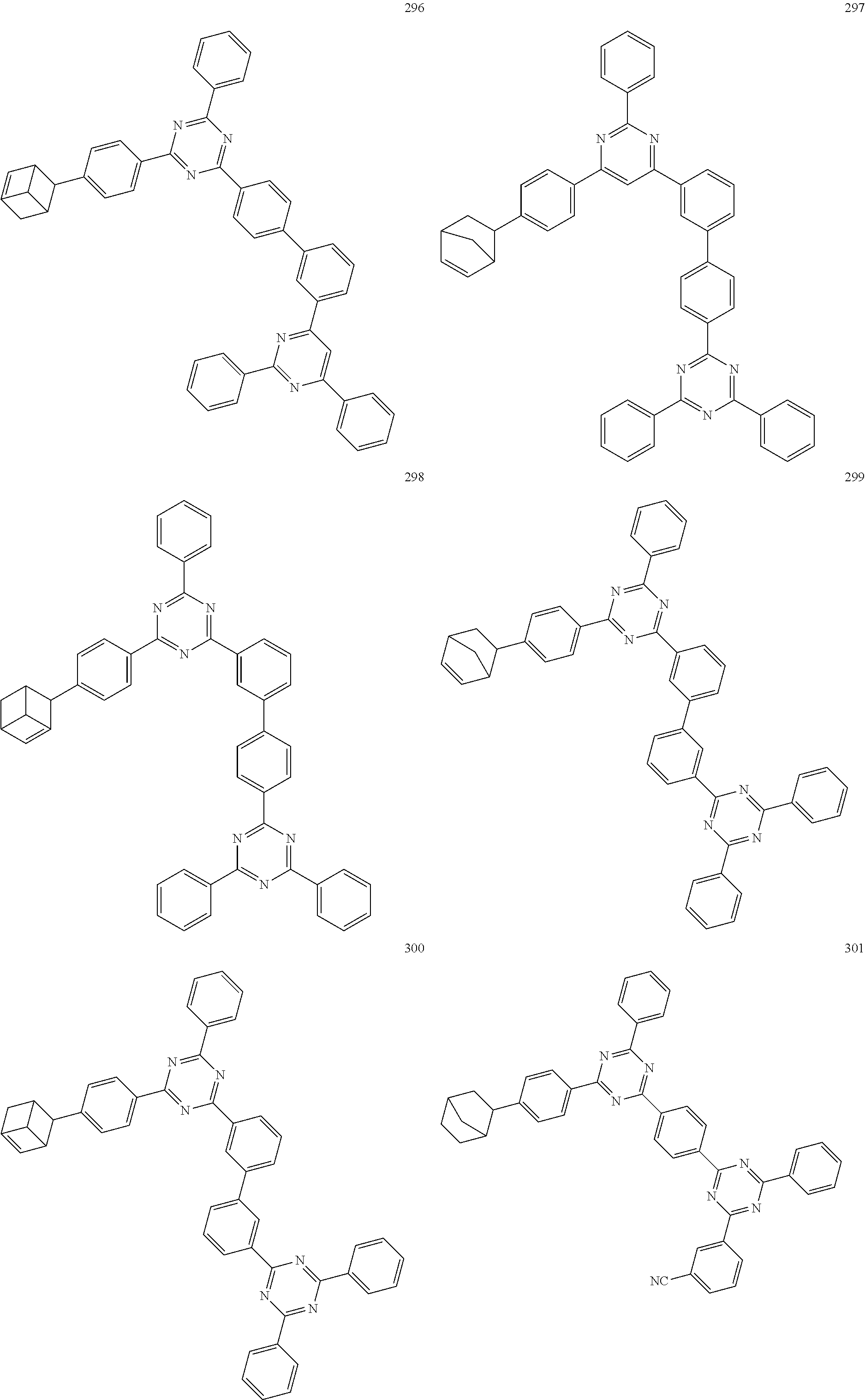

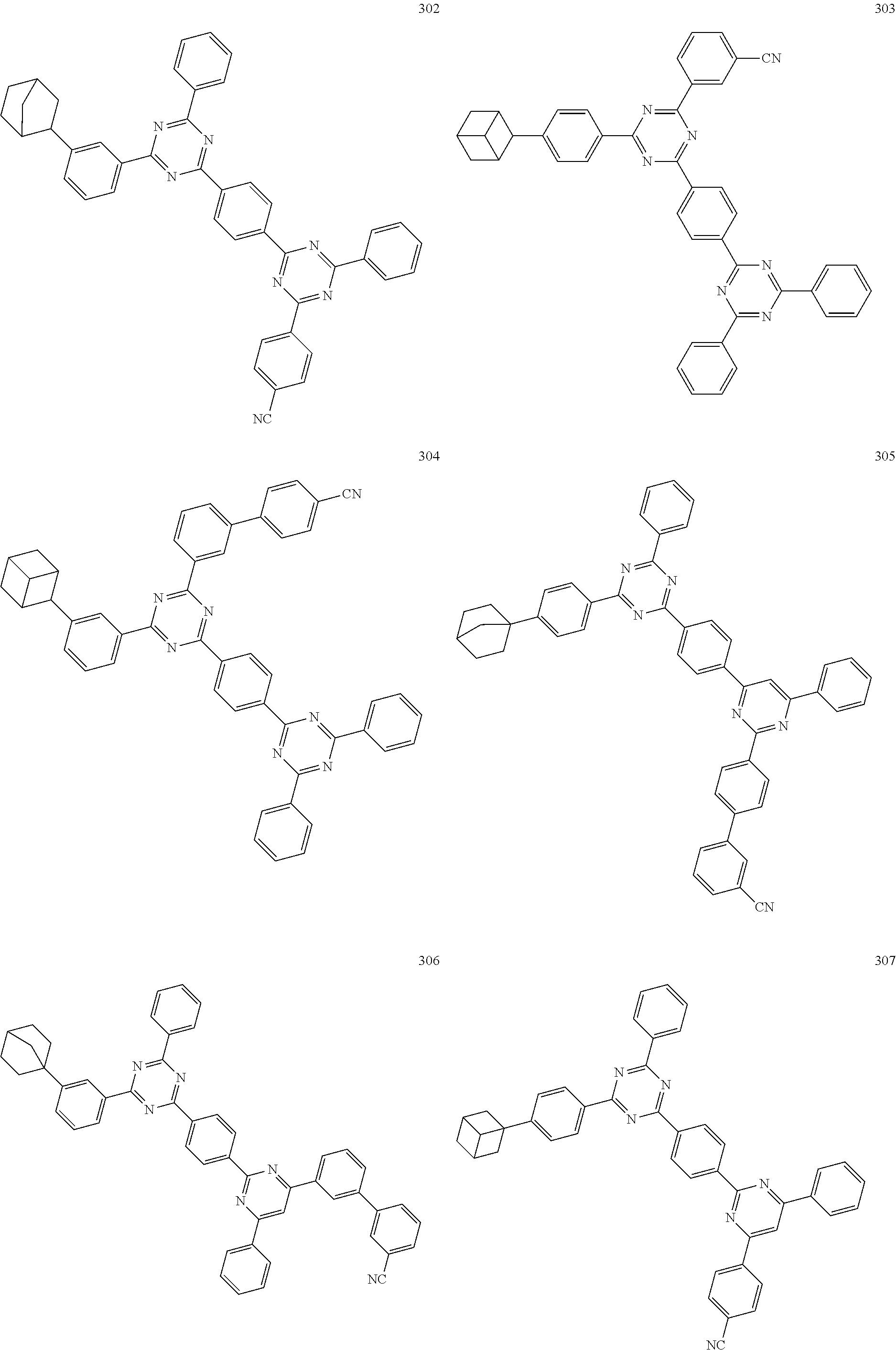

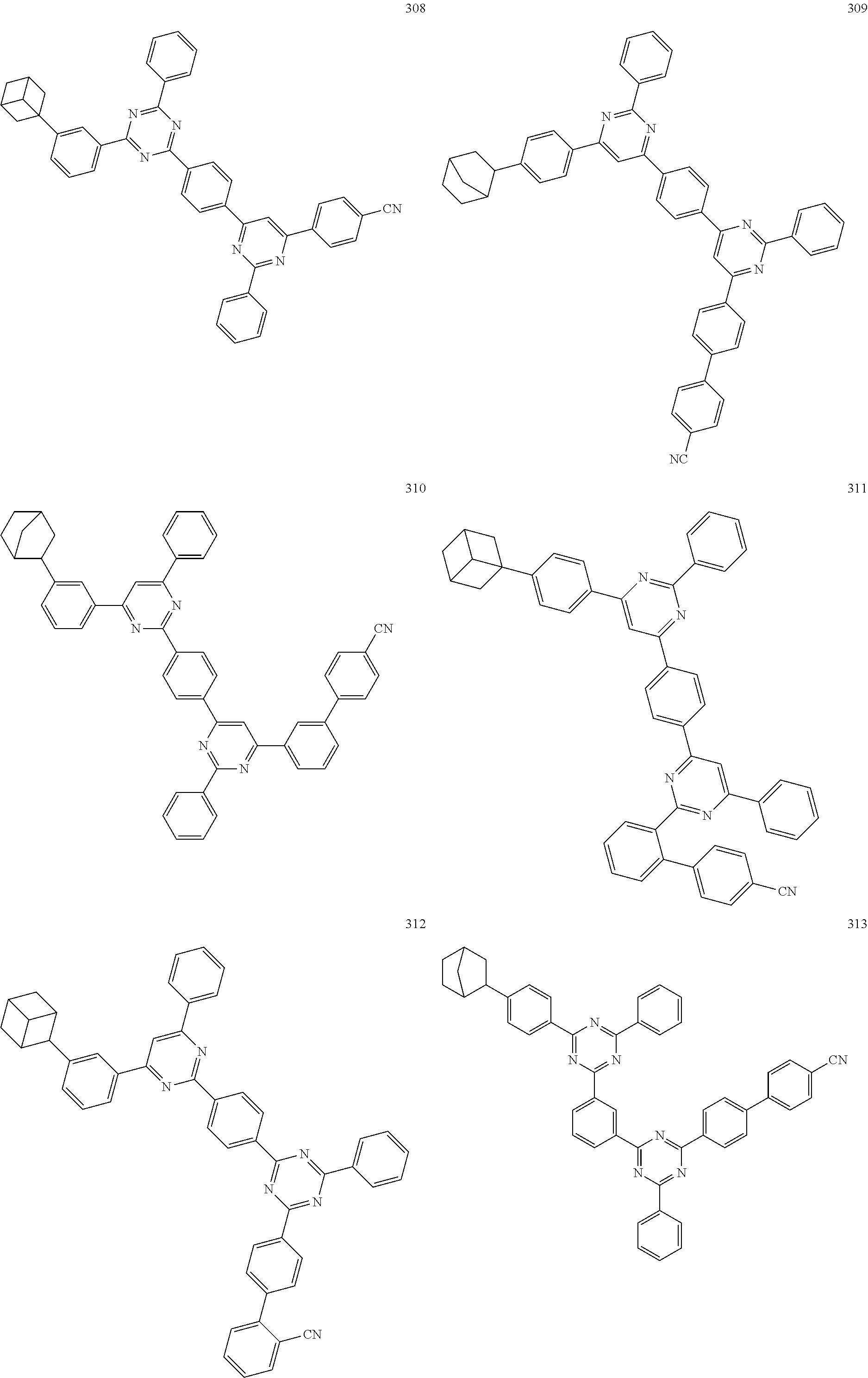

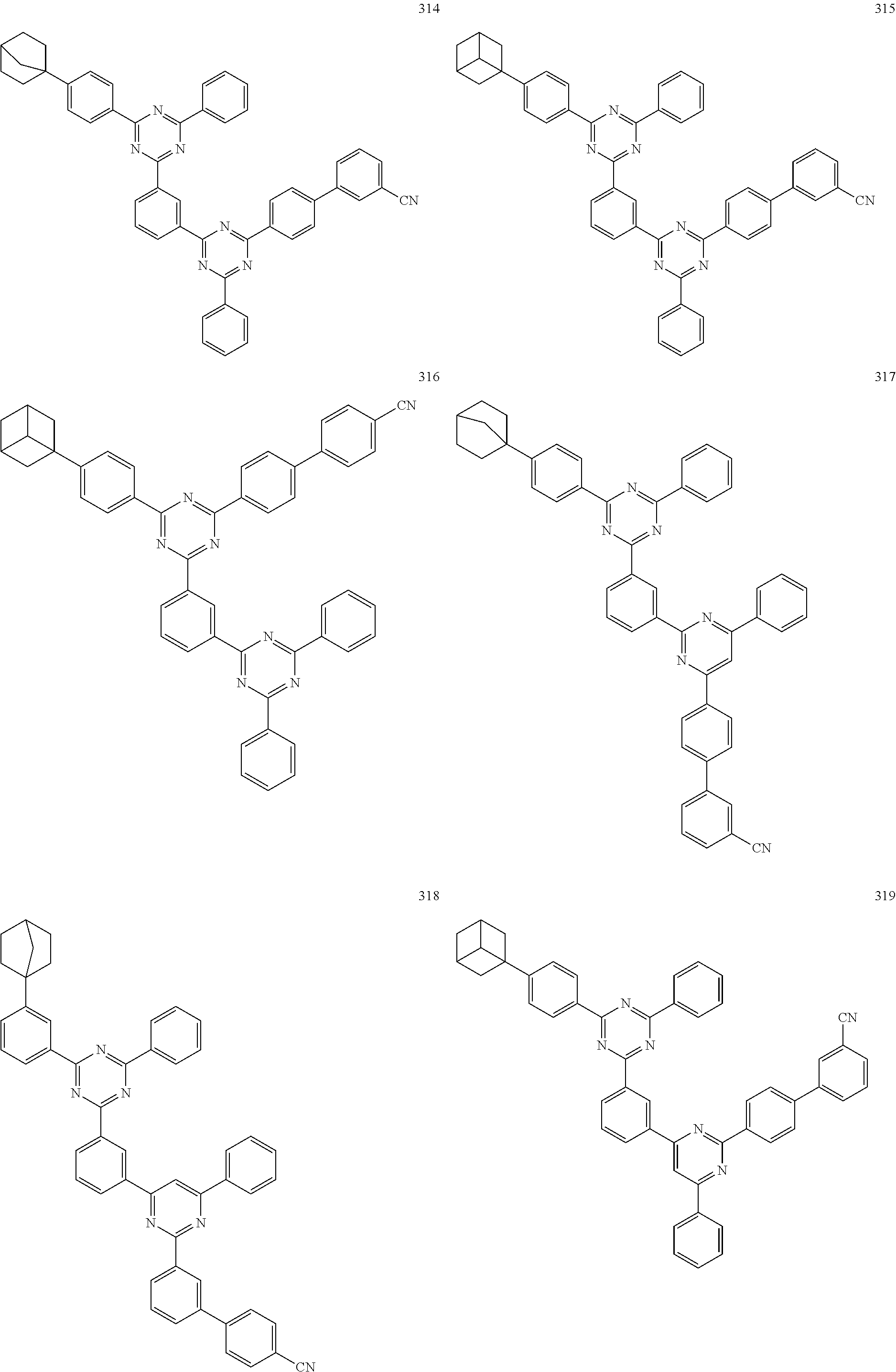

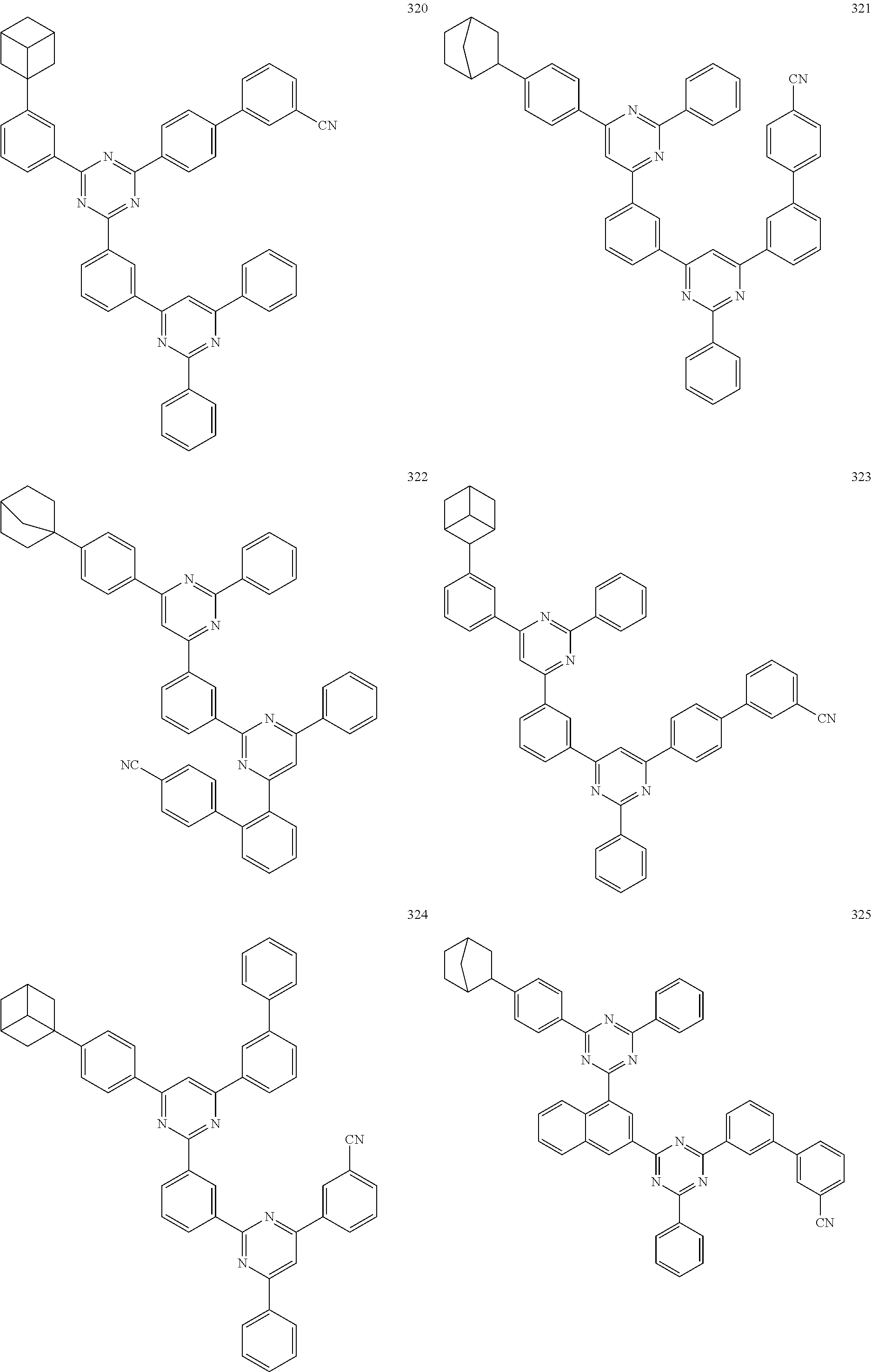

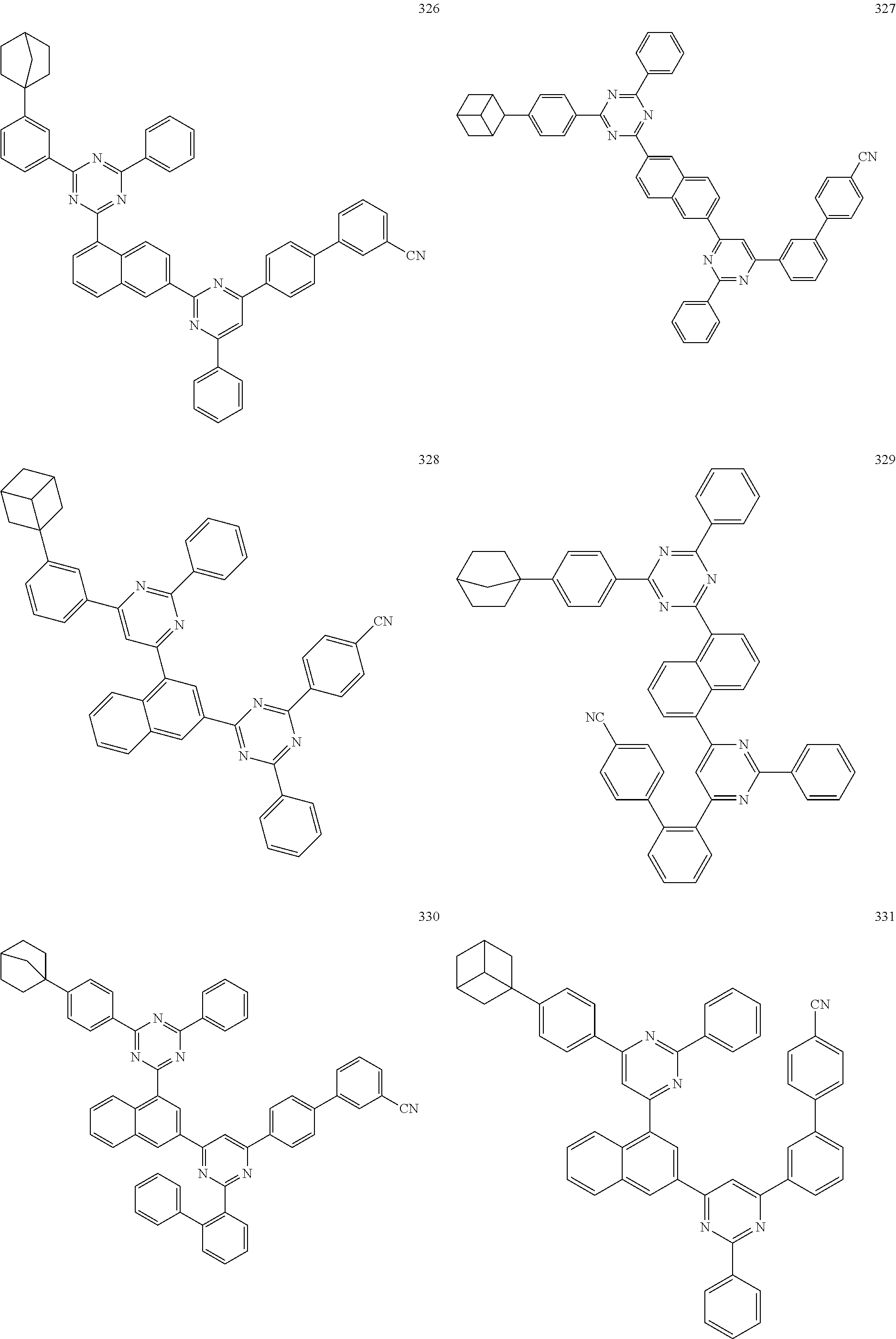

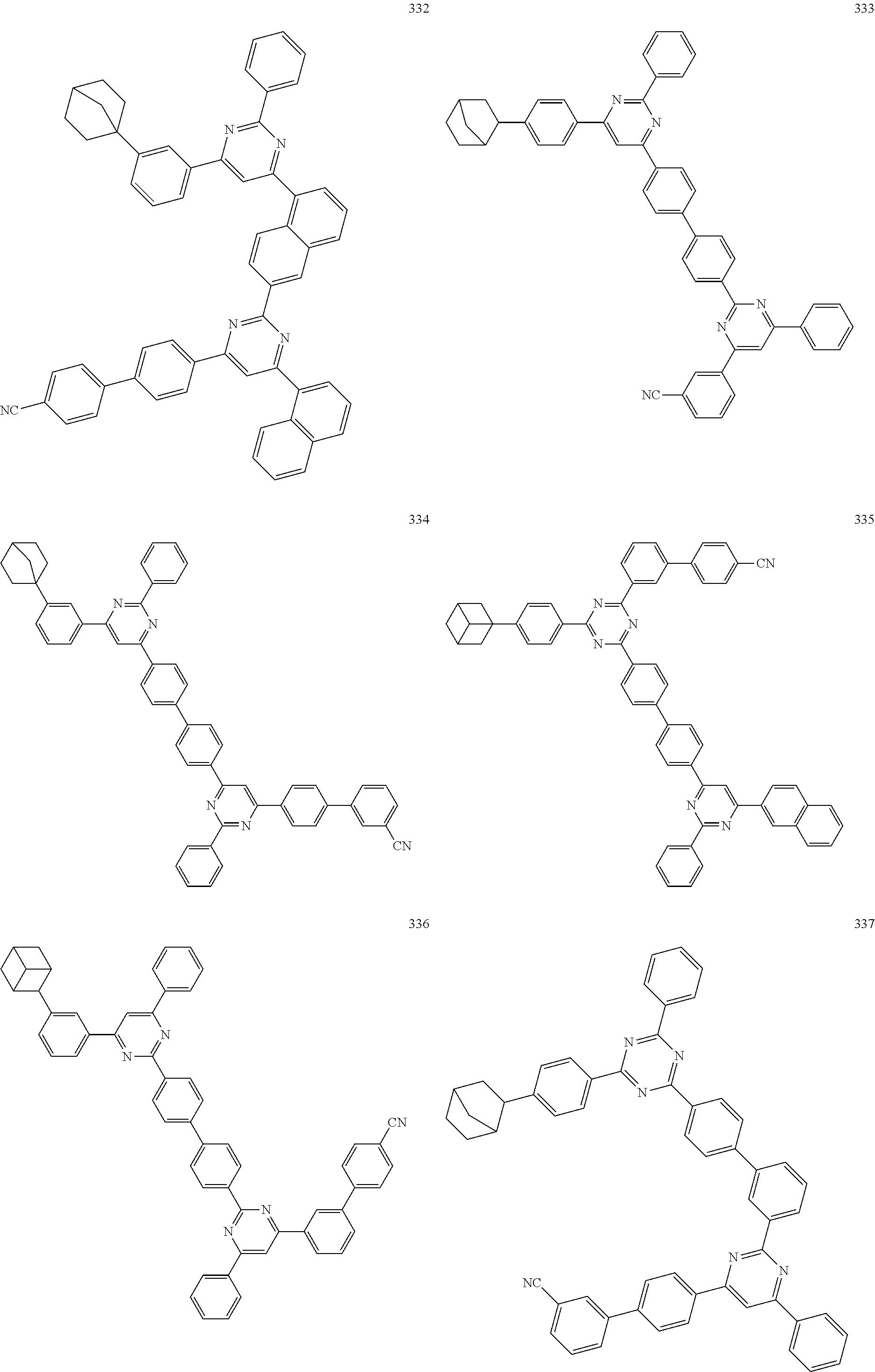

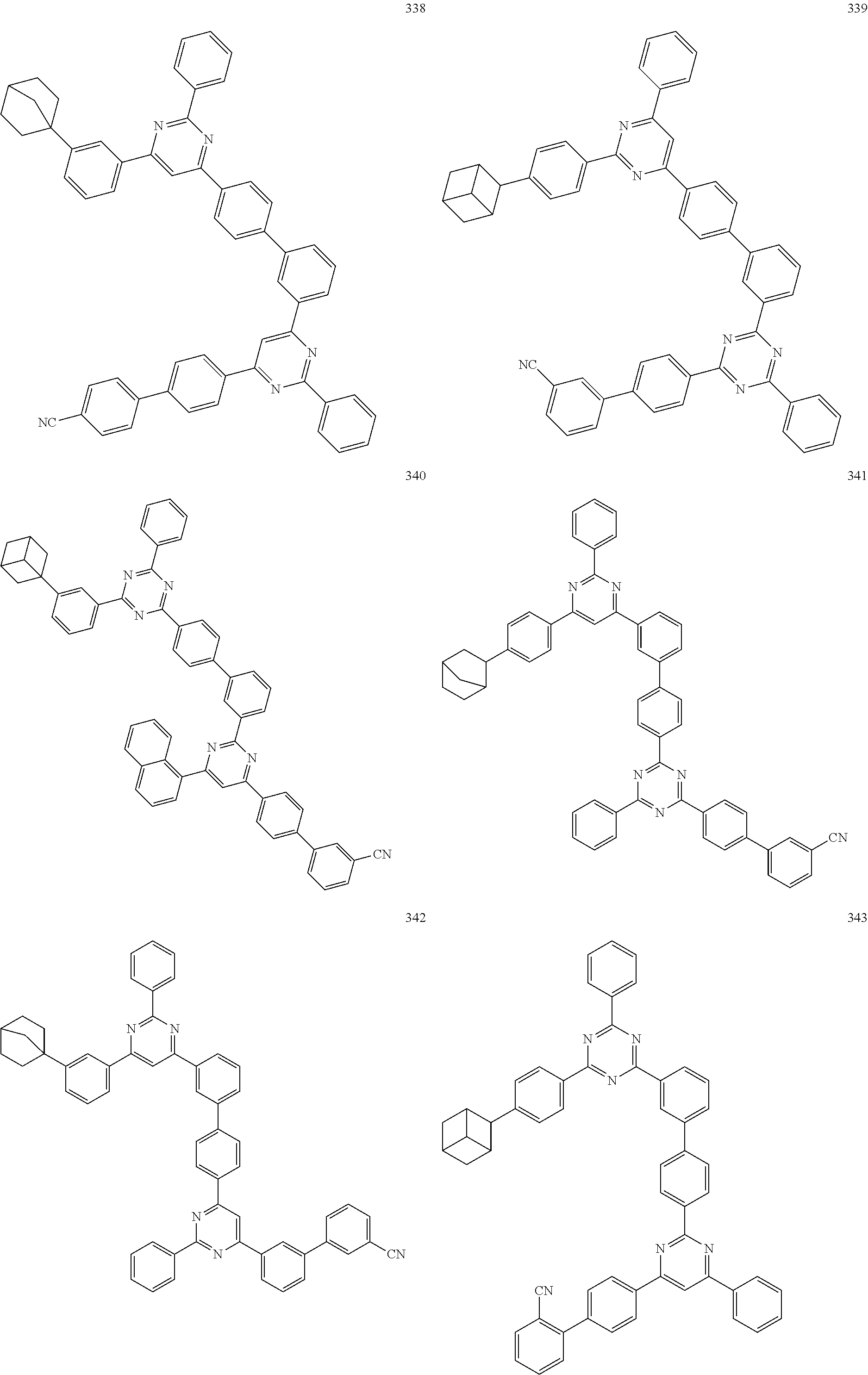

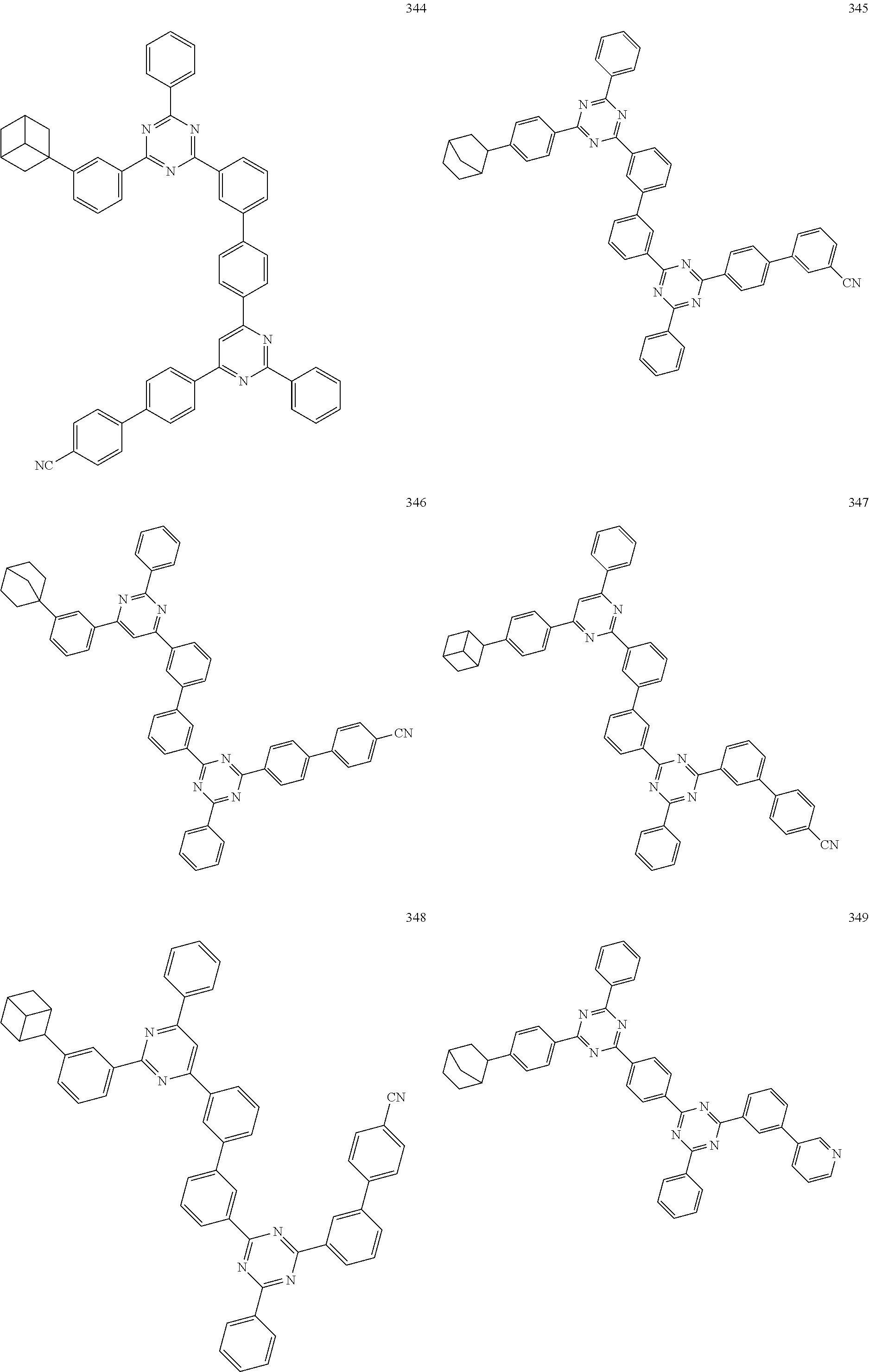

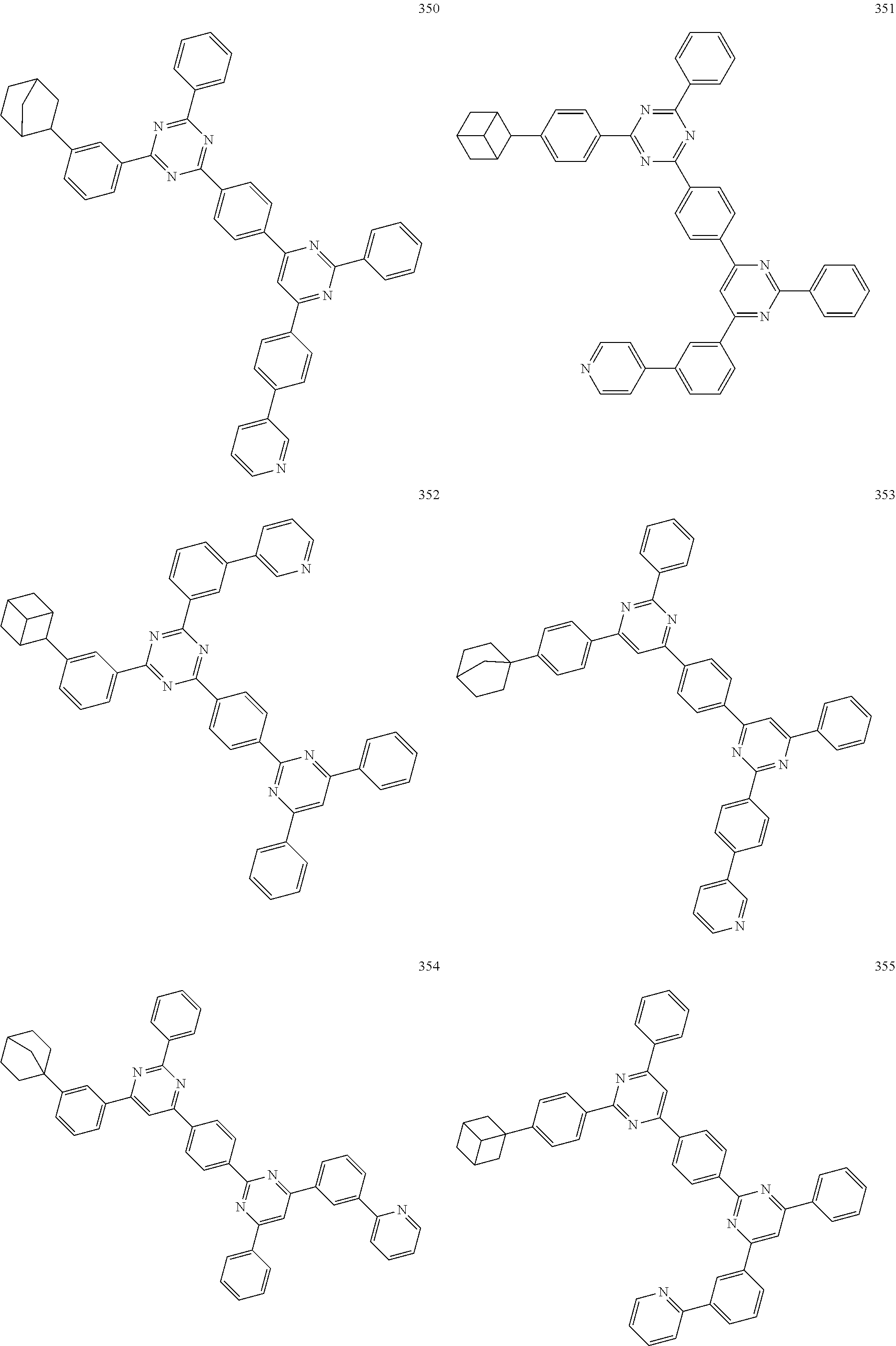

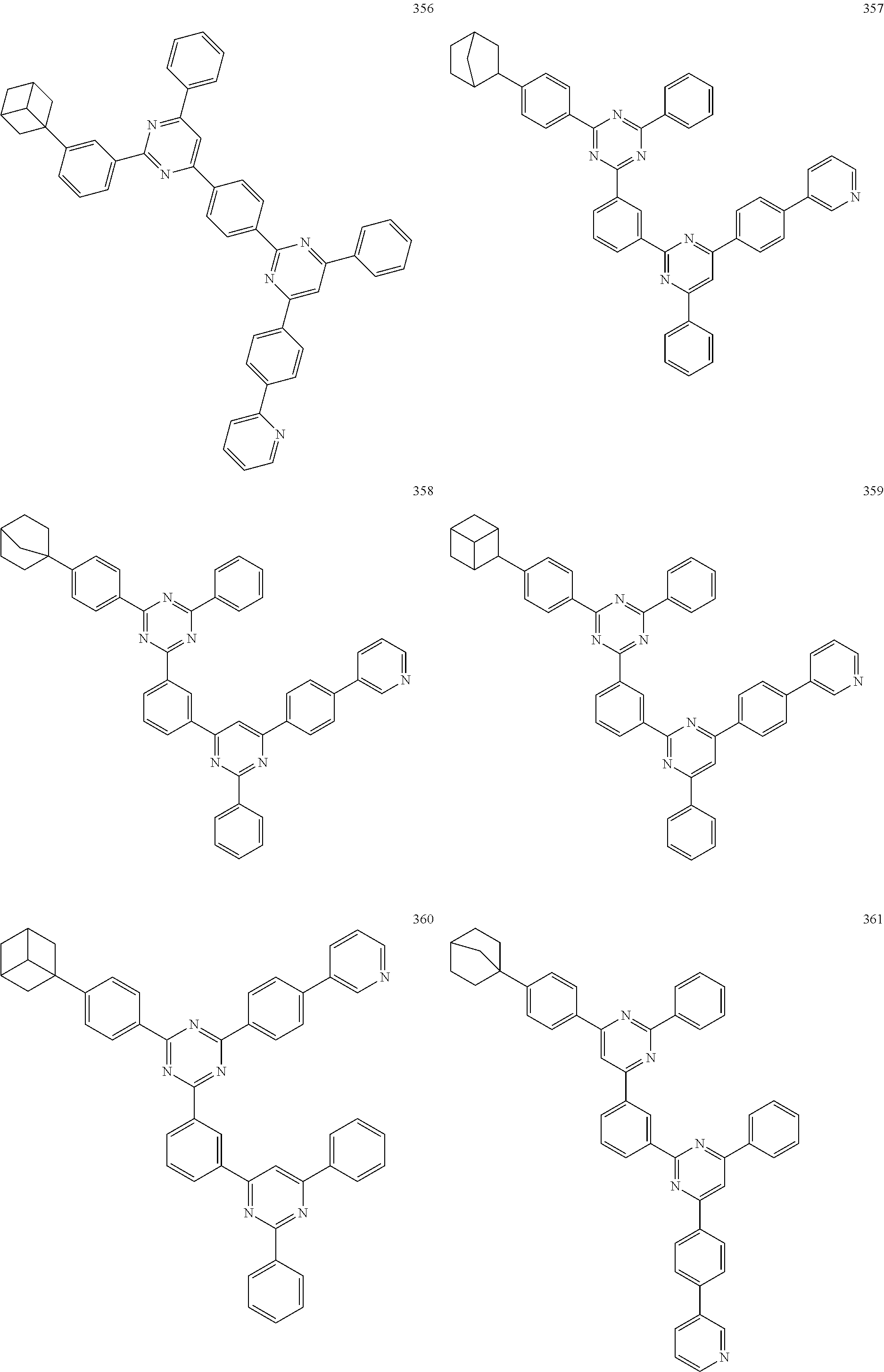

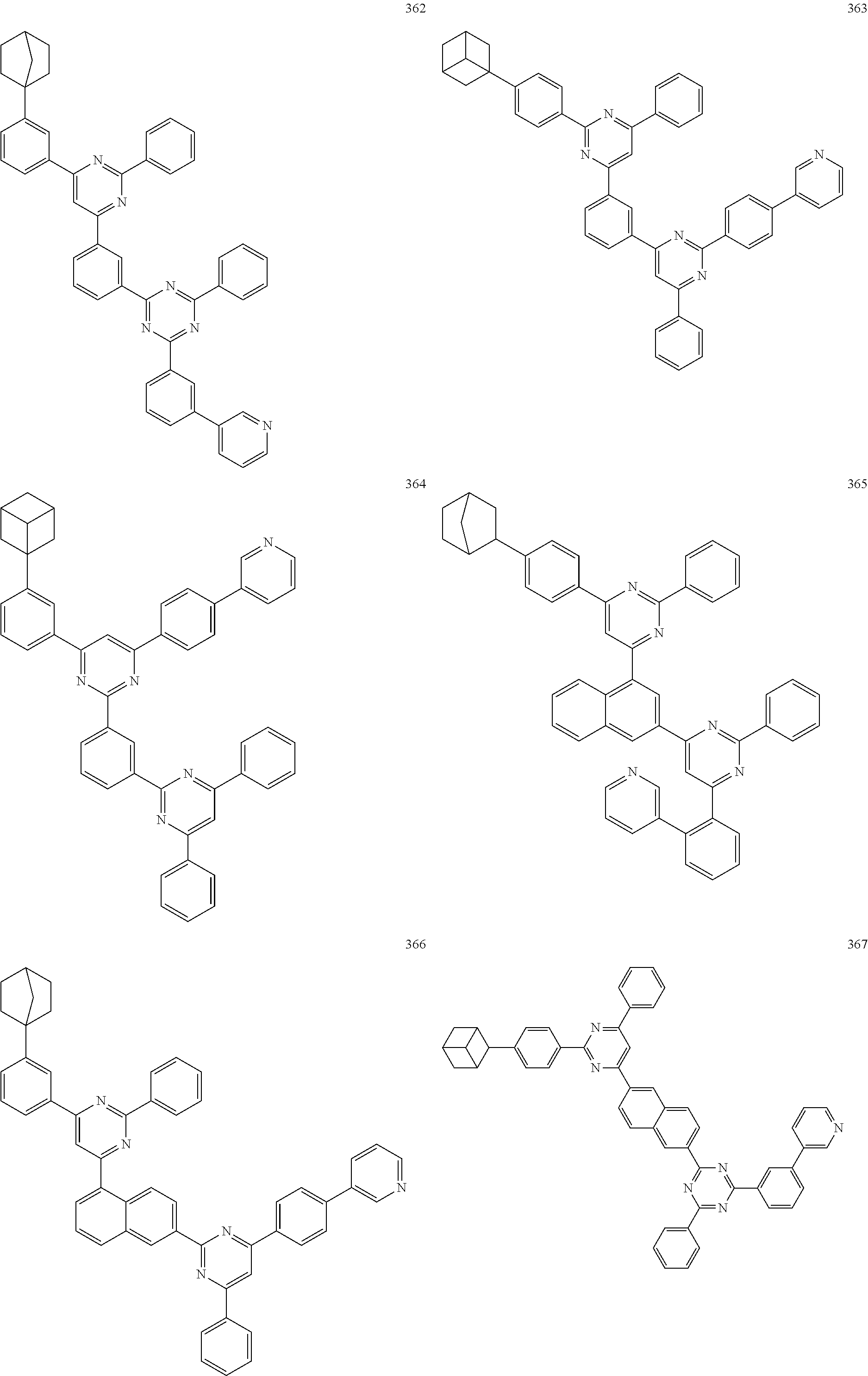

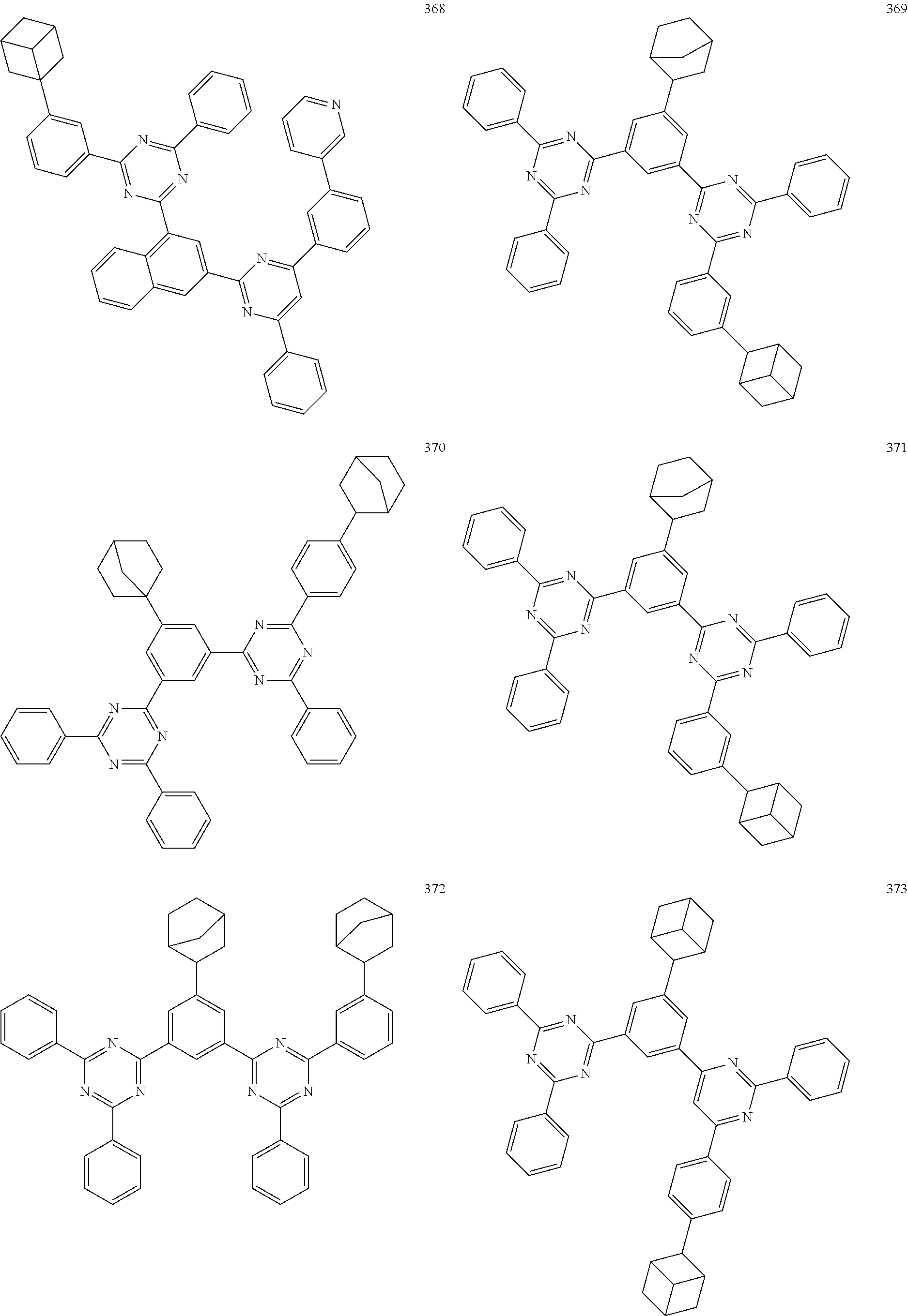

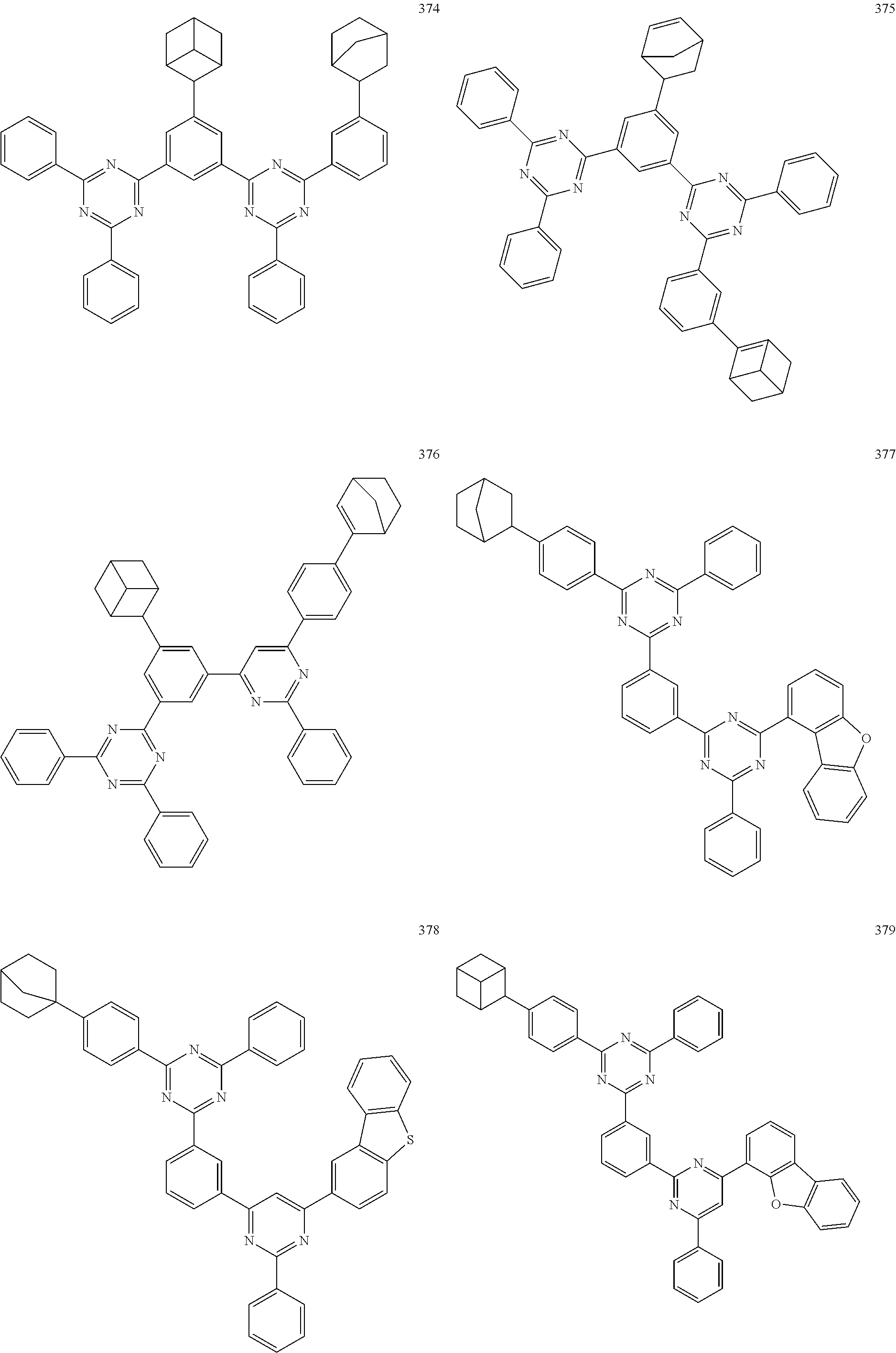

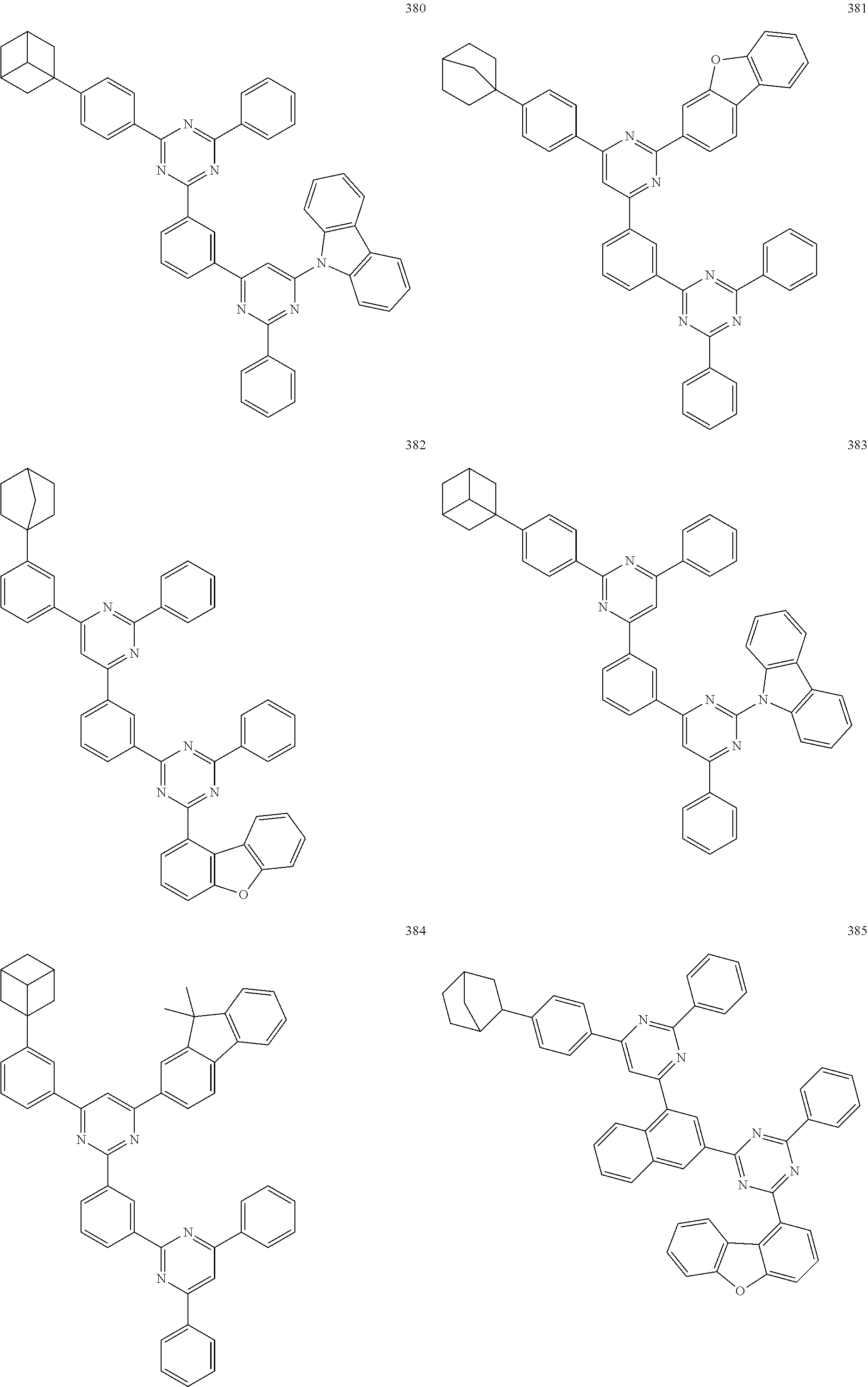

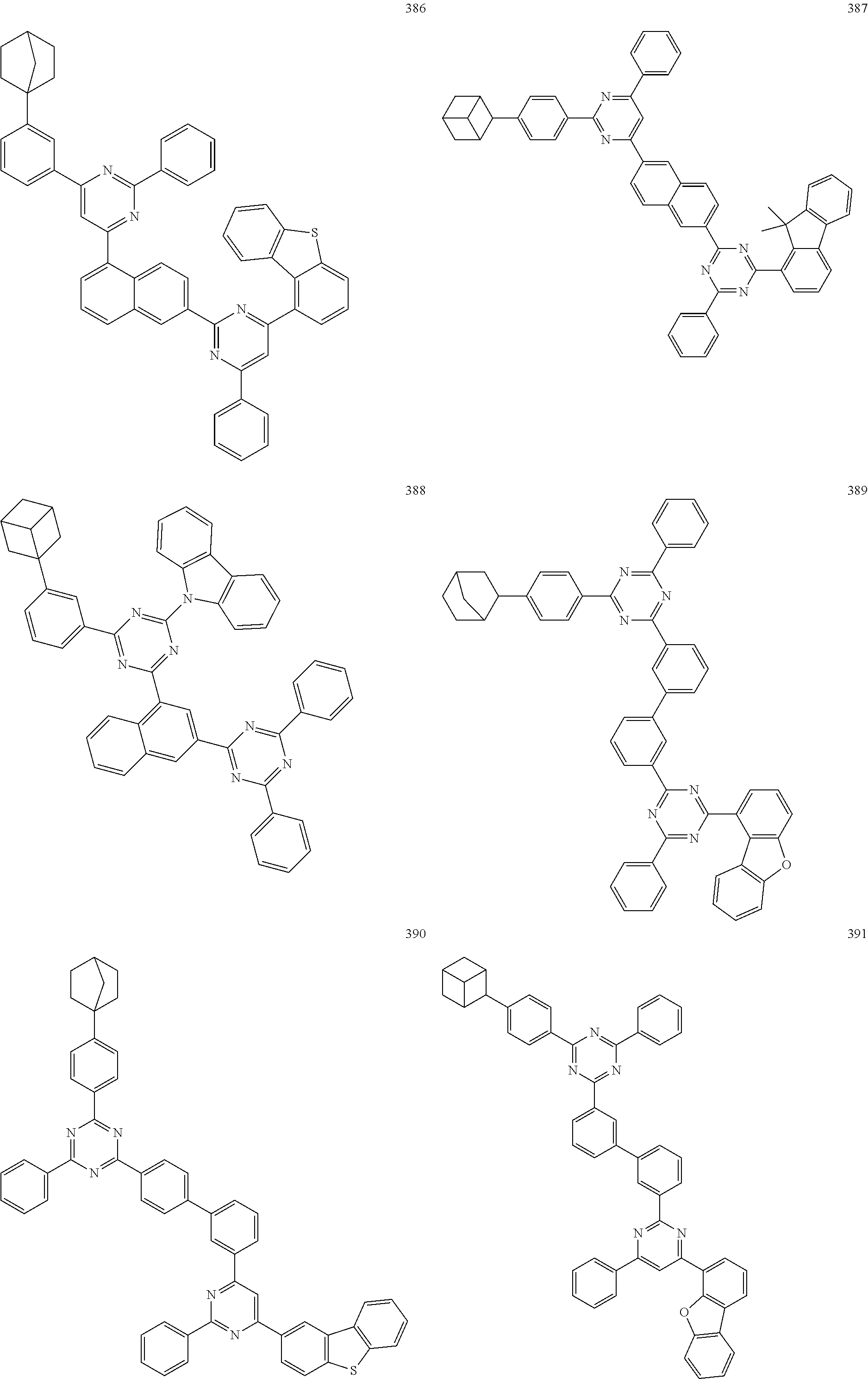

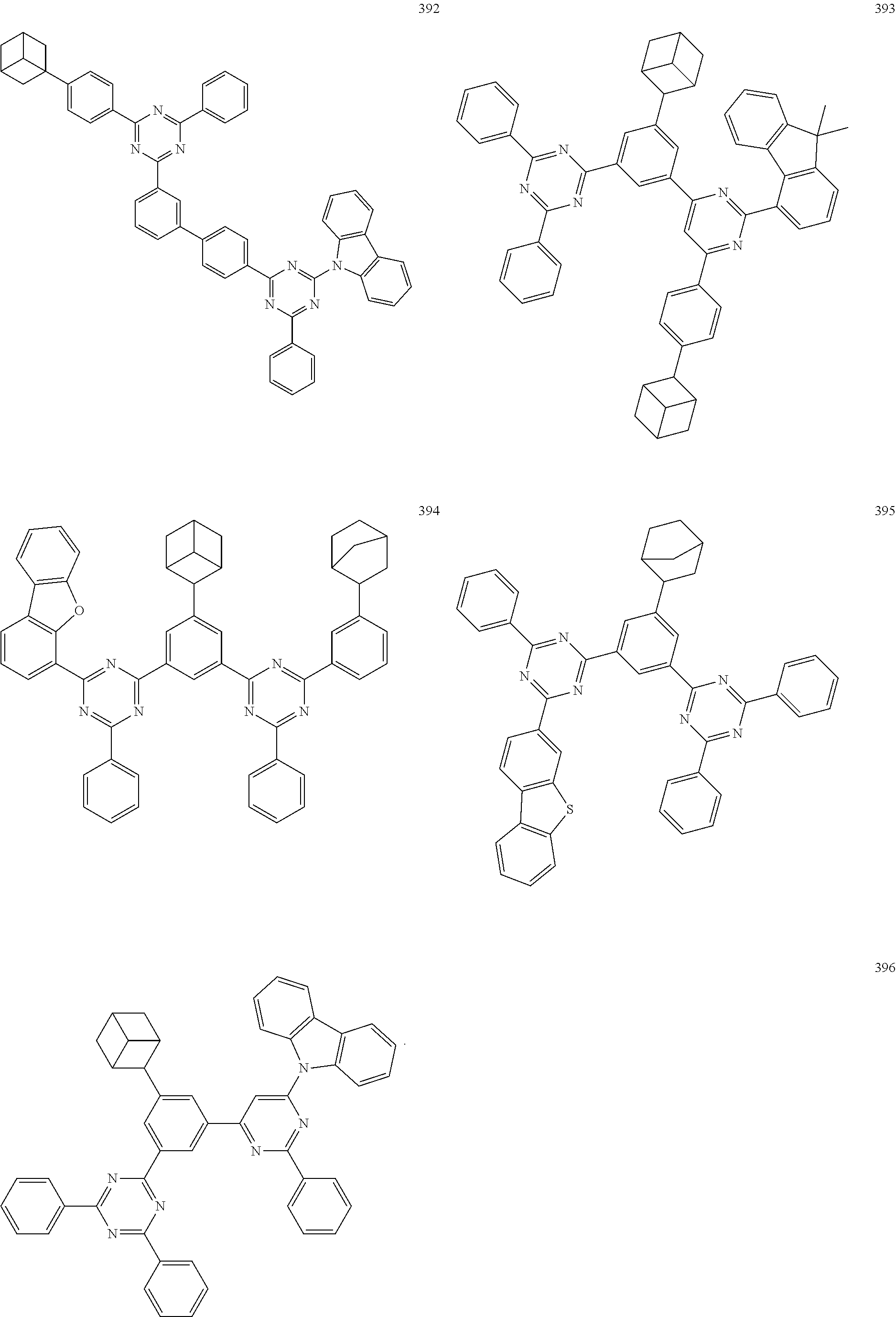

In the heterocyclic compound represented by Formula 1, at least one selected from R₁₁, R₁₂, R₂₁, R₂₂, and R₃ is a bridged C₅-C₁₂ polycyclic group unsubstituted or substituted with at least one R₁₀ₐ, and thus, due to a reduction in refractive index by introduction of the bridged polycyclic group, the light extraction effect of the light-emitting device is increased so that the current efficiency and/or light efficiency may be improved, thereby having excellent or suitable characteristics in terms of color coordinates. In some embodiments, due to the increased light efficiency, the lifespan characteristics may be improved by reducing the current required for the light-emitting device to realize the same luminance.

In some embodiments, the heterocyclic compound represented by Formula 1 may have improved electron transfer ability by combining two or more strong electron acceptors, such as a triazine group, a pyrimidine group, or a pyridine group, and accordingly, the light-emitting device may have an improved driving voltage and improved lifespan characteristics.

In some embodiments, by maintaining constant polarizability of a molecule by adjusting an appropriate or suitable dipole moment in the molecule, the change in refractive index may be minimized or reduced and the electrical characteristics of materials may be maintained, and accordingly the light efficiency of the device may be improved.

In this regard, when the heterocyclic compound represented by Formula 1 is included in the electron transport region (e.g., the electron transport layer or the electron transport auxiliary layer) of the light-emitting device, the driving voltage, current efficiency, color purity, device lifespan, and/or the like may be improved. Therefore, due to the utilization of the heterocyclic compound, an electronic apparatus (e.g., an organic light-emitting device) having low driving voltage, high efficiency, and long lifespan characteristics may be implemented.

Synthesis methods of the heterocyclic compound may be recognizable by one of ordinary skill in the art by referring to Synthesis Examples and/or Examples provided herein.

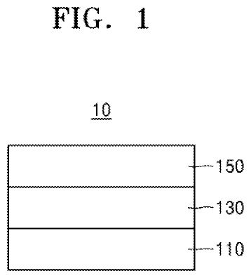

### Description of FIG. 1

FIG. 1 is a schematic cross-sectional view of a light-emitting device 10 according to one or more embodiments of the present disclosure. The light-emitting device 10 may include a first electrode 110, an interlayer 130, and a second electrode 150.

Hereinafter, the structure of the light-emitting device 10 according to one or more embodiments and a method of manufacturing the light-emitting device 10 will be described in more detail with reference to FIG. 1.

### First Electrode 110

In FIG. 1, in one or more embodiments, a substrate may be additionally provided and arranged under the first electrode 110 and/or on the second electrode 150. In some embodiments, as the substrate, a glass substrate or a plastic substrate may be utilized. In one or more embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. In one or more embodiments, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure including (e.g., consisting of) a single layer, or a multi-layered structure including multiple layers. For example, in some embodiments, the first electrode 110 may have a three-layered structure of ITO/Ag/ITO.

### Interlayer 130

The interlayer 130 may be arranged on the first electrode 110. The interlayer 130 may include an emission layer.

In one or more embodiments, the interlayer 130 may further include a hole transport region between the first electrode 110 and the emission layer, and an electron transport region between the emission layer and the second electrode 150.

In one or more embodiments, the interlayer 130 may further include, in addition to one or more suitable organic materials, a metal-containing compound such as an organometallic compound, an inorganic material such as quantum dots, and/or the like.

In one or more embodiments, the interlayer 130 may include i) two or more emitting units sequentially stacked between the first electrode 110 and the second electrode 150, and ii) a charge generation layer located between two neighboring emitting units. When the interlayer 130 includes emitting units and a charge generation layer as described above, the light-emitting device 10 may be a tandem light-emitting device.

### Hole Transport Region in Interlayer 130

The hole transport region may have: i) a single-layered structure including (e.g., consisting of) a single layer including (e.g., consisting of) a single material, ii) a single-layered structure including (e.g., consisting of) a single layer including (e.g., consisting of) multiple materials that are different from each other, or iii) a multi-layered structure including multiple layers including multiple materials that are different from each other.

The hole transport region may include a hole injection layer, a hole transport layer, an emission auxiliary layer, an electron blocking layer, or any combination thereof.

For example, in one or more embodiments, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein constituting layers in each structure are sequentially stacked from the first electrode 110 in each stated order.

In one or more embodiments, the hole transport region may include a compound represented by Formula 201, a compound represented by Formula 202, or any combination thereof:

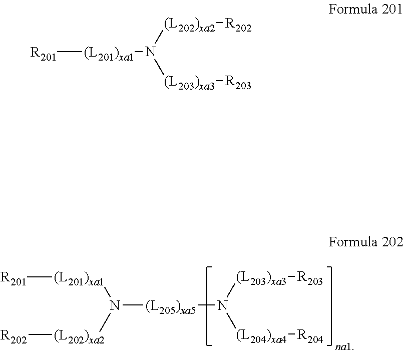

In Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—**, *—S—**, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group and/or the like) unsubstituted or substituted with at least one R₁₀ₐ (e.g., see Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
na1 may be an integer from 1 to 4.

For example, in some embodiments, each of Formulae 201 and 202 may include at least one selected from groups represented by Formulae CY201 to CY217:

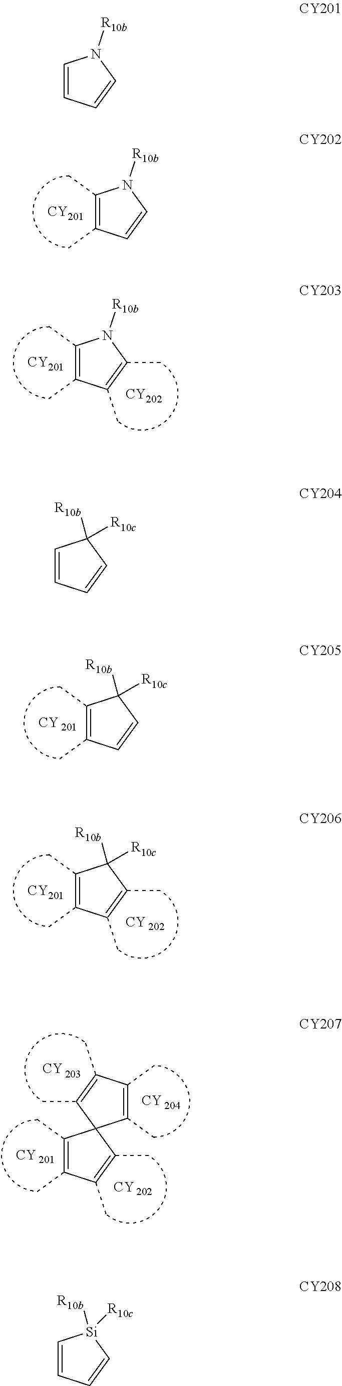

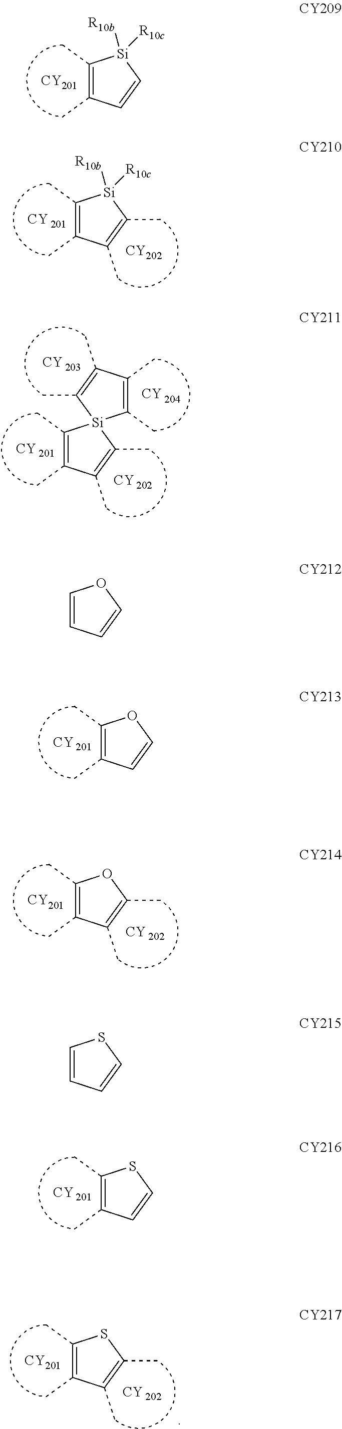

In Formulae CY201 to CY217, R_{10b} and R_{10c} may each be the same as described with respect to R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one selected from the groups represented by Formulae CY201 to CY203.

In one or more embodiments, Formula 201 may include at least one selected from the groups represented by Formulae CY201 to CY203 and at least one selected from the groups represented by Formulae CY204 to CY217.

In one or more embodiments, in Formula 201, xa1 may be 1, R₂₀₁ may be one selected from the groups represented by Formulae CY201 to CY203, xa2 may be 0, and R₂₀₂ may be one selected from the groups represented by one of Formulae CY204 to CY207.

In one or more embodiments, each of Formulae 201 and 202 may not include (e.g., may exclude) any of the groups represented by Formulae CY201 to CY203.

In one or more embodiments, each of Formulae 201 and 202 may not include (e.g., may exclude) any of the groups represented by Formulae CY201 to CY203, and may include at least one selected from the groups represented by Formulae CY204 to CY217.

In one or more embodiments, each of Formulae 201 and 202 may not include (e.g., may exclude) any of the groups represented by Formulae CY201 to CY217.

For example, in one or more embodiments, the hole transport region may include at least one selected from Compounds HT1 to HT46, 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), N, N′-di(naphthalen-1-yl)-N, N′-diphenyl-benzidine (NPB(NPD)), β-NPB, N,N′-bis(3-methylphenyl)-N,N′-diphenyl-[1,1′-biphenyl]-4,4′-diamine (TPD), spiro-TPD, spiro-NPB, methylated NPB, 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), and/or any combination thereof:

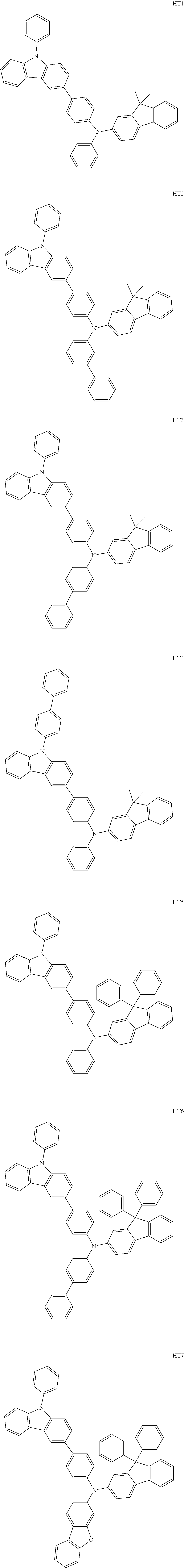

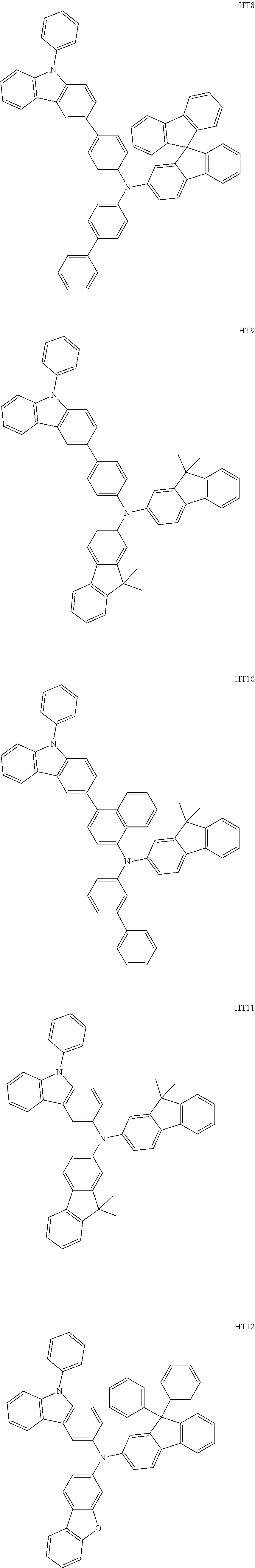 [SMILES: C1=CC(N(c2ccc(-c3ccccc3)cc2)c2ccc3c(c2)C2(c4ccccc4-c4ccccc42)c2ccccc2-3)CC=C1c1ccc2c(c1)c1ccccc1n2-c1ccccc1 ; CC1(C)C2=CC(N(c3ccc(-c4ccc5c(c4)c4ccccc4n5-c4ccccc4)cc3)c3ccc4c(c3)C(C)(C)c3ccccc3-4)CC=C2c2ccccc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

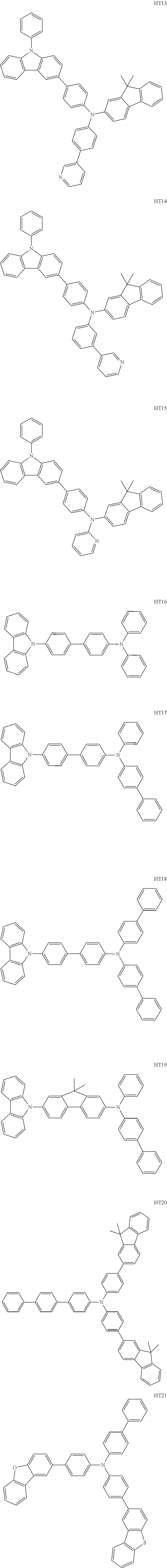

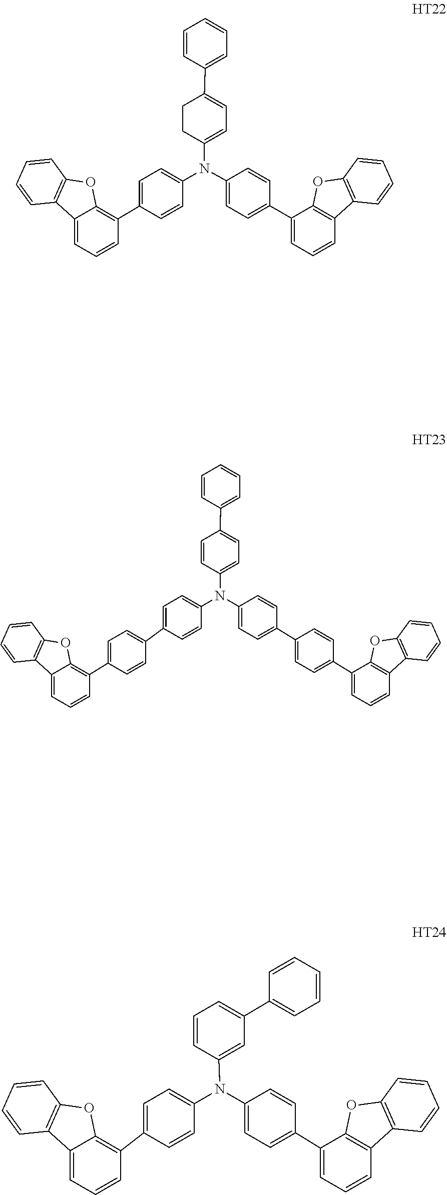 [SMILES: C1=C(c2ccccc2)CCC(N(c2ccc(-c3cccc4c3oc3ccccc34)cc2)c2ccc(-c3cccc4c3oc3ccccc34)cc2)=C1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1]

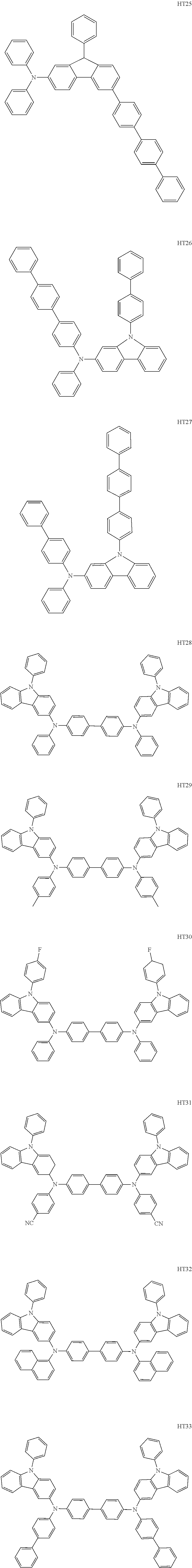

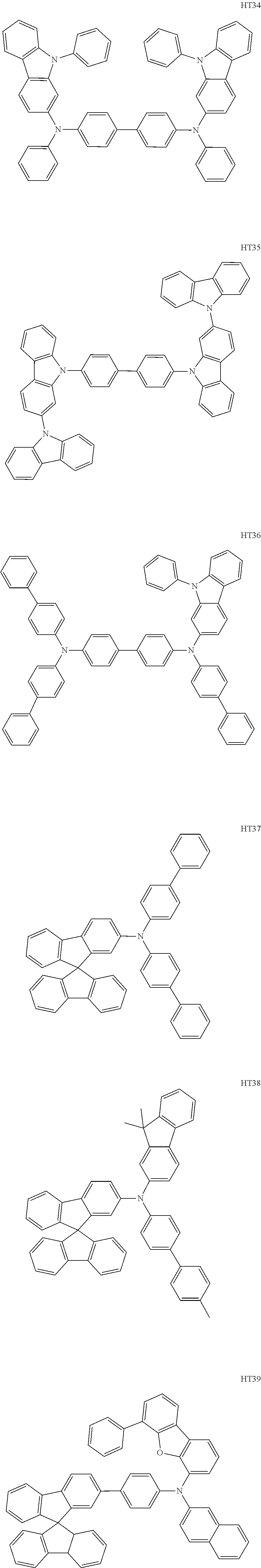

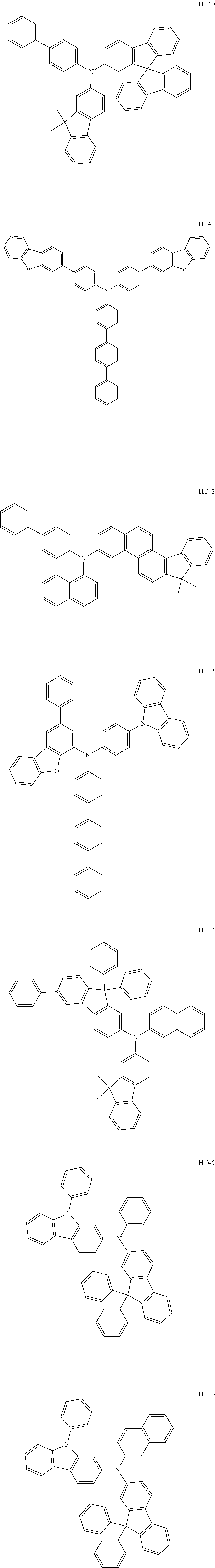

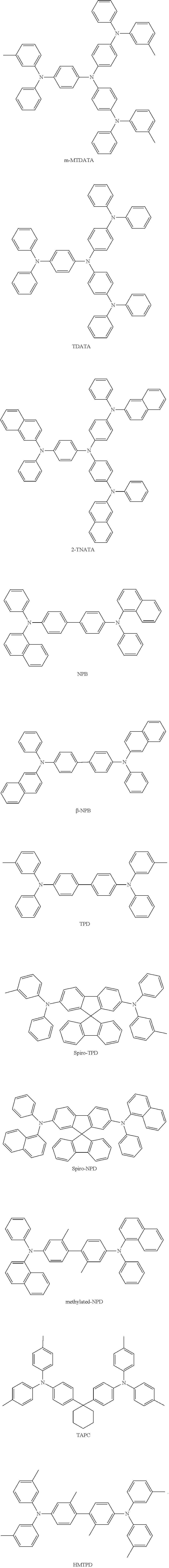

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron-blocking layer may block or reduce the leakage of electrons from the emission layer to the hole transport region. Materials that may be included in the hole transport region may be included in the emission auxiliary layer and the electron blocking layer.

p-Dopant

In one or more embodiments, the hole transport region may further include, in addition to the aforementioned materials, a charge-generation material for the improvement of conductive properties. The charge-generation material may be substantially uniformly or non-uniformly dispersed in the hole transport region (for example, in the form of a single layer including (e.g., consisting of) a charge-generation material).

The charge-generation material may be, for example, a p-dopant.

For example, in some embodiments, the lowest unoccupied molecular orbital (LUMO) energy level of the p-dopant may be −3.5 eV or less.

In one or more embodiments, the p-dopant may include a quinone derivative, a cyano group-containing compound, a compound including element EL1 and element EL2, or any combination thereof.

Non-limiting examples of the quinone derivative may be tetracyanoquinodimethane (TCNQ), 2,3,5,6-tetrafluoro-7,7,8,8-tetracyanoquinodimethane (F4-TCNQ), and/or the like.

Non-limiting examples of the cyano group-containing compound may be dipyrazino[2,3-f:2′,3′-h] quinoxaline-2,3,6,7,10,11-hexacarbonitrile (HAT-CN), a compound represented by Formula 221, and/or the like:

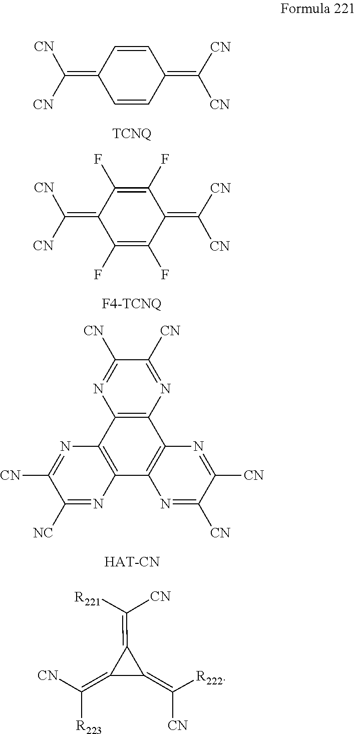

In Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be metal, metalloid, or any combination thereof, and element EL2 may be non-metal, metalloid, or any combination thereof.

Non-limiting examples of the metal may be: alkali metals (e.g., lithium (Li), sodium (Na), potassium (K), rubidium (Rb), cesium (Cs), etc.); alkaline earth metals (e.g., beryllium (Be), magnesium (Mg), calcium (Ca), strontium (Sr), barium (Ba), etc.); transition metals (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); post-transition metals (e.g., zinc (Zn), indium (In), tin (Sn), etc.); lanthanide metals (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and/or the like.

Non-limiting examples of the metalloid may be silicon (Si), antimony (Sb), tellurium (Te), and/or the like.

Non-limiting examples of the non-metal may be oxygen (O), a halogen (e.g., F, Cl, Br, I, etc.), and/or the like.

Non-limiting examples of the compound containing element EL1 and element EL2 may include metal oxides, metal halides (e.g., metal fluorides, metal chlorides, metal bromides, metal iodides, etc.), metalloid halides (e.g., metalloid fluorides, metalloid chlorides, metalloid bromides, metalloid iodides, etc.), metal tellurides, or any combination thereof.

Non-limiting examples of the metal oxide may be tungsten oxides (e.g., WO, W₂O₃, WO₂, WO₃, W₂O₅, etc.), vanadium oxides (e.g., VO, V₂O₃, VO₂, V₂O₅, etc.), molybdenum oxides (e.g., MoO, Mo₂O₃, MoO₂, MoO₃, Mo₂O₅, etc.), rhenium oxides (e.g., ReO₃, etc.), and/or the like.

Non-limiting examples of the metal halide may be alkali metal halides, alkaline earth metal halides, transition metal halides, post-transition metal halides, lanthanide metal halides, and/or the like.

Non-limiting examples of the alkali metal halide may be LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Non-limiting examples of the alkaline earth metal halide may be BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, BaI₂, and/or the like.

Non-limiting examples of the transition metal halide may be titanium halides (e.g., TiF₄, TiCl₄, TiBr₄, TiI₄, etc.), zirconium halides (e.g., ZrF₄, ZrCl₄, ZrBr₄, ZrI₄, etc.), hafnium halides (e.g., HfF₄, HfCl₄, HfBr₄, HfI₄, etc.), vanadium halides (e.g., VF₃, VCl₃, VBr₃, VI₃, etc.), niobium halides (e.g., NbF₃, NbCl₃, NbBr₃, NbI₃, etc.), tantalum halides (e.g., TaF₃, TaCl₃, TaBr₃, TaI₃, etc.), chromium halides (e.g., CrF₃, CrCl₃, CrBr₃, CrI₃, etc.), molybdenum halides (e.g., MoF₃, MoCl₃, MoBr₃, MoI₃, etc.), tungsten halides (e.g., WF₃, WCl₃, WBr₃, WI₃, etc.), manganese halides (e.g., MnF₂, MnCl₂, MnBr₂, MnI₂, etc.), technetium halides (e.g., TcF₂, TcCl₂, TcBr₂, TcI₂, etc.), rhenium halides (e.g., ReF₂, ReCl₂, ReBr₂, ReI₂, etc.), ferrous halides (e.g., FeF₂, FeCl₂, FeBr₂, FeI₂, etc.), ruthenium halides (e.g., RuF₂, RuCl₂, RuBr₂, RuI₂, etc.), osmium halides (e.g., OsF₂, OsCl₂, OsBr₂, OsI₂, etc.), cobalt halides (e.g., CoF₂, CoCl₂, CoBr₂, CoI₂, etc.), rhodium halides (e.g., RhF₂, RhCl₂, RhBr₂, RhI₂, etc.), iridium halides (e.g., IrF₂, IrCl₂, IrBr₂, IrI₂, etc.), nickel halides (e.g., NiF₂, NiCl₂, NiBr₂, NiI₂, etc.), palladium halides (e.g., PdF₂, PdCl₂, PdBr₂, PdI₂, etc.), platinum halides (e.g., PtF₂, PtCl₂, PtBr₂, PtI₂, etc.), cuprous halides (e.g., CuF, CuCl, CuBr, CuI, etc.), silver halides (e.g., AgF, AgCl, AgBr, AgI, etc.), gold halides (e.g., AuF, AuCl, AuBr, AuI, etc.), and/or the like.

Non-limiting examples of the post-transition metal halide may be zinc halides (e.g., ZnF₂, ZnCl₂, ZnBr₂, ZnI₂, etc.), indium halides (e.g., InI₃, etc.), tin halides (e.g., SnI₂, etc.), and/or the like.

Non-limiting examples of the lanthanide metal halide may be YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Non-limiting examples of the metalloid halide may be antimony halides (e.g., SbCl₅, etc.) and/or the like.

Non-limiting examples of the metal telluride may be alkali metal tellurides (e.g., Li₂Te, Na₂Te, K₂Te, Rb₂Te, Cs₂Te, etc.), alkaline earth metal tellurides (e.g., BeTe, MgTe, CaTe, SrTe, BaTe, etc.), transition metal tellurides (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal tellurides (e.g., ZnTe, etc.), lanthanide metal tellurides (e.g., LaTe, CeTe, PrTe, NdTe, PmTe, EuTe, GdTe, TbTe, DyTe, HoTe, ErTe, TmTe, YbTe, LuTe, etc.), and/or the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers selected from a red emission layer, a green emission layer, and a blue emission layer, in which the two or more layers contact each other or are separated from each other to emit white light (e.g., combined white light). In one or more embodiments, the emission layer may include two or more materials selected from a red light-emitting material, a green light-emitting material, and a blue light-emitting material, in which the two or more materials are mixed with each other in a single layer to emit white light (e.g., combined white light).

In one or more embodiments, the emission layer may include a host and a dopant (or emitter). The dopant may include a phosphorescent dopant, a fluorescent dopant, or any combination thereof. In one or more embodiments, the emission layer may further include an auxiliary dopant that promotes energy transfer to a dopant (or emitter), in addition to the host and the dopant (or emitter). When the emission layer includes the dopant (or emitter) and the auxiliary dopant, the dopant (or emitter) and the auxiliary dopant are different from each other.

An amount (weight) of the dopant (or emitter) in the emission layer may be in a range of about 0.01 parts by weight to about 15 parts by weight based on 100 parts by weight of the host.

In one or more embodiments, the emission layer may include a quantum dot.

In one or more embodiments, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within these ranges, excellent or suitable light-emission characteristics may be obtained without a substantial increase in driving voltage.

### Host

In one or more embodiments, the host may include a compound represented by Formula 301:

[Ar₃₀₁]_{xb11}-[(L₃₀₁)_{xb1}-R₃₀₁]_{xb21}, Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁) (Q₃₀₂)(Q₃₀₃), —N(Q₃₀₁) (Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each the same as described with respect to Q₁.

For example, in some embodiments, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

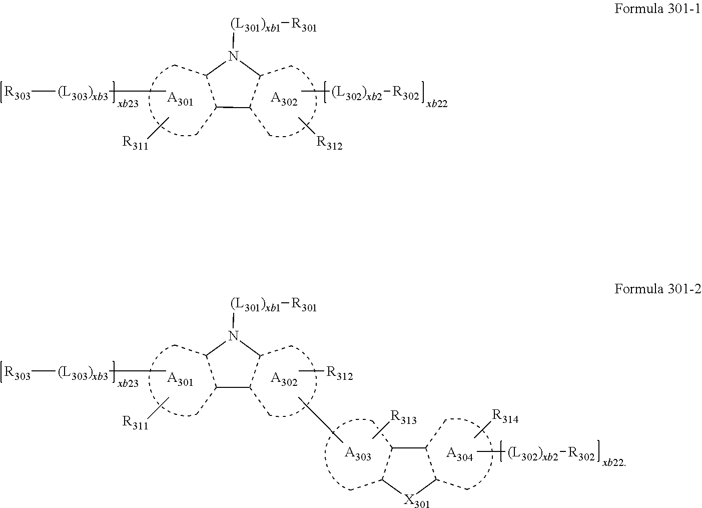

In Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described herein,
L₃₀₂ to L₃₀₄ may each independently be the same as described herein with respect to L₃₀₁,
xb2 to xb4 may each independently be the same as described with respect to xb1, and
R₃₀₂ to R₃₀₅ and R₃₁₁ to R₃₁₄ may each be the same as described herein with respect to R₃₀₁.

In one or more embodiments, the host may include an alkaline earth metal complex, a post-transition metal complex, or any combination thereof. For example, in some embodiments, the host may include a Be complex (for example, Compound H55), an Mg complex, a Zn complex, or any combination thereof.

In one or more embodiments, the host may include at least one selected from Compounds H1 to H130, 9,10-di(2-naphthyl)anthracene (ADN), 2-methyl-9,10-bis(naphthalen-2-yl)anthracene (MADN), 9,10-di(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl)benzene (TCP), and/or any combination thereof:

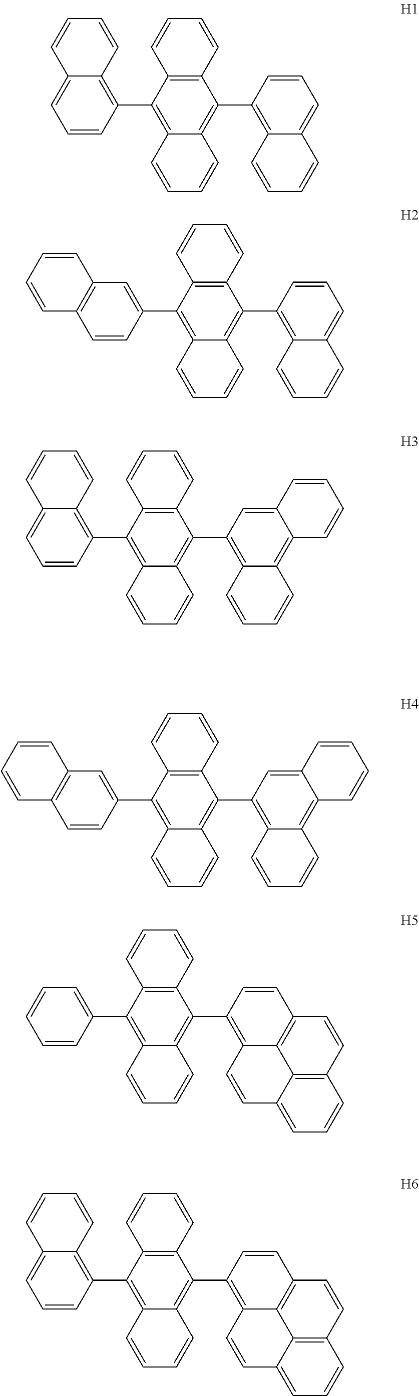

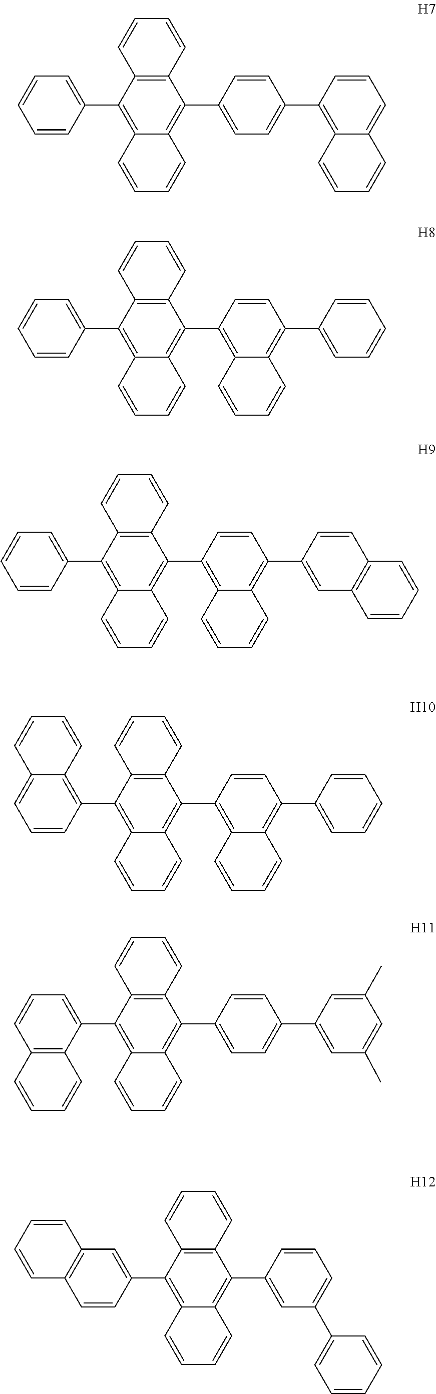

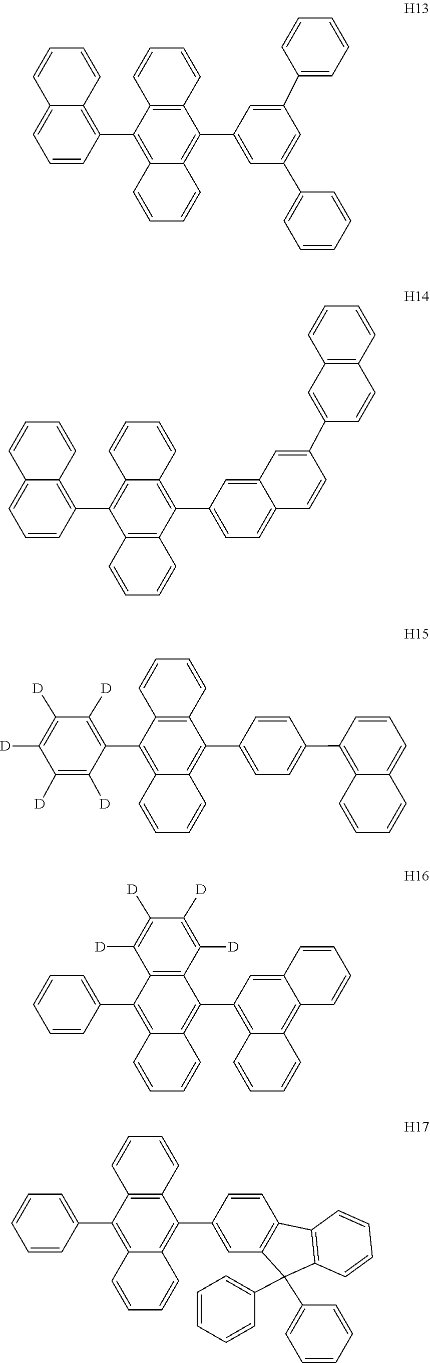

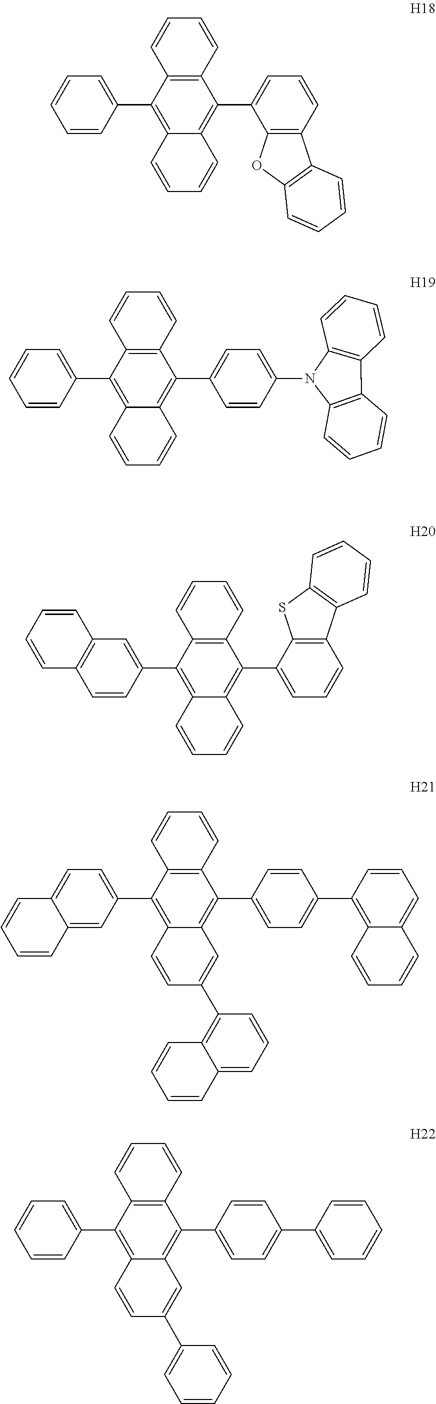

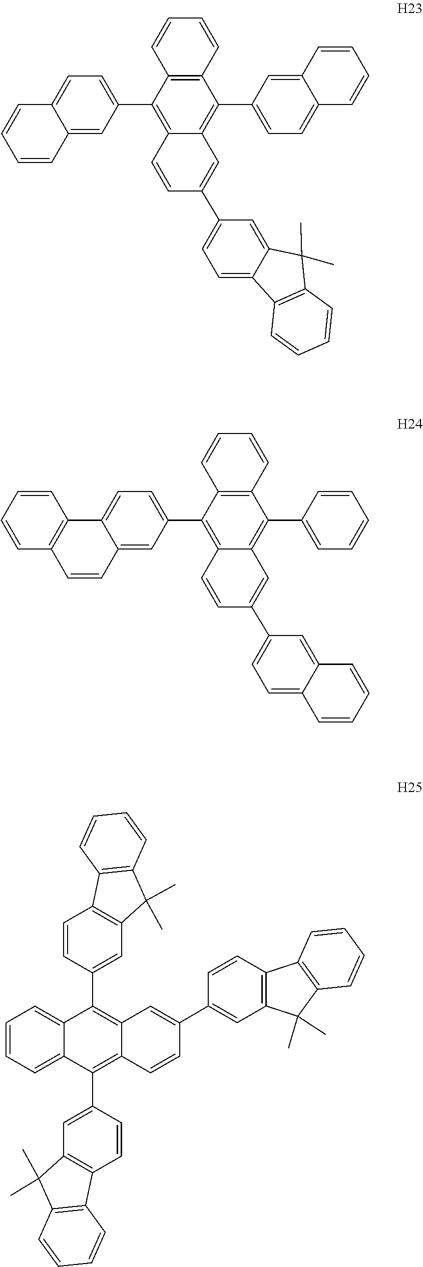

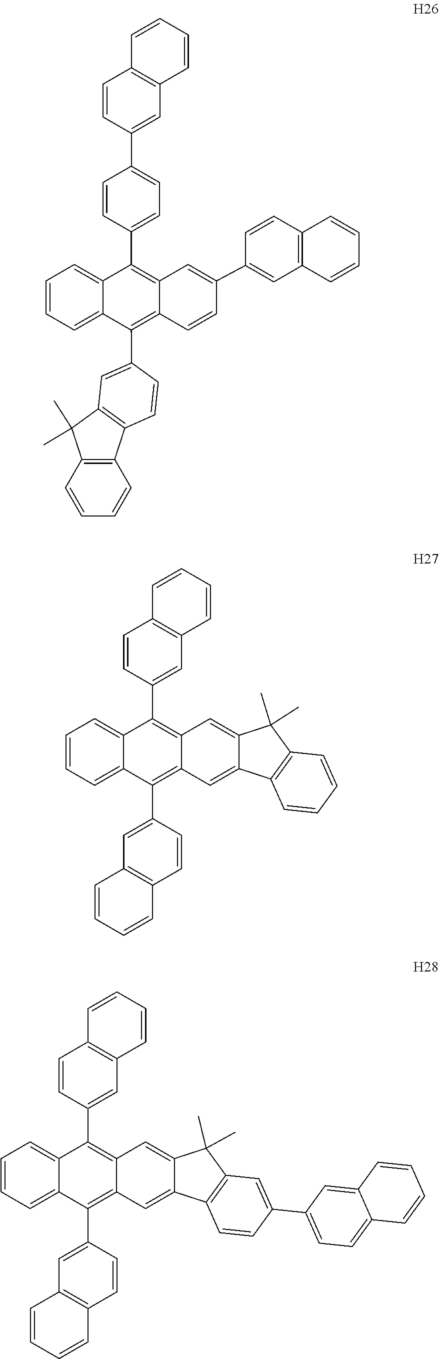

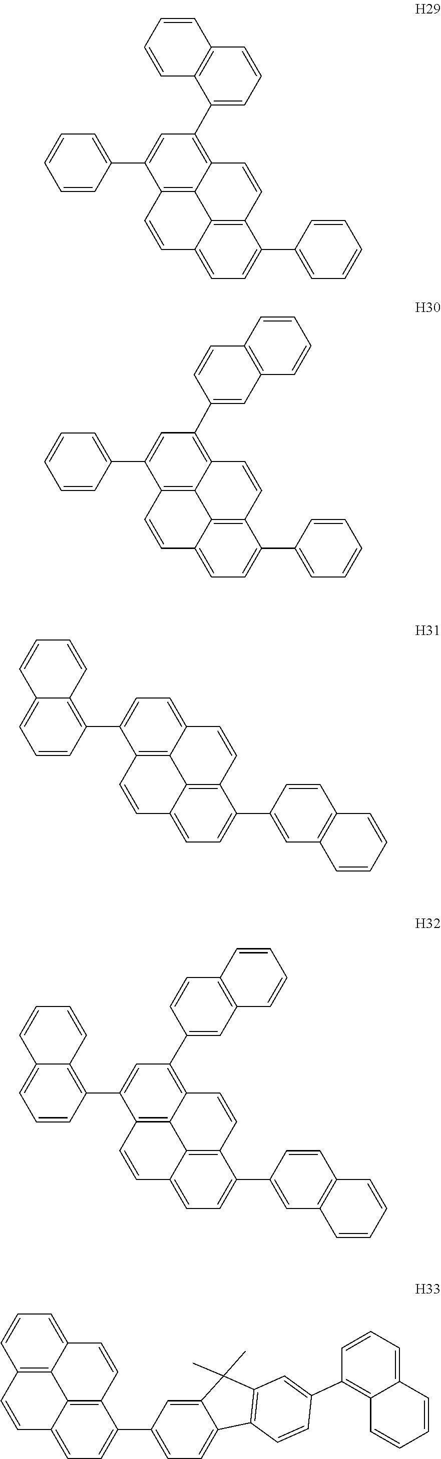

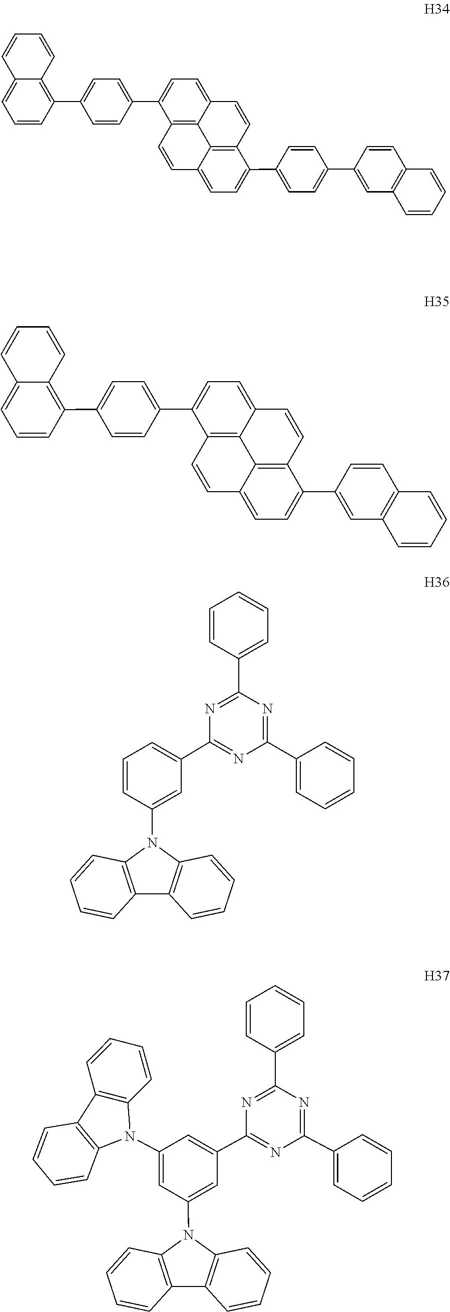

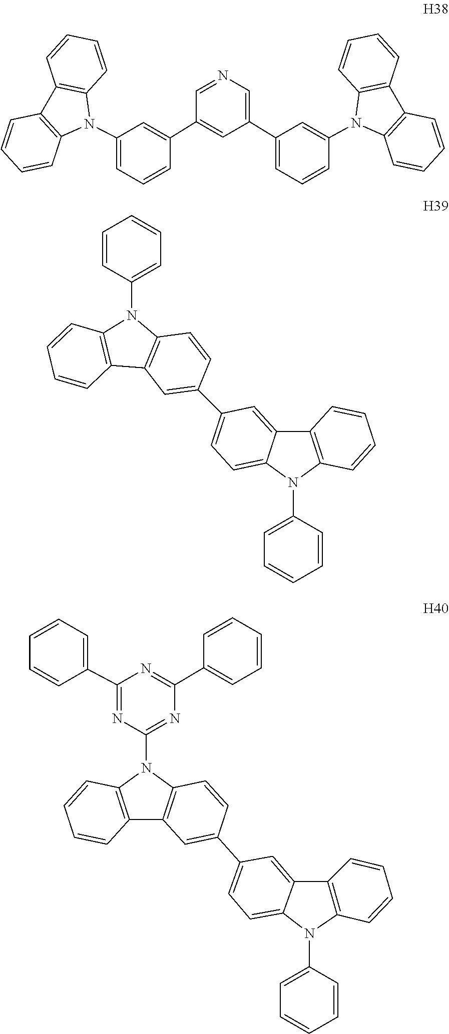

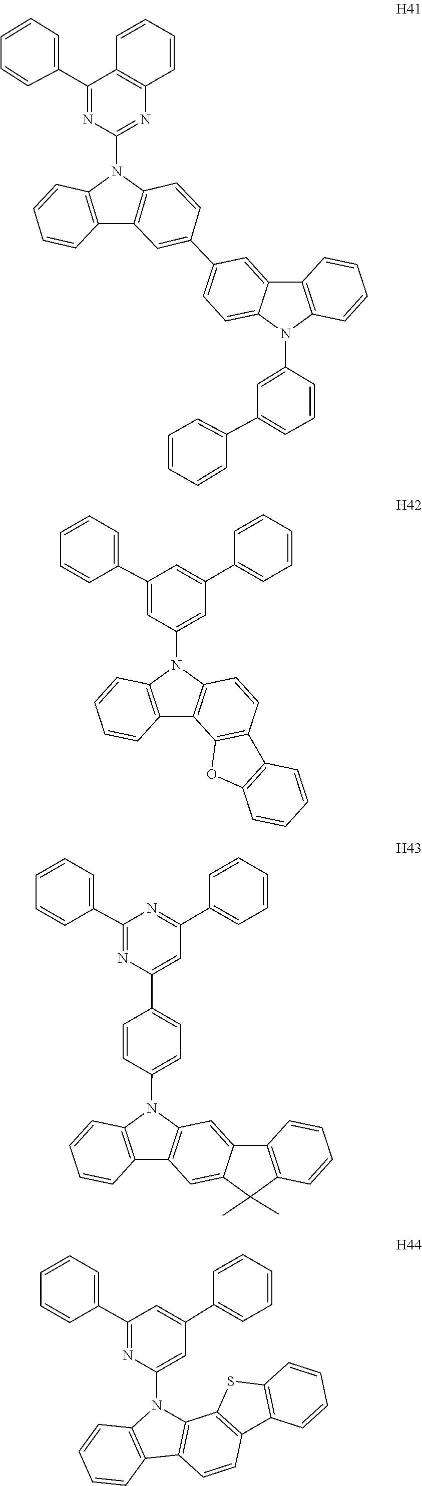

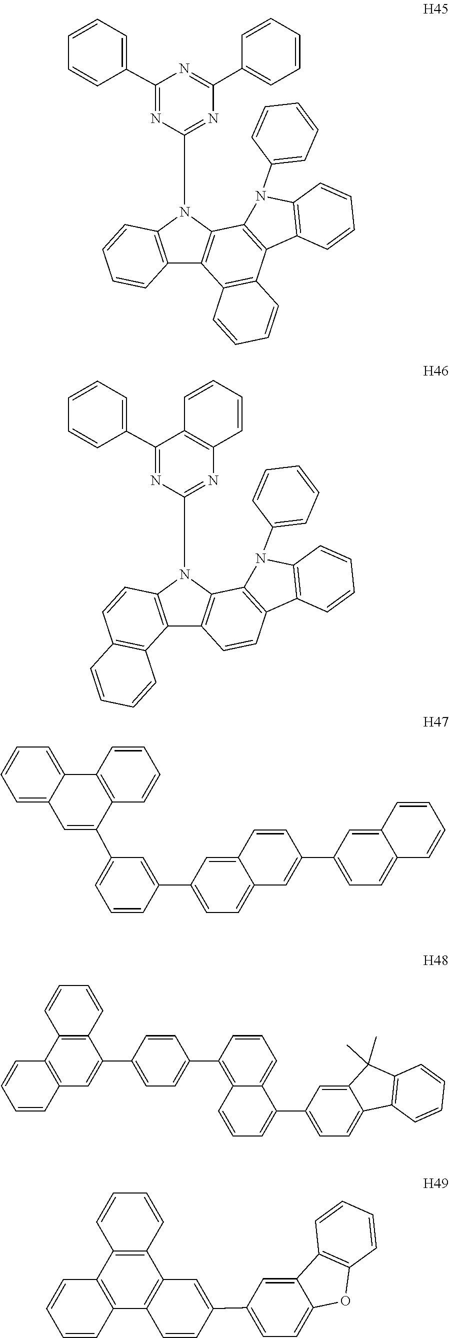

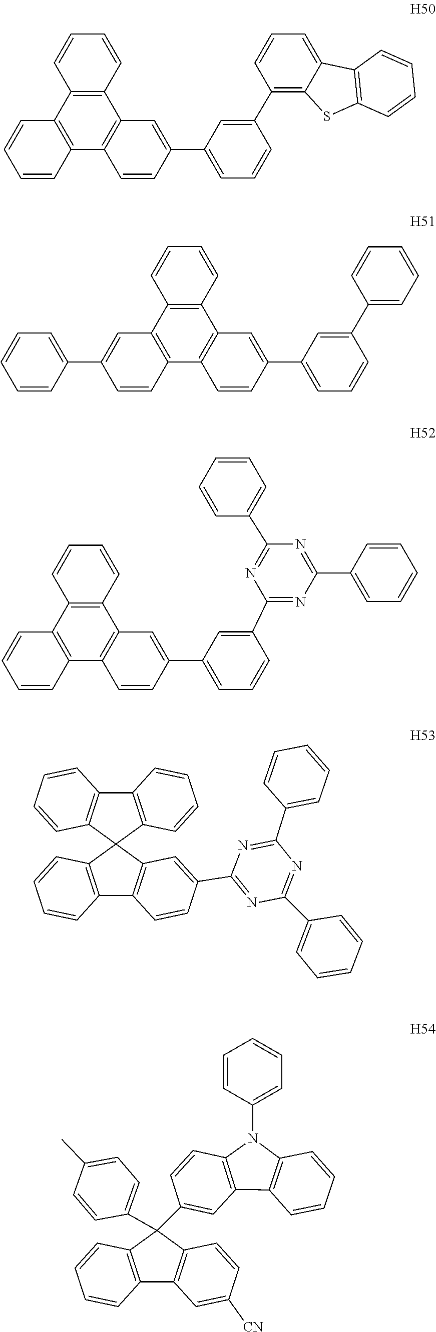

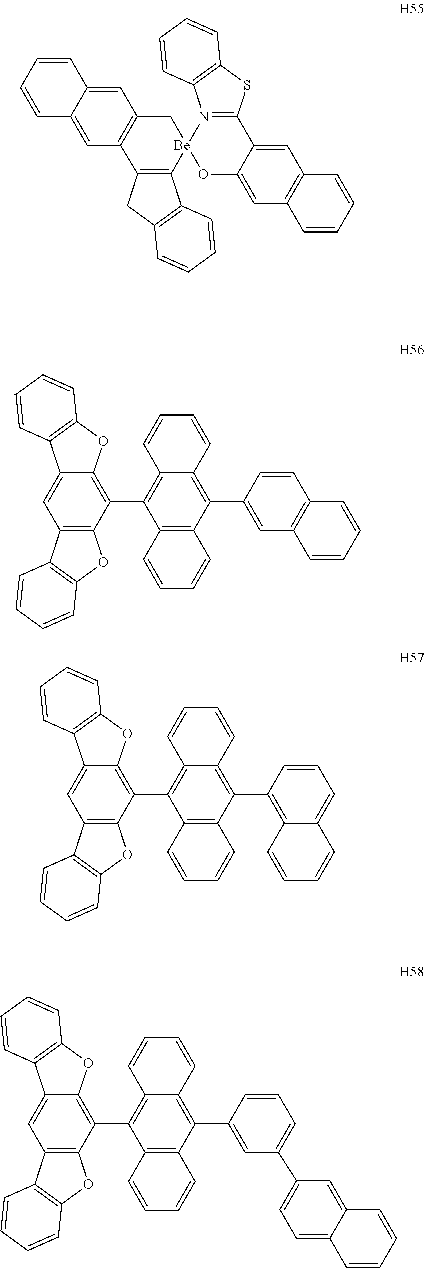

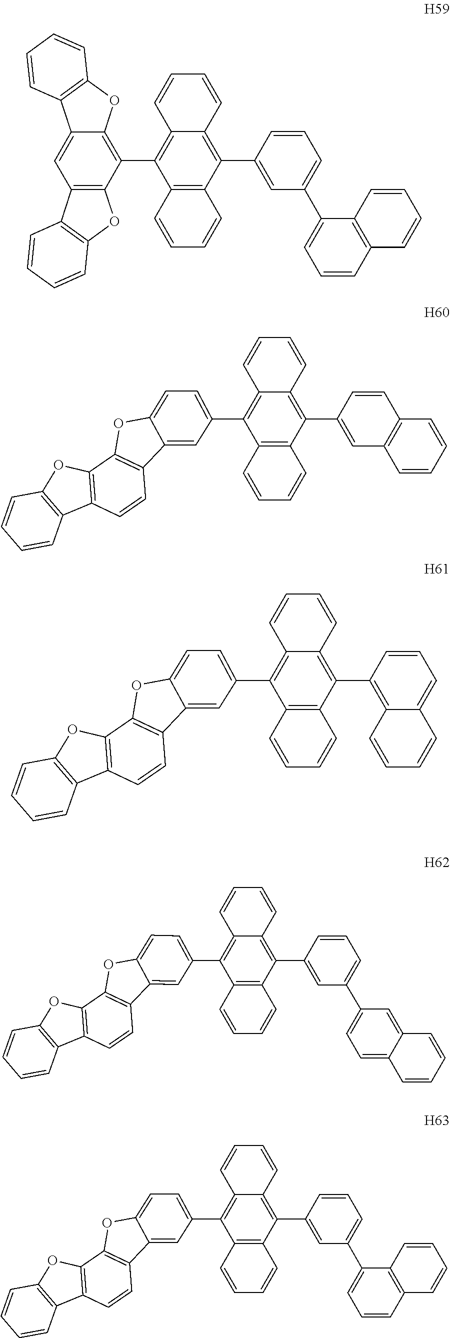 [SMILES: c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1]

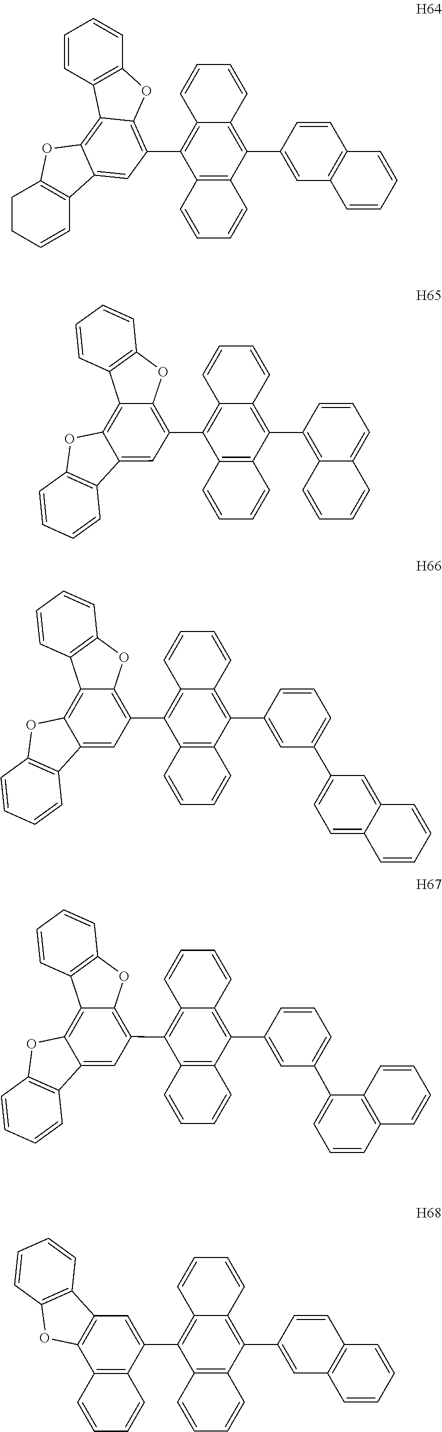 [SMILES: C1=Cc2c(oc3c2cc(-c2c4ccccc4c(-c4ccc5ccccc5c4)c4ccccc24)c2oc4ccccc4c23)CC1 ; c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1]

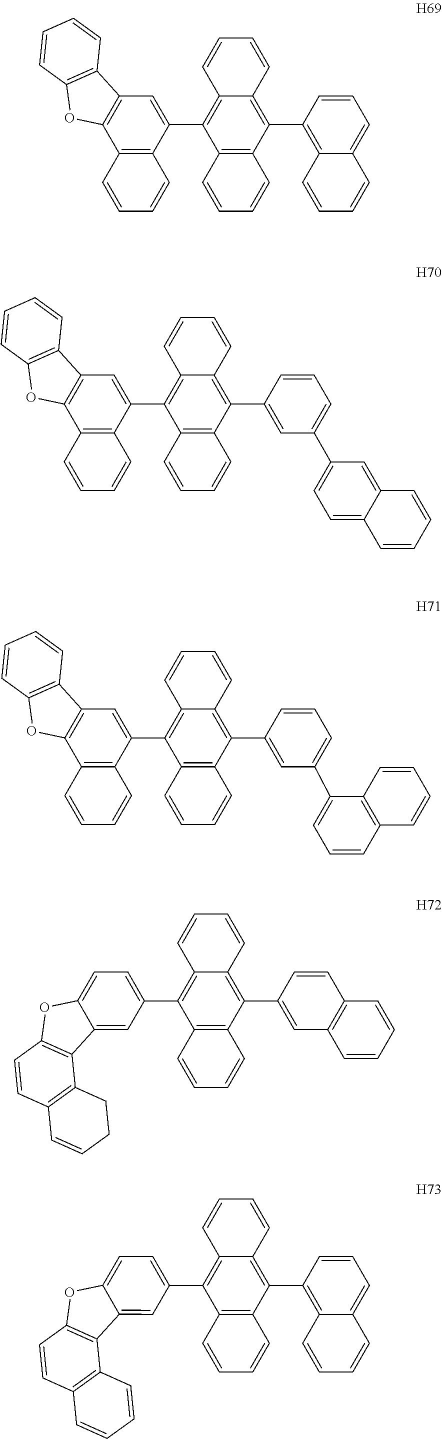 [SMILES: c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; C1=Cc2ccc3oc4ccc(-c5c6ccccc6c(-c6ccc7ccccc7c6)c6ccccc56)cc4c3c2CC1 ; c1ccc2c(-c3c4ccccc4c(-c4ccc5oc6ccc7ccccc7c6c5c4)c4ccccc34)cccc2c1]

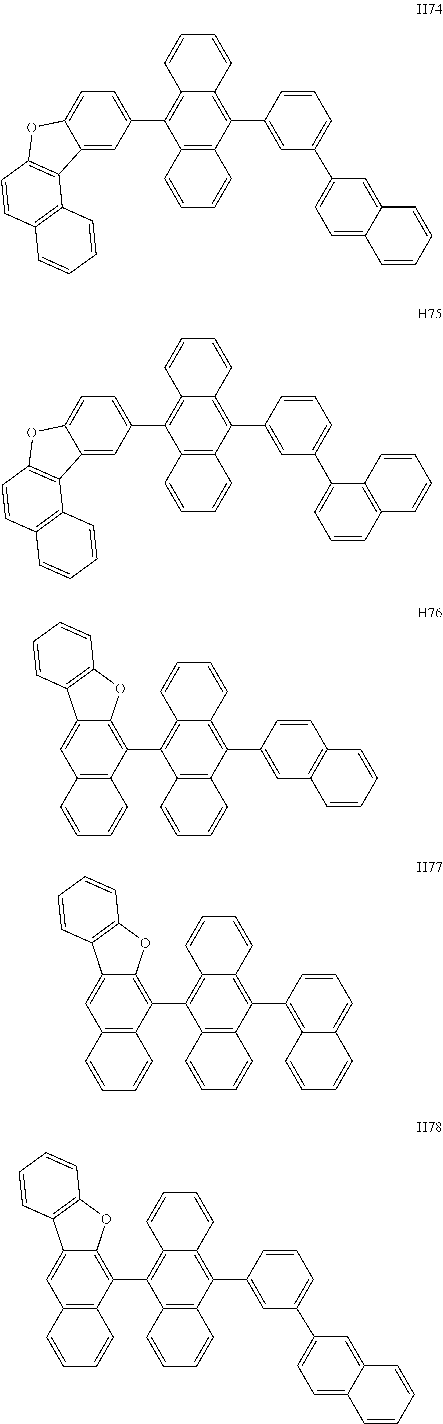

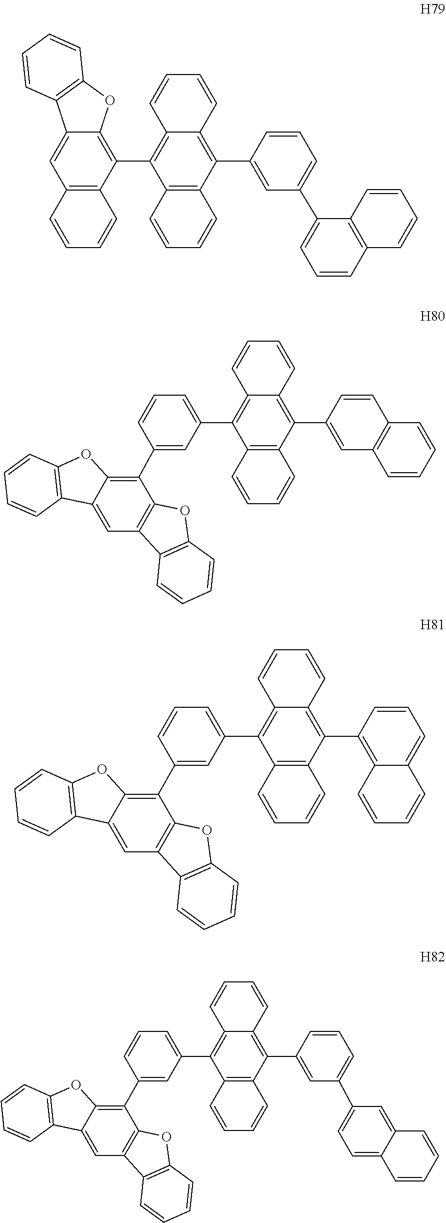 [SMILES: c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1]

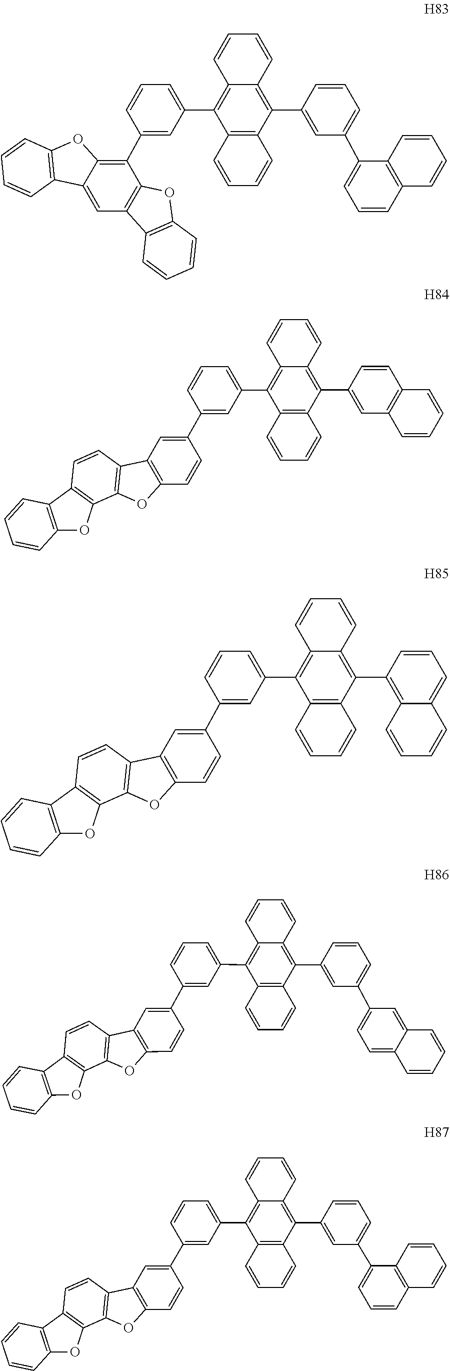 [SMILES: c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1]

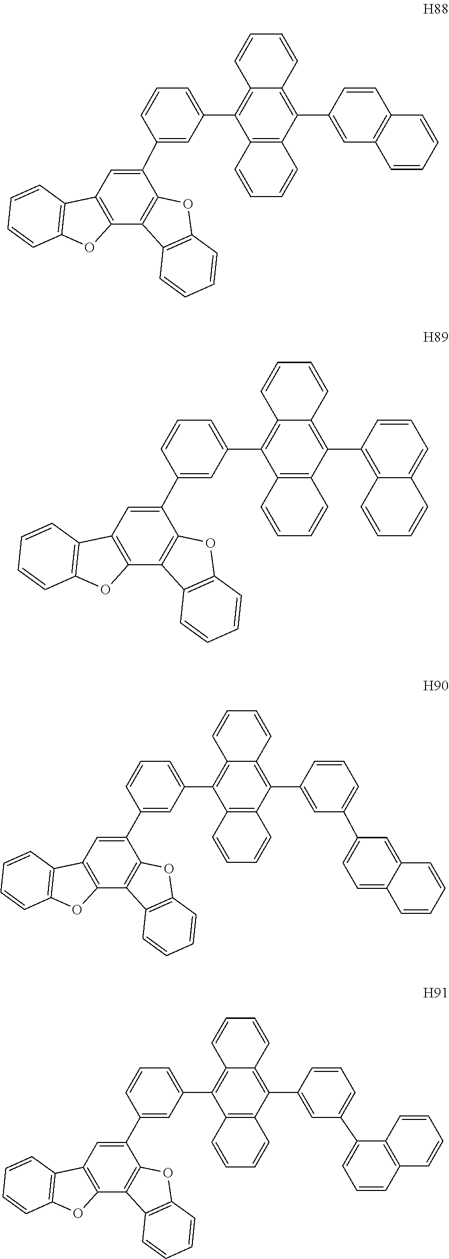 [SMILES: c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1]

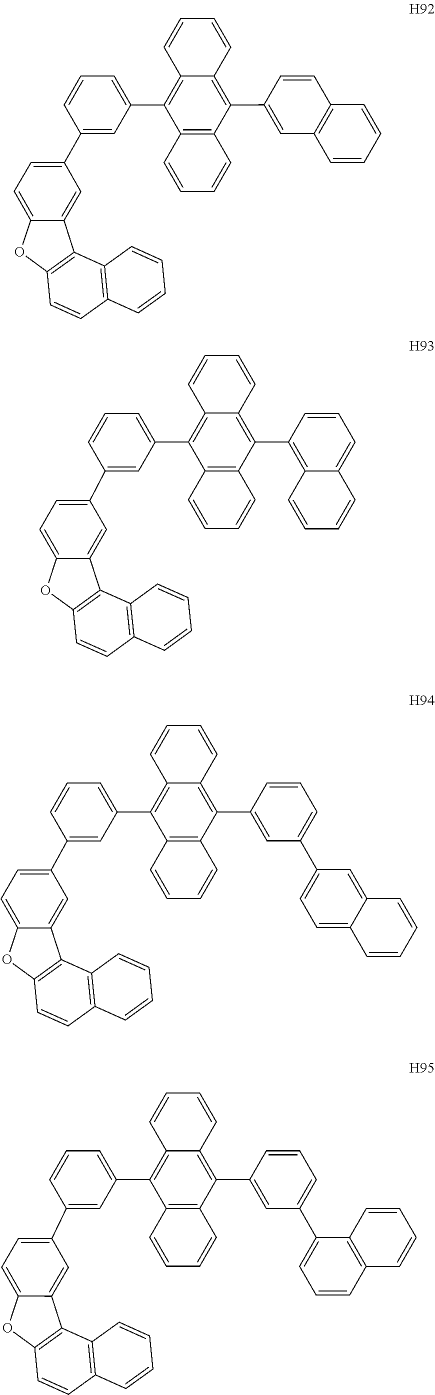 [SMILES: c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1]

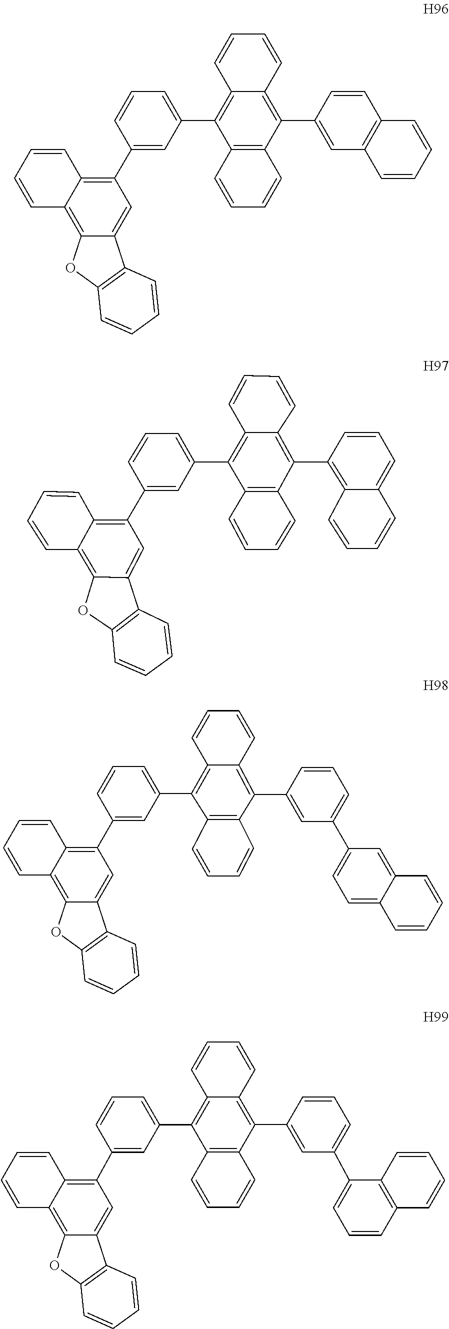 [SMILES: c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1]

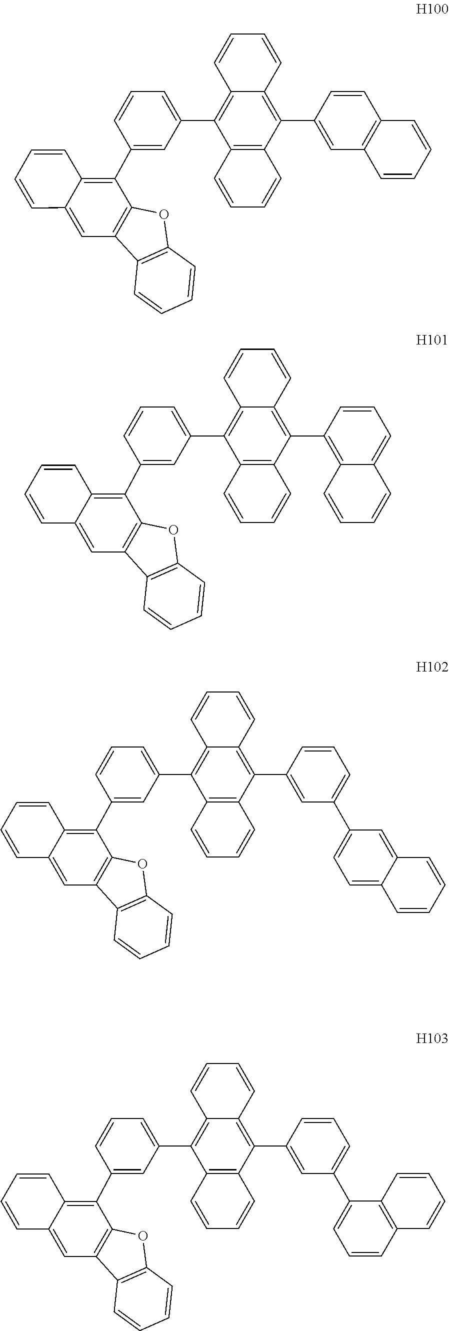 [SMILES: c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

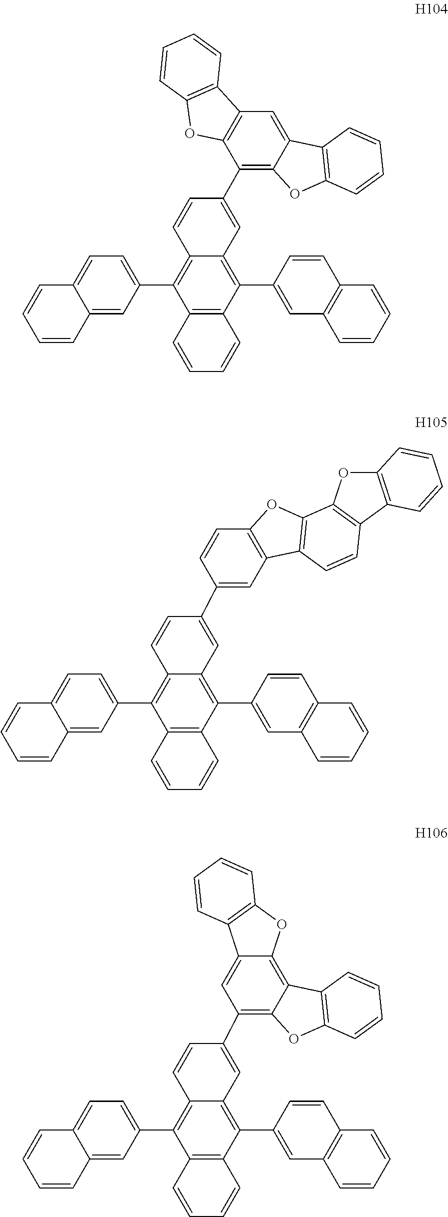 [SMILES: c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1]

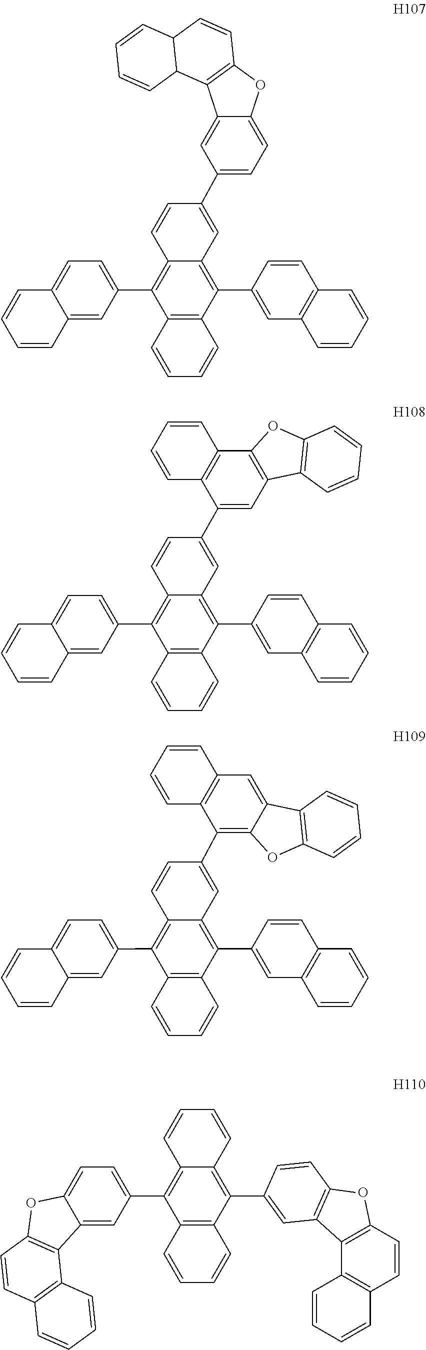

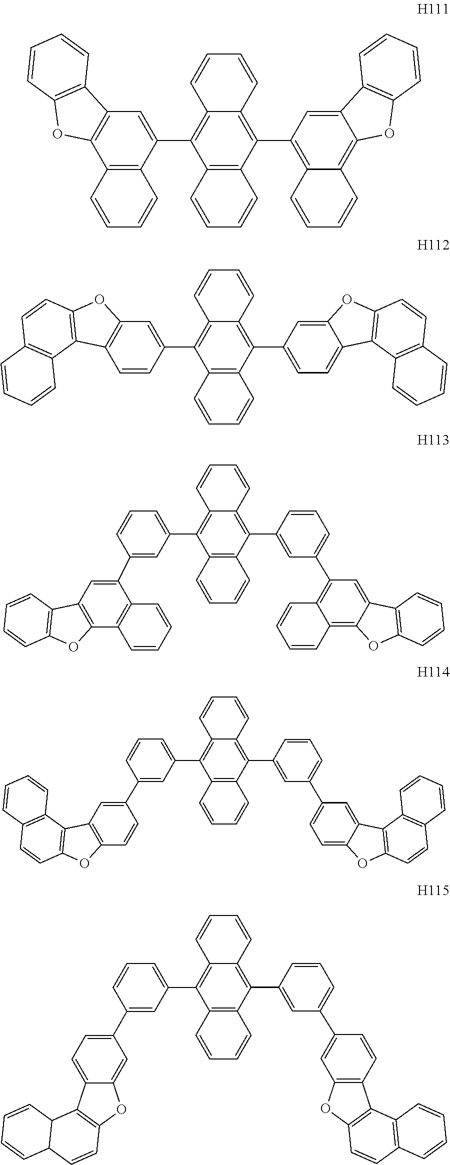 [SMILES: c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; C1=CC2C=Cc3oc4cc(-c5cccc(-c6c7ccccc7c(-c7cccc(-c8ccc9c(c8)oc8ccc%10ccccc%10c89)c7)c7ccccc67)c5)ccc4c3C2C=C1]

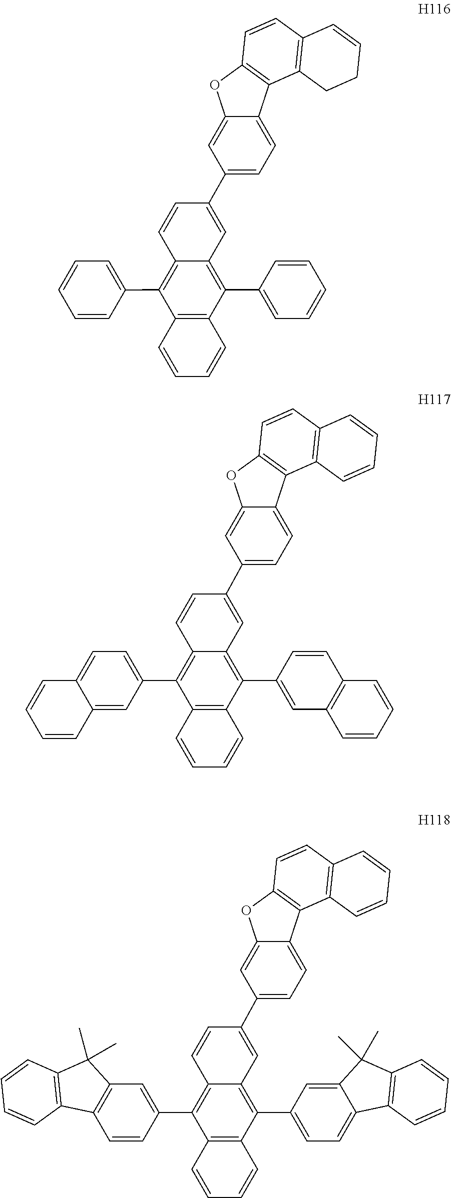 [SMILES: C1=Cc2ccc3oc4cc(-c5ccc6c(-c7ccccc7)c7ccccc7c(-c7ccccc7)c6c5)ccc4c3c2CC1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

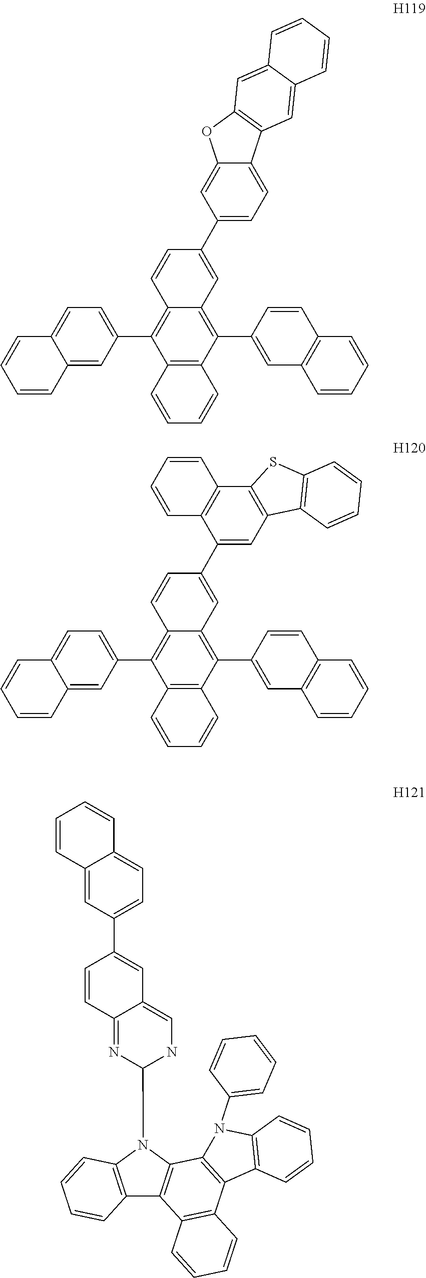

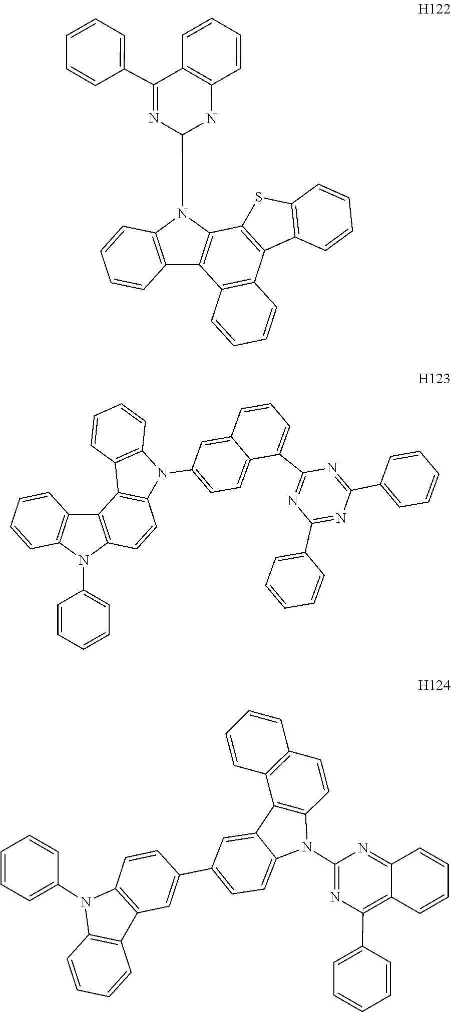

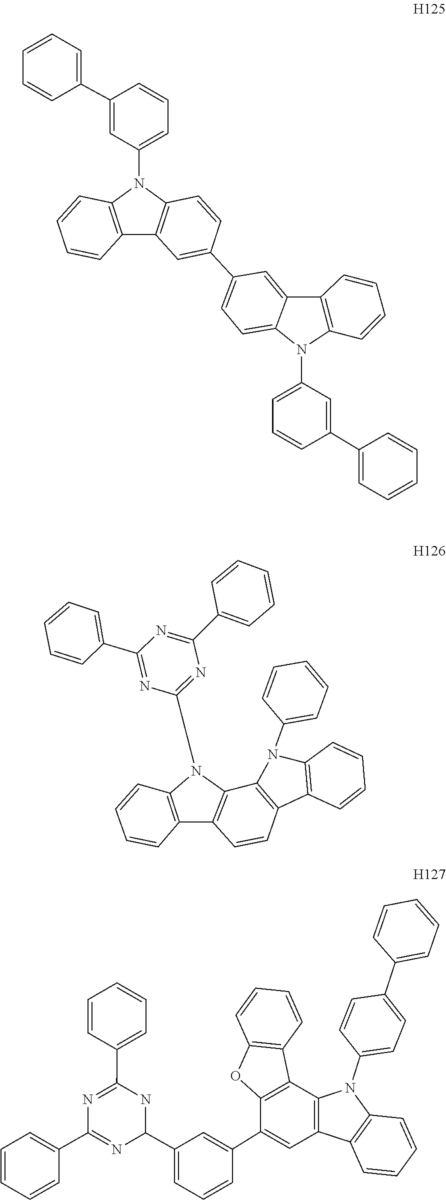

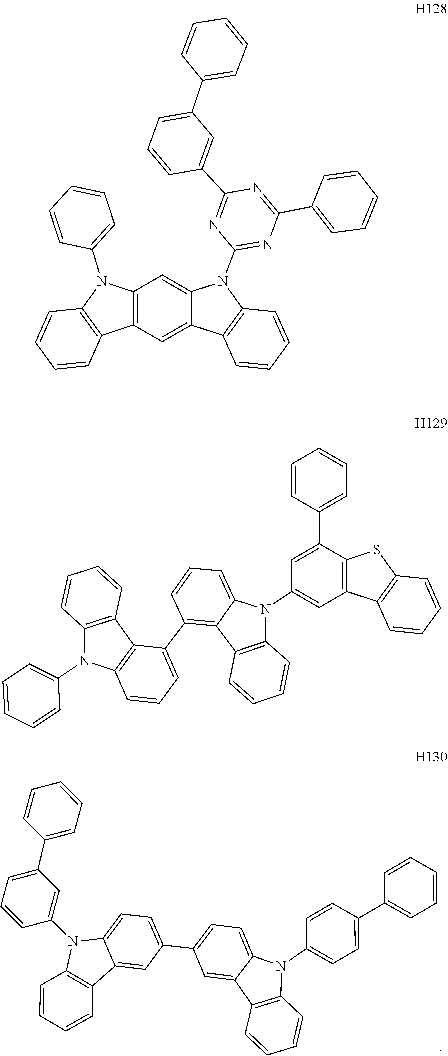 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

In one or more embodiments, the host may include a silicon-containing compound, a phosphine oxide-containing compound, or any combination thereof.

The host may have one or more suitable modifications. For example, the host may include only one kind of compound, or may include two or more kinds of different compounds.

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

In some embodiments, the phosphorescent dopant may be electrically neutral.

For example, in one or more embodiments, the phosphorescent dopant may include an organometallic compound represented by Formula 401:

M(L₄₀₁)_{xc1}(L₄₀₂)_{xc2} Formula 401

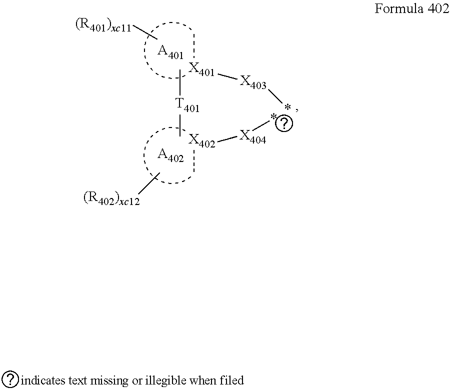

wherein, in Formulae 401 and 402,
M may be a transition metal (e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, when xc1 is 2 or more, two or more of L₄₀₁(s) may be identical to or different from each other,
L₄₀₂ may be an organic ligand, and xc2 may be 0, 1, 2, 3, or 4, and when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*, *—S—**, *—C(═O)—*, *—N(Q₄₁₁)-*, *—C(Q₄₁₁)(Q₄₁₂)-**, *—C(Q₄₁₁)=C(Q₄₁₂)-**, *—C(Q₄₁₁)=**, or *═C(Q₄₁₁)-**,
X₄₀₃ and X₄₀₄ may each independently be a chemical bond (e.g., a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be the same as described with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be the same as described with respect to Q₁,
xc11 and xc12 may each independently be an integer from 0 to 10, and
* and *′ in Formula 402 each indicate a binding site to M in Formula 401.

For example, in some embodiments, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two of ring A₄₀₁(s) among two or more of L₄₀₁(s) may be optionally linked to each other via T₄₀₂, which is a linking group, and/or two of ring A₄₀₂(s) among two or more of L₄₀₁(s) may be optionally linked to each other via T₄₀₃, which is a linking group (see Compounds PD1 to PD4 and PD7). T₄₀₂ and T₄₀₃ may each be the same as described with respect to T₄₀₁.

In Formula 401, L₄₀₂ may be an organic ligand. For example, L₄₀₂ may include a halogen, a diketone group (e.g., an acetylacetonate group), a carboxylic acid group (e.g., a picolinate group), —C(═O), an isonitrile group, a —CN group, a phosphorus-containing group (e.g., a phosphine group, a phosphite group, etc.), or any combination thereof.

In one or more embodiments, the phosphorescent dopant may include, for example, one selected from compounds PD1 to PD39, and/or any combination thereof:

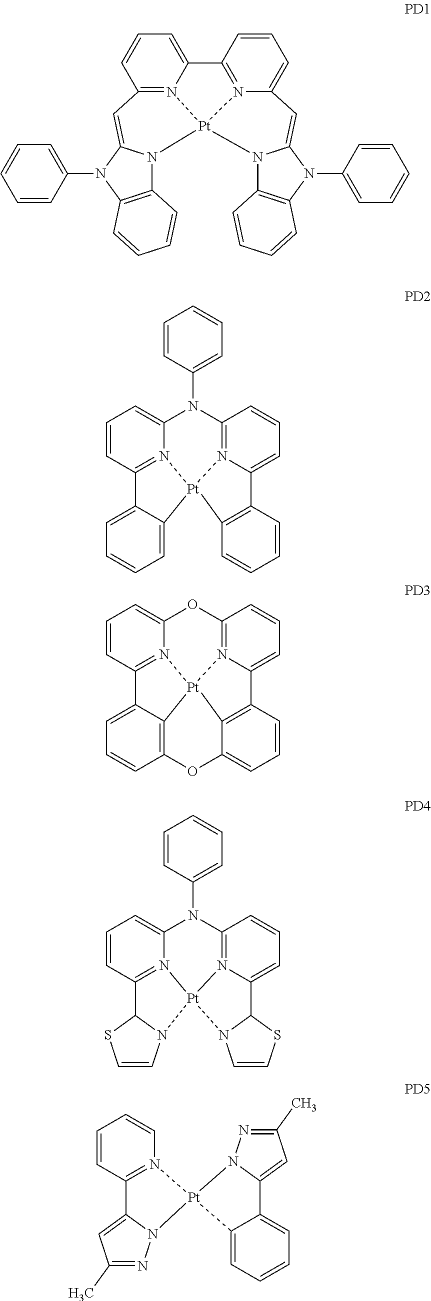

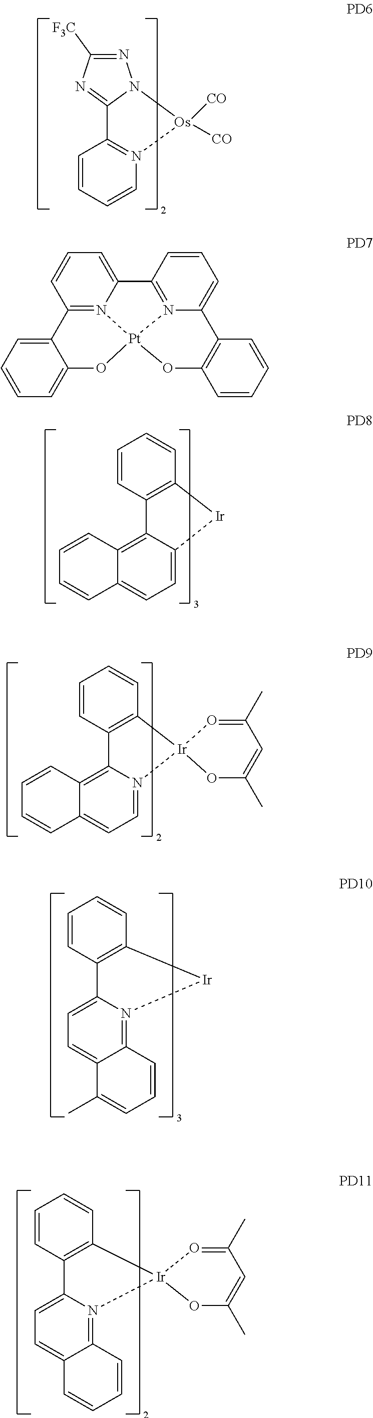

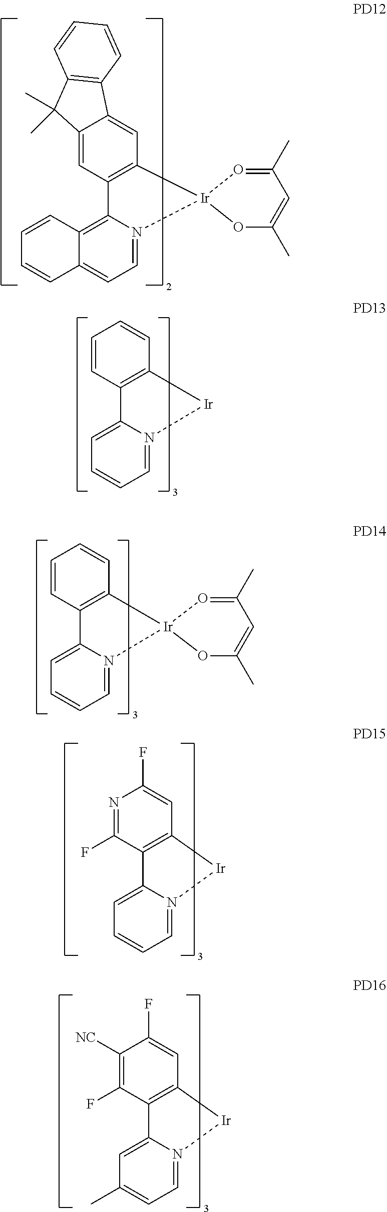

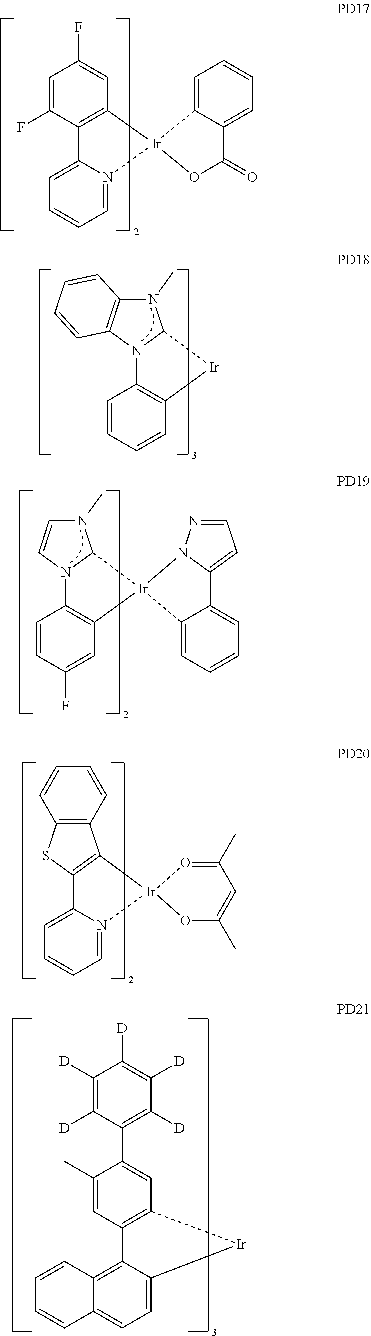

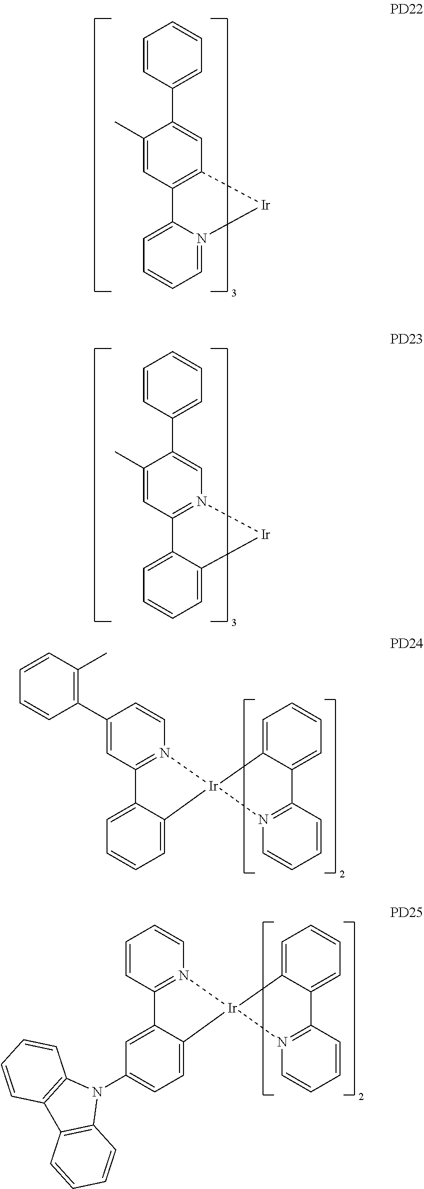

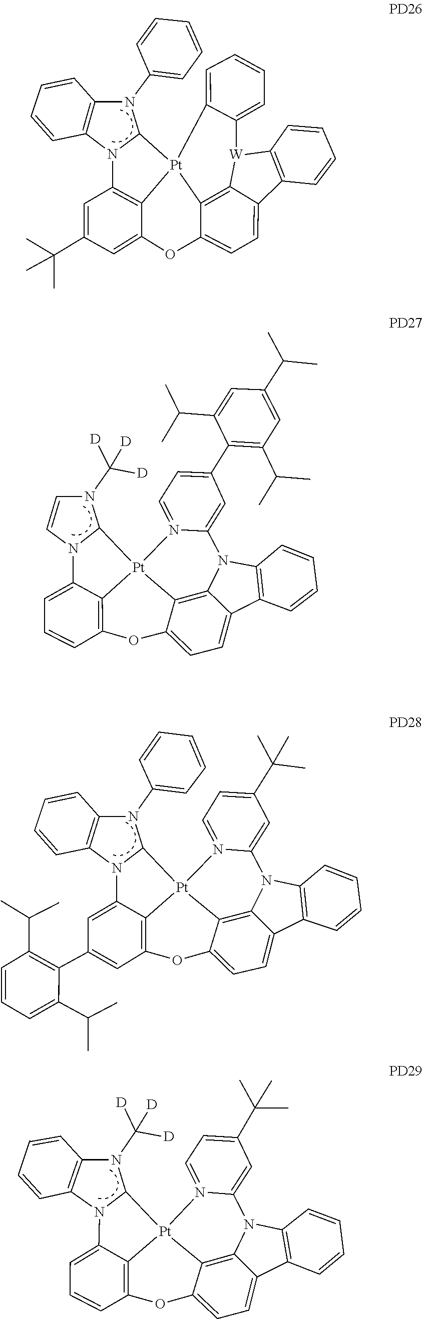

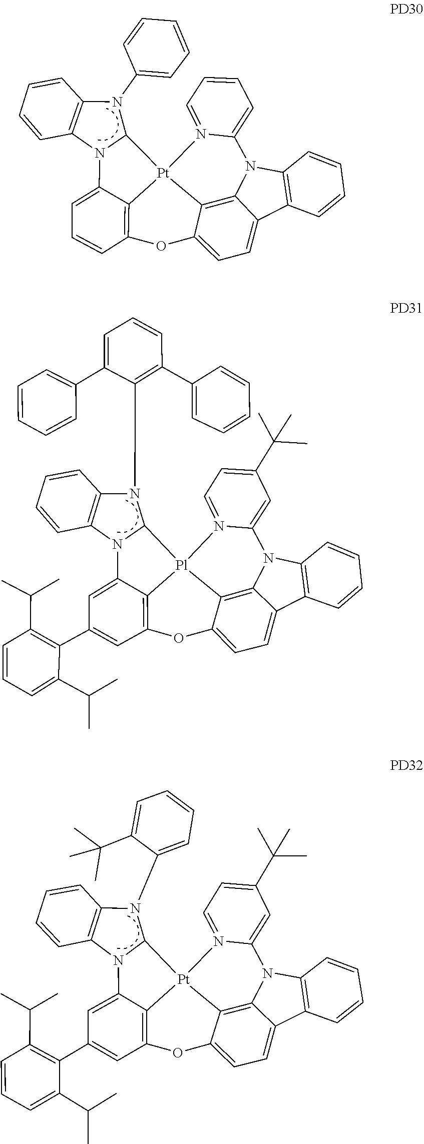

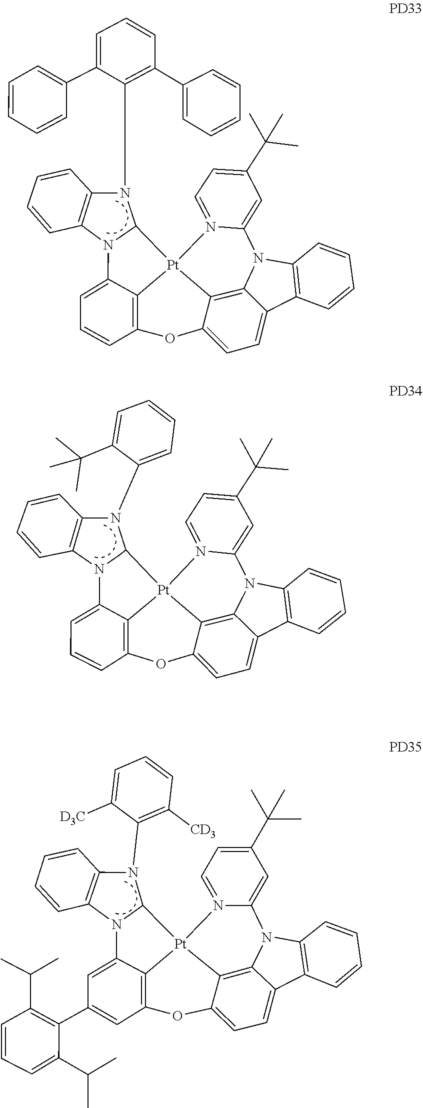

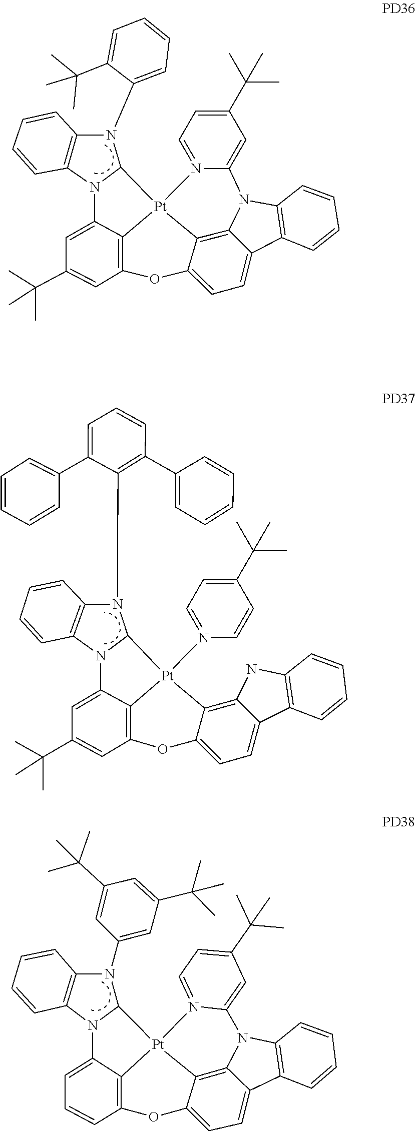

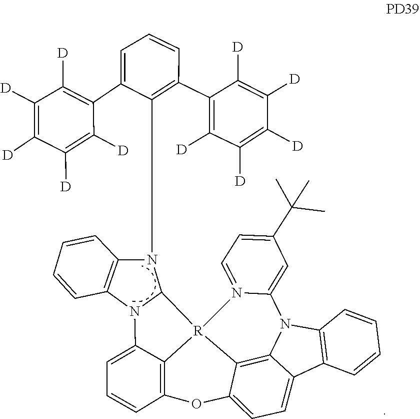

### Fluorescent Dopant

The fluorescent dopant may include an amine group-containing compound, a styryl group-containing compound, or any combination thereof.

For example, in one or more embodiments, the fluorescent dopant may include a compound represented by Formula 501:

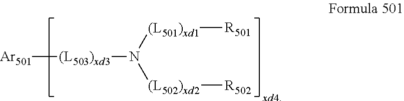

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, in some embodiments, Ar₅₀₁ in Formula 501 may be a condensed cyclic group (e.g., an anthracene group, a chrysene group, a pyrene group, etc.) in which three or more monocyclic groups are condensed together.

For example, in some embodiments, xd4 in Formula 501 may be 2.

For example, in some embodiments, the fluorescent dopant and the auxiliary dopant may each include at least one selected from Compounds FD1 to FD37, 4,4′-bis(2,2-diphenylvinyl)-1,1′-biphenyl (DPVBi), 4,4′-bis[4-(N,N-diphenylamino)styryl]biphenyl (DPAVBi), and/or any combination thereof:

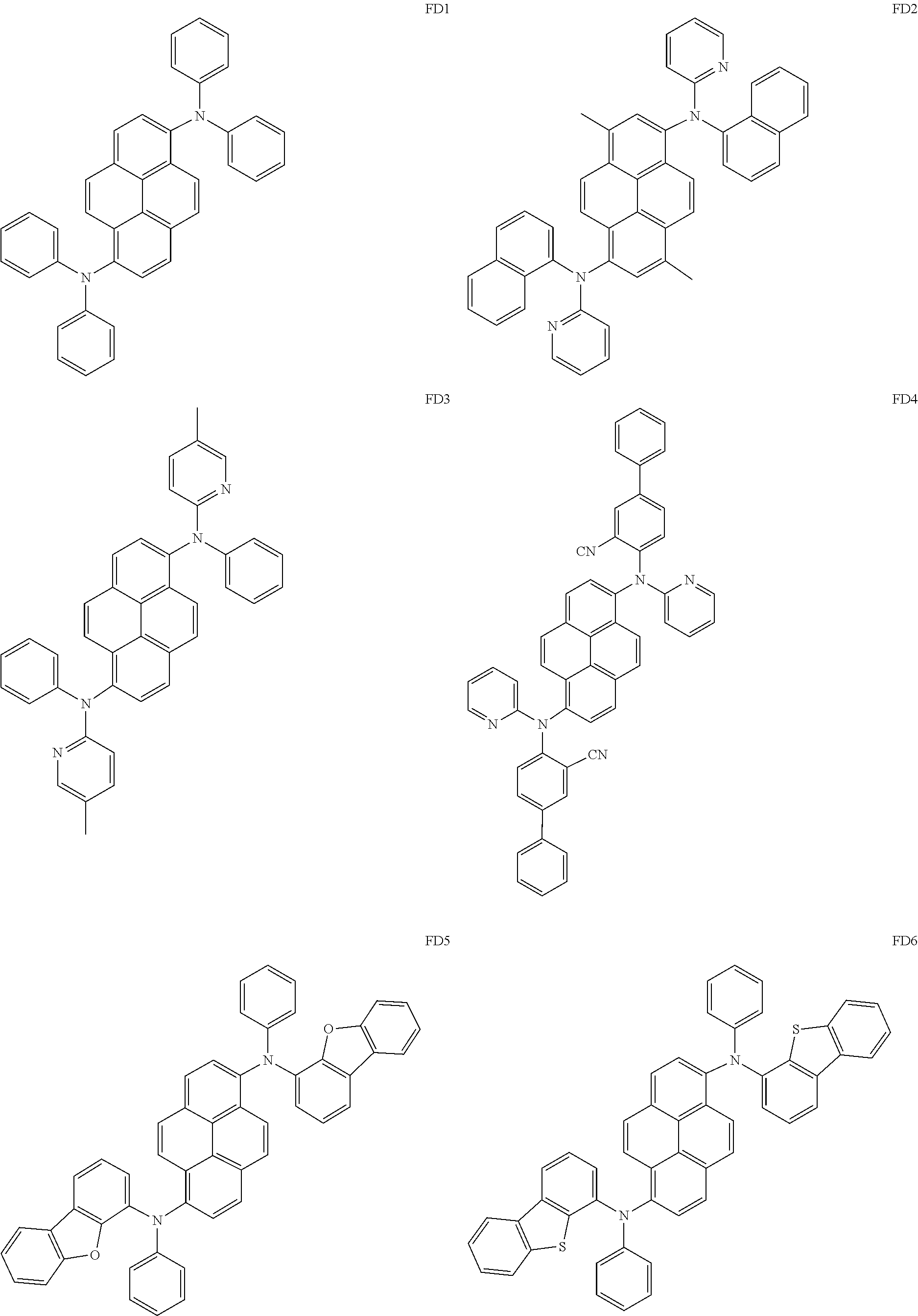

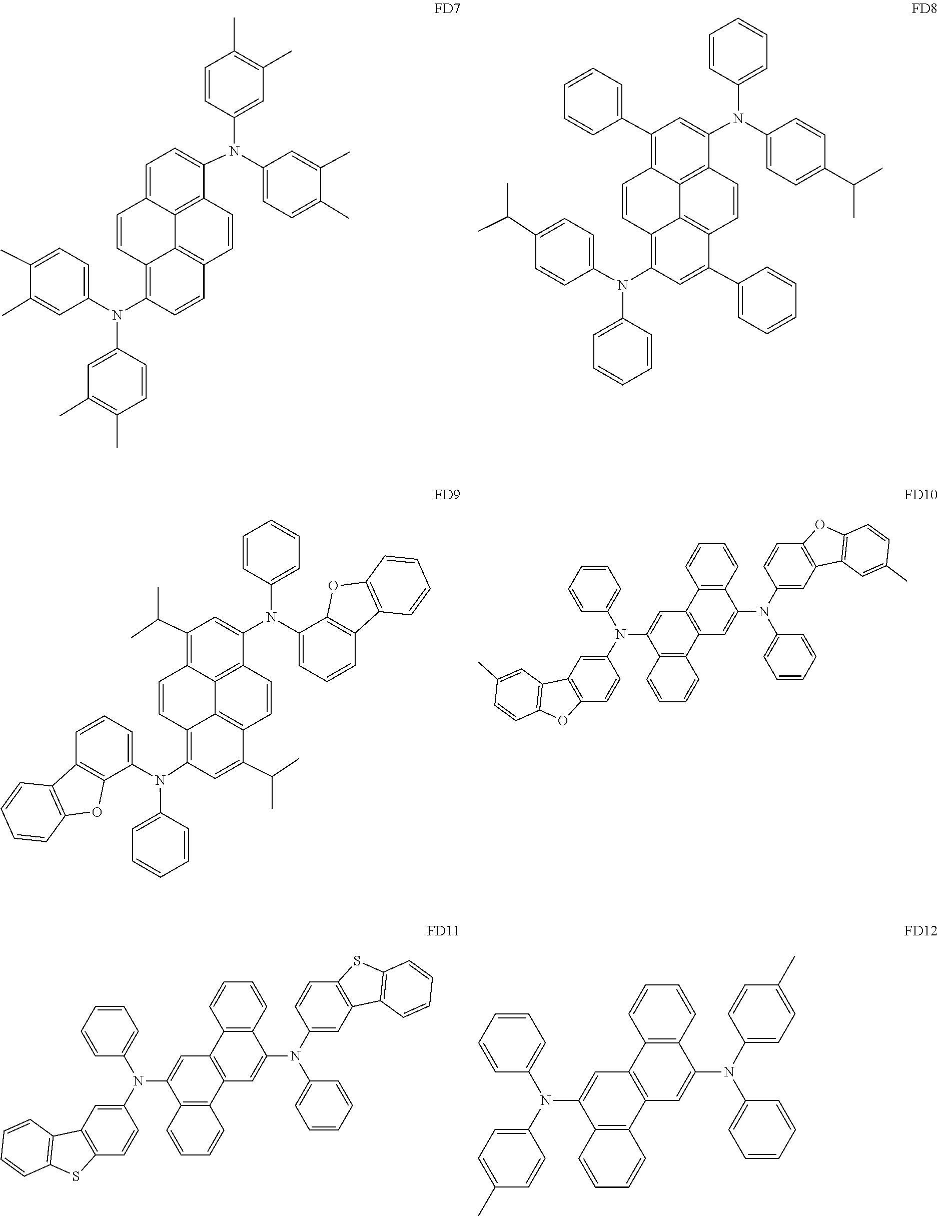

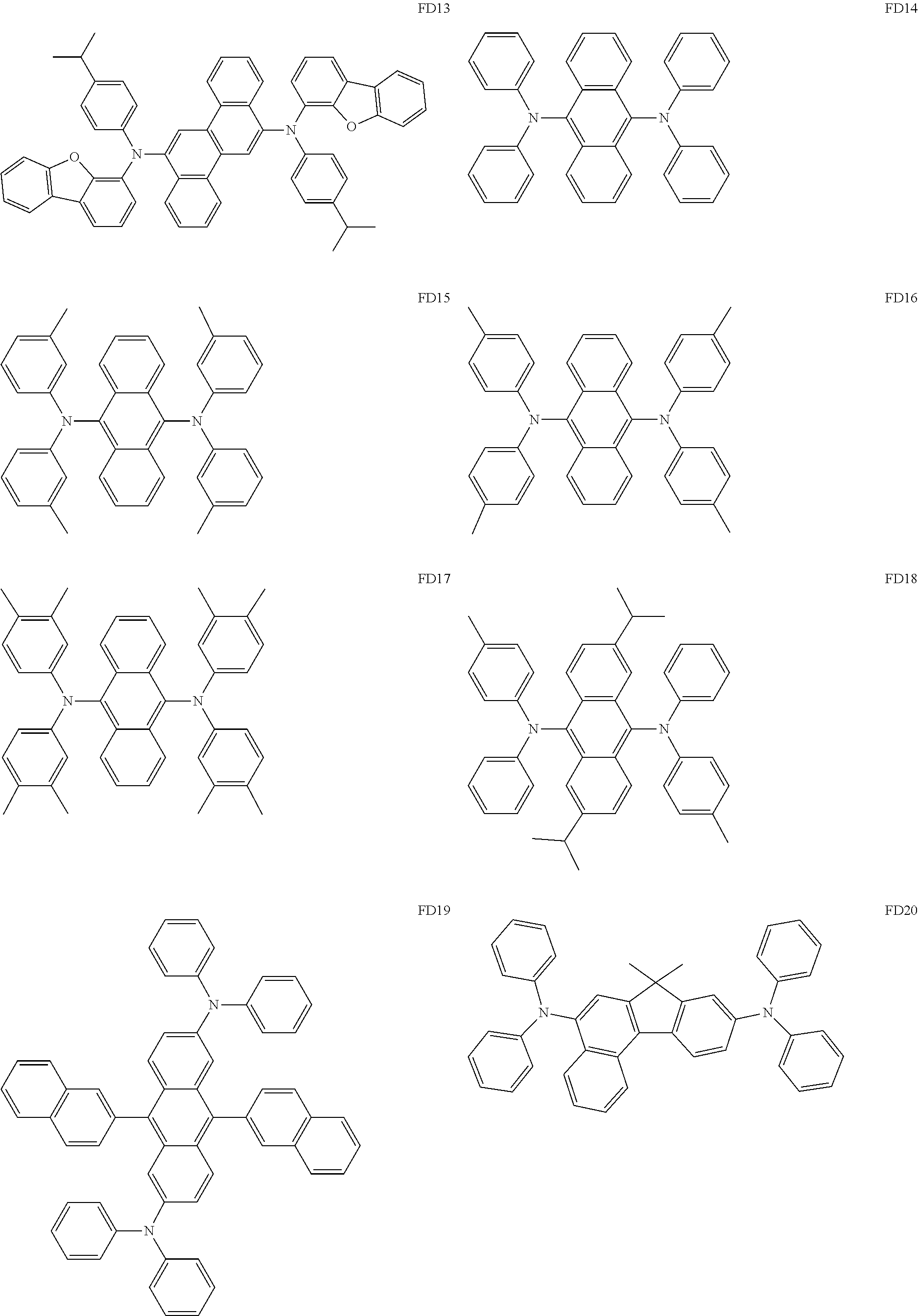

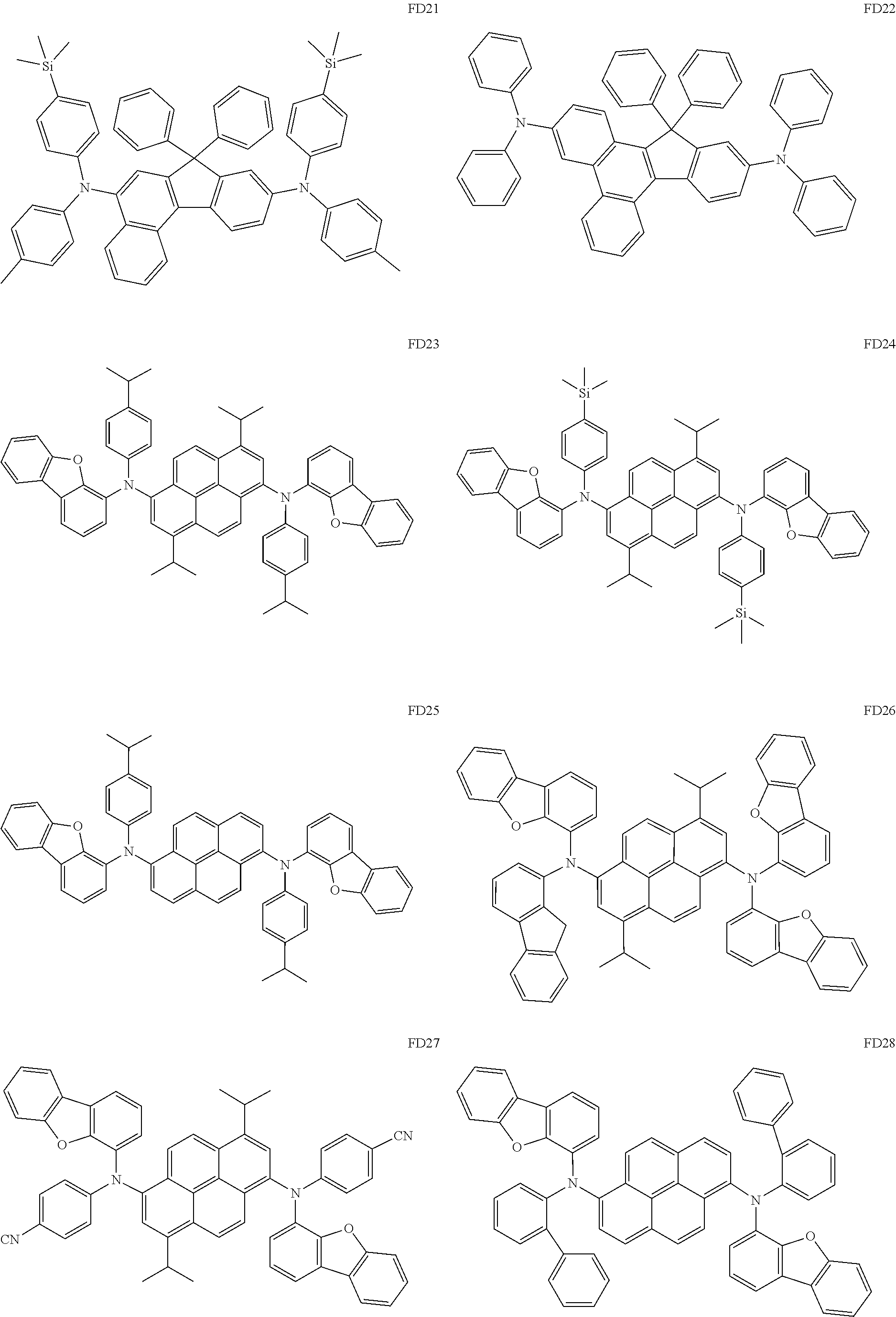

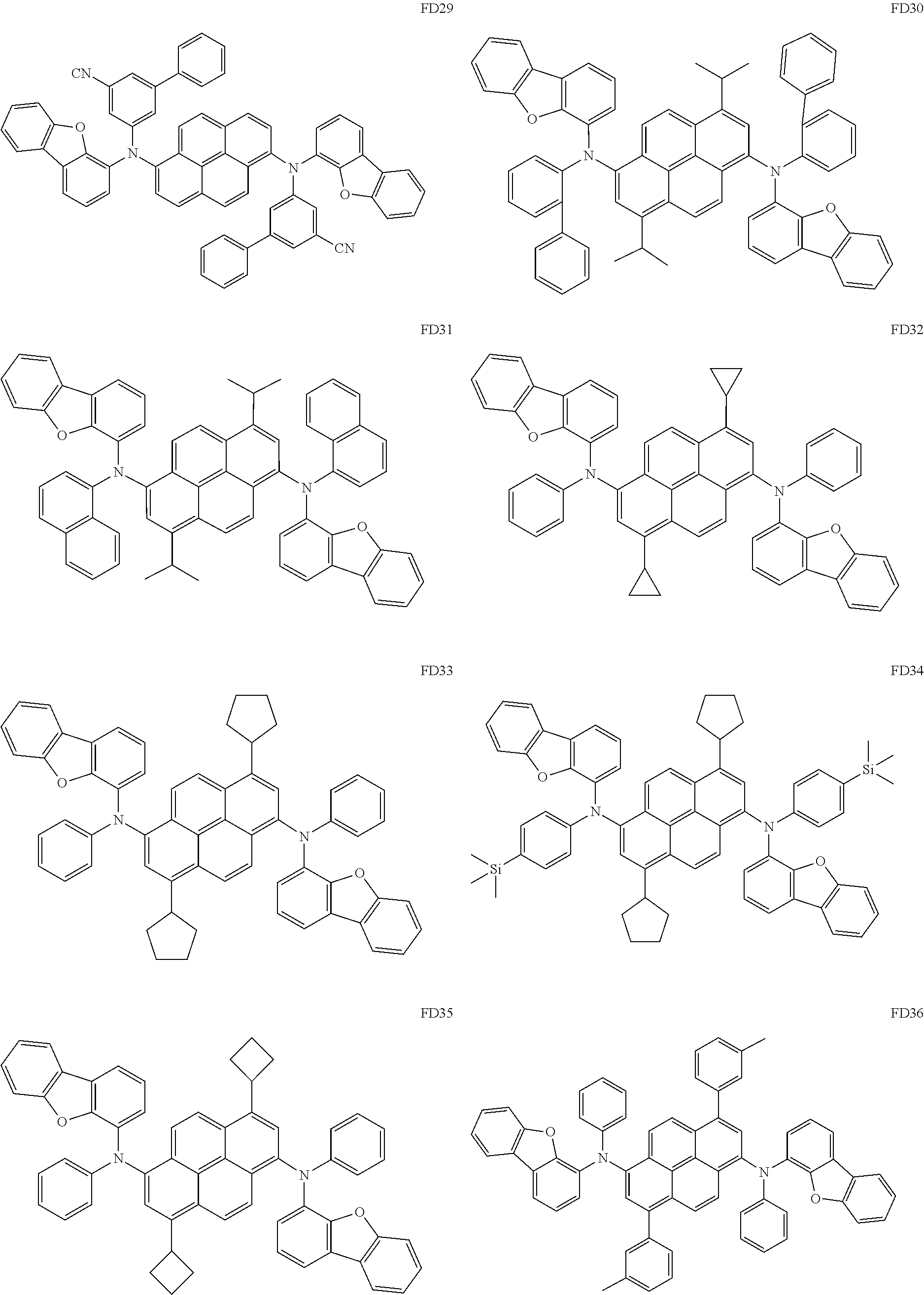

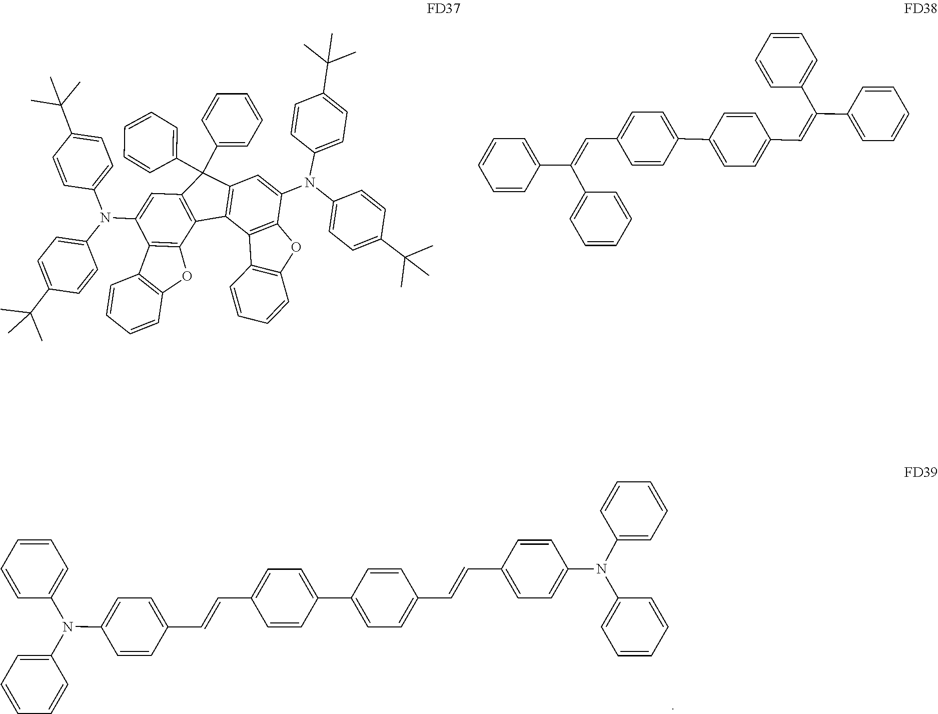

### Delayed Fluorescence Material

In one or more embodiments, the emission layer may include a delayed fluorescence material.

In the present disclosure, the delayed fluorescence material may be selected from compounds capable of emitting delayed fluorescent light based on a delayed fluorescence emission mechanism.

The delayed fluorescence material included in the emission layer may act as a host or a dopant depending on the type or kind of other materials included in the emission layer.

In one or more embodiments, a difference between a triplet energy level (eV) of the delayed fluorescence material and a singlet energy level (eV) of the delayed fluorescence material may be greater than or equal to 0 eV and less than or equal to 0.5 eV. When the difference between the triplet energy level of the delayed fluorescence material and the singlet energy level of the delayed fluorescence material is satisfied with the ranges above, up-conversion from the triplet state to the singlet state of the delayed fluorescence materials may effectively occur, and thus, the luminescence efficiency and/or the like of the light-emitting device 10 may be improved.

For example, in one or more embodiments, the delayed fluorescence material may include i) a material including at least one electron donor (e.g., a TT electron-rich C₃-C₆₀ cyclic group, such as a carbazole group, etc.) and at least one electron acceptor (e.g., a sulfoxide group, a cyano group, a TT electron-deficient nitrogen-containing C₁-C₆₀ cyclic group, etc.), and/or ii) a material including a C₈-C₆₀ polycyclic group in which two or more cyclic groups are condensed while sharing boron (B).

Non-limiting examples of the delayed fluorescence material may include at least one selected from Compounds DF1 to DF14:

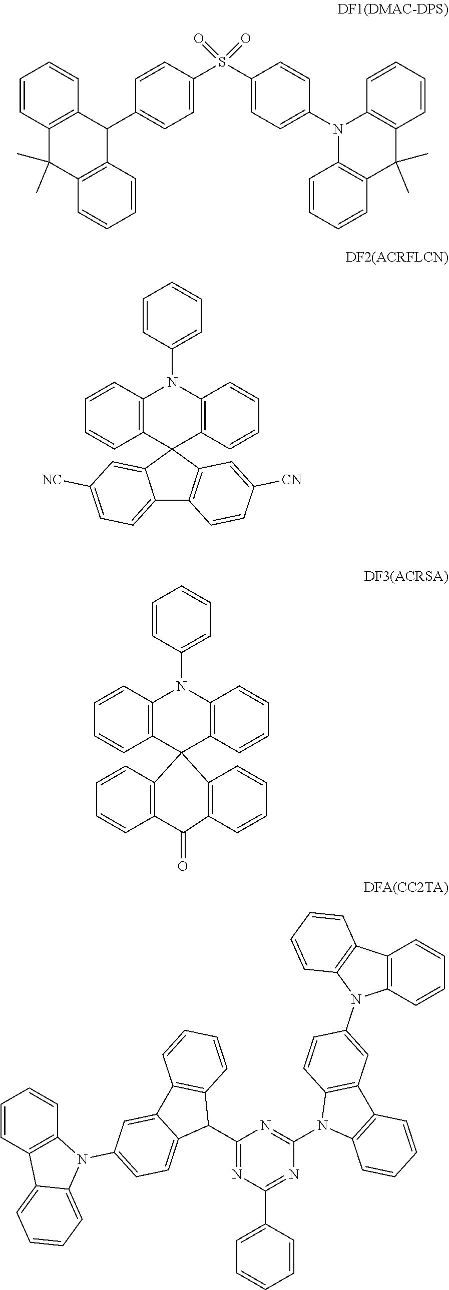

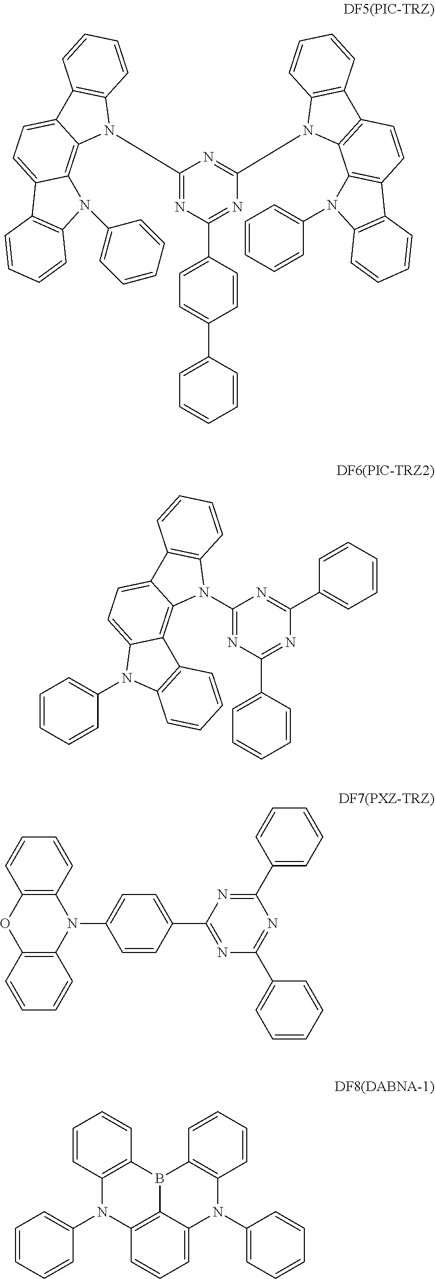 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1 ; c1ccc(N2c3ccccc3B3c4ccccc4N(c4ccccc4)c4cccc2c43)cc1]

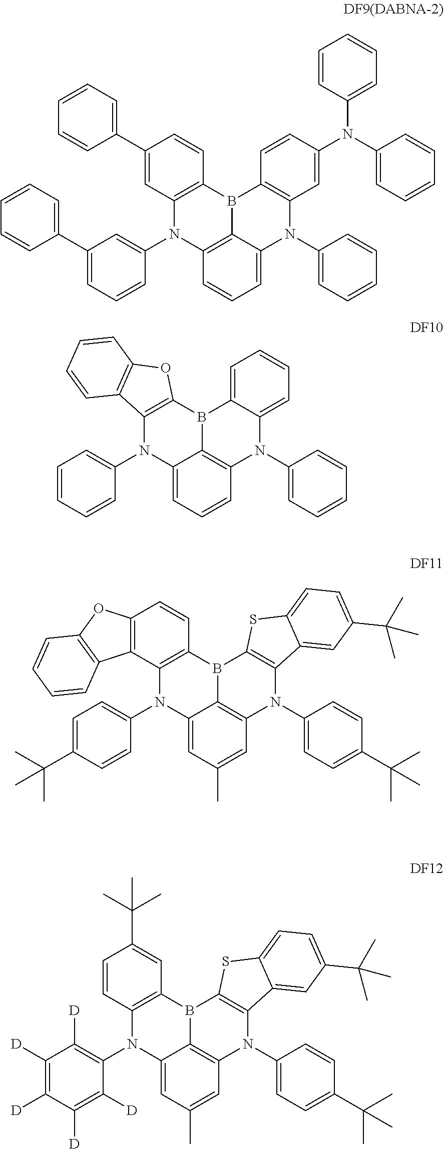

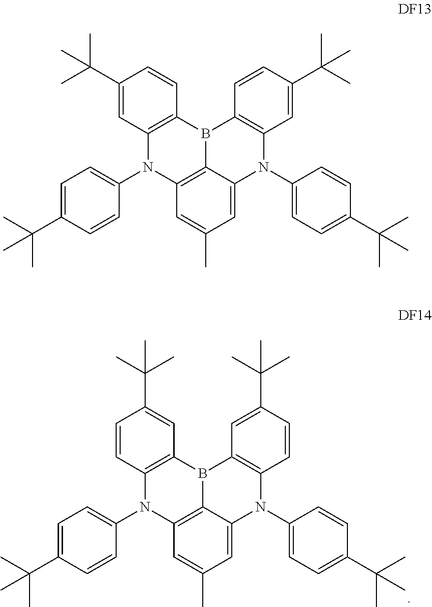

### Quantum Dot

In one or more embodiments, the emission layer may include quantum dots.

The term “quantum dot” as utilized herein refers to a crystal of a semiconductor compound, and may include any material capable of emitting light of one or more suitable emission wavelengths according to the size of the crystal.

A diameter of the quantum dots may be, for example, in a range of about 1 nm to about 10 nm.

The quantum dots may be synthesized by a wet chemical process, a metal organic chemical vapor deposition process, a molecular beam epitaxy process, or any process similar thereto.

The wet chemical process is a method including mixing a precursor material of a quantum dot with an organic solvent and then growing quantum dot particle crystals. When the crystals grow, the organic solvent naturally acts as a dispersant coordinated on the surface of the quantum dot crystals and controls the growth of the crystals so that the growth of quantum dot particles may be controlled or selected through a process which costs lower and is easier than vapor deposition methods, such as metal organic chemical vapor deposition (MOCVD) or molecular beam epitaxy (MBE).

The quantum dots may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Non-limiting examples of the Group II-VI semiconductor compound may include: a binary compound, such as CdS, CdSe, CdTe, ZnS, ZnSe, ZnTe, ZnO, HgS, HgSe, HgTe, MgSe, MgS, etc.; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, etc.; a quaternary compound, such as CdZnSeS, CdZnSeTe, CdZnSTe, CdHgSeS, CdHgSeTe, CdHgSTe, HgZnSeS, HgZnSeTe, HgZnSTe, etc.; or any combination thereof.

Non-limiting examples of the Group III-V semiconductor compound may include: a binary compound, such as GaN, GaP, GaAs, GaSb, AlN, AlP, AlAs, AlSb, InN, InP, InAs, InSb, etc.; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, etc.; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, etc.; or any combination thereof. In some embodiments, the Group III-V semiconductor compound may further include a Group II element. Non-limiting examples of the Group III-V semiconductor compound further including a Group II element may include InZnP, InGaZnP, InAlZnP, etc.

Non-limiting examples of the Group III-VI semiconductor compound may include: a binary compound, such as GaS, GaSe, Ga₂Se₃, GaTe, InS, InSe, In₂S₃, In₂Se₃, InTe, etc.; a ternary compound, such as InGaS₃, InGaSe₃, etc.; or any combination thereof.

Non-limiting examples of the Group I-III-VI semiconductor compound may include: a ternary compound, such as AgInS, AgInS₂, CuInS, CuInS₂, CuGaO₂, AgGaO₂, AgAlO₂, etc.; or any combination thereof.

Non-limiting examples of the Group IV-VI semiconductor compound may include: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, SnPbTe, etc.; a quaternary compound, such as SnPbSSe, SnPbSeTe, SnPbSTe, etc.; or any combination thereof.

The Group IV element or compound may include: a single element compound, such as Si, Ge, etc.; a binary compound, such as SiC, SiGe, etc.; or any combination thereof.

Each element included in a multi-element compound such as the binary compound, the ternary compound, and the quaternary compound may be present at a substantially uniform concentration or non-substantially uniform concentration in a particle.

In some embodiments, the quantum dots may (e.g., may each) have a single structure in which the concentration of each element in the quantum dots is substantially uniform, or a core-shell dual structure. For example, in some embodiments, materials included in the core and materials included in the shell may be different from each other.

The shell of (e.g., each of) the quantum dots may act as a protective layer that prevents chemical degeneration of the core to maintain semiconductor characteristics, and/or as a charging layer that imparts electrophoretic characteristics to the quantum dots. The shell may be a single layer or a multi-layer. The interface between the core and the shell may have a concentration gradient in which the concentration of an element existing in the shell decreases toward the center of the core.

Non-limiting examples of the shell of the quantum dots may include: oxides of metal, metalloid, or non-metal, semiconductor compounds: or any combination thereof. Non-limiting examples of the oxides of metal, metalloid, or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, etc.; a ternary compound, such as MgAl₂O₄, CoFe₂O₄, NiFe₂O₄, CoMn₂O₄, etc.; and/or any combination thereof. Non-limiting examples of the semiconductor compound may include: as described above, a Group II-VI semiconductor compound; a Group III-V semiconductor compound; a Group III-VI semiconductor compound; a Group I-III-VI semiconductor compound; a Group IV-VI semiconductor compound; or any combination thereof. For example, the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of the emission spectrum of the quantum dots may be less than or equal to about 45 nm, for example, less than or equal to about 40 nm, or for example, less than or equal to about 30 nm, and within these ranges, color purity or color reproducibility of the quantum dots may be improved. In some embodiments, because light emitted through the quantum dots is emitted in all directions, the wide viewing angle may be improved.

In some embodiments, the quantum dots may be in the form of a spherical particle, a pyramidal particle, a multi-arm particle, a cubic nanoparticle, a nanotube particle, a nanowire particle, a nanofiber particle, a nanoplate particle, and/or the like.

Because the energy band gap of the quantum dot may be adjusted by controlling the size of the quantum dots, light having one or more suitable wavelength bands may be obtained from a quantum dot-containing emission layer. Accordingly, by utilizing quantum dots of different sizes, a light-emitting device that emits light of one or more suitable wavelengths may be implemented. In one or more embodiments, the size of the quantum dots may be selected to emit red light, green light, and/or blue light. In some embodiments, the size of the quantum dots may be configured to emit white light by combination of light of one or more suitable colors.

### Electron Transport Region in Interlayer 130

The electron transport region may have: i) a single-layered structure including (e.g., consisting of) a single layer including (e.g., consisting of) a single material, ii) a single-layered structure including (e.g., consisting of) a single layer including (e.g., consisting of) multiple materials that are different from each other, or iii) a multi-layered structure including multiple layers including multiple materials that are different from each other.

The electron transport region may include a buffer layer, a hole blocking layer, an electron control layer, an electron transport layer, an electron injection layer, or any combination thereof.

For example, in one or more embodiments, the electron transport region may have an electron transport layer/electron injection layer structure, a hole-blocking layer/electron transport layer/electron injection layer structure, an electron control layer/electron transport layer/electron injection layer structure, a buffer layer/electron transport layer/electron injection layer structure, and/or the like, wherein constituting layers of each structure are sequentially stacked from the emission layer in the stated order.

In one or more embodiments, the electron transport region may further include an electron transport auxiliary layer. For example, the electron transport auxiliary layer may be in direct contact with the emission layer. For example, the electron transport region may have an electron transport auxiliary layer/electron transport layer structure, an electron transport auxiliary layer/electron transport layer/electron injection layer structure, and/or the like, wherein constituting layers of each structure are sequentially stacked from the emission layer in the stated order.

In one or more embodiments, the electron transport region (e.g., a buffer layer, a hole blocking layer, an electron control layer, or an electron transport layer in the electron transport region) may include a metal-free compound including at least one IT electron-deficient nitrogen-containing C₁-C₆₀ heterocyclic group.

For example, in one or more embodiments, the electron transport region may include a compound represented by Formula 601:

[Ar₆₀₁]ₓₑ₁₁-[(L₆₀₁)ₓₑ₁-R₆₀₁]ₓₑ₂₁, Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be the same as described with respect to Q₁,
xe21 may be 1, 2, 3, 4, or 5, and
at least one selected from among Ar₆₀₁, L₆₀₁, and R₆₀₁ may each independently be a TT electron-deficient nitrogen-containing C₁-C₆₀ cyclic group that is unsubstituted or substituted with at least one R₁₀ₐ.

For example, in some embodiments, when xe11 in Formula 601 is 2 or more, two or more of Ar₆₀₁(s) may be linked to each other via a single bond.

In one or more embodiments, Ar₆₀₁ in Formula 601 may be a substituted or unsubstituted anthracene group.

In one or more embodiments, the electron transport region may include a compound represented by Formula 601-1:

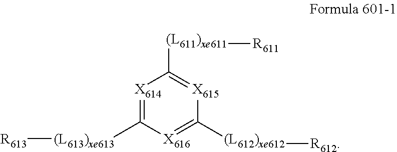

In Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each be the same as described with respect to L₆₀₁,
xe611 to xe613 may each be the same as described with respect to xe1,
R₆₁₁ to R₆₁₃ may each be the same as described with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, in some embodiments, xe1 and xe611 to xe613 in Formulae 601 and 601-1 may each independently be 0, 1, or 2.

In one or more embodiments, the electron transport region may include at least one selected from Compounds ET1 to ET46, 2,9-dimethyl-4,7-diphenyl-1,10-phenanthroline (BCP), 4,7-diphenyl-1,10-phenanthroline (Bphen), tris(8-hydroxyquinolinato)aluminum (Alq₃), bis(2-methyl-8-quinolinolato-N1,O8)-(1,1′-biphenyl-4-olato)aluminum (BAlq), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), and/or any combination thereof:

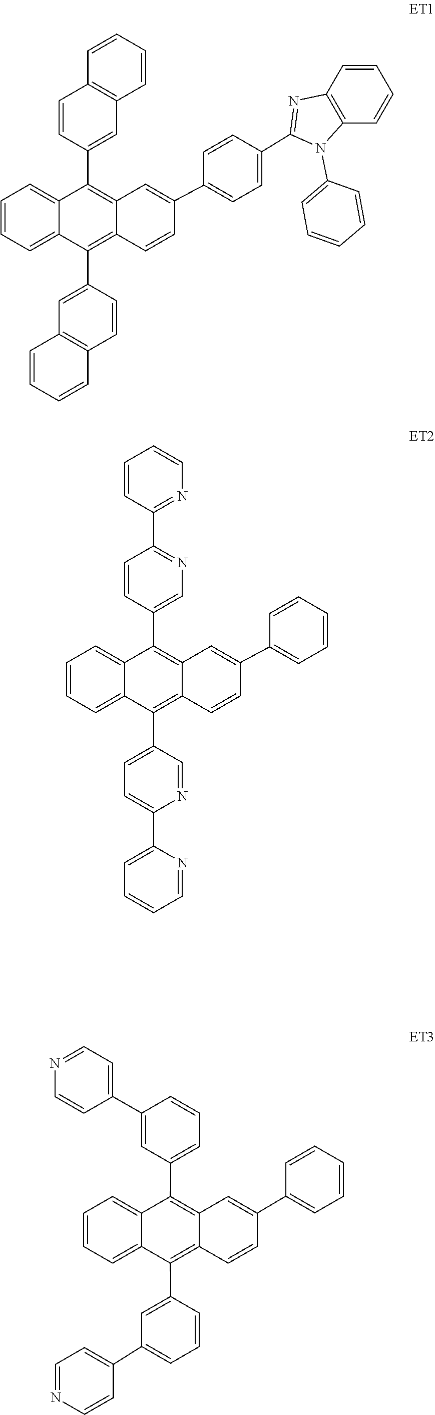 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

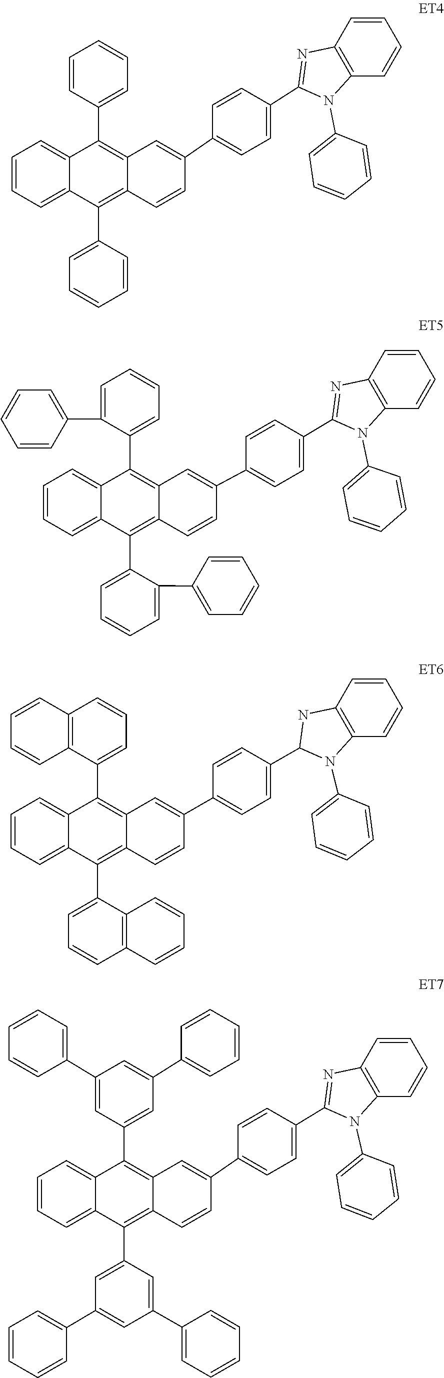

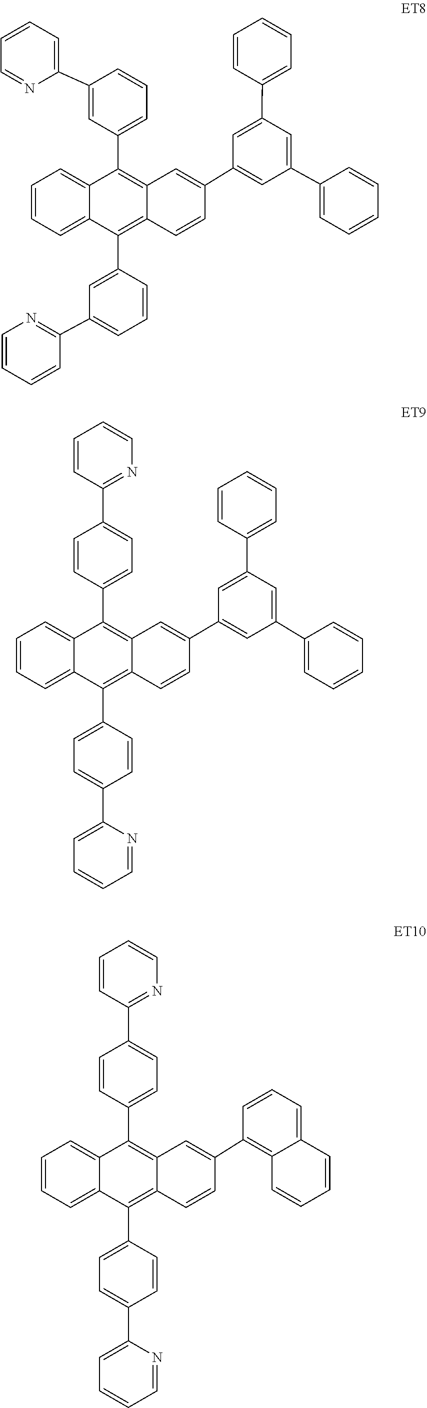 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

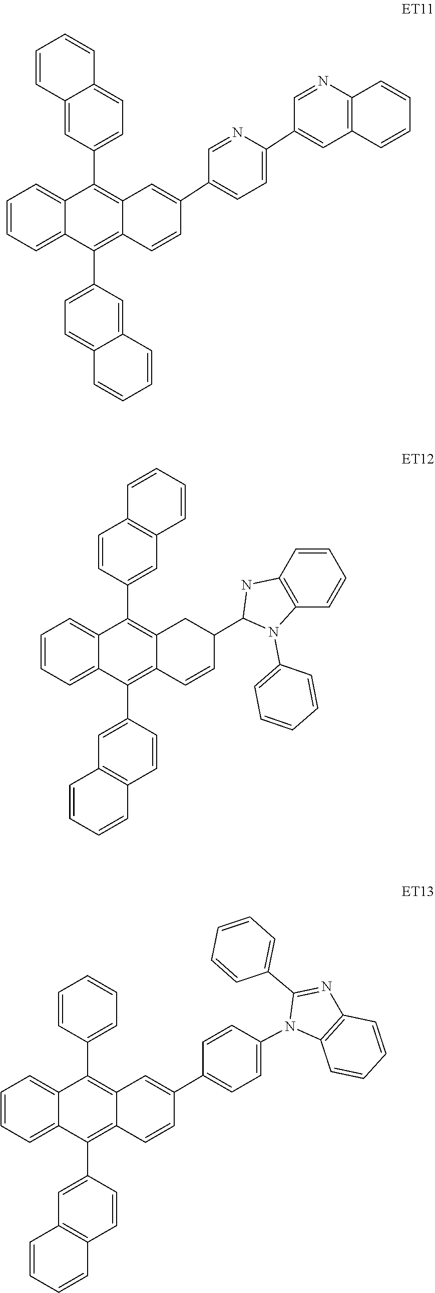

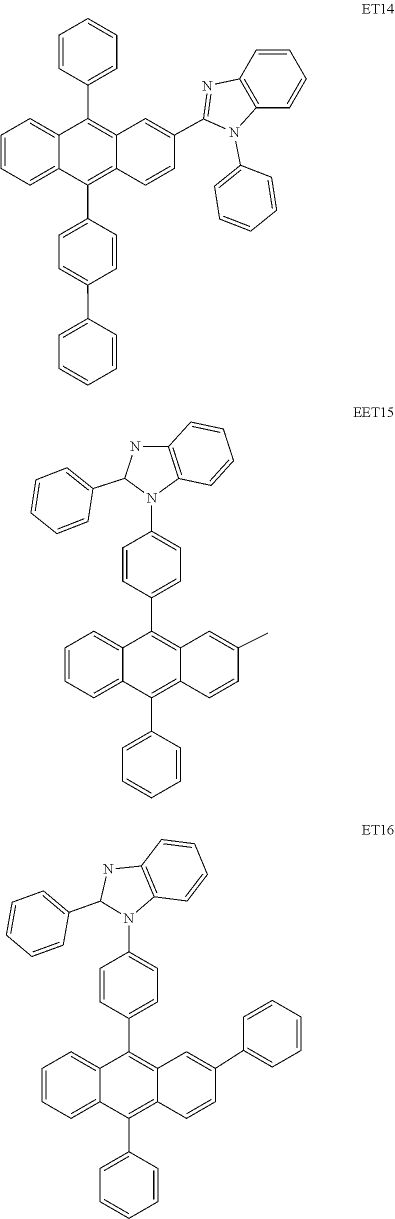

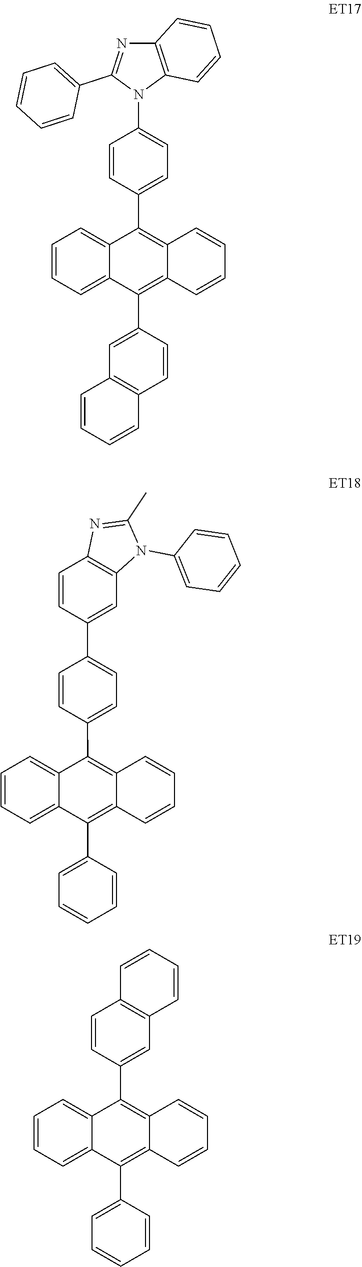

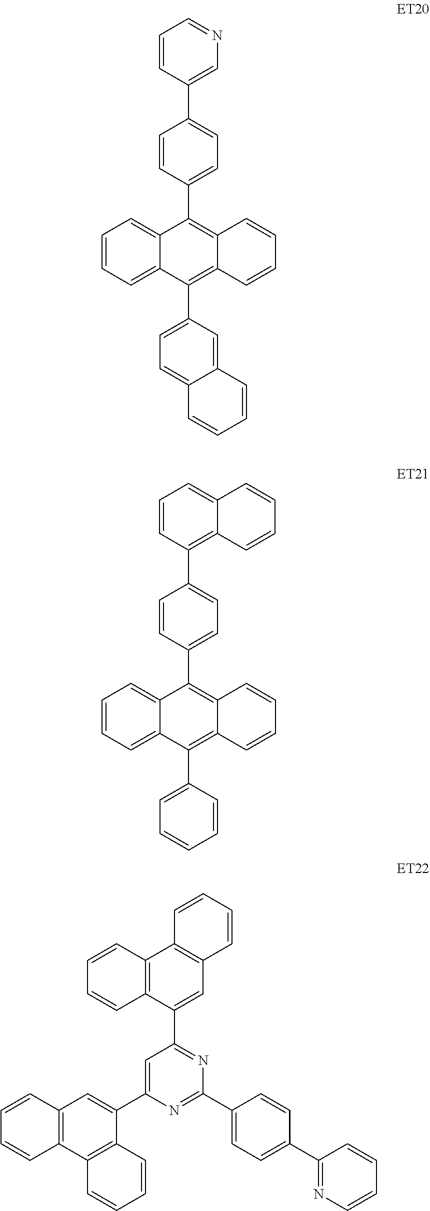

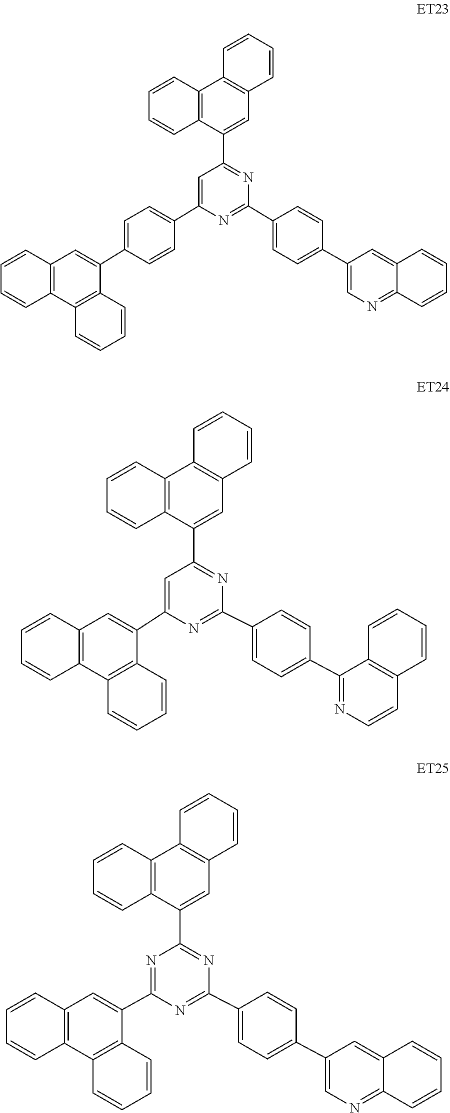 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

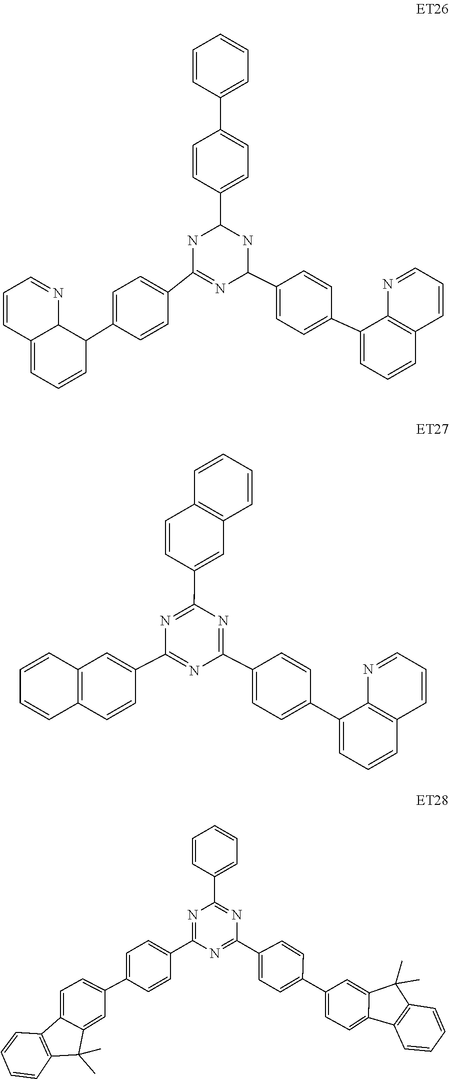

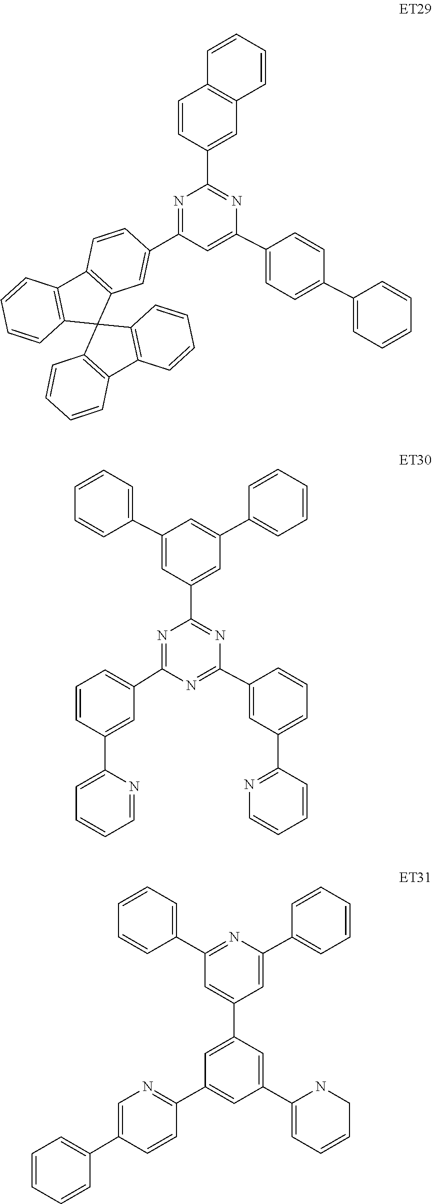

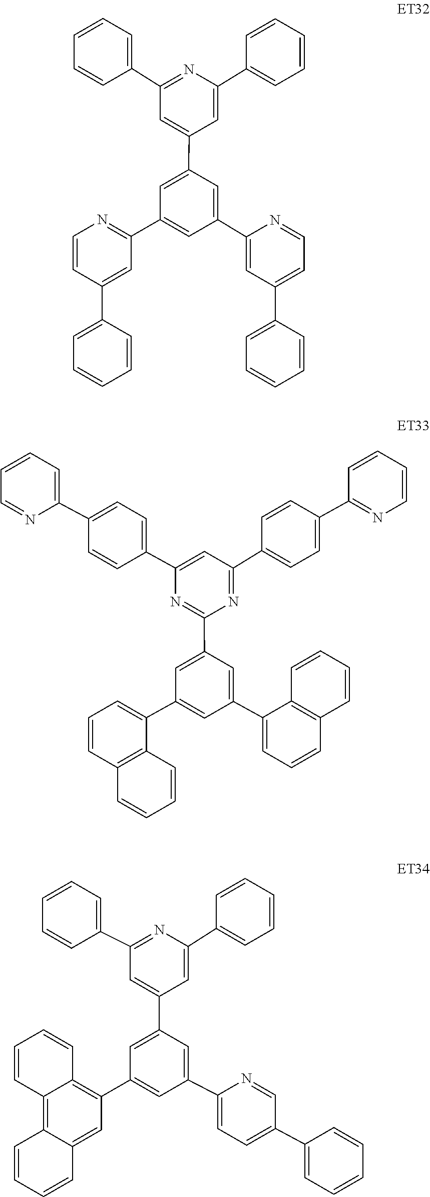 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

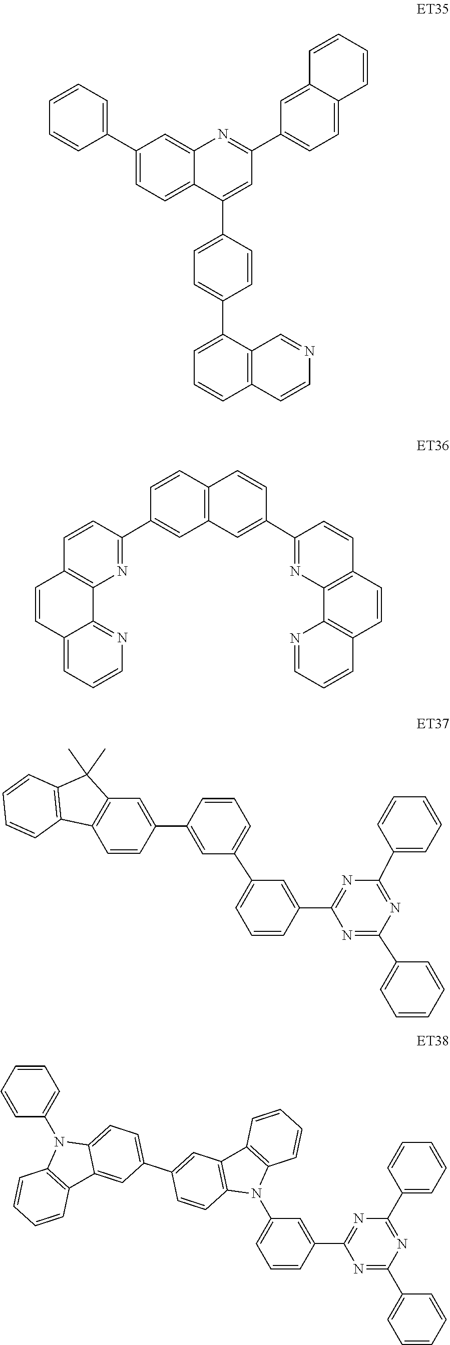 [SMILES: c1ccc(-c2ccc3c(-c4ccc(-c5cccc6ccncc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

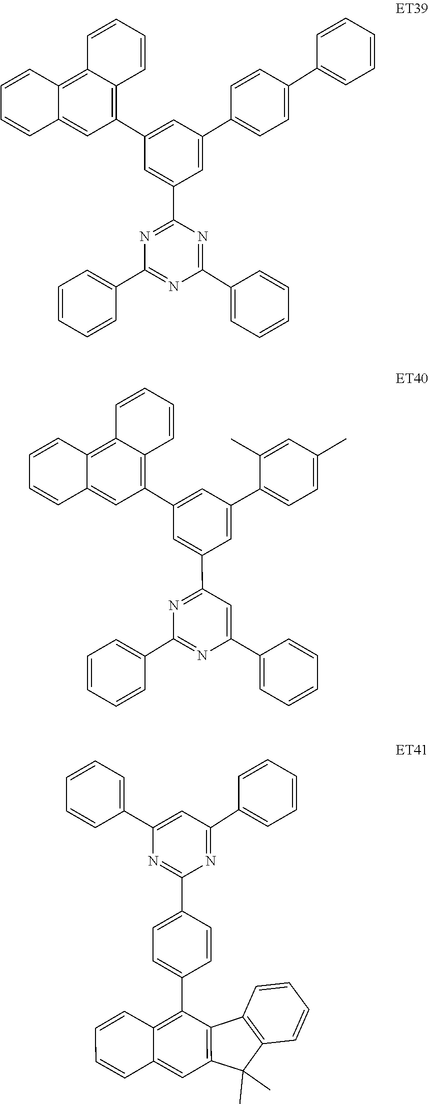 [SMILES: c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1]

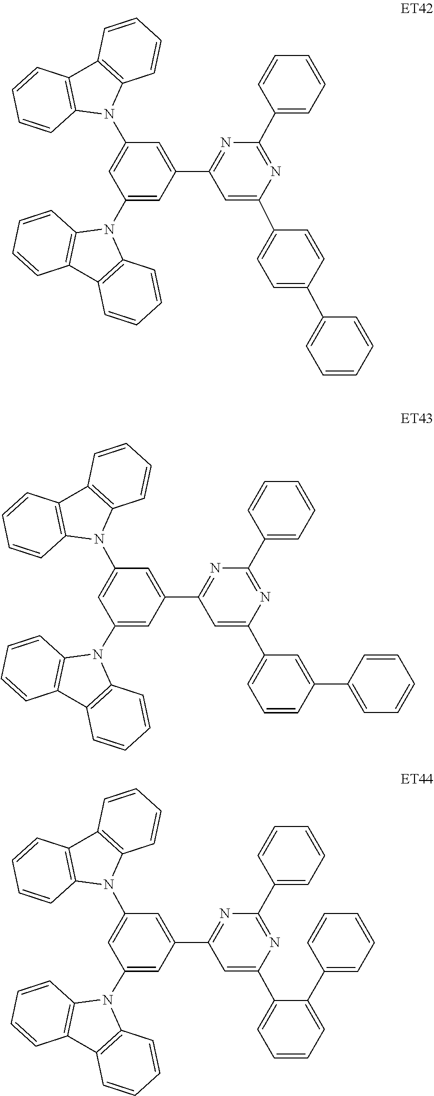

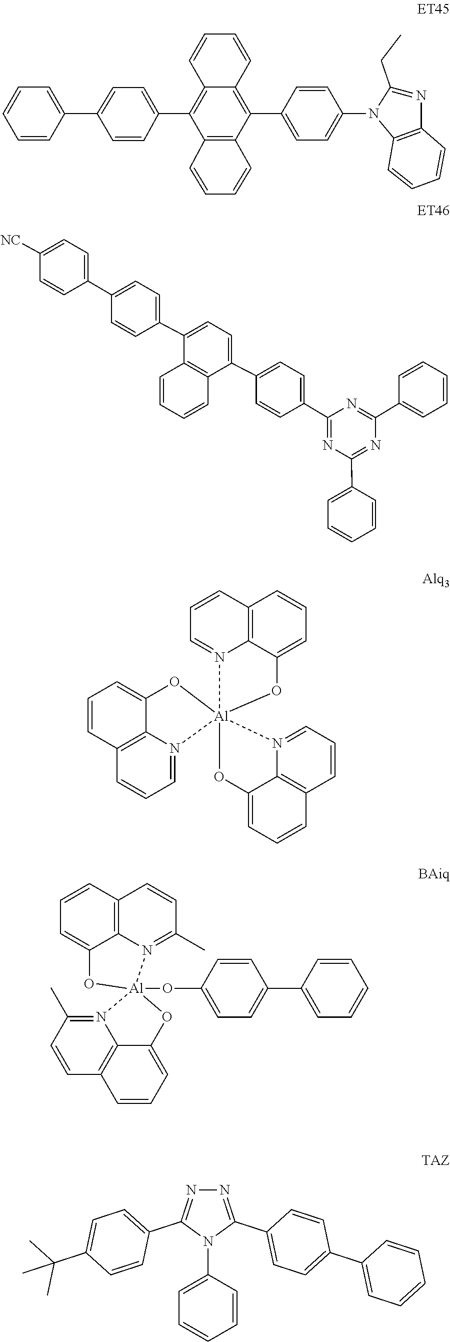

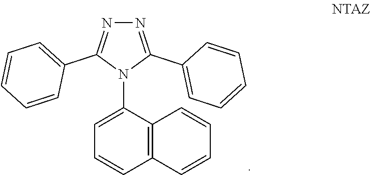 [SMILES: c1ccc(-c2nnc(-c3ccccc3)n2-c2cccc3ccccc23)cc1]

A thickness of the electron transport region may be from about 100 Å to about 5,000 Å, for example, about 160 Å to about 4,000 Å. When the electron transport region includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport layer are within these ranges, satisfactory electron transporting characteristics may be obtained without a substantial increase in driving voltage.

In one or more embodiments, the electron transport region (e.g., the electron transport layer in the electron transport region) may further include, in addition to the aforementioned materials, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the metal ion of the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, in some embodiments, the metal-containing material may include a Li complex. The Li complex may include, for example, Compound ET-D1 (LiQ) or ET-D2:

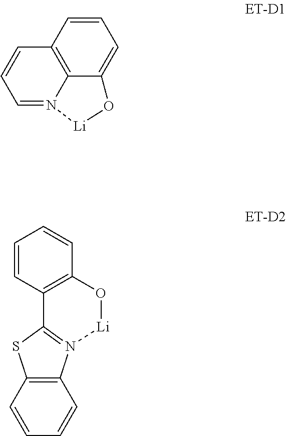

In one or more embodiments, the electron transport region may include an electron injection layer that facilitates the injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including (e.g., consisting of) multiple layers that are different from each other, or iii) a multi-layered structure including multiple layers including multiple materials that are different from each other.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may respectively be oxides, halides (e.g., fluorides, chlorides, bromides, or iodides), or tellurides of the alkali metal, the alkaline earth metal, and the rare earth metal, or any combination thereof.

The alkali metal-containing compound may include: alkali metal oxides, such as Li₂O, Cs₂O, K₂O, etc.; alkali metal halides, such as LiF, NaF, CsF, KF, LiI, NaI, CsI, KI, etc.; or any combination thereof. The alkaline earth metal-containing compound may include alkaline earth metal compounds, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal tellurides. Non-limiting examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) one of metal ions of the alkali metal, one of metal ions of the alkaline earth metal, and one of metal ions of the rare earth metal, respectively, and ii) a ligand bonded to the metal ion, for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (e.g., the compound represented by Formula 601).

In one or more embodiments, the electron injection layer may include (e.g., consist of) i) an alkali metal-containing compound (e.g., an alkali metal halide), or ii) a) an alkali metal-containing compound (e.g., an alkali metal halide), and b) an alkali metal, an alkaline earth metal, a rare earth metal, or any combination thereof. For example, in some embodiments, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be substantially uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within these ranges, satisfactory electron injection characteristics may be obtained without a substantial increase in driving voltage.

### Second Electrode 150

The second electrode 150 may be arranged on the interlayer 130 having the aforementioned structure. The second electrode 150 may be a cathode, which is an electron injection electrode, and as a material for forming the second electrode 150, a metal, an alloy, an electrically conductive compound, or any combination thereof, each having a low-work function, may be utilized.

The second electrode 150 may include Li, Ag, Mg, Al, Al—Li, Ca, Mg—In, Mg—Ag, Yb, Ag—Yb, ITO, IZO, or any combination thereof. The second electrode 150 may be a transmissive electrode, a semi-transmissive electrode, or a reflective electrode.

The second electrode 150 may have a single-layered structure or a multi-layered structure including multiple layers.

### Capping Layer

A first capping layer may be arranged outside (e.g., on) the first electrode 110, and/or a second capping layer may be arranged outside (e.g., on) the second electrode 150. In one or more embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

In some embodiments, light generated from the emission layer of the interlayer 130 of the light-emitting device 10 may be extracted toward the outside through the first electrode 110, which is a semi-transmissive electrode or a transmissive electrode, and the first capping layer. In some embodiments, light generated from the emission layer of the interlayer 130 of the light-emitting device 10 may be extracted toward the outside through the second electrode 150, which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, and the luminescence efficiency of the light-emitting device 10 may be improved.

Each of the first capping layer and the second capping layer may include a material having a refractive index of greater than or equal to 1.6 (e.g., at 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from among the first capping layer and the second capping layer may (e.g., the first capping layer and the second capping layer may each) independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may each optionally be substituted with a substituent including O, N, S, Se, Si, F, Cl, Br, I, or any combination thereof. In one or more embodiments, at least one selected from among the first capping layer and the second capping layer may (e.g., the first capping layer and the second capping layer may each) independently include an amine group-containing compound.

In one or more embodiments, at least one selected from among the first capping layer and the second capping layer may (e.g., the first capping layer and the second capping layer may each) independently include a compound represented by Formula 201, a compound represented by Formula 202, or any combination thereof.

In one or more embodiments, at least one selected from among the first capping layer and the second capping layer may (e.g., the first capping layer and the second capping layer may each) independently include at least one selected from Compounds HT28 to HT33, Compounds CP1 to CP6, β-NPB, and/or any combination thereof:

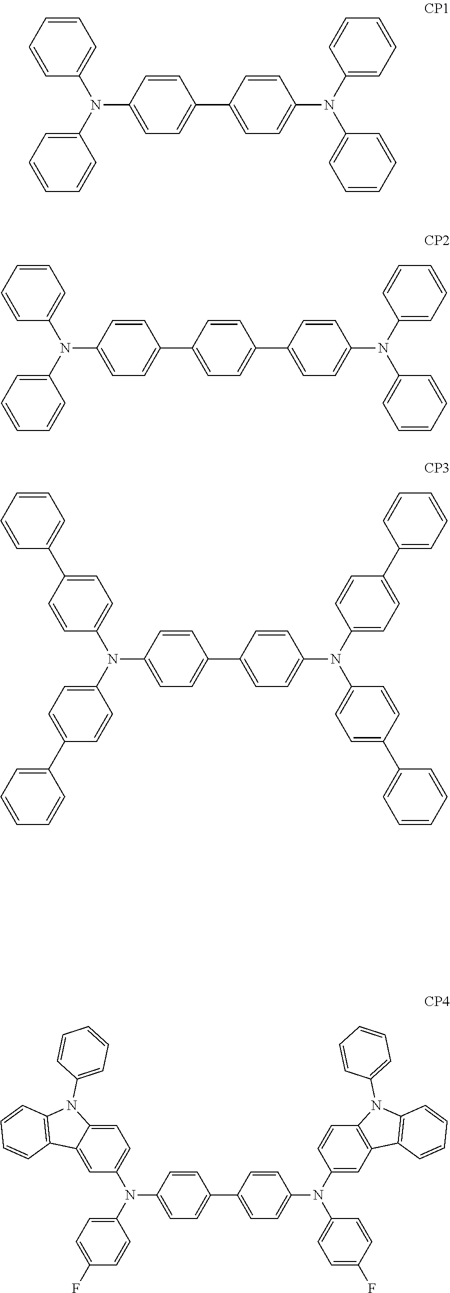 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

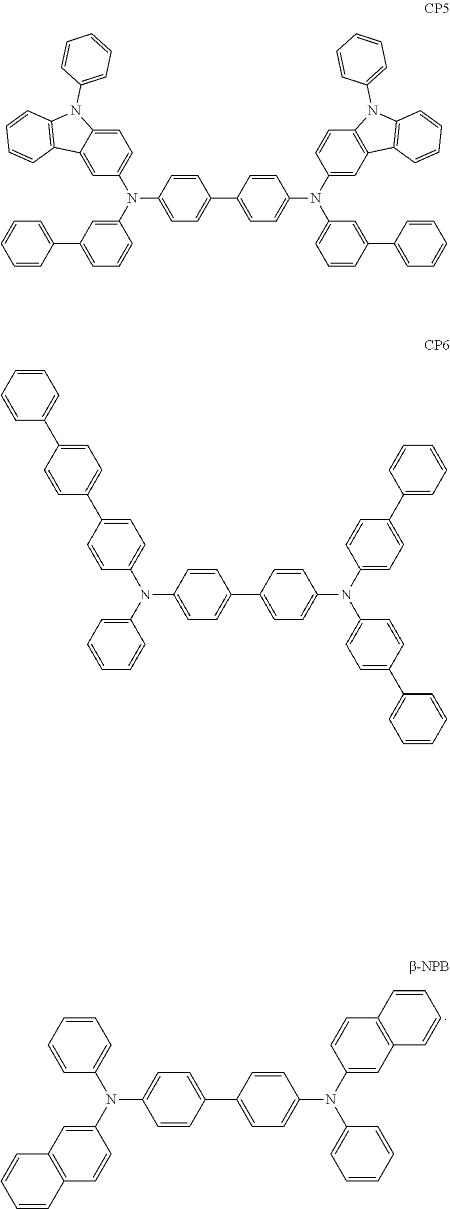 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The heterocyclic compound represented by Formula 1 may be included in one or more suitable films. Accordingly, one or more aspects of embodiments of the present disclosure are directed toward a film including the heterocyclic compound represented by Formula 1. The film may be, for example, an optical member (or a light control member) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, and/or the like), and/or a protective member (for example, an insulating layer, a dielectric layer, and/or the like).

### Electronic Apparatus

The light-emitting device may be included in one or more suitable electronic apparatuses. For example, in one or more embodiments, the electronic apparatus including the light-emitting device may be a light-emitting apparatus, an authentication apparatus, and/or the like.

In one or more embodiments, the electronic apparatus (e.g., a light-emitting apparatus) may further include, in addition to the light-emitting device, i) a color filter, ii) a color conversion layer, or iii) a color filter and a color conversion layer. The color filter and/or the color conversion layer may be arranged in at least one direction in which light emitted from the light-emitting device travels. For example, in some embodiments, light emitted from the light-emitting device may be blue light, green light, or white light (e.g., combined white light). Details on the light-emitting device may be the same as described herein. In some embodiments, the color conversion layer may include quantum dots.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns thereon, and the color conversion layer may further include a plurality of color conversion areas and light-shielding patterns thereon.

The plurality of color filter areas (or the plurality of color conversion areas) may include a first area configured to emit first color light, a second area configured to emit second color light, and/or a third area configured to emit third color light, wherein the first color light, the second color light, and/or the third color light may have different maximum emission wavelengths from one another. For example, in one or more embodiments, the first color light may be red light, the second color light may be green light, and the third color light may be blue light. For example, in one or more embodiments, the plurality of color filter areas (or the plurality of color conversion areas) may include quantum dots. In some embodiments, the first area may include red quantum dots to emit red light, and the second area may include green quantum dots to emit green light, whereas the third area may not include (e.g., may exclude) (e.g., any) quantum dots. Details on the quantum dot may be the same as described herein. The first area, the second area, and/or the third area may each further include a scatter.

For example, in one or more embodiments, the light-emitting device may be to emit first light, the first area may be to absorb the first light to emit first-first color light, the second area may be to absorb the first light to emit second-first color light, and the third area may be to absorb the first light to emit third-first color light. In this regard, the first-first color light, the second-first color light, and the third-first color light may have different maximum emission wavelengths from one another. In some embodiments, the first light may be blue light, the first-first color light may be red light, the second-first color light may be green light, and the third-first color light may be blue light.

In one or more embodiments, the electronic apparatus may further include a thin-film transistor, in addition to the aforementioned light-emitting device. The thin-film transistor may include a source electrode, a drain electrode, and an activation layer, wherein one selected from the source electrode and the drain electrode may be electrically connected to the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

In one or more embodiments, the electronic apparatus may further include a sealing portion for sealing the light-emitting device. The sealing portion may be arranged between the color filter and/or the color conversion layer and the light-emitting device. The sealing portion allows light from the light-emitting device to be extracted to the outside, and concurrently (e.g., simultaneously) prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin-film encapsulation layer, the electronic apparatus may be flexible.

Various functional layers may be additionally located on the sealing portion, in addition to the color filter and/or the color conversion layer, according to the utilization of the electronic apparatus. Non-limiting examples of the functional layers may include a touch screen layer, a polarizing layer, and/or the like. The touch screen layer may be a pressure-sensitive touch screen layer, a capacitive touch screen layer, or an infrared touch screen layer.

The authentication apparatus may be, for example, a biometric authentication apparatus that authenticates an individual by utilizing biometric information of a living body (e.g., fingertips, pupils, etc.). The authentication apparatus may further include, in addition to the light-emitting device as described above, a biometric information collector.

The electronic apparatus may be applied to one or more of displays, light sources, lighting, personal computers (e.g., mobile personal computers), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (e.g., meters for a vehicle, an aircraft, and a vessel), projectors, and/or the like.

### Electronic Equipment

The light-emitting device may be included in one or more suitable electronic equipment.

For example, the electronic equipment including the light-emitting device may be at least one of a flat panel display, a curved display, a computer monitor, a medical monitor, a television, a billboard, a light for indoor or outdoor lighting and/or signaling, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented-reality display, a vehicle, a video wall including multiple displays tiled together, a theater or stadium screen, a phototherapy device, or a signboard.

Because the light-emitting device has excellent or suitable effects in terms of luminescence efficiency long lifespan, the electronic equipment including the light-emitting device may have characteristics with high luminance, high resolution, and low power consumption.

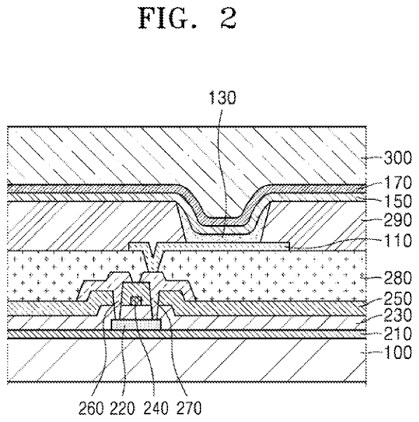

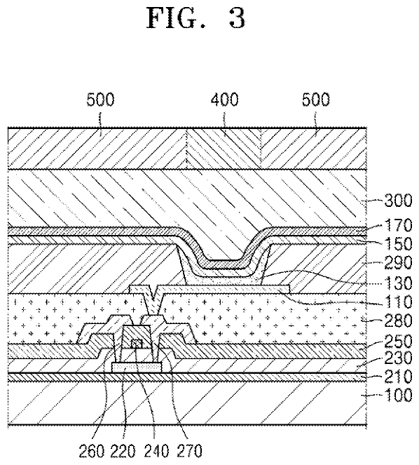

### [Description of FIG. 2 and FIG. 3]

FIG. 2 is a cross-sectional view showing a light-emitting apparatus as an example of the electronic apparatus according to one or more embodiments of the present disclosure.

The light-emitting apparatus of FIG. 2 may include a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be on the substrate 100. The buffer layer 210 may prevent or reduce penetration of impurities through the substrate 100, and provide a flat surface on the substrate 100.

The TFT may be on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be arranged between the gate electrode 240 and the source electrode 260 and between the gate electrode 240 and the drain electrode 270, to insulate these electrodes from one another.

The source electrode 260 and the drain electrode 270 may be on the interlayer insulating film 250. The interlayer insulating film 250 and the gate insulating film 230 may be formed to expose the source region and the drain region of the activation layer 220, and the source electrode 260 and the drain electrode 270 may be located in contact with the exposed portions of the source region and the drain region of the activation layer 220, respectively.

The TFT is electrically connected to the light-emitting device to drive the light-emitting device, and may be covered and protected by a passivation layer 280. The passivation layer 280 may include an inorganic insulating film, an organic insulating film, or any combination thereof. The light-emitting device may be provided on the passivation layer 280. The light-emitting device may include a first electrode 110, an interlayer 130, and a second electrode 150.

The first electrode 110 may be on the passivation layer 280. The passivation layer 280 may be arranged to expose a portion of the drain electrode 270 without fully covering the drain electrode 270, and the first electrode 110 may be arranged to be connected to the exposed portion of the drain electrode 270.

A pixel defining layer 290 including an insulating material may be on the first electrode 110. The pixel defining layer 290 may expose a certain region of the first electrode 110, and the interlayer 130 may be formed in the exposed region of the first electrode 110. The pixel defining layer 290 may be a polyimide-based organic film or a polyacrylic-based organic film. In some embodiments, at least some layers of the interlayer 130 may extend beyond the upper portion of the pixel defining layer 290 to be arranged in the form of a common layer.

The second electrode 150 may be on the interlayer 130, and a second capping layer 170 may be additionally formed on the second electrode 150. The second capping layer 170 may be formed to cover the second electrode 150.

The encapsulation portion 300 may be on the second capping layer 170. The encapsulation portion 300 may be arranged on the light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or any combination of the inorganic films and the organic films.

FIG. 3 is a cross-sectional view of a light-emitting apparatus as an example of the electronic apparatus according to one or more embodiments of the present disclosure.

The light-emitting apparatus of FIG. 3 is substantially the same as the light-emitting apparatus of FIG. 2, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the light-emitting apparatus of FIG. 3 may be a tandem light-emitting device.

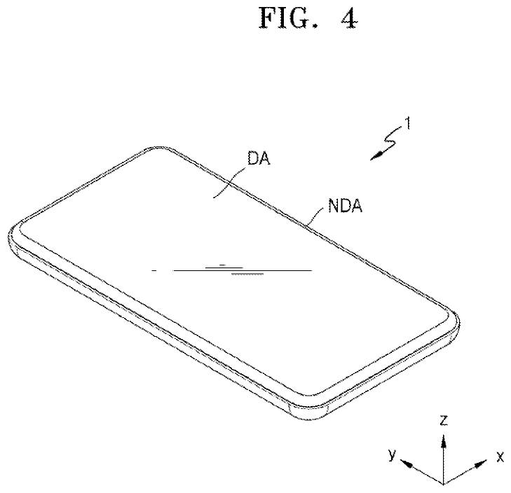

### Description of FIG. 4

FIG. 4 is a schematic perspective view of electronic equipment 1 including a light-emitting device according to one or more embodiments. The electronic equipment 1 may be, as a device apparatus that displays a moving image or a still image, portable electronic equipment, such as a mobile phone, a smart phone, a tablet personal computer (PC), a mobile communication terminal, an electronic notebook, an electronic book, a portable multimedia player (PMP), a navigation, or a ultra-mobile PC (UMPC), as well as one or more suitable products or a portion thereof, such as a television, a laptop, a monitor, a billboard, or an Internet of things (IoT). In some embodiments, the electronic equipment 1 may be a wearable device, such as a smart watch, a watch phone, a glasses-type or kind display, or a head mounted display (HMD), or a part of the wearable device. However, embodiments of the disclosure are not limited thereto. For example, in one or more embodiments, the electron equipment 1 may include a dashboard of a vehicle, a center information display on a center fascia or dashboard of a vehicle, a room mirror display replacing a side mirror of a vehicle, an entertainment display arranged for the rear seat of a vehicle or arranged on the back of the front seat, a head-up display (HUD) installed at the front of a vehicle or projected on a front window glass, and/or a computer generated hologram augmented reality head up display (CGH AR HUD). FIG. 4 illustrates an embodiment in which the electronic equipment 1 is a smartphone for convenience of explanation.

The electronic equipment 1 may include a display area DA and a non-display area NDA outside the display area DA. A display device of the electronic equipment 1 may implement an image through an array of a plurality of pixels that are two-dimensionally arranged in the display area DA.

The non-display area NDA is an area that does not display an image, and may entirely surround the display area DA. On the non-display area NDA, a driver for providing electrical signals or power to display devices arranged on the display area DA may be arranged. On the non-display area NDA, a pad, which is an area to which an electronic element or a printing circuit board may be electrically connected, may be arranged.

In the electronic equipment 1, a length in the x-axis direction and a length in the y-axis direction may be different from each other. In one or more embodiments, as shown in FIG. 4, the length in the x-axis direction may be shorter than the length in the y-axis direction. In one or more embodiments, the length in the x-axis direction may be substantially the same as the length in the y-axis direction. In one or more embodiments, the length in the x-axis direction may be longer than the length in the y-axis direction.

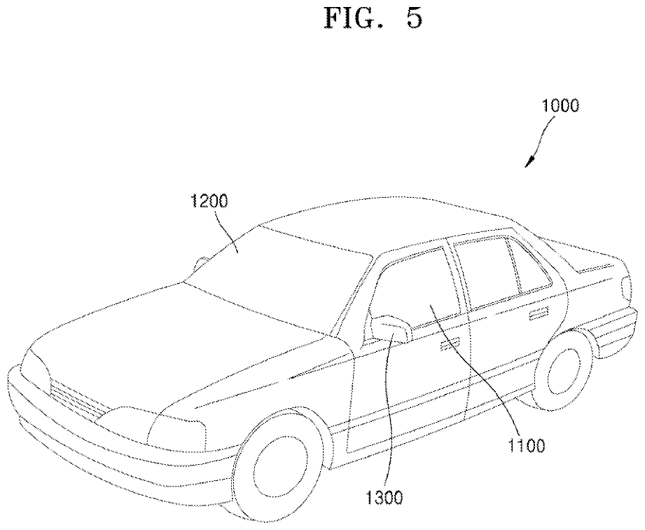

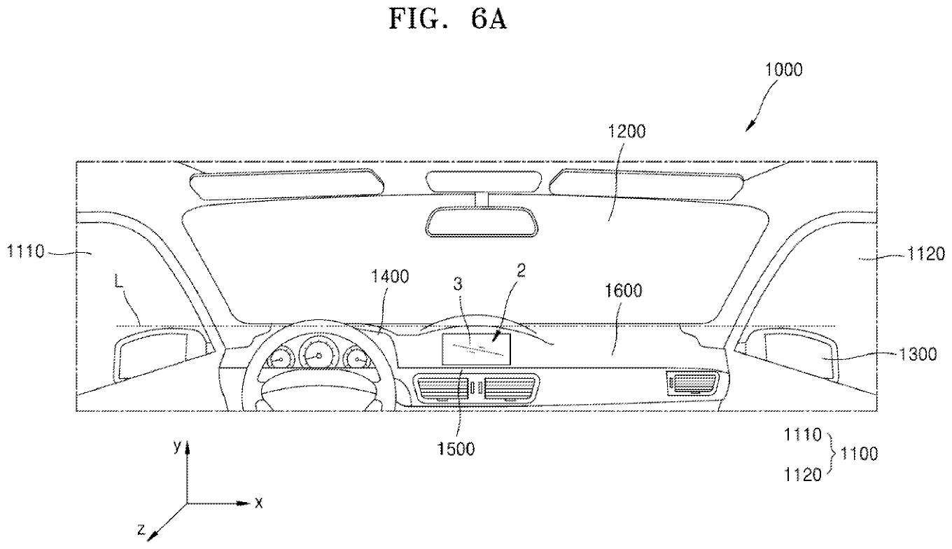

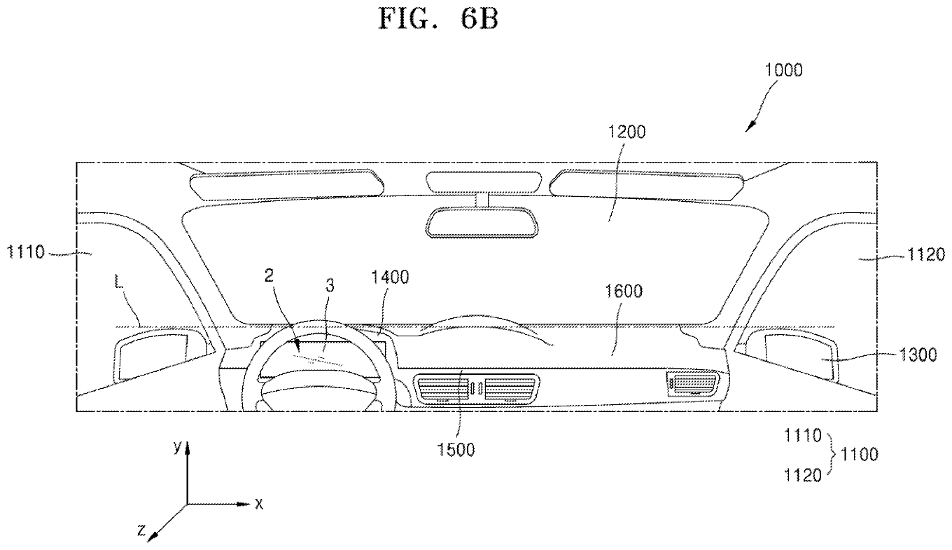

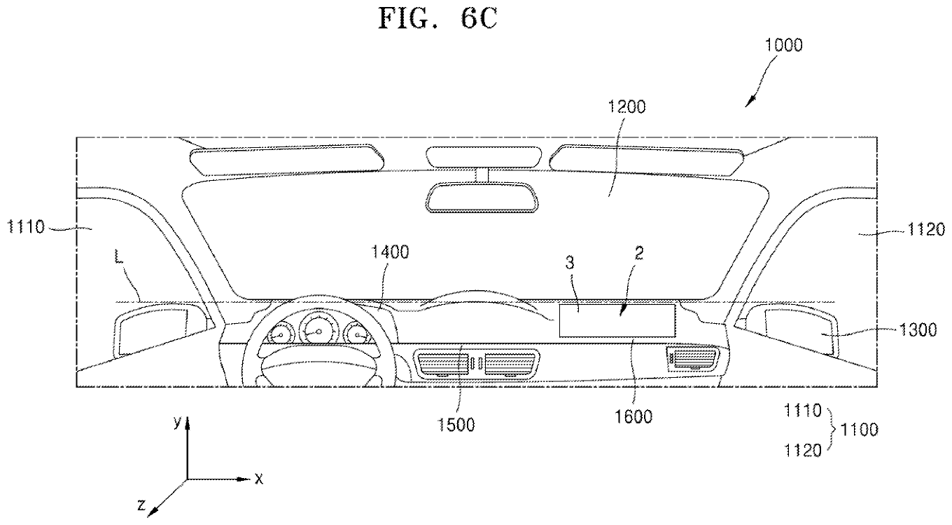

### Descriptions of FIGS. 5 and 6A to 6C

FIG. 5 is a schematic view of an exterior of a vehicle 1000 as an electronic apparatus including a light-emitting device according to one or more embodiments. FIGS. 6A to 6C are each a schematic view of an interior of the vehicle 1000 according to one or more embodiments.

Referring to FIGS. 5 and 6A to 6C, the vehicle 1000 may refer to one or more suitable apparatuses for moving an object to be transported, such as a human, an object, or an animal, from a departure point to a destination. The vehicle 1000 may include a vehicle traveling on a road or a track, a vessel moving over the sea or a river, an airplane flying in the sky utilizing the action of air, and/or the like.

In one or more embodiments, the vehicle 1000 may travel on a road or a track. The vehicle 1000 may move in a set or predetermined direction according to the rotation of at least one wheel thereof. For example, the vehicle 1000 may include a three-wheeled or four-wheeled vehicle, a construction machine, a two-wheeled vehicle, a prime mover device, a bicycle, or a train running on a track.

The vehicle 1000 may include a body having an interior and an exterior, and a chassis in which mechanical apparatuses necessary for driving are installed as other parts except for the body. The exterior of the vehicle body may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a pillar arranged between the side window glass 1100 and the front window glass 1200.

The side window glass 1100 may be installed on a side of the vehicle 1000. In some embodiments, the side window glass 1100 may be installed on a door of the vehicle 1000. A plurality of side window glasses 1100 may be provided and may face each other. In some embodiments, the side window glass 1100 may include a first side window glass 1110 and a second side window glass 1120. In some embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced apart from each other in the x-direction or the −x-direction. For example, in some embodiments, the first side window glass 1110 and the second side window glass 1120 may be spaced apart from each other in the x direction or the −x direction. In other words, an imaginary straight line L connecting the side window glasses 1100 may extend in the x-direction or the −x-direction. For example, in some embodiments, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In one embodiment, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning light, a seat belt warning light, an odometer, a hodometer, an automatic shift selector indicator, a door open warning light, an engine oil warning light, and/or a low fuel warning light.

The center fascia 1500 may include a control panel on which a plurality of buttons for adjusting an audio device, an air conditioning device, and/or a heater of a seat are disposed. The center fascia 1500 may be arranged on one side of the cluster 1400.

The passenger seat dashboard 1600 may be spaced apart from the cluster 1400 with the center fascia 1500 arranged therebetween. In some embodiments, the cluster 1400 may be arranged to correspond to a driver seat, and the passenger seat dashboard 1600 may be disposed to correspond to a passenger seat. In some embodiments, the cluster 1400 may be adjacent to the first side window glass 1110, and the passenger seat dashboard 1600 may be adjacent to the second side window glass 1120.

In one or more embodiments, the display device 2 may include a display panel 3, and the display panel 3 may display an image. The display device 2 may be arranged inside the vehicle 1000. In some embodiments, the display device 2 may be arranged between the side window glasses 1100 facing each other. The display device 2 may be arranged on at least one of the cluster 1400, the center fascia 1500, or the passenger seat dashboard 1600.

The display device 2 may include an organic light-emitting display device, an inorganic electroluminescent display device, a quantum dot display device, and/or the like. Hereinafter, as the display device 2 according to one or more embodiments, an 1 organic light-emitting display apparatus including the aforementioned light-emitting device will be described as an example, but one or more suitable types (kinds) of the aforementioned display apparatus may be utilized in embodiments of the present disclosure.

Referring to FIG. 6A, in one or more embodiments, the display device 2 may be arranged on the center fascia 1500. In some embodiments, the display device 2 may display navigation information. In some embodiments, the display device 2 may display audio, video, and/or information regarding vehicle settings.

Referring to FIG. 6B, in one or more embodiments, the display device 2 may be arranged on the cluster 1400. When the display device 2 is arranged on the cluster 1400, the cluster 1400 may display driving information and/or the like through the display device 2. For example, in some embodiments, the cluster 1400 may be implemented digitally. The digital cluster 1400 may display vehicle information and driving information as images. For example, a needle and a gauge of a tachometer and one or more suitable warning light icons may be displayed by a digital signal.

Referring to FIG. 6C, in one or more embodiments, the display device 2 may be arranged on the passenger seat dashboard 1600. The display device 2 may be embedded in the passenger seat dashboard 1600 or arranged on the passenger seat dashboard 1600. In some embodiments, the display device 2 arranged on the passenger seat dashboard 1600 may display an image related to information displayed on the cluster 1400 and/or information displayed on the center fascia 1500. In one or more embodiments, the display device 2 arranged on the passenger seat dashboard 1600 may display information different from information displayed on the cluster 1400 and/or information displayed on the center fascia 1500.

### Manufacturing Method

The layers constituting the hole transport region, the emission layer, and the layers constituting the electron transport region may each be formed in a certain region by utilizing one or more suitable methods such as vacuum deposition, spin coating, casting, Langmuir-Blodgett (LB) deposition, ink-jet printing, laser-printing, laser-induced thermal imaging, and/or the like.

When the layers constituting the hole transport region, the emission layer, and the layers constituting the electron transport region are each formed by vacuum deposition, the deposition may be performed at a deposition temperature in a range of about 100° C. to about 500° C., at a vacuum degree in a range of about 10-8 torr to about 10-3 torr, and at a deposition speed in a range of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group including (e.g., consisting of) carbon only as a ring-forming atom and having three to sixty carbon atoms, and the term “C₁-C₆₀ heterocyclic group” as utilized herein refers to a cyclic group that has 1 to 60 carbon atoms and further has, in addition to carbon, a heteroatom as a ring-forming atom. The C₃-C₆₀ carbocyclic group and the C₁-C₆₀ heterocyclic group may each be: a monocyclic group including (e.g., consisting of) one (e.g., only one) ring; or a polycyclic group in which two or more rings are condensed with each other. For example, the number of ring-forming atoms of the C₁-C₆₀ heterocyclic group may be from 3 to 61.

The term “cyclic group” as utilized herein may include both (e.g., simultaneously) the C₃-C₆₀ carbocyclic group and the C₁-C₆₀ heterocyclic group.

The term “π electron-rich C₃-C₆₀ cyclic group” as utilized herein refers to a cyclic group that has 3 to 60 carbon atoms and does not include *—N═*′ as a ring-forming moiety, and the term “π electron-deficient nitrogen-containing C₁-C₆₀ heterocyclic group” as utilized herein refers to a heterocyclic group that has 1 to 60 carbon atoms and includes *—N═*′ as a ring-forming moiety.

For example,
the C₃-C₆₀ carbocyclic group may be i) Group T1 or ii) a condensed cyclic group in which two or more of Group T1 are condensed with each other (e.g., a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 and at least one Group T1 are condensed with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (e.g., the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),
the π electron-deficient nitrogen-containing C₁-C₆₀ heterocyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
Group T3 may be a furan group, a thiophene group, a 1H-pyrrole group, a silole group, or a borole group, and
Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆₀ heterocyclic group,” “TT electron-rich C₃-C₆₀ cyclic group,” or “TT electron-deficient nitrogen-containing C₁-C₆₀ heterocyclic group” as utilized herein may refer to a group condensed to any cyclic group, a monovalent group, or a polyvalent group (e.g., a divalent group, a trivalent group, a tetravalent group, etc.) according to the structure of a formula for which the corresponding term is utilized. For example, the “benzene group” may be a benzo group, a phenyl group, a phenylene group, and/or the like, which may be easily understood by one of ordinary skill in the art according to the structure of a formula including the “benzene group.”

Depending on context, in the present disclosure, a divalent group may refer or be a polyvalent group (e.g., trivalent, tetravalent, etc., and not just divalent) per, e.g., the structure of a formula in connection with which of the terms are utilized.

Non-limiting examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₅-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Non-limiting examples of the divalent C₅-C₆₀ carbocyclic group and the divalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as utilized herein refers to a linear or branched aliphatic hydrocarbon monovalent group that has 1 to 60 carbon atoms, and non-limiting examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as utilized herein refers to a divalent group having substantially the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of a C₂-C₆₀ alkyl group, and non-limiting examples thereof may include an ethenyl group, a propenyl group, a butenyl group, and/or the like. The term “C₂-C₆₀ alkenylene group” as utilized herein refers to a divalent group having substantially the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of a C₂-C₆₀ alkyl group, and non-limiting examples thereof may include an ethynyl group, a propynyl group, and/or the like. The term “C₂-C₆₀ alkynylene group” as utilized herein refers to a divalent group having substantially the same structure as the C₂-C₆₀ alkynyl group.

The term “C₁-C₆₀ alkoxy group” as utilized herein refers to a monovalent group represented by —OA₁₀₁ (wherein A₁₀₁ is a C₁-C₆₀ alkyl group), and non-limiting examples thereof may include a methoxy group, an ethoxy group, an isopropyloxy group, and/or the like.

The term “C₃-C₁₀ cycloalkyl group” as utilized herein refers to a monovalent saturated hydrocarbon cyclic group having 3 to 10 carbon atoms, and non-limiting examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and/or the like. The term “C₃-C₁₀ cycloalkylene group” as utilized herein refers to a divalent group having substantially the same structure as the C₃-C₁₀ cycloalkyl group.

The term “C₁-C₁₀ heterocycloalkyl group” as utilized herein refers to a monovalent cyclic group of 1 to 10 carbon atoms, further including, in addition to carbon atoms, at least one heteroatom as ring-forming atoms, and non-limiting examples thereof may include a 1,2,3,4-oxatriazolidinyl group, a tetrahydrofuranyl group, a tetrahydrothiophenyl group, and/or the like. The term “C₁-C₁₀ heterocycloalkylene group” as utilized herein refers to a divalent group having substantially the same structure as the C₁-C₁₀ heterocycloalkyl group.

The term “C₃-C₁₀ cycloalkenyl group” as utilized herein refers to a monovalent cyclic group that 3 to 10 carbon atoms, at least one carbon-carbon double bond in the ring thereof, and no aromaticity, and non-limiting examples thereof may include a cyclopentenyl group, a cyclohexenyl group, a cycloheptenyl group, and/or the like. The term “C₃-C₁₀ cycloalkenylene group” as utilized herein refers to a divalent group having substantially the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom as ring-forming atoms and at least one double bond in the cyclic structure thereof. Non-limiting examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and/or the like. The term “C₁-C₁₀ heterocycloalkenylene group” as utilized herein refers to a divalent group having substantially the same structure as the C₁-C₁₀ heterocycloalkenyl group.

The term “C₆-C₆₀ aryl group” as utilized herein refers to a monovalent group having a carbocyclic aromatic system of 6 to 60 carbon atoms, and the term “C₆-C₆₀ arylene group” as utilized herein refers to a divalent group having a carbocyclic aromatic system of 6 to 60 carbon atoms. Non-limiting examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and/or the like. When the C₆-C₆₀ aryl group and the C₆-C₆₀ arylene group each include two or more rings, the rings may be condensed with each other.

The term “C₁-C₆₀ heteroaryl group” as utilized herein refers to a monovalent group having a heterocyclic aromatic system of 1 to 60 carbon atoms, further including, in addition to carbon atoms, at least one heteroatom as ring-forming atoms. The term “C₁-C₆₀ heteroarylene group” as utilized herein refers to a divalent group having a heterocyclic aromatic system of 1 to 60 carbon atoms, further including, in addition to carbon atoms, at least one heteroatom as ring-forming atoms. Non-limiting examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and/or the like. When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group (for example, having 8 to 60 carbon atoms) having two or more rings condensed to each other, only carbon atoms as ring-forming atoms, and no aromaticity in the entire molecular structure as a whole. Non-limiting examples of the monovalent non-aromatic condensed polycyclic group may include an indenyl group, a fluorenyl group, a spiro-bifluorenyl group, a benzofluorenyl group, an indenophenanthrenyl group, an indeno anthracenyl group, and/or the like. The term “divalent non-aromatic condensed polycyclic group” as utilized herein refers to a divalent group having substantially the same structure as the monovalent non-aromatic condensed polycyclic group described above.

The term “monovalent non-aromatic condensed heteropolycyclic group” as utilized herein refers to a monovalent group (for example, having 1 to 60 carbon atoms) having two or more rings condensed to each other, further including, in addition to carbon atoms, at least one heteroatom as ring-forming atoms, and no aromaticity in the entire molecular structure as a whole. Non-limiting examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and/or the like. The term “divalent non-aromatic condensed heteropolycyclic group” as utilized herein refers to a divalent group having substantially the same structure as the monovalent non-aromatic condensed heteropolycyclic group.

The term “C₆-C₆₀ aryloxy group” as utilized herein indicates —OA₁₀₂ (wherein A₁₀₂ is a C₆-C₆₀ aryl group), and the term “C₆-C₆₀ arylthio group” as utilized herein indicates —SA₁₀₃ (wherein A₁₀₃ is a C₆-C₆₀ aryl group).

The term “C₇-C₆₀ aryl alkyl group” utilized herein refers to -A₁₀₄A₁₀₅ (where A₁₀₄ may be a C₁-C₅₄ alkylene group, and A₁₀₅ may be a C₆-C₅₉ aryl group), and the term C₂-C₆₀ heteroaryl alkyl group” utilized herein refers to -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as utilized herein may be:
deuterium, —F, —Cl, —Br, —I, a hydroxyl group, a cyano group, a nitro group, —SCN;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, —SCN, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₅-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, —SCN, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In the present disclosure, Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; —SCN; or a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as utilized herein refers to any atom other than a carbon atom. Examples of the heteroatom may include O, S, N, P, Si, B, Ge, Se, and any combination thereof.

The term “third-row transition metal” as utilized herein may include hafnium (Hf), tantalum (Ta), tungsten (W), rhenium (Re), osmium (Os), iridium (Ir), platinum (Pt), gold (Au), and/or the like.

“Ph” as utilized herein refers to a phenyl group, “Me” as utilized herein refers to a methyl group, “Et” as utilized herein refers to an ethyl group, “tert-Bu” or “But” as utilized herein refers to a tert-butyl group, and “OMe” as utilized herein refers to a methoxy group.

The term “biphenyl group” as utilized herein refers to “a phenyl group substituted with a phenyl group.” In some embodiments, the “biphenyl group” is a substituted phenyl group having a C₆-C₆₀ aryl group as a substituent.

The term “terphenyl group” as utilized herein refers to “a phenyl group substituted with a biphenyl group”. In some embodiments, the “terphenyl group” is a substituted phenyl group having, as a substituent, a C₆-C₆₀ aryl group substituted with a C₆-C₆₀ aryl group.

*, *′, and *″ as utilized herein, unless defined otherwise, each refer to a binding site to a neighboring atom in a corresponding formula or moiety.

In the present disclosure, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to one or more embodiments and light-emitting devices according to one or more embodiments will be described in more detail with reference to the following Synthesis Examples and Examples. The wording “B was utilized instead of A” utilized in describing Synthesis Examples refers to that an identical molar equivalent of B was utilized in place of A.

### EXAMPLES

### Synthesis Example 1: Synthesis of Compound 9

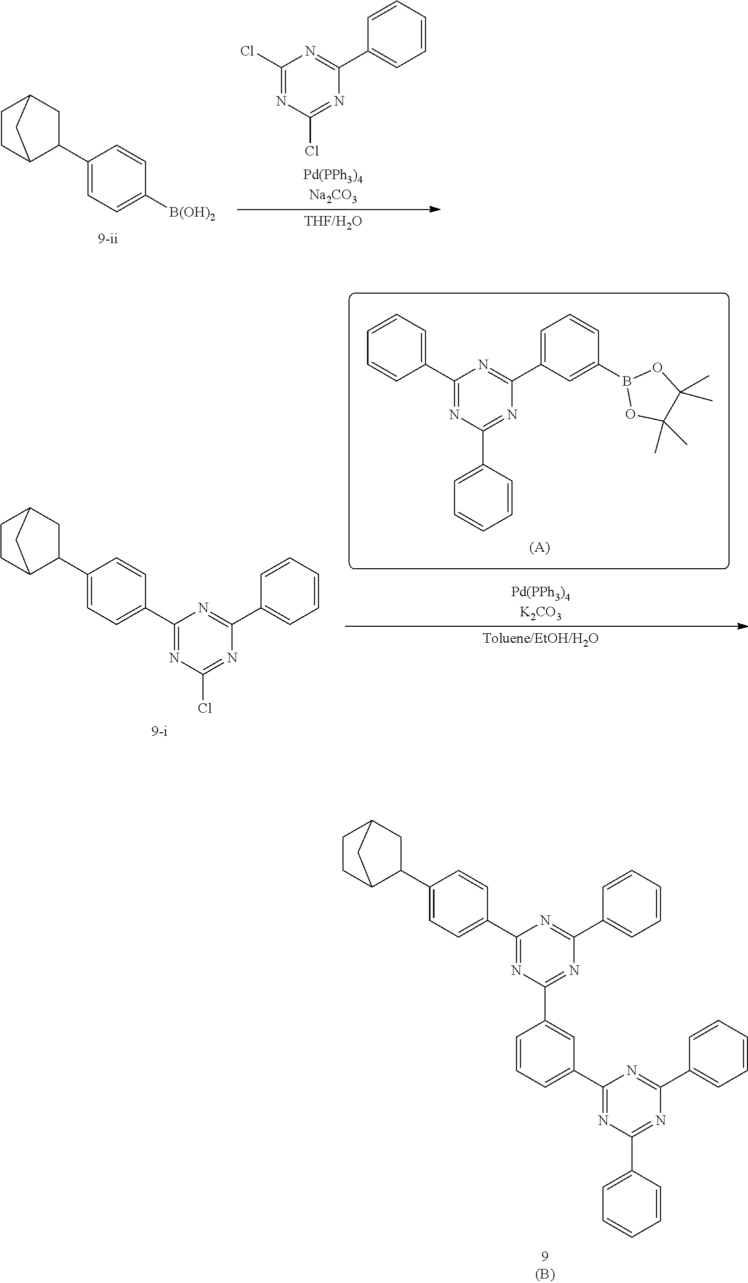

### 1) Synthesis of Compound 9-i

100 g (462.8 mmol, 1 eq.) of (4-(bicyclo[2.2.1]heptan-2-yl)phenyl)boronic acid, 209.2 g (925.5 mmol, 2 eq.) of 2,4-dichloro-6-phenyl-1,3,5-triazine, 21.4 g (18.5 mmol, 0.04 eq.) of Pd(PPh₃)₄, and 73.6 g (694.2 mmol, 1.5 eq.) of Na₂CO₃ were added to 1,500 mL of THF and 400 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 9-i, which is 2-(4-(bicyclo[2.2.1]heptan-2-yl)phenyl)-4-chloro-6-phenyl-1,3,5-triazine (103.8 g, yield of 62%).

Mass: [(M+H)⁺]: 361.87

NMR(¹H): σ=8.57 (2H, d), 8.36 (2H, d), 7.53-7.50 (5H, m), 2.62(1H, t), 2.19 (1H, s), 2.10-1.99 (2H, m), 1.82 (1H, s), 1.70-1.64 (2H, m), 1.44-1.36 (4H, m)

### 2) Synthesis of Compound 9

10 g (27.6 mmol, 1 eq.) of 2-(4-(bicyclo[2.2.1]heptan-2-yl)phenyl)-4-chloro-6-phenyl-1,3,5-triazine, 14.4 g (33.2 mmol, 1.2 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.6 g (55.3 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 9 (12.8 g, yield of 73%).

Mass: [(M+H)⁺]: 634.79

### Synthesis of Compounds 29, 45, 73, 85, 97, 313, 357, 377, and 389

Target Compound B was synthesized in substantially the same manner as in the synthesis of Compound 9 according to Synthesis Example 1, except that the intermediate (A) was changed to Intermediate A in Table 1.

TABLE 1

| | | | Yield | Mass |
|---|---|---|---|---|
| Com- | | | (9-i to | [(M + |
| pound | Intermediate A | Target Compound B | B) | H)⁺] |
| Com- pound 29 | 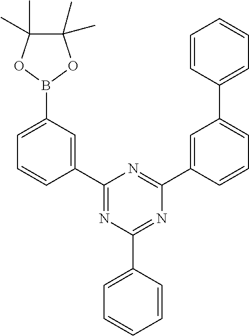 [SMILES: CC1(C)OB(c2cccc(-c3nc(-c4ccccc4)nc(-c4cccc(-c5ccccc5)c4)n3)c2)OC1(C)C] | 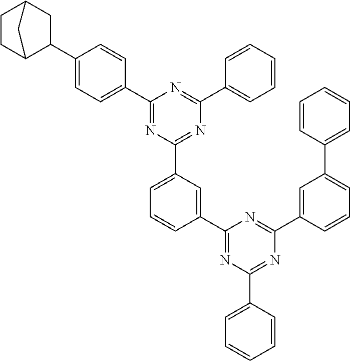 [SMILES: c1ccc(-c2cccc(-c3nc(-c4ccccc4)nc(-c4cccc(-c5nc(-c6ccccc6)nc(-c6ccc(C7CC8CCC7C8)cc6)n5)c4)n3)c2)cc1] | 75% | 710.89 |
| Com- pound 45 | 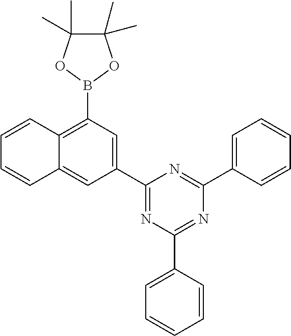 [SMILES: CC1(C)OB(c2cc(-c3nc(-c4ccccc4)nc(-c4ccccc4)n3)cc3ccccc23)OC1(C)C] | 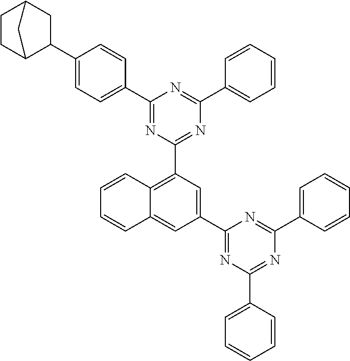 [SMILES: c1ccc(-c2nc(-c3ccccc3)nc(-c3cc(-c4nc(-c5ccccc5)nc(-c5ccc(C6CC7CCC6C7)cc5)n4)c4ccccc4c3)n2)cc1] | 74% | 684.85 |
| Com- pound 73 | 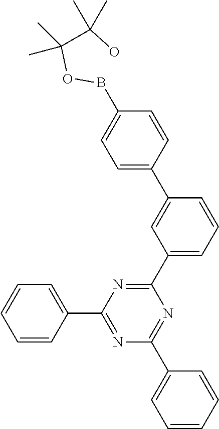 | 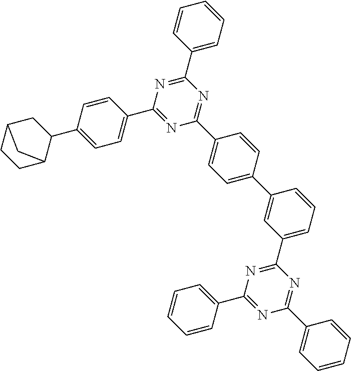 [SMILES: c1ccc(-c2nc(-c3ccc(-c4cccc(-c5nc(-c6ccccc6)nc(-c6ccccc6)n5)c4)cc3)nc(-c3ccc(C4CC5CCC4C5)cc3)n2)cc1] | 70% | 710.89 |
| Com- pound 85 | 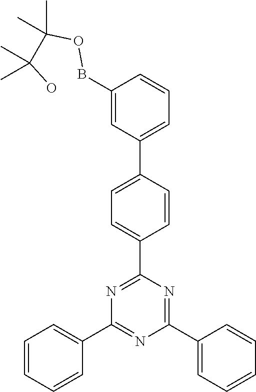 | 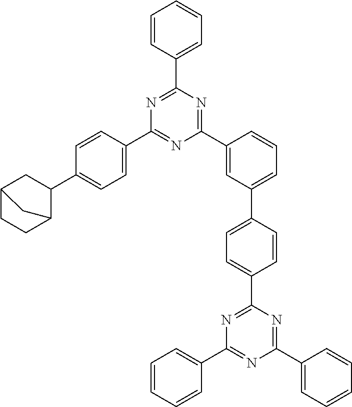 [SMILES: c1ccc(-c2nc(-c3ccccc3)nc(-c3ccc(-c4cccc(-c5nc(-c6ccccc6)nc(-c6ccc(C7CC8CCC7C8)cc6)n5)c4)cc3)n2)cc1] | 74% | 710.89 |
| Com- pound 97 | 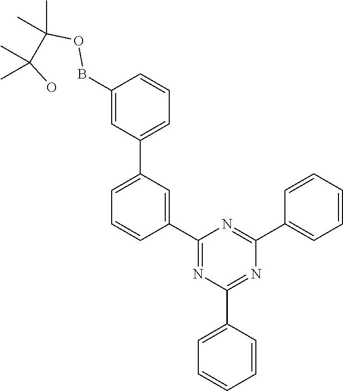 | 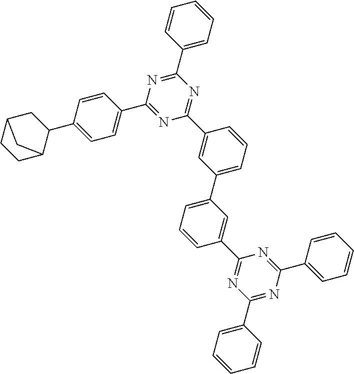 [SMILES: c1ccc(-c2nc(-c3ccccc3)nc(-c3cccc(-c4cccc(-c5nc(-c6ccccc6)nc(-c6ccc(C7CC8CCC7C8)cc6)n5)c4)c3)n2)cc1] | 74% | 710.89 |
| Com- pound 313 | 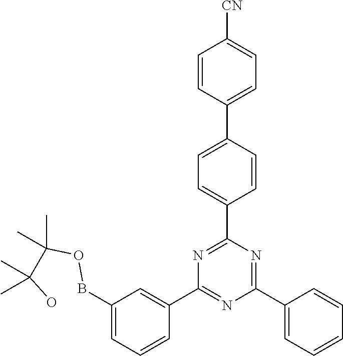 | 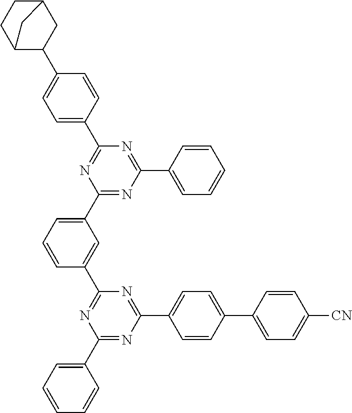 [SMILES: N#Cc1ccc(-c2ccc(-c3nc(-c4ccccc4)nc(-c4cccc(-c5nc(-c6ccccc6)nc(-c6ccc(C7CC8CCC7C8)cc6)n5)c4)n3)cc2)cc1] | 66% | 735.90 |
| Com- pound 357 | 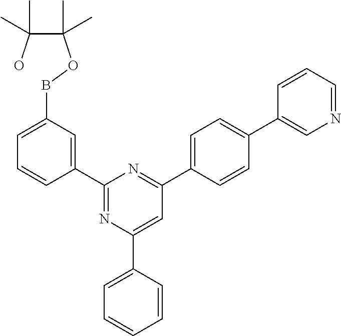 | 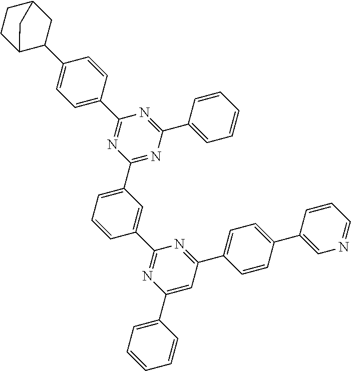 [SMILES: c1ccc(-c2cc(-c3ccc(-c4cccnc4)cc3)nc(-c3cccc(-c4nc(-c5ccccc5)nc(-c5ccc(C6CC7CCC6C7)cc5)n4)c3)n2)cc1] | 64% | 710.89 |
| Com- pound 377 | 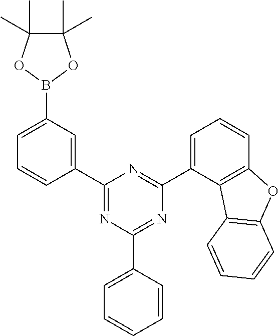 [SMILES: CC1(C)OB(c2cccc(-c3nc(-c4ccccc4)nc(-c4cccc5oc6ccccc6c45)n3)c2)OC1(C)C] | 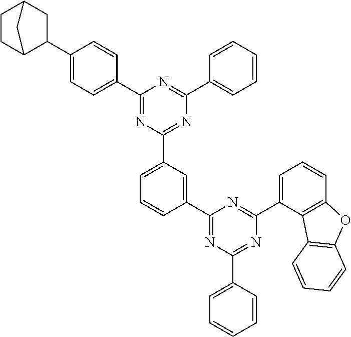 [SMILES: c1ccc(-c2nc(-c3ccc(C4CC5CCC4C5)cc3)nc(-c3cccc(-c4nc(-c5ccccc5)nc(-c5cccc6oc7ccccc7c56)n4)c3)n2)cc1] | 72% | 724.87 |
| Com- pound 389 | 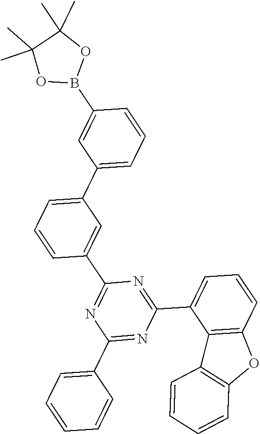 [SMILES: CC1(C)OB(c2cccc(-c3cccc(-c4nc(-c5ccccc5)nc(-c5cccc6oc7ccccc7c56)n4)c3)c2)OC1(C)C] | 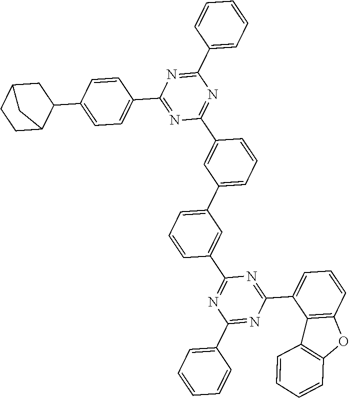 [SMILES: c1ccc(-c2nc(-c3ccc(C4CC5CCC4C5)cc3)nc(-c3cccc(-c4cccc(-c5nc(-c6ccccc6)nc(-c6cccc7oc8ccccc8c67)n5)c4)c3)n2)cc1] | 74% | 800.97 |

### Synthesis Example 2: Synthesis of Compound 10

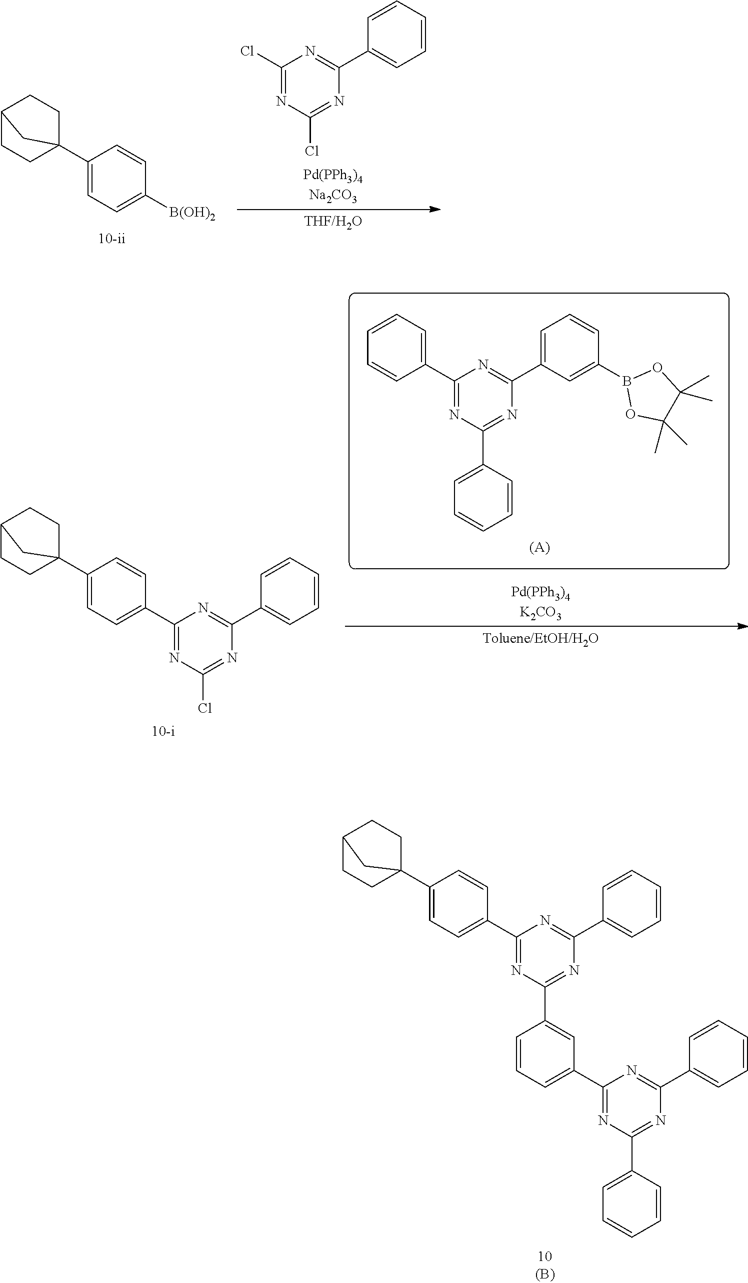

### 1) Synthesis of Compound 10-i

20 g (92.6 mmol, 1 eq.) of (4-(bicyclo[2.2.1]heptan-1-yl)phenyl)boronic acid, 41.8 g (185.1 mmol, 2 eq.) of 2,4-dichloro-6-phenyl-1,3,5-triazine, 4.3 g (3.7 mmol, 0.04 eq.) of Pd(PPh₃)₄, and 14.7 g (138.8 mmol, 1.5 eq.) of Na₂CO₃ were added to 300 mL of THF and 80 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 10-i, which is 2-(4-(bicyclo[2.2.1]heptan-1-yl)phenyl)-4-chloro-6-phenyl-1,3,5-triazine (21.4 g, yield of 64%).

Mass: [(M+H)⁺]: 361.87

NMR(¹H): σ=8.49 (2H, d), 8.36 (2H, d), 7.50 (3H, s), 7.38 (2H, s) 2.19 (1H, s), 1.82 (2H, s), 1.70-1.64 (4H, m), 1.44-1.36 (4H, m)

### 2) Synthesis of Compound 10

10 g (27.6 mmol, 1 eq.) of 2-(4-(bicyclo[2.2.1]heptan-1-yl)phenyl)-4-chloro-6-phenyl-1,3,5-triazine, 15.5 g (30.4 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.6 g (55.3 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 10 (12.3 g, yield of 70%).

Mass: [(M+H)⁺]: 634.79

### Synthesis of Compound 358

Target Compound B was synthesized in substantially the same manner as in the synthesis of Compound 10 according to Synthesis Example 2, except that the intermediate (A) was changed to Intermediate A in Table 2.

TABLE 2

| | | | Yield | Mass |
|---|---|---|---|---|
| Com- | | | (10-i to | [(M + |
| pound | Intermediate A | Target Compound B | B) | H)⁺] |
| Com- pound 358 | 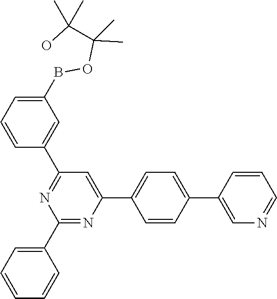 | 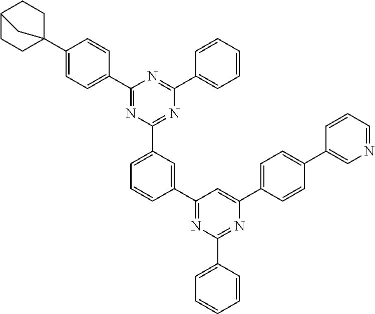 [SMILES: c1ccc(-c2nc(-c3ccc(-c4cccnc4)cc3)cc(-c3cccc(-c4nc(-c5ccccc5)nc(-c5ccc(C67CCC(CC6)C7)cc5)n4)c3)n2)cc1] | 62% | 710.89 |

### Synthesis Example 3: Synthesis of Compound 11

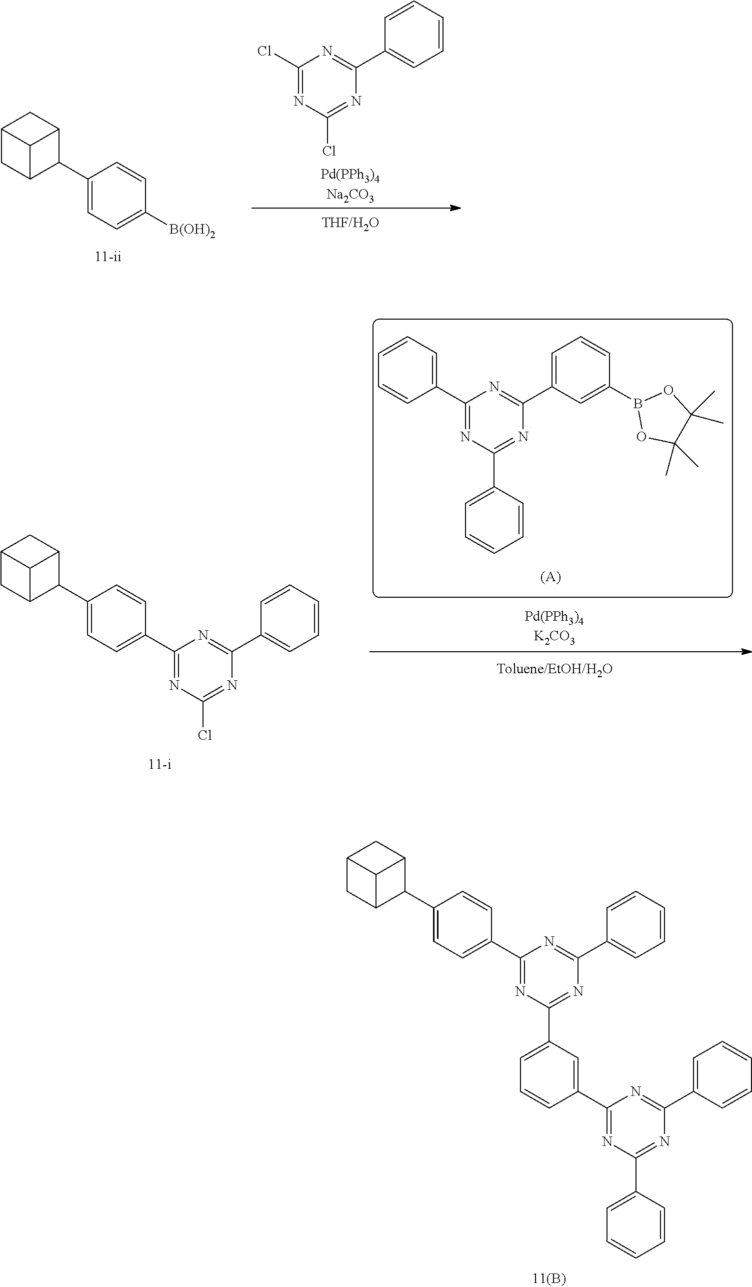

### 1) Synthesis of Compound 11-i

20 g (92.6 mmol, 1 eq.) of (4-((1S,5R,6S)-tricyclo[3.1.1.03,6]heptan-2-yl)phenyl) boronic acid, 41.8 g (185.1 mmol, 2 eq.) of 2,4-dichloro-6-phenyl-1,3,5-triazine, 4.3 g (3.7 mmol, 0.04 eq.) of Pd(PPh₃)₄, and 14.7 g (138.8 mmol, 1.5 eq.) of Na₂CO₃ were added to 300 mL of THF and 80 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 11-i, which is 2-chloro-4-phenyl-6-(4-((1S,5R,6S)-tricyclo[3.1.1.03,6]heptan-2-yl)phenyl)-1,3,5-triazine (20.1 g, yield of 60%).

Mass: [(M+H)⁺]: 361.87

NMR(¹H): σ=8.57 (2H, d), 8.36 (2H, d), 7.50 (5H, m), 4.39 (2H, t) 4.01 (1H, t), 3.81 (1H, t), 2.61 (1H, t), 1.50-1.25 (4H, m)

### 2) Synthesis of Compound 11

10 g (27.8 mmol, 1 eq.) of 2-chloro-4-phenyl-6-(4-((1S,5R,6S)-tricyclo[3.1.1.03,6]heptan-2-yl)phenyl)-1,3,5-triazine, 13.3 g (30.6 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.7 g (55.6 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 11 (12.7 g, yield of 72%).

Mass: [(M+H)⁺]: 632.77

### Synthesis of Compound 87 and 315

Target Compound B was synthesized in substantially the same manner as in the synthesis of Compound 11 according to Synthesis Example 3, except that the intermediate (A) was changed to Intermediate A in Table 3.

TABLE 3

| | | | Yield | Mass |
|---|---|---|---|---|
| Compound | Intermediate A | Target Compound B | (11-i to B) | [(M + H)⁺] |
| Compound 87 | 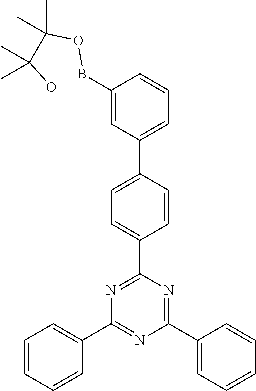 | 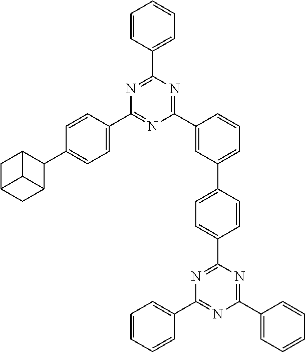 [SMILES: c1ccc(-c2nc(-c3ccccc3)nc(-c3ccc(-c4cccc(-c5nc(-c6ccccc6)nc(-c6ccc(C7C8CC9CC7C98)cc6)n5)c4)cc3)n2)cc1] | 75% | 708.87 |
| Compound 315 | 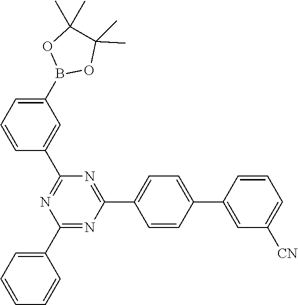 [SMILES: CC1(C)OB(c2cccc(-c3nc(-c4ccccc4)nc(-c4ccc(-c5cccc(C#N)c5)cc4)n3)c2)OC1(C)C] | 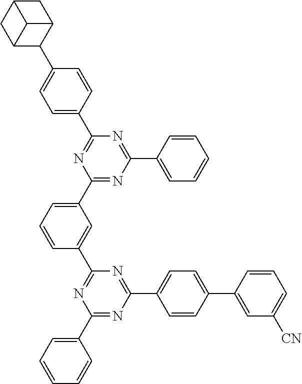 [SMILES: N#Cc1cccc(-c2ccc(-c3nc(-c4ccccc4)nc(-c4cccc(-c5nc(-c6ccccc6)nc(-c6ccc(C7C8CC9CC7C98)cc6)n5)c4)n3)cc2)c1] | 70% | 733.88 |

### Synthesis Example 4: Synthesis of Compound 12

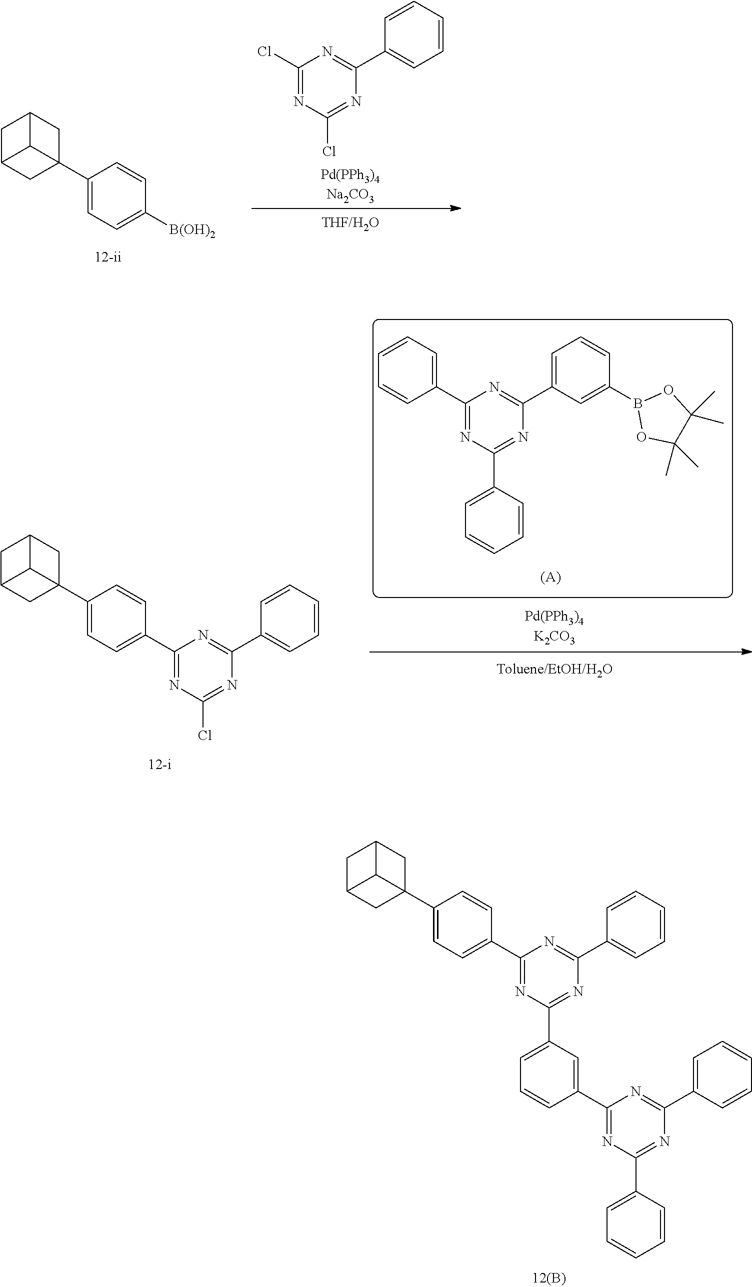

### 1) Synthesis of Compound 12-i

20 g (93.4 mmol, 1 eq.) of (4-((1R,3R,6R)-tricyclo[3.1.1.03,6]heptan-1-yl)phenyl)boronic acid, 42.2 g (186.9 mmol, 2 eq.) of 2,4-dichloro-6-phenyl-1,3,5-triazine, 4.3 g (3.7 mmol, 0.04 eq.) of Pd(PPh₃)₄, and 14.9 g (140.1 mmol, 1.5 eq.) of Na₂CO₃ were added to 300 mL of THF and 80 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 12-i, which is 2-chloro-4-phenyl-6-(4-((1R,3R,6R)-tricyclo[3.1.1.03,6]heptan-1-yl)phenyl)-1,3,5-triazine (20.3 g, yield of 60%).

Mass: [(M+H)⁺]: 359.86

NMR(¹H): σ=8.49 (2H, d), 8.36 (2H, d), 7.50 (3H, d), 7.38 (2H, d), 4.1(1H, m), 4.01 (2H, m), 1.8-1.5(4H, m), 1.5-1.3(2H, m)

### 2) Synthesis of Compound 12

10 g (27.8 mmol, 1 eq.) of 2-chloro-4-phenyl-6-(4-((1R,3R,6R)-tricyclo[3.1.1.03,6]heptan-1-yl)phenyl)-1,3,5-triazine, 13.3 g (30.6 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.7 g (55.6 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 12 (12.7 g, yield of 72%).

Mass: [(M+H)⁺]: 632.77

### Synthesis Example 5: Synthesis of Compound 13

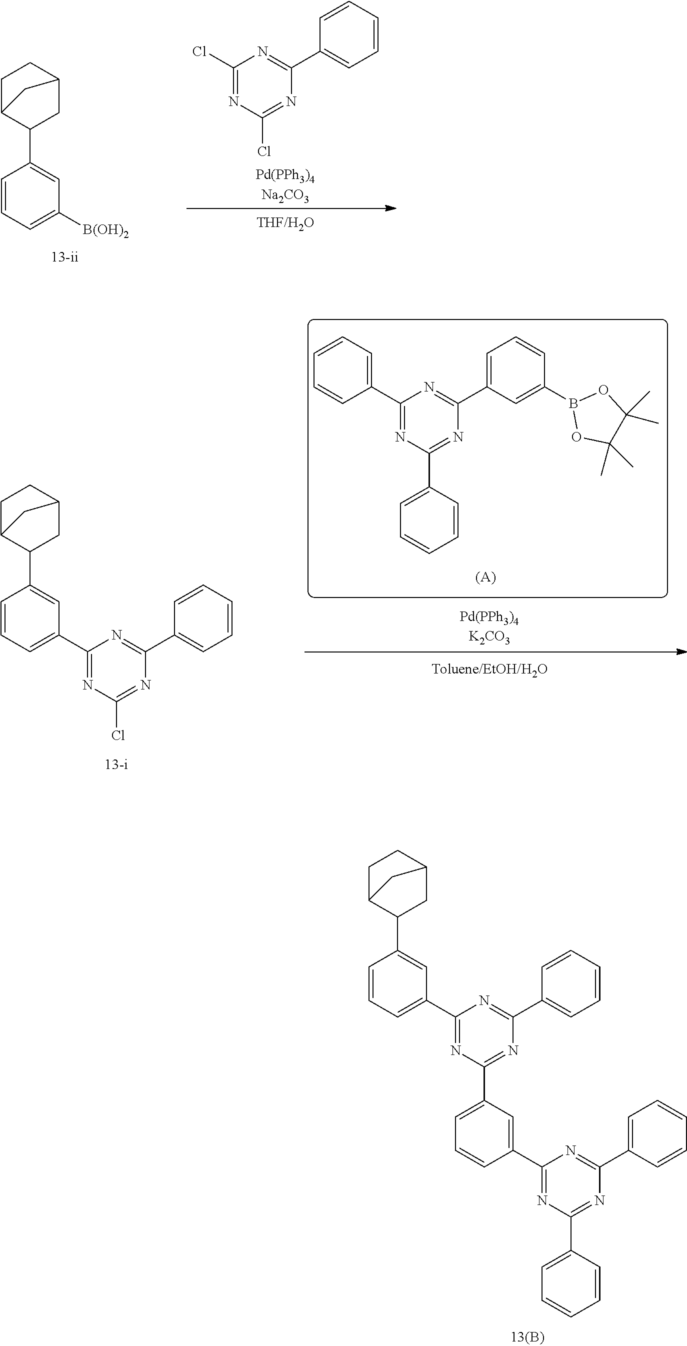

### 1) Synthesis of Compound 13-i

50 g (231.4 mmol, 1 eq.) of (3-(bicyclo[2.2.1]heptan-2-yl)phenyl)boronic acid, 104.6 g (462.8 mmol, 2 eq.) of 2,4-dichloro-6-phenyl-1,3,5-triazine, 10.8 g (9.3 mmol, 0.04 eq.) of Pd(PPh₃)₄, and 36.8 g (347.1 mmol, 1.5 eq.) of Na₂CO₃ were added to 750 mL of THF and 200 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 13-i, which is 2-(3-(bicyclo[2.2.1]heptan-2-yl)phenyl)-4-chloro-6-phenyl-1,3,5-triazine (51.9 g, yield of 62%).

Mass: [(M+H)⁺]: 361.87

NMR(¹H): σ=8.36 (2H, d), 8.18 (1H, s), 7.94 (1H, s), 7.54-7.50 (4H, m), 7.38 (1H, d), 2.62 (1H, d), 2.19 (1H, s), 1.88 (2H, m), 1.82-1.79 (3H, m), 1.56-1.31 (4H, m)

### 2) Synthesis of Compound 13

10 g of 2-(3-(bicyclo[2.2.1]heptan-2-yl)phenyl)-4-chloro-6-phenyl-1,3,5-triazine, 13.2 g (30.4 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.6 g (55.3 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 13 (12.3 g, yield of 70%).

Mass: [(M+H)⁺]: 634.79

### Synthesis of Compounds 74 and 98

Target Compound B was synthesized in substantially the same manner as in the synthesis of Compound 13 according to Synthesis Example 5, except that the intermediate (A) was changed to Intermediate A in Table 4.

TABLE 4

| | | | Yield | Mass |
|---|---|---|---|---|
| Compound | Intermediate A | Target Compound B | (13-i to B) | [(M + H)⁺] |
| Compound 74 | 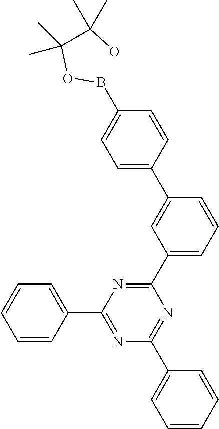 | 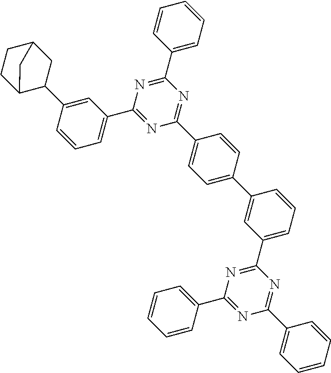 [SMILES: c1ccc(-c2nc(-c3ccccc3)nc(-c3cccc(-c4ccc(-c5nc(-c6ccccc6)nc(-c6cccc(C7CC8CCC7C8)c6)n5)cc4)c3)n2)cc1] | 73% | 710.89 |
| Compound 98 | 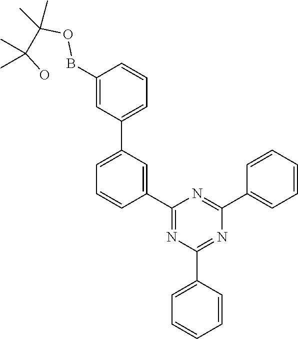 | 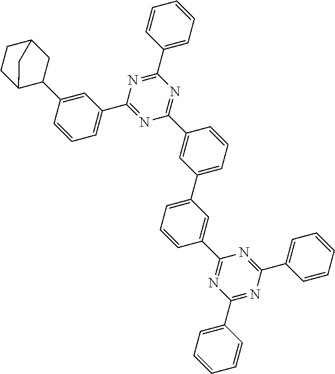 [SMILES: c1ccc(-c2nc(-c3ccccc3)nc(-c3cccc(-c4cccc(-c5nc(-c6ccccc6)nc(-c6cccc(C7CC8CCC7C8)c6)n5)c4)c3)n2)cc1] | 74% | 710.89 |

### Synthesis Example 6: Synthesis of Compound 14

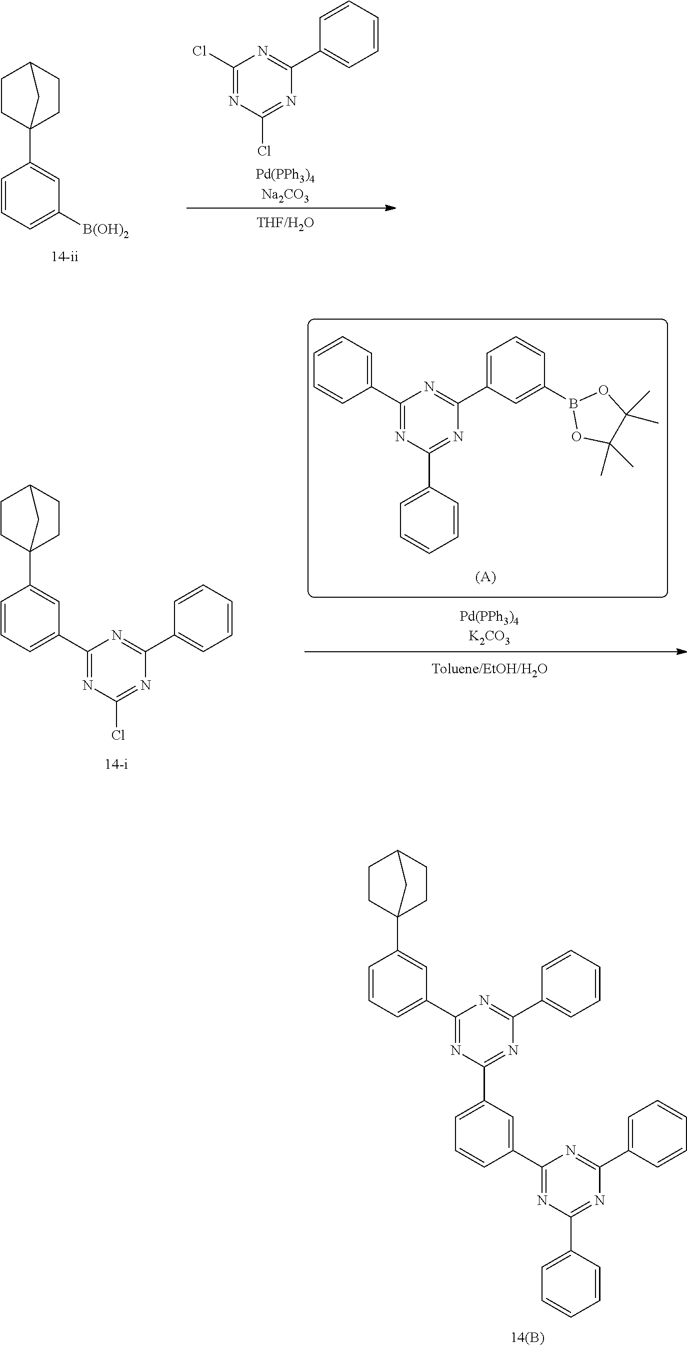

### 1) Synthesis of Compound 14-i

50 g (231.4 mmol, 1 eq.) of (3-(bicyclo[2.2.1]heptan-1-yl)phenyl)boronic acid, 104.6 g (462.8 mmol, 2 eq.) of 2,4-dichloro-6-phenyl-1,3,5-triazine, 10.8 g (9.3 mmol, 0.04 eq.) of Pd(PPh₃)₄, and 36.8 g (347.1 mmol, 1.5 eq.) of Na₂CO₃ were added to 750 mL of THF and 200 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 14-i, which is 2-(3-(bicyclo[2.2.1]heptan-1-yl)phenyl)-4-chloro-6-phenyl-1,3,5-triazine (46.1 g, yield of 55%).

Mass: [(M+H)⁺]: 361.87

NMR(¹H): σ=8.36 (2H, d), 8.08 (1H, d), 7.93 (1H, s), 7.55-7.46 (5H, m), 2.19 (1H, m), 1.80-1.54(6H, m), 1.57-1.32 (4H, m)

### 2) Synthesis of Compound 14

10 g (27.6 mmol, 1.0 eq.) of 2-(3-(bicyclo[2.2.1]heptan-1-yl)phenyl)-4-chloro-6-phenyl-1,3,5-triazine, 13.2 g (30.4 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.6 g (55.3 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 14 (13.0 g, yield of 74%).

Mass: [(M+H)⁺]: 634.79

### Synthesis of Compound 26

Target Compound B was synthesized in substantially the same manner as in the synthesis of Compound 14 according to Synthesis Example 6, except that the intermediate (A) was changed to Intermediate A in Table 5.

TABLE 5

| | | | Yield | |
|---|---|---|---|---|
| | | | (14-i | Mass |
| Com- | | | to | [(M + |
| pound | Intermediate A | Target Compound B | B) | H)⁺] |
| Com- pound 26 | 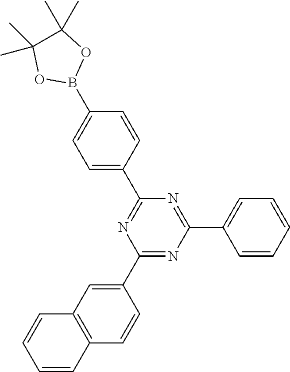 [SMILES: CC1(C)OB(c2ccc(-c3nc(-c4ccccc4)nc(-c4ccc5ccccc5c4)n3)cc2)OC1(C)C] | 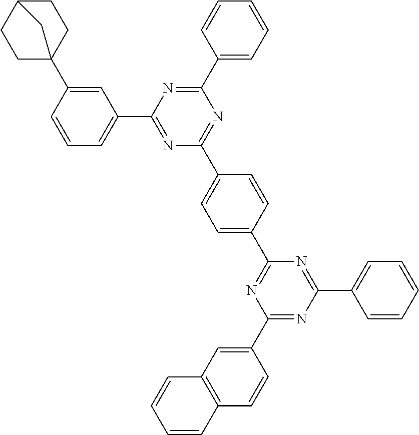 [SMILES: c1ccc(-c2nc(-c3ccc(-c4nc(-c5ccccc5)nc(-c5ccc6ccccc6c5)n4)cc3)nc(-c3cccc(C45CCC(CC4)C5)c3)n2)cc1] | 76% | 684.85 |

### Synthesis Example 7: Synthesis of Compound 15

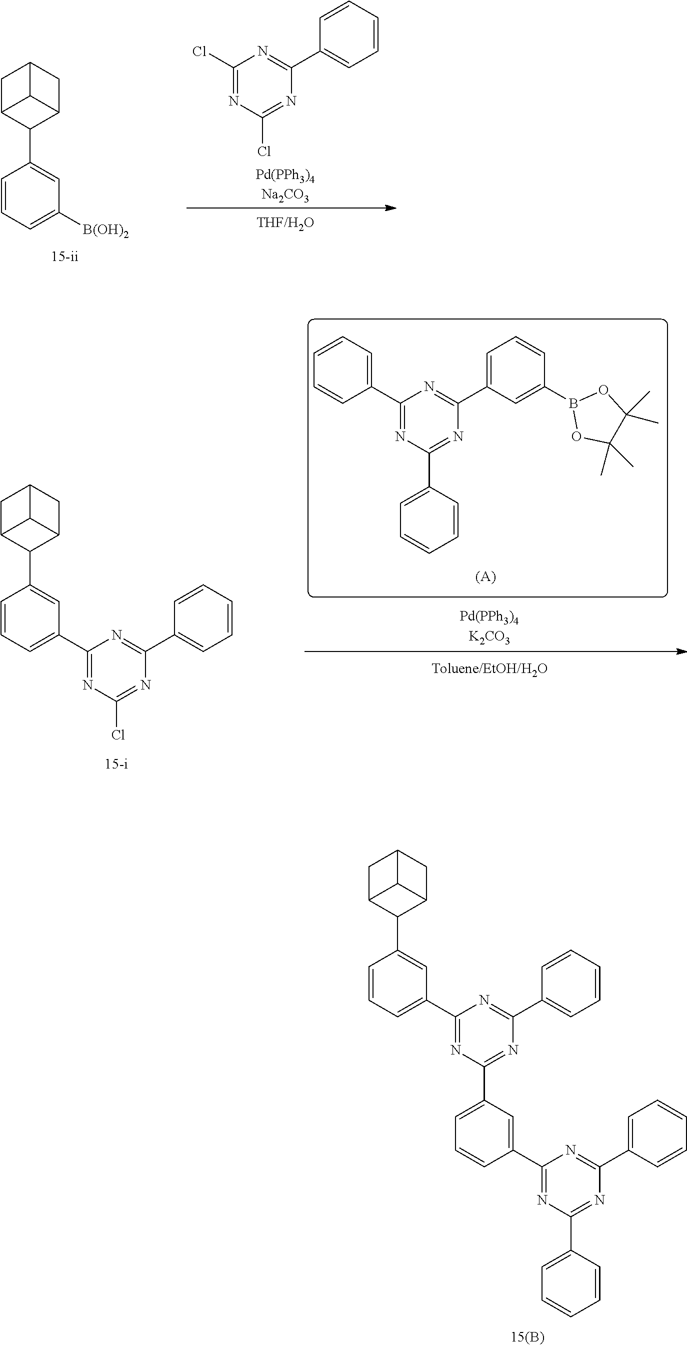

### 1) Synthesis of Compound 15-i

50 g (233.6 mmol, 1 eq.) of (3-((1R,2r,3S,6r)-tricyclo[3.1.1.03,6]heptan-2-yl)phenyl)boronic acid, 105.6 g (467.1 mmol, 2 eq.) of 2,4-dichloro-6-phenyl-1,3,5-triazine, 10.8 g (9.3 mmol, 0.04 eq.) of Pd(PPh₃)₄, and 37.1 g (350.4 mmol, 1.5 eq.) of Na₂CO₃ were added to 750 mL of THF and 200 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 15-i, which is 2-chloro-4-phenyl-6-(3-((1R,2r,3S,6r)-tricyclo[3.1.1.03,6]heptan-2-yl)phenyl)-1,3,5-triazine (47.9 g, yield of 57%).

Mass: [(M+H)⁺]: 359.86

NMR(¹H): σ=8.36 (2H, d), 8.18 (1H, d), 7.94 (1H, s), 7.54-7.40 (5H, m), 4.39 (2H, s), 4.01 (1H, s), 3.81(1H, s), 2.60 (1H, s), 1.5-1.25 (4H, m)

### 2) Synthesis of Compound 15

10 g (27.8 mmol, 1.0 eq.) of 2-chloro-4-phenyl-6-(3-((1R,2r,3S,6r)-tricyclo[3.1.1.03,6]heptan-2-yl)phenyl)-1,3,5-triazine, 13.3 g (30.6 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.7 g (55.6 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 15 (12.8 g, yield of 73%).

Mass: [(M+H)⁺]: 632.77

### Synthesis of Compound 76

Target Compound B was synthesized in substantially the same manner as in the synthesis of Compound 15 according to Synthesis Example 7, except that the intermediate (A) was changed to Intermediate A in Table 6.

TABLE 6

| Com- | | | Yield | Mass |
|---|---|---|---|---|
| pound | Intermediate A | Target Compound B | (15-i to B) | [(M + H)⁺] |
| Com- pound 76 | 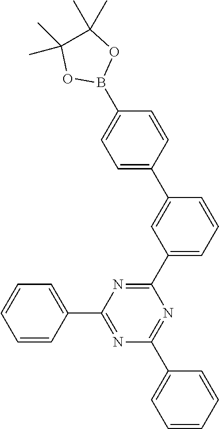 [SMILES: CC1(C)OB(c2ccc(-c3cccc(-c4nc(-c5ccccc5)nc(-c5ccccc5)n4)c3)cc2)OC1(C)C] | 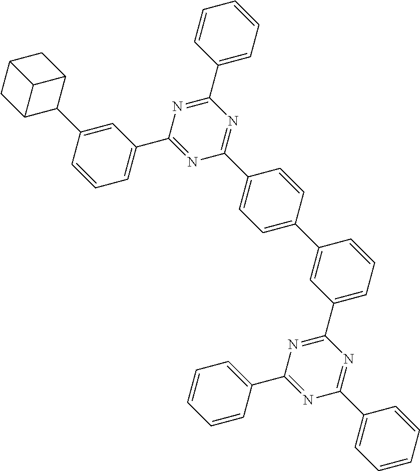 [SMILES: c1ccc(-c2nc(-c3ccccc3)nc(-c3cccc(-c4ccc(-c5nc(-c6ccccc6)nc(-c6cccc(C7C8CC9CC7C98)c6)n5)cc4)c3)n2)cc1] | 73% | 708.87 |

### Synthesis Example 8: Synthesis of Compound 16

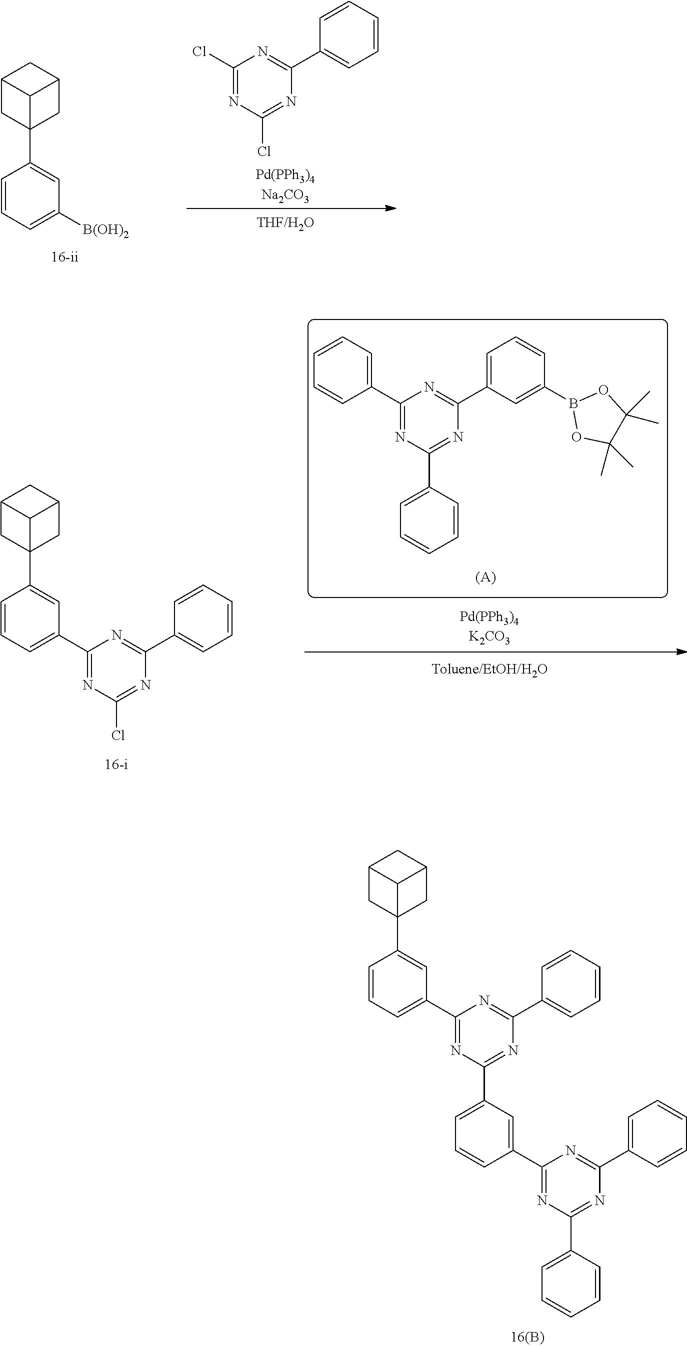

### 1) Synthesis of Compound 16-i

50 g (233.6 mmol, 1 eq.) of (3-((3R,5S,6r)-tricyclo[3.1.1.03,6]heptan-1-yl)phenyl)boronic acid, 105.6 g (467.1 mmol, 2 eq.) of 2,4-dichloro-6-phenyl-1,3,5-triazine, 10.8 g (9.3 mmol, 0.04 eq.) of Pd(PPh₃)₄, and 37.1 g (350.4 mmol, 1.5 eq.) of Na₂CO₃ were added to 750 mL of THF and 200 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 16-i, which is 2-chloro-4-phenyl-6-(3-((3R,5S,6r)-tricyclo[3.1.1.03,6]heptan-1-yl)phenyl)-1,3,5-triazine (46.2 g, yield of 55%).

Mass: [(M+H)⁺]: 359.86

NMR(¹H): σ=8.36 (2H, d), 8.08 (1H, s), 7.93 (1H, s), 7.55-7.46 (5H, m) 4.19 (1H, s), 4.01 (2H, s), 1.79-1.50 (4H, m), 1.40-1.30 (2H, m)

### 2) Synthesis of Compound 16

10 g (27.8 mmol, 1.0 eq.) of 2-chloro-4-phenyl-6-(3-((3R,5S,6r)-tricyclo[3.1.1.03,6]heptan-1-yl)phenyl)-1,3,5-triazine, 13.3 g (30.6 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.7 g (55.6 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 16 (12.3 g, yield of 70%).

Mass: [(M+H)⁺]: 632.77

### Synthesis of Compounds 60 and 100

Target Compound B was synthesized in substantially the same manner as in the synthesis of Compound 16 according to Synthesis Example 8, except that the intermediate (A) was changed to Intermediate A in Table 7.

TABLE 7

| | | | Yield | |
|---|---|---|---|---|
| | | | (16-i to | Mass |
| Compound | Intermediate A | Target Compound B | B) | [(M + H)⁺] |
| Compound 60 | 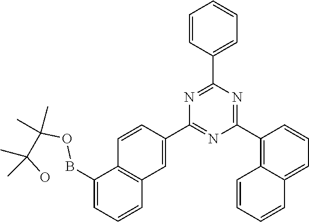 | 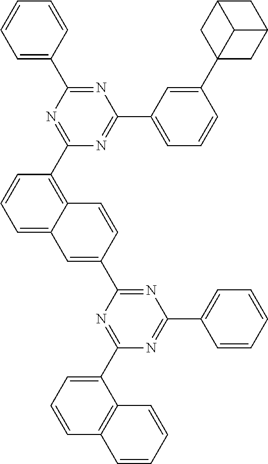 [SMILES: c1ccc(-c2nc(-c3ccc4c(-c5nc(-c6ccccc6)nc(-c6cccc(C78CC9CC(C7)C98)c6)n5)cccc4c3)nc(-c3cccc4ccccc34)n2)cc1] | 69% | 732.89 |
| Compound 100 | 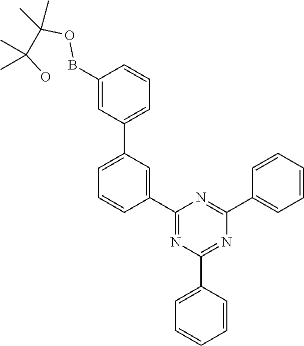 | 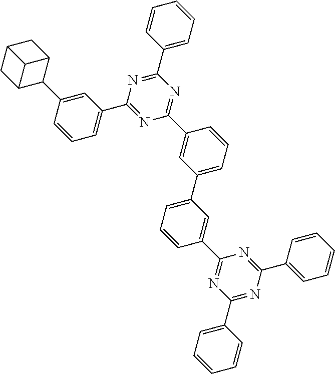 [SMILES: c1ccc(-c2nc(-c3ccccc3)nc(-c3cccc(-c4cccc(-c5nc(-c6ccccc6)nc(-c6cccc(C7C8CC9CC7C98)c6)n5)c4)c3)n2)cc1] | 75% | 708.87 |

### Synthesis Example 9: Synthesis of Compound 117

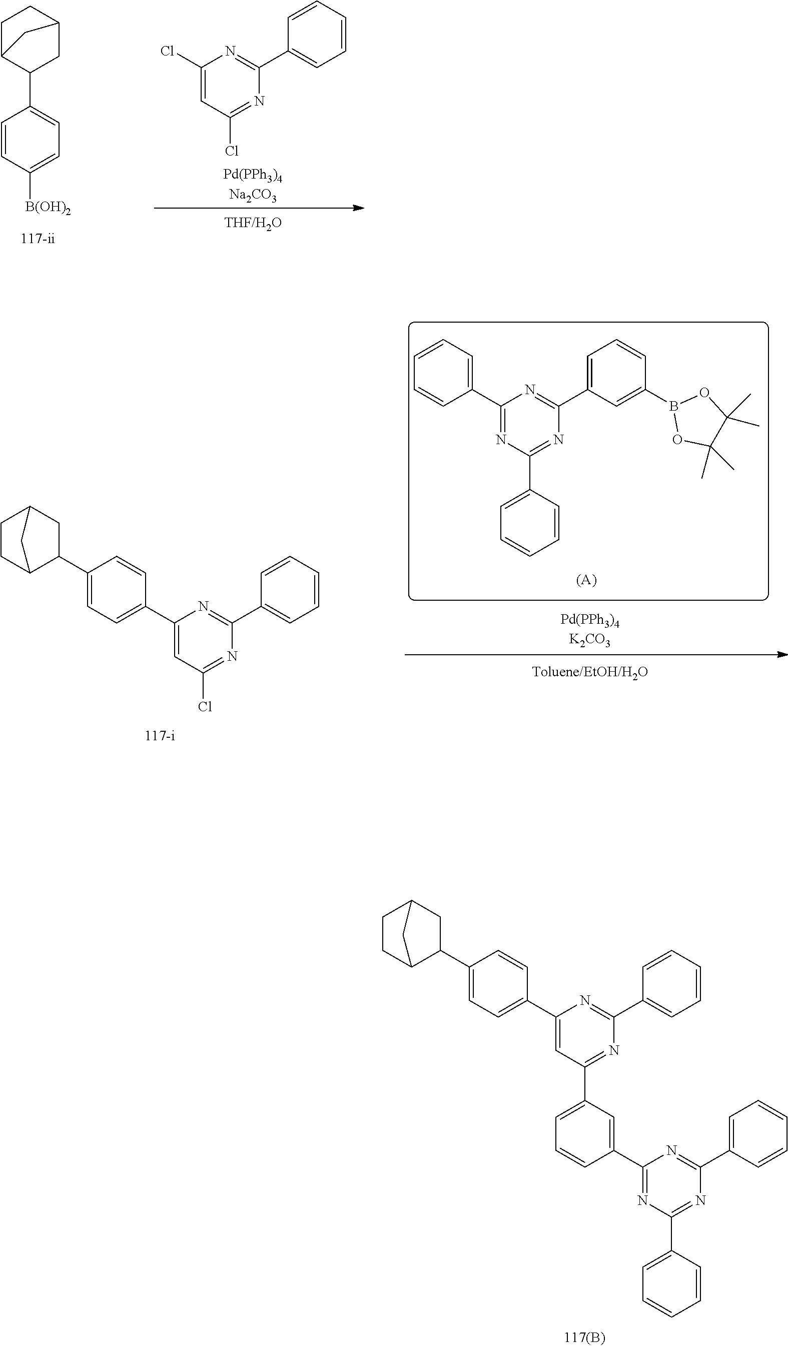

### 1) Synthesis of Compound 117-i

150 g (694.2 mmol, 1 eq.) of (4-(bicyclo[2.2.1]heptan-2-yl)phenyl)boronic acid, 312.5 g (1388.3 mmol, 2 eq.) of 4,6-dichloro-2-phenylpyrimidine, 32.1 g (27.8 mmol, 0.04 eq.) of Pd(PPh₃)₄, and 110.4 g (1041.2 mmol, 1.5 eq.) of Na₂CO₃ were added to 2,000 mL of THF and 600 mL of H₂O, and the mixed solution was heated under reflux for 8 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 117-i, which is 4-(4-(bicyclo[2.2.1]heptan-2-yl)phenyl)-6-chloro-2-phenylpyrimidine (155.3 g, yield of 62%).

Mass: [(M+H)⁺]: 360.89

NMR(¹H): σ=8.36 (2H, d), 7.64 (2H, d), 7.54-7.48 (5H, m), 7.03 (1H, s), 2.62 (1H, s), 2.19-2.05 (3H, m), 1.82-1.72 (3H, m), 1.55-1.32 (4H, m)

### 2) Synthesis of Compound 117

10 g (27.7 mmol, 1.0 eq.) of 4-(4-(bicyclo[2.2.1]heptan-2-yl)phenyl)-6-chloro-2-phenylpyrimidine, 13.3 g (30.5 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.7 g (55.4 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 117 (12.5 g, yield of 71%).

Mass: [(M+H)⁺]: 633.80

### Synthesis of Compounds 137, 145, 153, 181, 193, 205, and 225

Target Compound B was synthesized in substantially the same manner as in the synthesis of Compound 117 according to Synthesis Example 9, except that the intermediate (A) was changed to Intermediate A in Table 8.

TABLE 8

| | | | Yield | Mass |
|---|---|---|---|---|
| Com- | | | (117-i to | [(M + |
| pound | Intermediate A | Target Compound B | B) | H)⁺] |
| Com- pound 137 | 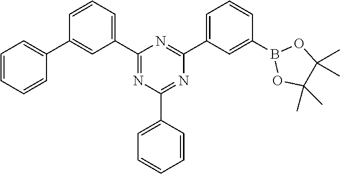 [SMILES: CC1(C)OB(c2cccc(-c3nc(-c4ccccc4)nc(-c4cccc(-c5ccccc5)c4)n3)c2)OC1(C)C] | 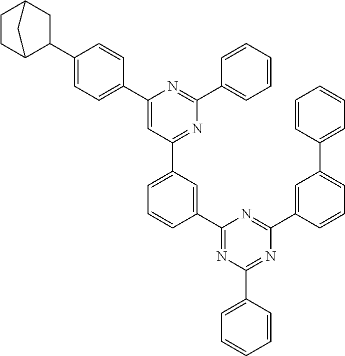 [SMILES: c1ccc(-c2cccc(-c3nc(-c4ccccc4)nc(-c4cccc(-c5cc(-c6ccc(C7CC8CCC7C8)cc6)nc(-c6ccccc6)n5)c4)n3)c2)cc1] | 72% | 709.90 |
| Com- pound 145 | 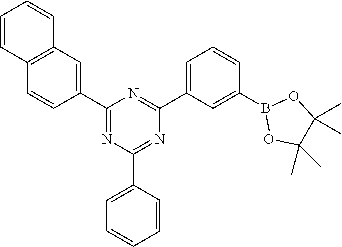 [SMILES: CC1(C)OB(c2cccc(-c3nc(-c4ccccc4)nc(-c4ccc5ccccc5c4)n3)c2)OC1(C)C] | 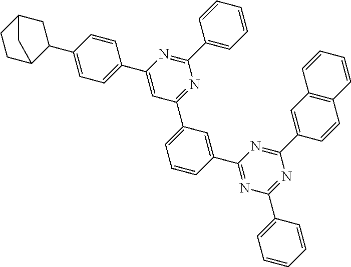 [SMILES: c1ccc(-c2nc(-c3ccc(C4CC5CCC4C5)cc3)cc(-c3cccc(-c4nc(-c5ccccc5)nc(-c5ccc6ccccc6c5)n4)c3)n2)cc1] | 70% | 683.86 |
| Com- pound 153 | 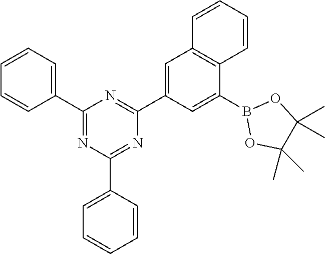 [SMILES: CC1(C)OB(c2cc(-c3nc(-c4ccccc4)nc(-c4ccccc4)n3)cc3ccccc23)OC1(C)C] | 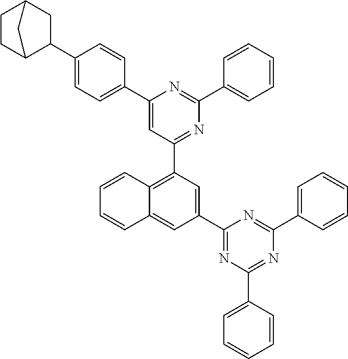 [SMILES: c1ccc(-c2nc(-c3ccc(C4CC5CCC4C5)cc3)cc(-c3cc(-c4nc(-c5ccccc5)nc(-c5ccccc5)n4)cc4ccccc34)n2)cc1] | 69% | 638.86 |
| Com- pound 181 | 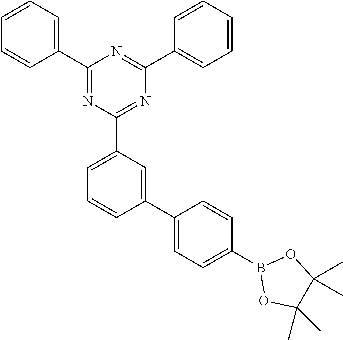 [SMILES: CC1(C)OB(c2ccc(-c3cccc(-c4nc(-c5ccccc5)nc(-c5ccccc5)n4)c3)cc2)OC1(C)C] | 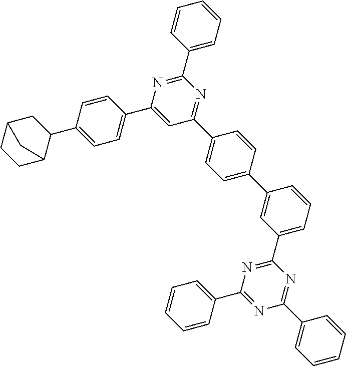 [SMILES: c1ccc(-c2nc(-c3ccc(-c4cccc(-c5nc(-c6ccccc6)nc(-c6ccccc6)n5)c4)cc3)cc(-c3ccc(C4CC5CCC4C5)cc3)n2)cc1] | 74% | 709.90 |
| Com- pound 193 | 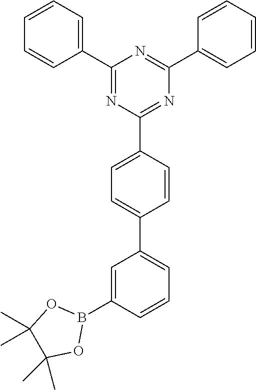 [SMILES: CC1(C)OB(c2cccc(-c3ccc(-c4nc(-c5ccccc5)nc(-c5ccccc5)n4)cc3)c2)OC1(C)C] | 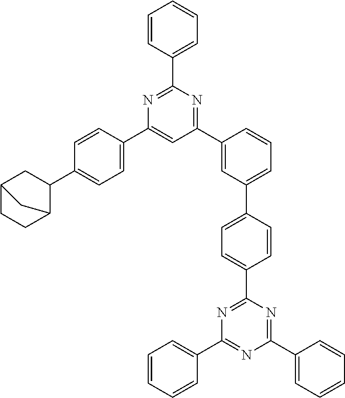 [SMILES: c1ccc(-c2nc(-c3ccc(C4CC5CCC4C5)cc3)cc(-c3cccc(-c4ccc(-c5nc(-c6ccccc6)nc(-c6ccccc6)n5)cc4)c3)n2)cc1] | 75% | 709.90 |
| Com- pound 205 | 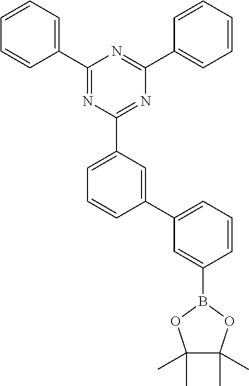 [SMILES: CC1(C)OB(c2cccc(-c3cccc(-c4nc(-c5ccccc5)nc(-c5ccccc5)n4)c3)c2)OC1(C)C] | 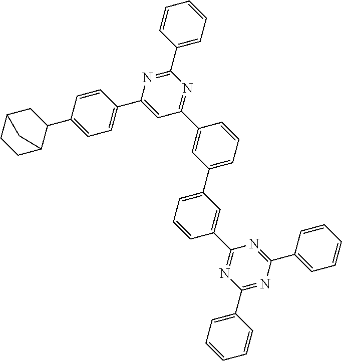 [SMILES: c1ccc(-c2nc(-c3ccc(C4CC5CCC4C5)cc3)cc(-c3cccc(-c4cccc(-c5nc(-c6ccccc6)nc(-c6ccccc6)n5)c4)c3)n2)cc1] | 75% | 709.90 |
| Com- pound 225 | 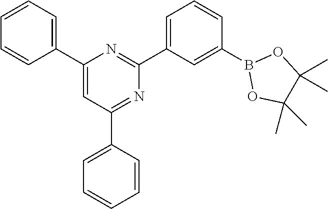 [SMILES: CC1(C)OB(c2cccc(-c3nc(-c4ccccc4)cc(-c4ccccc4)n3)c2)OC1(C)C] | 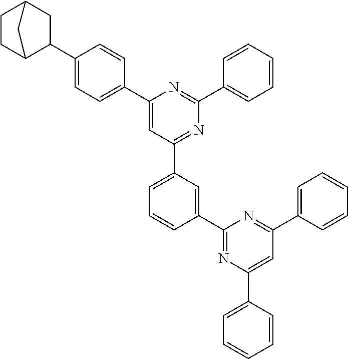 [SMILES: c1ccc(-c2cc(-c3ccccc3)nc(-c3cccc(-c4cc(-c5ccc(C6CC7CCC6C7)cc5)nc(-c5ccccc5)n4)c3)n2)cc1] | 72% | 632.81 |

### Synthesis Example 10: Synthesis of Compound 118

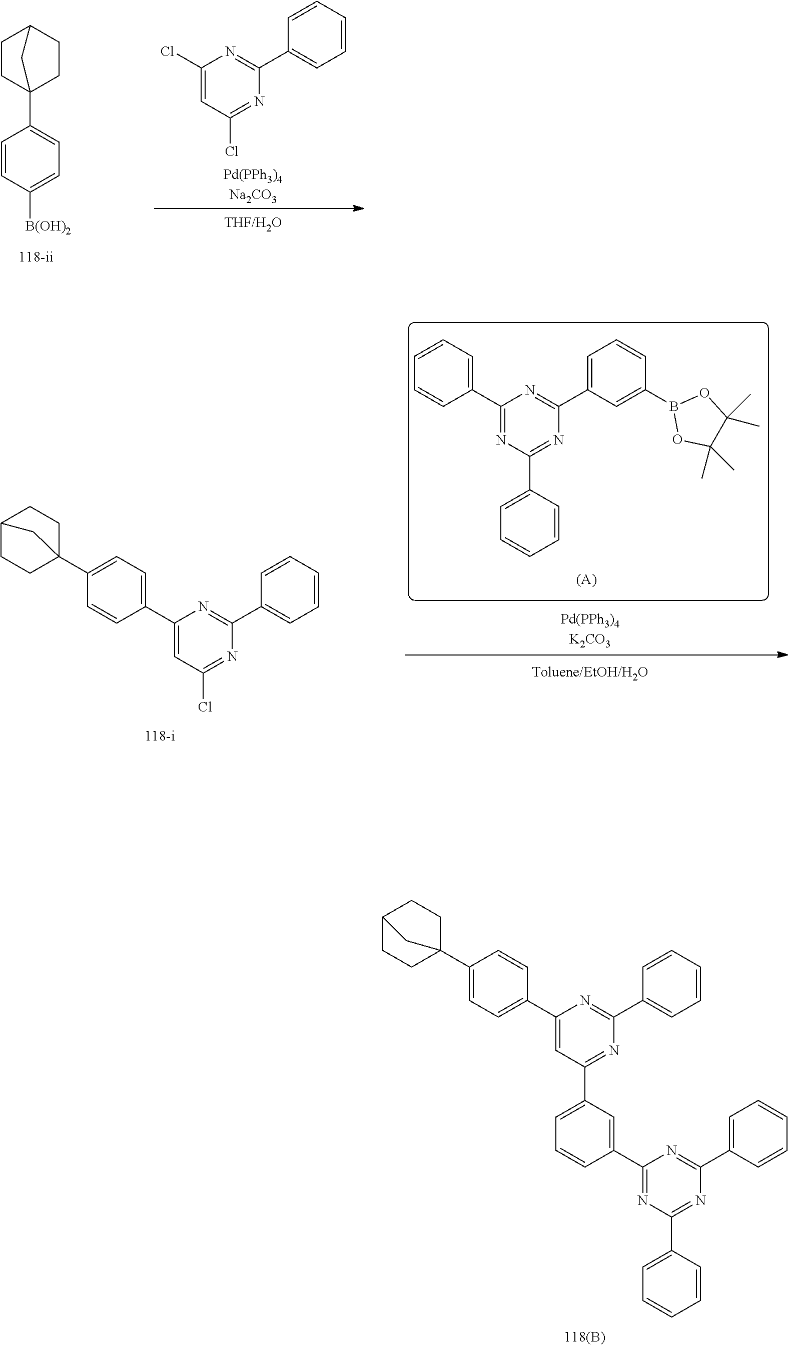

### 1) Synthesis of Compound 118-i

50 g (231.4 mmol, 1 eq.) of (4-(bicyclo[2.2.1]heptan-1-yl)phenyl)boronic acid, 104.2 g (462.8 mmol, 2 eq.) of 4,6-dichloro-2-phenylpyrimidine, 10.7 g (9.3 mmol, 0.04 eq.) of Pd(PPh₃)₄, and 36.8 g (347.1 mmol, 1.5 eq.) of Na₂CO₃ were added to 750 mL of THF and 200 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 118-i, which is 4-(4-(bicyclo[2.2.1]heptan-1-yl)phenyl)-6-chloro-2-phenylpyrimidine (50.9 g, yield of 61%).

Mass: [(M+H)⁺]: 360.89

NMR(¹H): σ=8.36 (2H, d), 7.56-7.46 (5H, m), 7.38 (2H, d), 7.03 (1H, s), 2.19 (1H, s), 1.80-1.65 (6H, m), 1.57-1.34 (4H, m)

### 2) Synthesis of Compound 118

10 g (27.7 mmol, 1.0 eq.) of 4-(4-(bicyclo[2.2.1]heptan-1-yl)phenyl)-6-chloro-2-phenylpyrimidine, 13.3 g (30.5 mmol, 1.1 eq.) of 2,4-diphenyl-6-(3-(4,4,5,5-tetramethyl-1,3,2-dioxaborolan-2-yl)phenyl)-1,3,5-triazine (A), 1.3 g (1.1 mmol, 0.04 eq.) of Pd(PPh₃)₄, and 7.7 g (55.4 mmol, 2 eq.) of K₂CO₃ were added to 150 ml of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 118 (12.5 g, yield of 71%).

Mass: [(M+H)⁺]: 633.80

### Synthesis of Compound 226

Target Compound B was synthesized in substantially the same manner as in the synthesis of Compound 118 according to Synthesis Example 10, except that the intermediate (A) was changed to Intermediate A in Table 9.

TABLE 9

| | | | Yield | Mass |
|---|---|---|---|---|
| Com- | | | (118-i to | [(M + |
| pound | Intermediate A | Target Compound B | B) | H)⁺] |
| Com- pound 226 | 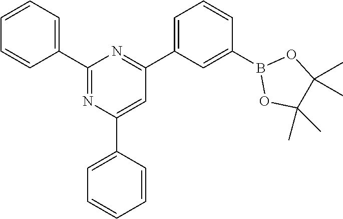 [SMILES: CC1(C)OB(c2cccc(-c3cc(-c4ccccc4)nc(-c4ccccc4)n3)c2)OC1(C)C] | 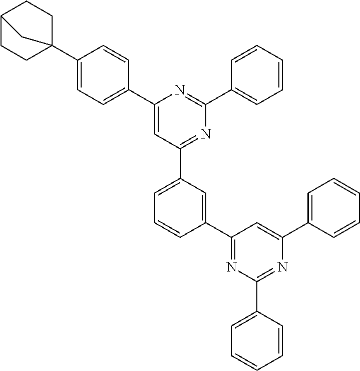 [SMILES: c1ccc(-c2cc(-c3cccc(-c4cc(-c5ccc(C67CCC(CC6)C7)cc5)nc(-c5ccccc5)n4)c3)nc(-c3ccccc3)n2)cc1] | 66% | 632.81 |

### Synthesis Example 11: Synthesis of Compound 139

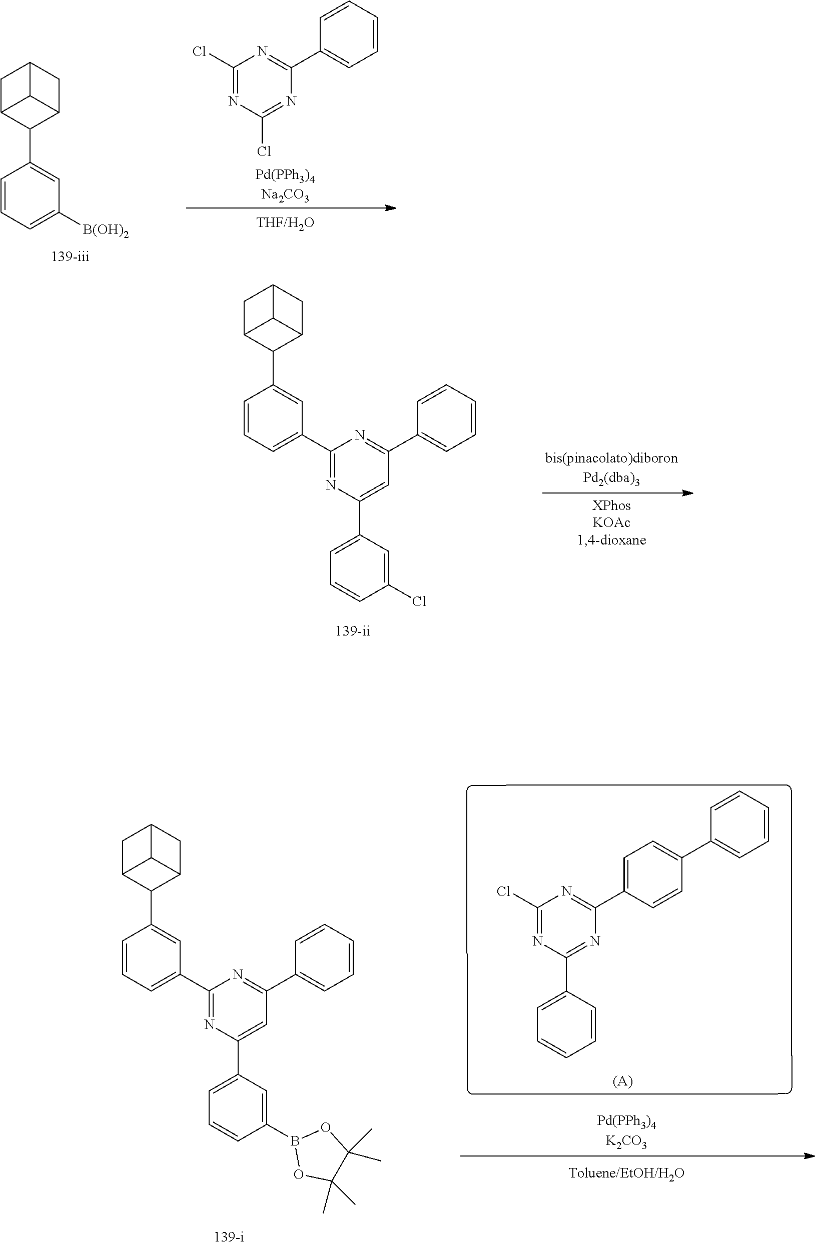

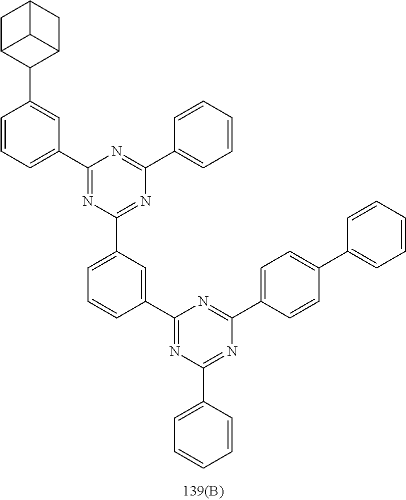 [SMILES: c1ccc(-c2ccc(-c3nc(-c4ccccc4)nc(-c4cccc(-c5nc(-c6ccccc6)nc(-c6cccc(C7C8CC9CC7C98)c6)n5)c4)n3)cc2)cc1]

### 1) Synthesis of Compound 139-ii

50 g (231.4 mmol, 1 eq.) of (3-((3R,5S,6r)-tricyclo[3.1.1.03,6]heptan-2-yl)phenyl)boronic acid, 77.1 g (256.9 mmol, 1.1 eq.) of 2-chloro-4-(3-chlorophenyl)-6-phenylpyrimidine, 10.8 g (9.3 mmol, 0.04 eq.) of Pd(PPh₃)₄, and 64.6 g (467.1 mmol, 2.0 eq.) of Na₂CO₃ were added to 200 mL of THF, and 200 ml of H₂O, and the mixed solution was heated under reflux for 5 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 139-ii, which is 4-(3-chlorophenyl)-6-phenyl-2-(3-((1R,2r,3S,6r)-tricyclo[3.1.1.03,6]heptan-2-yl)phenyl)pyrimidine (83.3 g, yield of 82%).

Mass: [(M+H)⁺]: 434.97

NMR(¹H): σ=8.23-8.18 (2H, m), 7.97-7.91 (4H, m), 7.72 (1H, d), 7.55-7.38 (7H, m), 4.39 (2H, s), 4.01 (1H, s), 3.81 (1H, s), 2.60 (1H, s), 1.50-1.33 (4H, m)

### 2) Synthesis of Compound 139-i

83 g (190.8 mmol, 1 eq.) of 4-(3-chlorophenyl)-6-phenyl-2-(3-((1R,2r,3S,6r)-tricyclo[3.1.1.03,6]heptan-2-yl)phenyl)pyrimidine, 63 g (248.1 mmol, 1.3 eq.) of bis(pinacolato)diboron, 8.7 g (9.5 mmol, 0.05 eq.) of tris(dibenzylideneacetone)dipalladium(0) (Pd₂(dba)₃), 9.1 g (19.1 mmol, 0.1 eq.) of 2-dicyclohexylphosphino-2′,4′,6′-triisopropylbiphenyl (Xphos), and 48.7 g (496.1 mmol, 2.0 eq.) of KOAc were added to 800 mL of 1,4-dioxane, and the mixed solution was heated under reflux for 4 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 139-i, which is 4-phenyl-6-(3-(4,4,5,5-tetramethyl-1,3,2-dioxaborolan-2-yl)phenyl)-2-(3-((1R,2r,3S,6r)-tricyclo[3.1.1.03,6]heptan-2-yl)phenyl)pyrimidine (71.3 g, yield of 71%).

Mass: [(M+H)⁺]: 526.49

### 3) Synthesis of Compound 139

15 g (28.5 mmol, 1.0 eq.) of 4-phenyl-6-(3-(4,4,5,5-tetramethyl-1,3,2-dioxaborolan-2-yl)phenyl)-2-(3-((1R,2r,3S,6r)-tricyclo[3.1.1.03,6]heptan-2-yl)phenyl)pyrimidine, 9.8 g (28.5 mmol, 1.0 eq.) of 2-([1,1′-biphenyl]-4-yl)-4-chloro-6-phenyl-1,3,5-triazine (A), 1.3 g (1.1 mmol, 0.04 eq.) of Pd(PPh₃)₄, and 7.9 g (57.0 mmol, 2 eq.) of K₂CO₃ were added to 230 mL of toluene, 60 mL of EtOH, and 60 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 139 (15.7 g, yield of 78%).

Mass: [(M+H)⁺]: 707.88

### Synthesis of Compound 236

Target Compound B was synthesized in substantially the same manner as in the synthesis of Compound 139 according to Synthesis Example 11, except that Intermediate (A) was changed to Intermediate A in Table 10.

TABLE 10

| | | | Yield | Mass |
|---|---|---|---|---|
| | | | (139-i to | [(M + |
| Compound | Intermediate A | Target Compound B | B) | H)⁺] |
| Compound 236 | 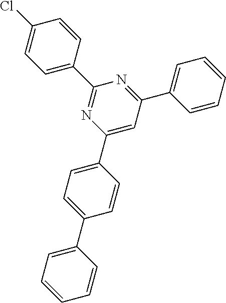 [SMILES: Clc1ccc(-c2nc(-c3ccccc3)cc(-c3ccc(-c4ccccc4)cc3)n2)cc1] | 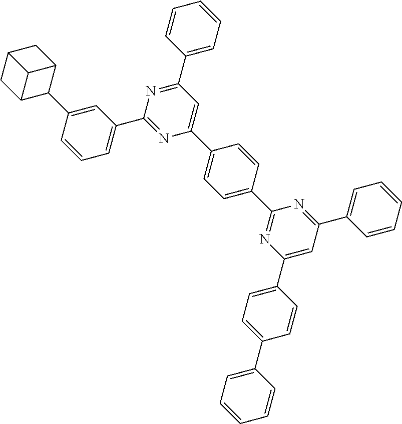 [SMILES: c1ccc(-c2ccc(-c3cc(-c4ccccc4)nc(-c4ccc(-c5cc(-c6ccccc6)nc(-c6cccc(C7C8CC9CC7C98)c6)n5)cc4)n3)cc2)cc1] | 76% | 706.89 |

### Synthesis Example 12: Synthesis of Compound 207

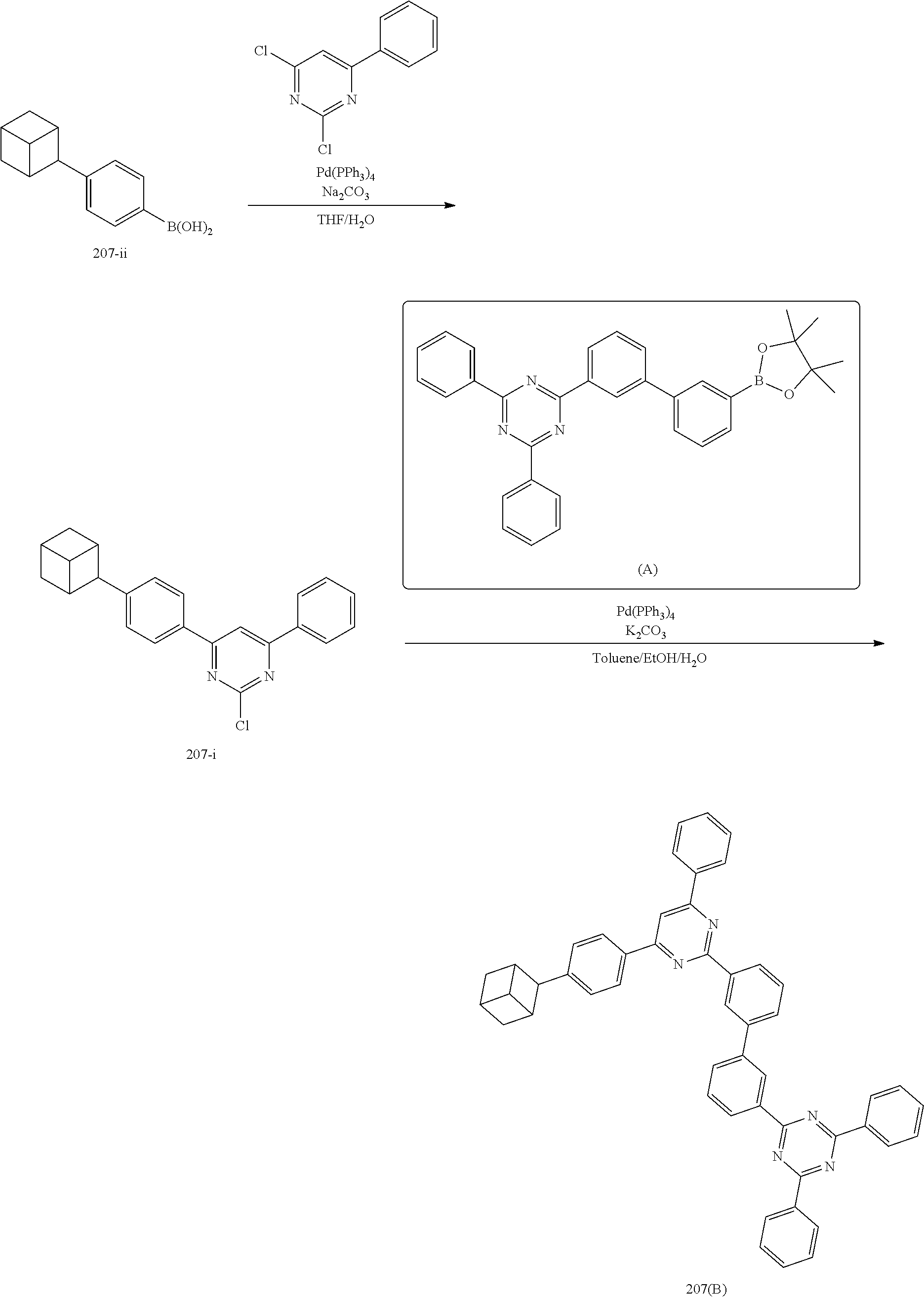 [SMILES: OB(O)c1ccc(C2C3CC4CC2C43)cc1 ; Clc1cc(-c2ccccc2)nc(Cl)n1 ; Clc1nc(-c2ccccc2)cc(-c2ccc(C3C4CC5CC3C54)cc2)n1 ; CC1(C)OB(c2cccc(-c3cccc(-c4nc(-c5ccccc5)nc(-c5ccccc5)n4)c3)c2)OC1(C)C ; "(A)" ; c1ccc(-c2cc(-c3ccc(C4C5CC6CC4C65)cc3)nc(-c3cccc(-c4cccc(-c5nc(-c6ccccc6)nc(-c6ccccc6)n5)c4)c3)n2)cc1]

### 1) Synthesis of Compound 207-i

50 g (233.6 mmol, 1 eq.) of (4-((1S,5R,6S)-tricyclo[3.1.1.03,6]heptan-2-yl)phenyl)boronic acid, 105.1 g (467.1 mmol, 2 eq.) of 2,4-dichloro-6-phenylpyrimidine, 10.8 g (9.3 mmol, 0.04 eq.) of Pd(PPh₃)₄, and 37.1 g (350.4 mmol, 1.5 eq.) of Na₂CO₃ were added to 750 mL of THF and 200 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 207-i, which is 2-chloro-4-phenyl-6-(4-((1S,5R,6S)-tricyclo[3.1.1.03,6]heptan-2-yl)phenyl)pyrimidine (50.3 g, yield of 60%).

Mass: [(M+H)⁺]: 358.87

NMR(¹H): σ=8.33 (1H, s), 7.94 (2H, d), 7.55-7.49 (7H, m), 4.39 (2H, s), 4.01 (1H, s), 3.81 (1H, s), 2.60 (1H, s), 1.50-1.30 (4H, m)

### 2) Synthesis of Compound 207

10 g (27.9 mmol, 1.0 eq.) of 2-chloro-4-phenyl-6-(4-((1S,5R,6S)-tricyclo[3.1.1.03,6]heptan-2-yl)phenyl)pyrimidine, 15.7 g (30.7 mmol, 1.1 eq.) of 2,4-diphenyl-6-(3′-(4,4,5,5-tetramethyl-1,3,2-dioxaborolan-2-yl)-[1,1′-biphenyl]-3-yl)-1,3,5-triazine (A), 1.3 g (1.1 mmol, 0.04 eq) of Pd(PPh₃)₄, and 7.7 g (55.7 mmol, 2 eq.) of K₂CO₃ were added to 150 mL of toluene, 40 mL of EtOH, and 40 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 207 (14.6 g, yield of 71%).

Mass: [(M+H)⁺]: 707.88

### Synthesis of Compound 227

Target Compound B was synthesized in substantially the same manner as in the synthesis of Compound 207 according to Synthesis Example 12, except that the intermediate (A) was changed to Intermediate A in Table 11.

TABLE 11

| | | | Yield | |
|---|---|---|---|---|
| Compound | Intermediate A | Target Compound B | (207-i to B) | Mass [(M + H)⁺] |
| Compound 227 | 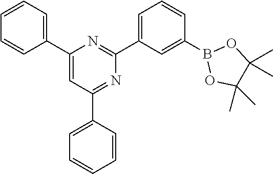 [SMILES: CC1(C)OB(c2cccc(-c3nc(-c4ccccc4)cc(-c4ccccc4)n3)c2)OC1(C)C] | 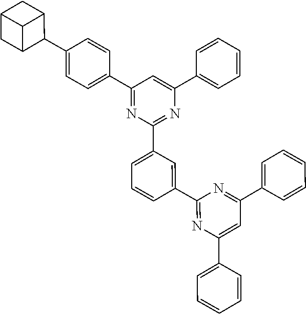 [SMILES: c1ccc(-c2cc(-c3ccccc3)nc(-c3cccc(-c4nc(-c5ccccc5)cc(-c5ccc(C6C7CC8CC6C87)cc5)n4)c3)n2)cc1] | 76% | 630.80 |

### Synthesis Example 13: Synthesis of Compound 281

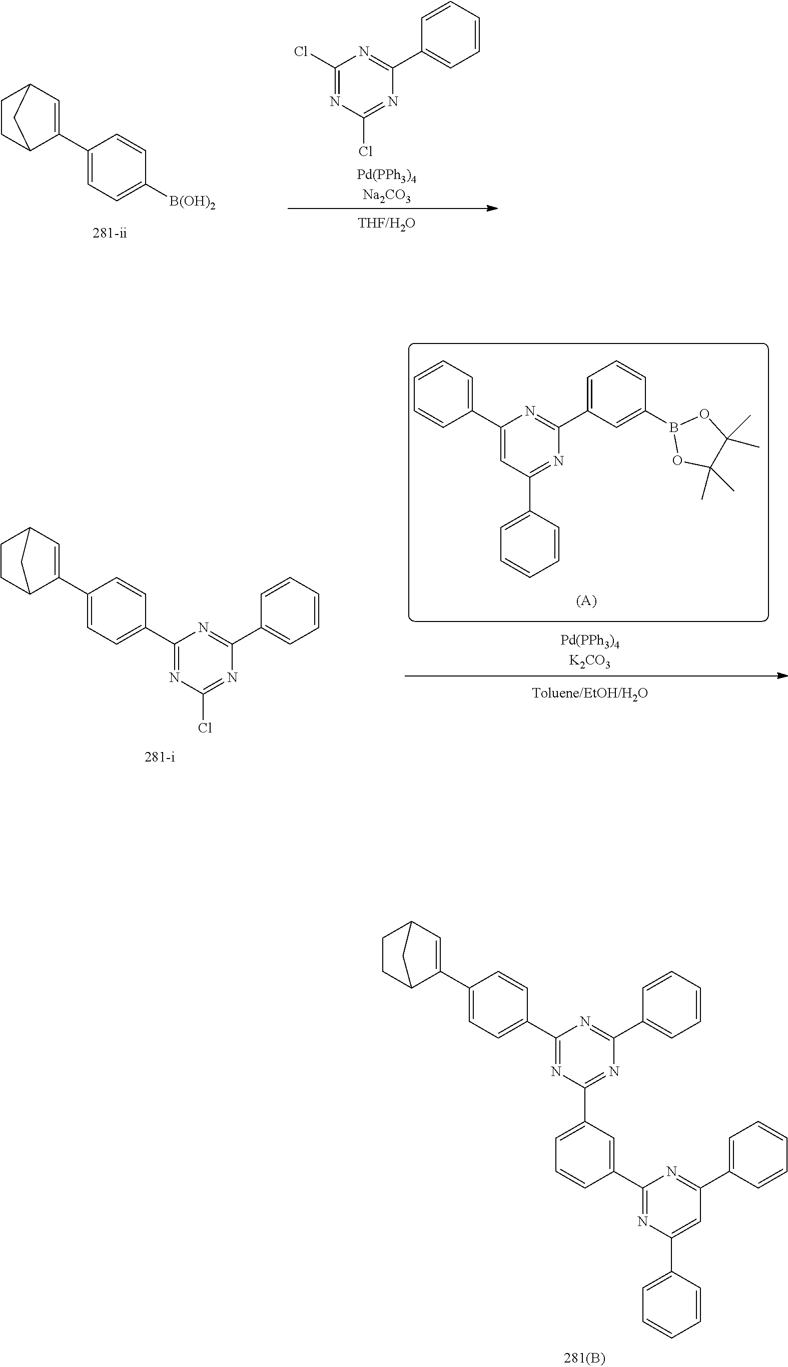

### 1) Synthesis of Compound 281-i

50 g (233.6 mmol, 1 eq.) of (4-(bicyclo[2.2.1]hept-2-en-2-yl)phenyl)boronic acid, 105.6 g (467.1 mmol, 2 eq.) of 2,4-dichloro-6-phenyl-1,3,5-triazine, 10.8 g (9.3 mmol, 0.04 eq.) of Pd(PPh₃)₄, and 37.1 g (350.4 mmol, 1.5 eq.) of Na₂CO₃ were added to 750 mL of THF and 200 mL of H₂O, and the mixed solution was heated under reflux for 5 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 281-i, which is 2-(4-(bicyclo[2.2.1]hept-2-en-2-yl)phenyl)-4-chloro-6-phenyl-1,3,5-triazine (48.8 g, yield of 58%).

Mass: [(M+H)⁺]: 359.86

NMR(¹H): σ=8.36 (2H, d), 7.72 (2H, d), 7.54-7.48 (5H, m), 5.96 (1H, s), 2.84 (1H, s), 2.18 (1H, s), 1.75-1.63 (2H, m), 1.55-1.35 (4H, m)

### 2) Synthesis of Compound 281

10 g (27.8 mmol, 1.0 eq.) of 2-(4-(bicyclo[2.2.1]hept-2-en-2-yl)phenyl)-4-chloro-6-phenyl-1,3,5-triazine, 13.3 g (30.6 mmol, 1.1 eq.) of 4,6-diphenyl-2-(3-(4,4,5,5-tetramethyl-1,3,2-dioxaborolan-2-yl)phenyl)pyrimidine (A), 1.3 g (1.1 mmol, 0.04 eq.) of Pd(PPh₃)₄, and 7.7 g (55.6 mmol, 2 eq.) of K₂CO₃ were added to 150 mL of toluene, 40 mL of EtOH, and 40 mL of H₂O, and the mixed solution was heated under reflux for 5 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 281 (12.6 g, yield of 72%).

Mass: [(M+H)⁺]: 631.78

### Synthesis Example 14: Synthesis of Compound 283

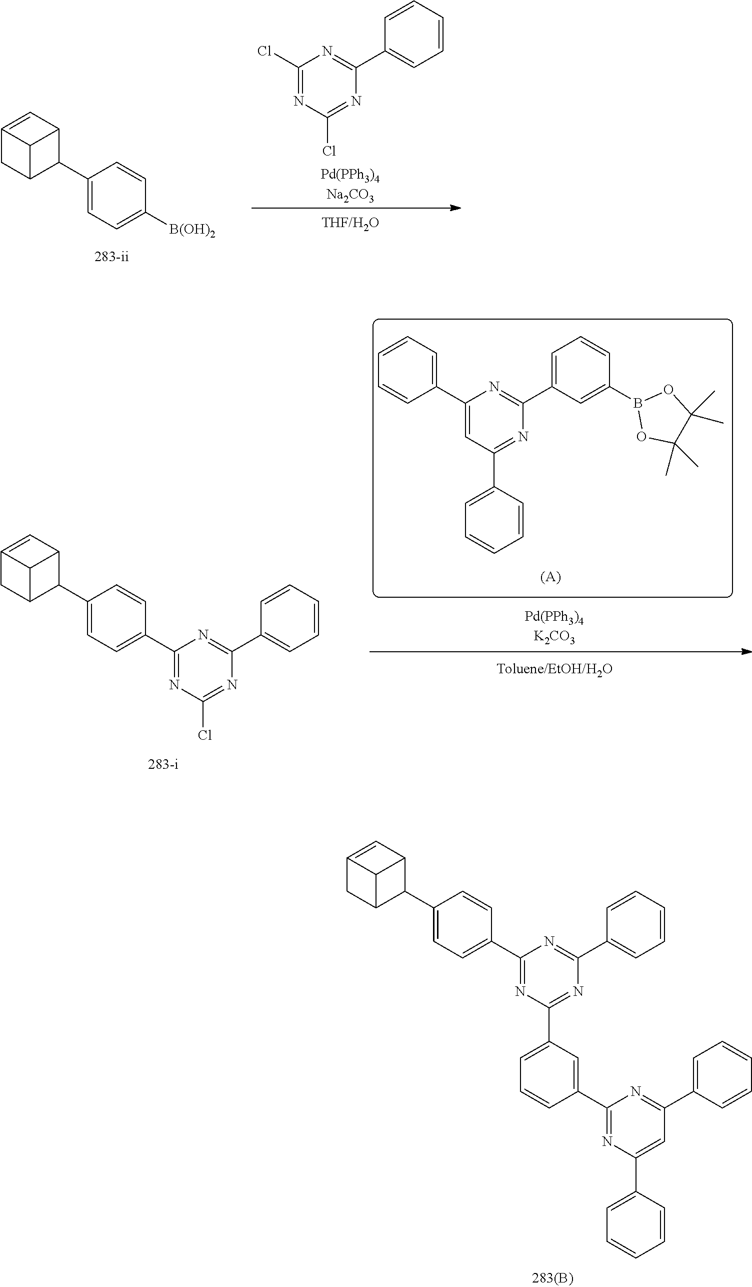

### 1) Synthesis of Compound 283-i

50 g (235.8 mmol, 1 eq.) of (4-((3S,6S)-tricyclo[3.1.1.03,6]hept-4-en-2-yl)phenyl)boronic acid, 106.6 g (471.6 mmol, 2 eq.) of 2,4-dichloro-6-phenyl-1,3,5-triazine, 10.9 g (9.4 mmol, 0.04 eq.) of Pd(PPh₃)₄, and 37.5 g (353.7 mmol, 1.5 eq.) of Na₂CO₃ were added to 750 mL of THF and 200 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 283-i, which is 2-chloro-4-phenyl-6-(4-((3S,6S)-tricyclo[3.1.1.03,6]hept-4-en-2-yl)phenyl)-1,3,5-triazine (49.8 g, yield of 59%).

Mass: [(M+H)⁺]: 357.84

NMR(¹H): σ=8.57 (2H, d), 8.36 (2H, d), 7.51-7.45 (5H, m), 5.7(1H, s), 3.2(1H, s), 2.8(1H, s), 2.58 (1H, s), 2.3(1H, s), 2.02-1.77 (2H, m)

### 2) Synthesis of Compound 283

10 g (27.9 mmol, 1.0 eq.) of 2-chloro-4-phenyl-6-(4-((3S,6S)-tricyclo[3.1.1.03,6]hept-4-en-2-yl)phenyl)-1,3,5-triazine, 13.4 g (30.7 mmol, 1.1 eq.) of 4,6-diphenyl-2-(3-(4,4,5,5-tetramethyl-1,3,2-dioxaborolan-2-yl)phenyl)pyrimidine (A), 1.3 g (1.1 mmol, 0.04 eq.) of Pd(PPh₃)₄, and 7.7 g (55.9 mmol, 2 eq.) of K₂CO₃ were added to 150 mL of toluene, 40 mL of EtOH, and 40 mL of H₂O, and the mixed solution was heated under reflux for 5 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 283 (13.6 g, yield of 77%).

Mass: [(M+H)⁺]: 629.77

### Synthesis Example 15: Synthesis of Compound 297

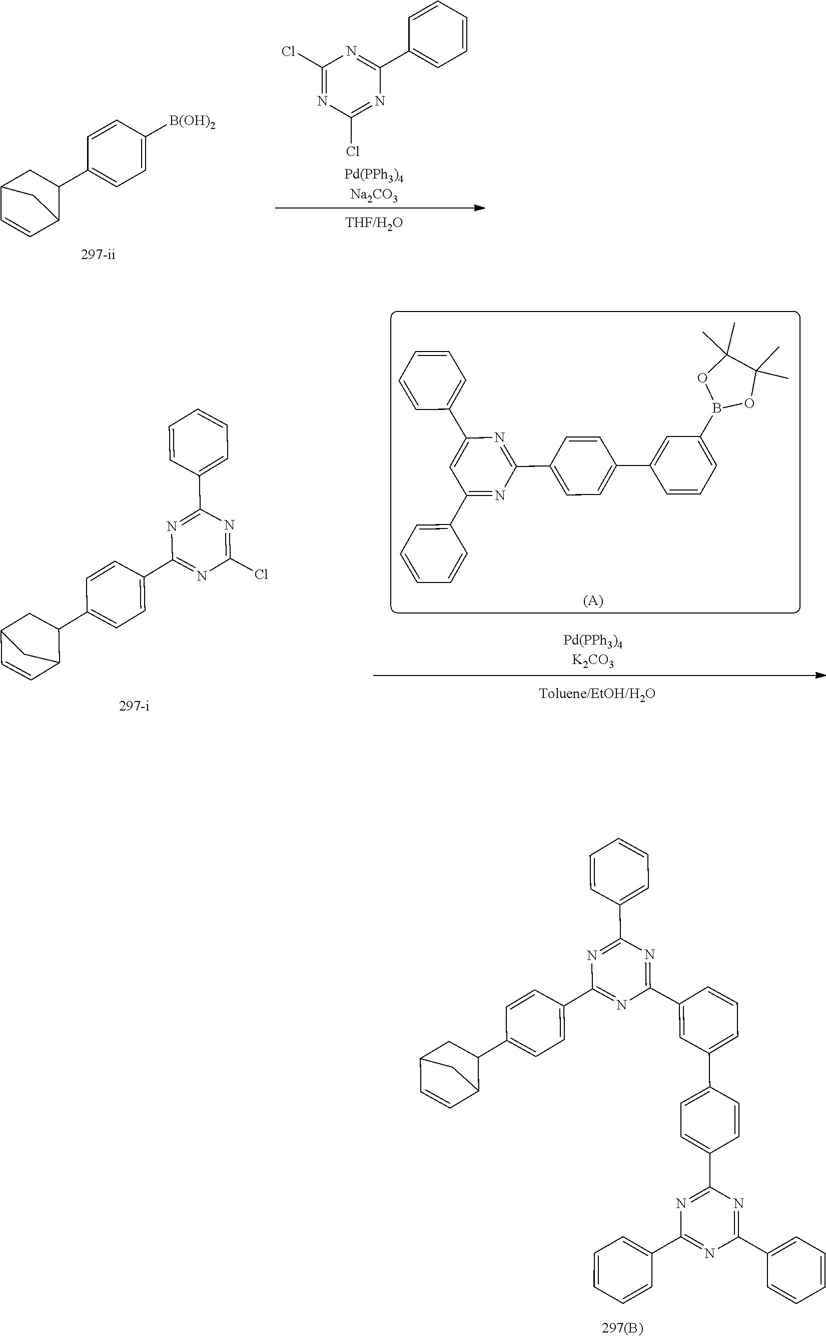 [SMILES: OB(O)c1ccc(C2CC3C=CC2C3)cc1 ; Clc1nc(Cl)nc(-c2ccccc2)n1 ; Clc1nc(-c2ccccc2)nc(-c2ccc(C3CC4C=CC3C4)cc2)n1 ; CC1(C)OB(c2cccc(-c3ccc(-c4nc(-c5ccccc5)cc(-c5ccccc5)n4)cc3)c2)OC1(C)C ; "(A)" ; C1=CC2CC1CC2c1ccc(-c2nc(-c3ccccc3)nc(-c3cccc(-c4ccc(-c5nc(-c6ccccc6)nc(-c6ccccc6)n5)cc4)c3)n2)cc1]

### 1) Synthesis of Compound 297-i

50 g (235.8 mmol, 1 eq.) of (4-(bicyclo[2.2.1]hept-5-en-2-yl)phenyl)boronic acid, 106.6 g (471.6 mmol, 2 eq.) of 2,4-dichloro-6-phenyl-1,3,5-triazine, 10.9 g (9.4 mmol, 0.04 eq.) of Pd(PPh₃)₄, and 37.5 g (353.7 mmol, 1.5 eq.) of Na₂CO₃ were added to 750 mL of THF and 200 mL of H₂O, and the mixed solution was heated under reflux for 6 hours. After completion of the reaction, the reaction solution was deactivated with a sufficient amount of water, and solids precipitated at room temperature were filtered through a filter. The solids thus obtained were sufficiently dried, dissolved in mono-chlorobenzene, and purified by column chromatography, so as to obtain Compound 297-i, which is 2-(4-(bicyclo[2.2.1]hept-5-en-2-yl)phenyl)-4-chloro-6-phenyl-1,3,5-triazine (49.8 g, yield of 59%).

Mass: [(M+H)⁺]: 357.84

NMR(¹H): σ=8.57 (2H, d), 8.36 (2H, d), 7.57-7.51 (5H, m), 6.23 (1H, s), 5.98 (1H, s), 2.84 (2H, s), 2.56 (1H, s), 1.93-1.50 (4H, m)

### 2) Synthesis of Compound 297

10 g (27.9 mmol, 1.0 eq.) of 2-(4-(bicyclo[2.2.1]hept-5-en-2-yl)phenyl)-4-chloro-6-phenyl-1,3,5-triazine, 15.7 g (30.7 mmol, 1.1 eq.) of 2,4-diphenyl-6-(3′-(4,4,5,5-tetramethyl-1,3,2-dioxaborolan-2-yl)-[1,1′-biphenyl]-4-yl)-1,3,5-triazine (A), 1.3 g (1.1 mmol, 0.04 eq) of Pd(PPh₃)₄, and 7.7 g (55.9 mmol, 2 eq.) of K₂CO₃ were added to 150 mL of toluene, 40 mL of EtOH, and 40 mL of H₂O, and the mixed solution was heated under reflux for 5 hours. After completion of the reaction, the reaction solution was deactivated with a sufficient amount of water, and solids precipitated at room temperature were filtered through a filter. The solids thus obtained were sufficiently dried, dissolved in mono-chlorobenzene, filtered through a silica gel, and then, concentrated. The resulting solids were sufficiently dried, dissolved in mono-chlorobenzene, and purified by column chromatography. Next, the resulting solids were dissolved again in mono-chlorobenzene and recrystallized, so as to obtain Compound 297 (14.5 g, yield of 73%).

Mass: [(M+H)⁺]: 708.87

### Synthesis of Compound 299

Target Compound B was synthesized in substantially the same manner as in the synthesis of Compound 297 according to Synthesis Example 15, except that Intermediate (A) was changed to Intermediate A in Table 12.

TABLE 12

| | | | Yield | Mass |
|---|---|---|---|---|
| Compound | Intermediate A | Target Compound B | (297-i to B) | [(M + H)⁺] |
| Compound 299 | 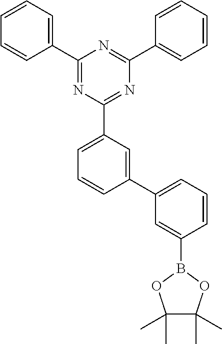 [SMILES: CC1(C)OB(c2cccc(-c3cccc(-c4nc(-c5ccccc5)nc(-c5ccccc5)n4)c3)c2)OC1(C)C] | 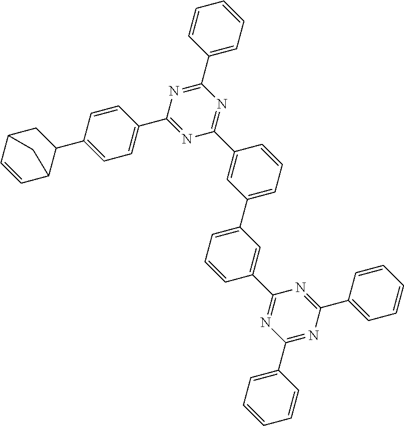 [SMILES: C1=CC2CC1CC2c1ccc(-c2nc(-c3ccccc3)nc(-c3cccc(-c4cccc(-c5nc(-c6ccccc6)nc(-c6ccccc6)n5)c4)c3)n2)cc1] | 70% | 708.87 |

Synthesis methods of compounds other than the compounds synthesized in Synthesis Examples above may be easily recognized by those skilled in the art by referring to the synthesis paths and source materials.

### Example 1

After the compounds synthesized in Synthesis Examples above were each subjected to high-purity sublimation purification by methods suitable in the art, a blue organic light-emitting device was manufactured according to the following process.

First, as an anode, a glass substrate on which indium tin oxide (ITO) was coated in the form of a thin film having a thickness of 1,200 Å was cleaned by ultrasonication in distilled water. After the cleaning with distilled water was finished, the resulting substrate was subjected to ultrasonication with a solvent, such as isopropyl alcohol, acetone, methanol, etc., dried, placed in a UV OZONE cleaning device (Power sonic 405, Hwashin Tech.) to be cleaned with UV for 5 minutes, and then, transferred to a vacuum evaporator.

On the ITO transparent electrode thus prepared, HI and 2% HAT-CN6 were co-deposited to form a hole injection layer having a thickness of 10 nm. Next, HI was deposited on the hole injection layer to form a hole transport layer having a thickness of 140 nm, and EB was deposited on the hole transport layer to form an electron blocking layer having a thickness of 5 nm.

BH as a host and 2% BD as a dopant were co-deposited on the electron blocking layer to form an emission layer having a thickness of 20 nm.

Subsequently, Compound 9 and LiQ were co-deposited at a weight ratio of 1:1 on the emission layer to form an electron transport layer having a thickness of 30 nm. Next, LiF was deposited on the electron transport layer to form an electron injection layer having a thickness of 1 nm, and Al was vacuum-deposited on the electron injection layer to a thickness of 100 nm to form an LiF/Al electrode, thereby manufacturing an organic light-emitting device.

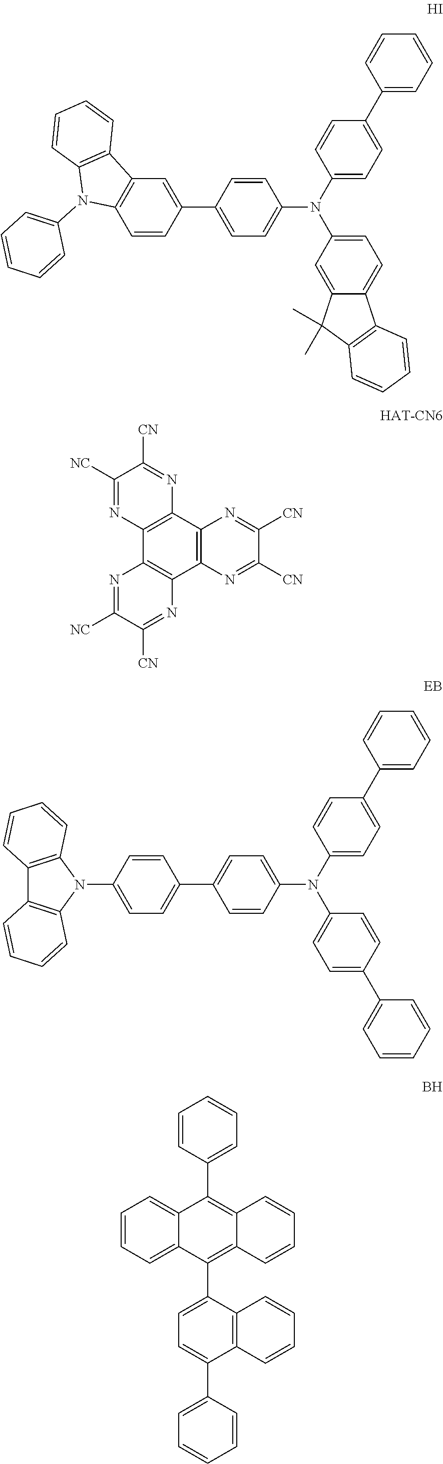

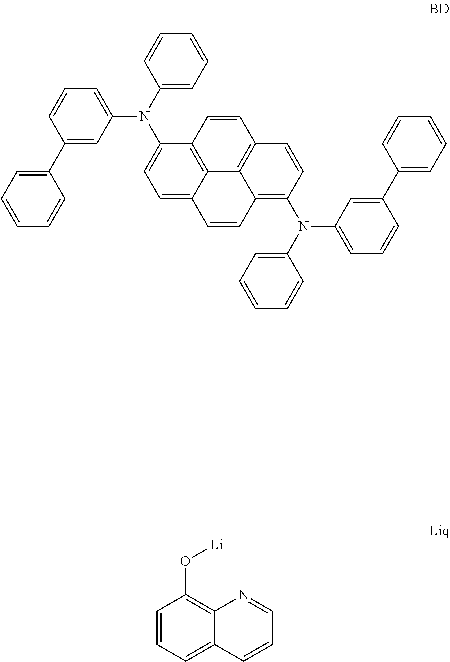

### Examples 2 to 44 and Comparative Examples 1 to 6

Organic light-emitting devices were each manufactured in substantially the same manner as in Example 1, except that Compound 9 was replaced by compounds shown in Table 13 in the formation of a respective electron transport layer.

### Evaluation Example 1

Regarding each of the organic light-emitting devices of Examples 1 to 44 and Comparative Examples 1 to 6, the driving voltage at current density of 10 mA/cm², emission wavelength, and current efficiency were measured.

The driving voltage, current efficiency, and emission color of each of the organic light-emitting devices were measured by utilizing a source meter (2400 series by Keithley Instrument) and a luminance meter PR650.

The evaluation results of the characteristics of each of the organic light-emitting devices are shown in Table 13.

TABLE 13

| | | Driving | Emission | Current |
|---|---|---|---|---|
| | Electron transport | voltage | peak | efficiency |
| Sample | layer material | (V) | (nm) | (cd/A) |
| Example 1 | Compound 9 | 3.4 | 454 | 8.2 |
| Example 2 | Compound 10 | 3.3 | 455 | 8.0 |
| Example 3 | Compound 11 | 3.3 | 455 | 8.1 |
| Example 4 | Compound 12 | 3.2 | 453 | 8.4 |
| Example 5 | Compound 13 | 3.3 | 454 | 8.3 |
| Example 6 | Compound 14 | 3.4 | 454 | 7.9 |
| Example 7 | Compound 15 | 3.2 | 453 | 8.2 |
| Example 8 | Compound 16 | 3.4 | 455 | 8.3 |
| Example 9 | Compound 26 | 3.3 | 454 | 8.1 |
| Example 10 | Compound 29 | 3.3 | 455 | 8.2 |
| Example 11 | Compound 45 | 3.4 | 454 | 8.3 |
| Example 12 | Compound 60 | 3.2 | 455 | 8.3 |
| Example 13 | Compound 73 | 3.4 | 456 | 7.8 |
| Example 14 | Compound 74 | 3.2 | 454 | 8.2 |
| Example 15 | Compound 76 | 3.2 | 453 | 8.2 |
| Example 16 | Compound 85 | 3.2 | 453 | 8.1 |
| Example 17 | Compound 87 | 3.2 | 454 | 8.0 |
| Example 18 | Compound 97 | 3.2 | 455 | 8.0 |
| Example 19 | Compound 98 | 3.2 | 454 | 8.1 |
| Example 20 | Compound 100 | 3.2 | 455 | 8.1 |
| Example 21 | Compound 117 | 3.4 | 456 | 8.2 |
| Example 22 | Compound 118 | 3.3 | 453 | 8.1 |
| Example 23 | Compound 137 | 3.3 | 453 | 8.3 |
| Example 24 | Compound 139 | 3.2 | 454 | 8.2 |
| Example 25 | Compound 145 | 3.3 | 454 | 8.0 |
| Example 26 | Compound 153 | 3.4 | 453 | 8.3 |
| Example 27 | Compound 181 | 3.2 | 456 | 7.9 |
| Example 28 | Compound 193 | 3.4 | 455 | 8.1 |
| Example 29 | Compound 205 | 3.3 | 453 | 8.3 |
| Example 30 | Compound 207 | 3.3 | 453 | 8.2 |
| Example 31 | Compound 225 | 3.4 | 455 | 8.0 |
| Example 32 | Compound 226 | 3.2 | 453 | 8.1 |
| Example 33 | Compound 227 | 3.4 | 454 | 8.1 |
| Example 34 | Compound 236 | 3.2 | 455 | 7.9 |
| Example 35 | Compound 281 | 3.2 | 455 | 8.1 |
| Example 36 | Compound 283 | 3.4 | 456 | 7.8 |
| Example 37 | Compound 297 | 3.2 | 456 | 8.0 |
| Example 38 | Compound 299 | 3.3 | 454 | 8.1 |
| Example 39 | Compound 313 | 3.4 | 453 | 8.2 |
| Example 40 | Compound 315 | 3.3 | 453 | 8.1 |
| Example 41 | Compound 357 | 3.3 | 455 | 8.0 |
| Example 42 | Compound 358 | 3.2 | 454 | 8.2 |
| Example 43 | Compound 377 | 3.2 | 454 | 8.3 |
| Example 44 | Compound 389 | 3.3 | 455 | 8.4 |
| Comparative | Alq₃ | 4.6 | 457 | 5.6 |
| Example 1 | | | | |
| Comparative | E-1 | 4.8 | 459 | 6.4 |
| Example 2 | | | | |
| Comparative | E-2 | 3.5 | 455 | 7.1 |
| Example 3 | | | | |
| Comparative | E-3 | 3.4 | 455 | 6.4 |
| Example 4 | | | | |
| Comparative | E-4 | 3.7 | 455 | 7.0 |
| Example 5 | | | | |
| Comparative | E-5 | 4.8 | 455 | 6.9 |
| Example 6 | | | | |

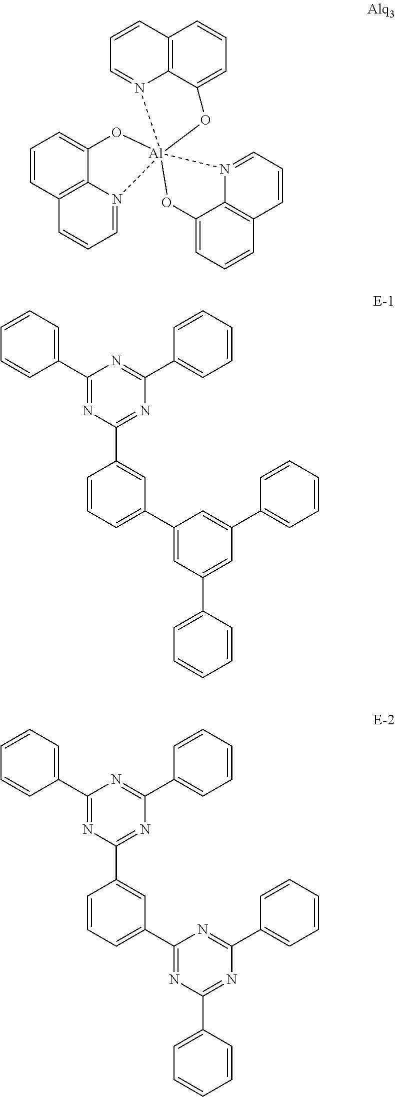

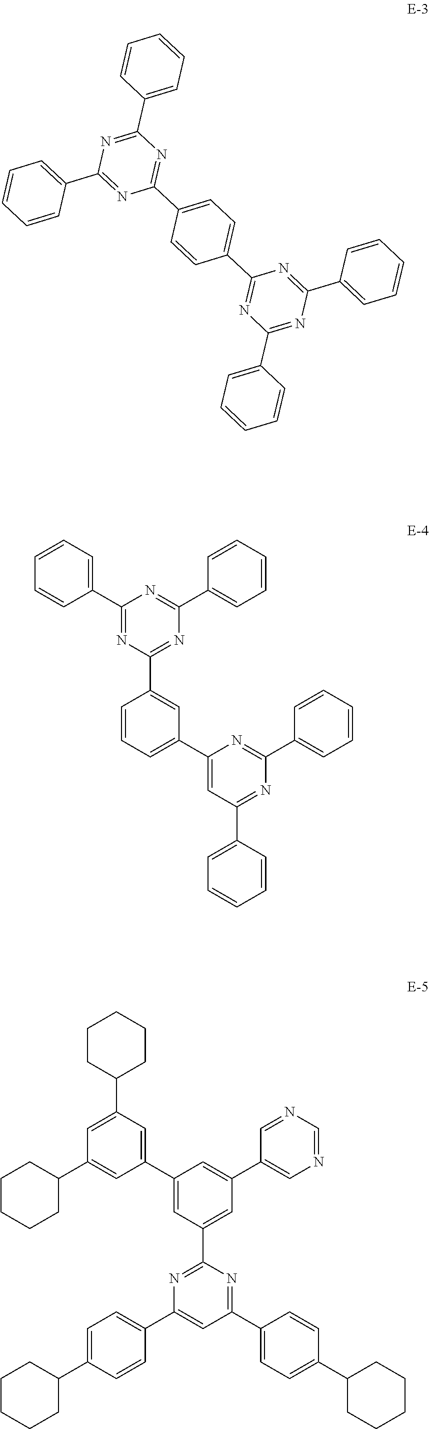 [SMILES: c1ccc(-c2nc(-c3ccccc3)nc(-c3ccc(-c4nc(-c5ccccc5)nc(-c5ccccc5)n4)cc3)n2)cc1 ; c1ccc(-c2cc(-c3cccc(-c4nc(-c5ccccc5)nc(-c5ccccc5)n4)c3)nc(-c3ccccc3)n2)cc1 ; c1ncc(-c2cc(-c3cc(C4CCCCC4)cc(C4CCCCC4)c3)cc(-c3nc(-c4ccc(C5CCCCC5)cc4)cc(-c4ccc(C5CCCCC5)cc4)n3)c2)cn1]

Referring to Table 13, the organic light-emitting devices of Examples 1 to 44 each had excellent or suitable characteristics in terms of driving voltage and current efficiency, compared to the organic light-emitting devices of Comparative Examples 1 to 6.

### Example 45

After the compounds synthesized in Synthesis Examples above were each subjected to high-purity sublimation purification by methods suitable in the art, a blue organic light-emitting device was manufactured according to the following process.

First, as an anode, a glass substrate on which ITO was coated in the form of a thin film having a thickness of 1,200 Å was cleaned by ultrasonication in distilled water. After the cleaning with distilled water was finished, the resulting substrate was subjected to ultrasonication with a solvent, such as isopropyl alcohol, acetone, methanol, etc., dried, placed in a UV OZONE cleaning device (Power sonic 405, Hwashin Tech.) to be cleaned with UV for 5 minutes, and then, transferred to a vacuum evaporator.

On the ITO transparent electrode thus prepared, HI and 2% HAT-CN6 were co-deposited to form a hole injection layer having a thickness of 10 nm. Next, HI was deposited on the hole injection layer to form a hole transport layer having a thickness of 140 nm, and EB was deposited on the hole transport layer to form an electron blocking layer having a thickness of 5 nm.

BH as a host and 2% BD as a dopant were co-deposited on the electron blocking layer to form an emission layer having a thickness of 20 nm.

Next, Compound 9 was deposited on the emission layer to form an electron transport auxiliary layer having a thickness of 5 nm. ET and LiQ were co-deposited at a weight ratio of 1:1 on the electron transport auxiliary layer to form an electron transport layer having a thickness of 30 nm. Next, LiF was deposited on the electron transport layer to form an electron injection layer having a thickness of 1 nm, and Al was vacuum-deposited on the electron injection layer to a thickness of 100 nm to form an LiF/Al electrode, thereby manufacturing an organic light-emitting device.

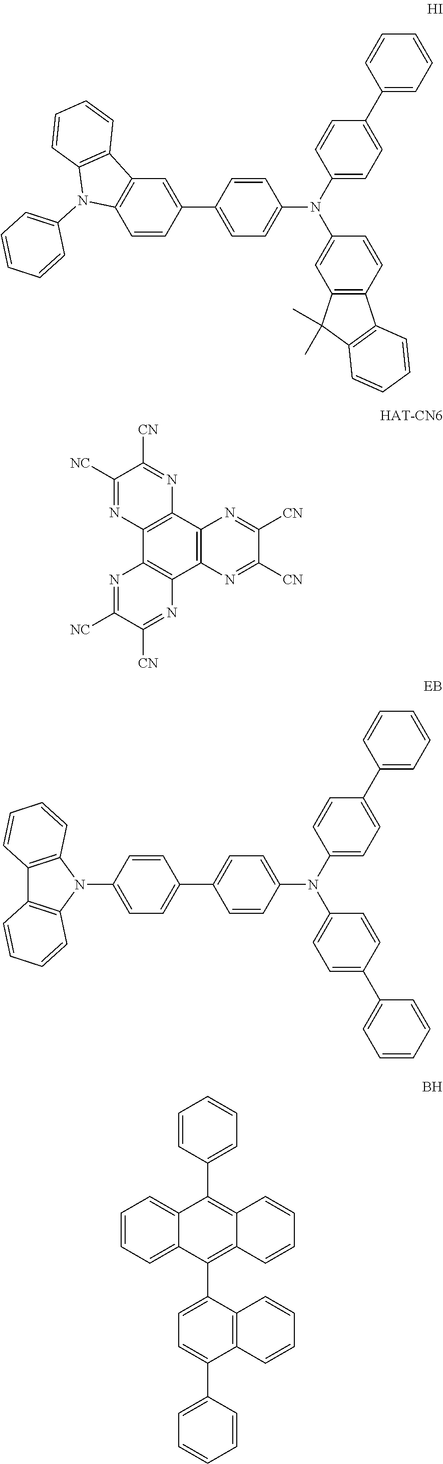

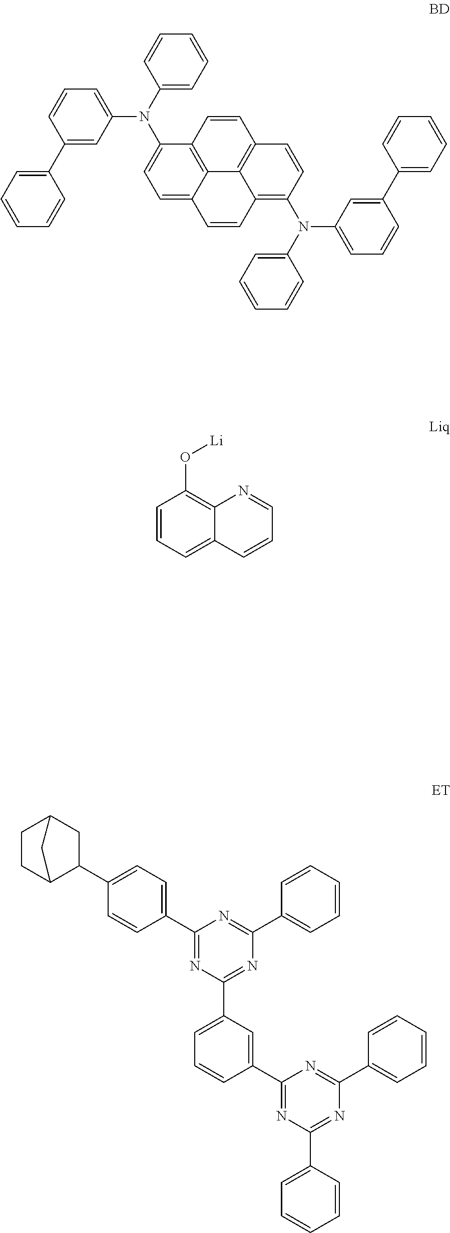

### Examples 46 to 88 and Comparative Examples 7 to 12

Organic light-emitting devices were each manufactured in substantially the same manner as in Example 45, except that Compound 9 was replaced by compounds shown in Table 14 in the formation of a respective electron transport auxiliary layer.

### Evaluation Example 2

Regarding each of the organic light-emitting devices of Examples 45 to 88 and Comparative Examples 7 to 12, the driving voltage at current density of 10 mA/cm², emission wavelength, and current efficiency were measured.

The driving voltage, current efficiency, and emission color of each of the organic light-emitting devices were measured by utilizing a source meter (2400 series by Keithley Instrument) and a luminance meter PR650.

The evaluation results of the characteristics of each of the organic light-emitting devices are shown in Table 14.

TABLE 14

| | Electron transport | Driving | Emission | Current |
|---|---|---|---|---|
| | auxiliary layer | voltage | peak | efficiency |
| Sample | material | (V) | (nm) | (cd/A) |
| Example 45 | Compound 9 | 3.3 | 454 | 8.4 |
| Example 46 | Compound 10 | 3.2 | 455 | 8.2 |
| Example 47 | Compound 11 | 3.2 | 455 | 8.3 |
| Example 48 | Compound 12 | 3.3 | 453 | 8.5 |
| Example 49 | Compound 13 | 3.2 | 454 | 8.5 |
| Example 50 | Compound 14 | 3.3 | 454 | 8.0 |
| Example 51 | Compound 15 | 3.2 | 453 | 8.2 |
| Example 52 | Compound 16 | 3.3 | 455 | 8.1 |
| Example 53 | Compound 26 | 3.4 | 454 | 8.3 |
| Example 54 | Compound 29 | 3.3 | 455 | 8.3 |
| Example 55 | Compound 45 | 3.2 | 454 | 8.4 |
| Example 56 | Compound 60 | 3.3 | 455 | 8.4 |
| Example 57 | Compound 73 | 3.3 | 456 | 7.9 |
| Example 58 | Compound 74 | 3.1 | 454 | 8.3 |
| Example 59 | Compound 76 | 3.2 | 453 | 8.1 |
| Example 60 | Compound 85 | 3.1 | 453 | 8.4 |
| Example 61 | Compound 87 | 3.3 | 454 | 8.4 |
| Example 62 | Compound 97 | 3.2 | 455 | 8.3 |
| Example 63 | Compound 98 | 3.1 | 454 | 8.2 |
| Example 64 | Compound 100 | 3.1 | 455 | 8.3 |
| Example 65 | Compound 117 | 3.3 | 456 | 8.2 |
| Example 66 | Compound 118 | 3.4 | 453 | 8.0 |
| Example 67 | Compound 137 | 3.2 | 453 | 8.2 |
| Example 68 | Compound 139 | 3.2 | 454 | 8.4 |
| Example 69 | Compound 145 | 3.1 | 454 | 8.4 |
| Example 70 | Compound 153 | 3.2 | 453 | 8.3 |
| Example 71 | Compound 181 | 3.3 | 456 | 8.0 |
| Example 72 | Compound 193 | 3.3 | 455 | 8.2 |
| Example 73 | Compound 205 | 3.2 | 453 | 8.4 |
| Example 74 | Compound 207 | 3.1 | 453 | 8.1 |
| Example 75 | Compound 225 | 3.3 | 455 | 8.3 |
| Example 76 | Compound 226 | 3.1 | 453 | 8.2 |
| Example 77 | Compound 227 | 3.3 | 454 | 8.2 |
| Example 78 | Compound 236 | 3.3 | 455 | 8.0 |
| Example 79 | Compound 281 | 3.4 | 455 | 8.2 |
| Example 80 | Compound 283 | 3.3 | 456 | 7.9 |
| Example 81 | Compound 297 | 3.2 | 456 | 8.2 |
| Example 82 | Compound 299 | 3.1 | 454 | 8.0 |
| Example 83 | Compound 313 | 3.3 | 453 | 8.1 |
| Example 84 | Compound 315 | 3.4 | 453 | 8.3 |
| Example 85 | Compound 357 | 3.2 | 455 | 8.2 |
| Example 86 | Compound 358 | 3.3 | 454 | 8.0 |
| Example 87 | Compound 377 | 3.2 | 453 | 8.4 |
| Example 88 | Compound 389 | 3.2 | 456 | 8.4 |
| Comparative | — | 4.9 | 457 | 5.3 |
| Example 7 | | | | |
| Comparative | E-1 | 5.0 | 459 | 6.7 |
| Example 8 | | | | |
| Comparative | E-2 | 3.4 | 455 | 7.2 |
| Example 9 | | | | |
| Comparative | E-3 | 3.7 | 455 | 6.5 |
| Example 10 | | | | |
| Comparative | E-4 | 3.6 | 455 | 7.3 |
| Example 11 | | | | |
| Comparative | E-5 | 4.9 | 459 | 6.9 |
| Example 12 | | | | |

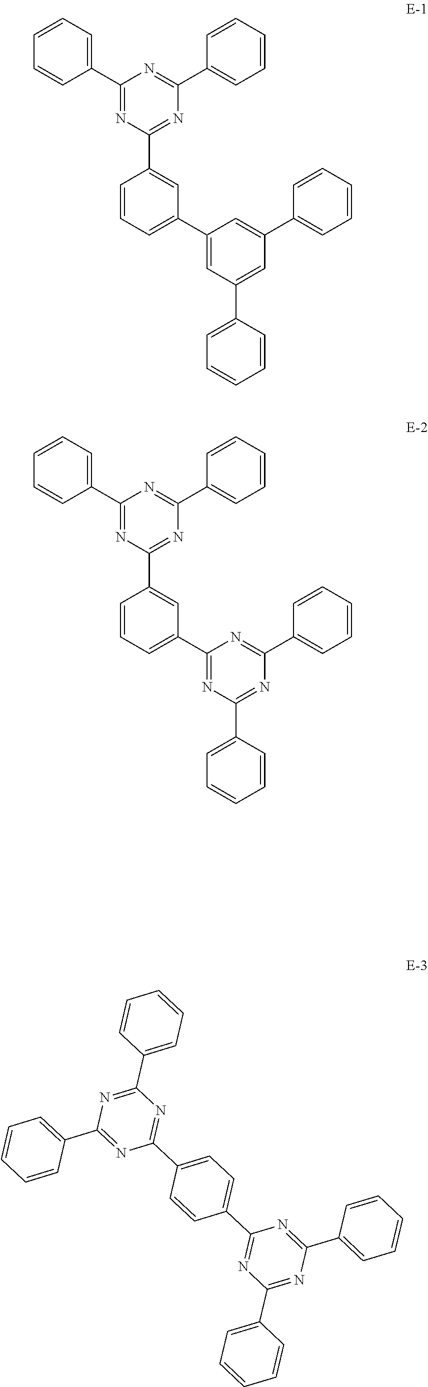 [SMILES: c1ccc(-c2cc(-c3ccccc3)cc(-c3cccc(-c4nc(-c5ccccc5)nc(-c5ccccc5)n4)c3)c2)cc1 ; c1ccc(-c2nc(-c3ccccc3)nc(-c3cccc(-c4nc(-c5ccccc5)nc(-c5ccccc5)n4)c3)n2)cc1 ; c1ccc(-c2nc(-c3ccccc3)nc(-c3ccc(-c4nc(-c5ccccc5)nc(-c5ccccc5)n4)cc3)n2)cc1]

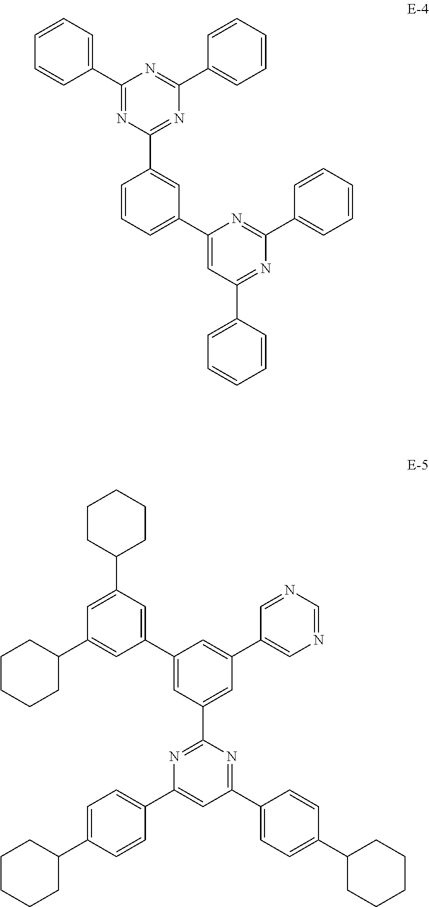 [SMILES: c1ccc(-c2cc(-c3cccc(-c4nc(-c5ccccc5)nc(-c5ccccc5)n4)c3)nc(-c3ccccc3)n2)cc1 ; c1ncc(-c2cc(-c3cc(C4CCCCC4)cc(C4CCCCC4)c3)cc(-c3nc(-c4ccc(C5CCCCC5)cc4)cc(-c4ccc(C5CCCCC5)cc4)n3)c2)cn1]

Referring to Table 14, the organic light-emitting devices of Examples 45 to 88 each had excellent or suitable characteristics in terms of driving voltage and current efficiency, compared to the organic light-emitting devices of Comparative Examples 7 to 12.

According to the one or more embodiments, when a heterocyclic compound represented by formula 1 is utilized, a light-emitting device having reduced driving voltage and improved luminescence efficiency and long lifespan and a high-quality electronic apparatus including the light-emitting device may be manufactured.

In the present disclosure, it will be understood that the terms “comprise(s),” “include(s),” or “have/has” specify the presence of stated features, integers, steps, operations, elements, and/or components, but do not preclude the presence or addition of one or more other features, integers, steps, operations, elements, components, and/or groups thereof.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within +30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in the present disclosure is intended to include all higher numerical limitations subsumed therein. Accordingly, Applicant reserves the right to amend the disclosure, including the claims, to expressly recite any sub-range subsumed within the ranges expressly recited herein.

In the present disclosure, when particles are spherical, “size” indicates a particle diameter or an average particle diameter, and when the particles are non-spherical, the “size” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The light-emitting device, the light-emitting apparatus, the display device, the electronic apparatus, the electronic equipment, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprising:
a first electrode;
a second electrode facing the first electrode;
an interlayer between the first electrode and the second electrode and comprising an emission layer; and
a heterocyclic compound represented by Formula 1: 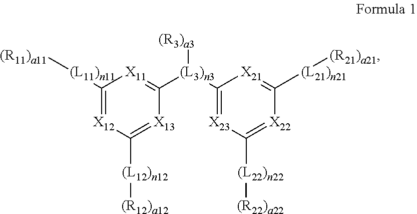
wherein, in Formula 1,
X₁₁ is C(Y₁₁) or N, X₁₂ is C(Y₁₂) or N, X₁₃ is C(Y₁₃) or N, X₂₁ is C(Y₂₁) or N, X₂₂ is C(Y₂₂) or N, and X₂₃ is C(Y₂₃) or N,
at least one selected from among X₁₁ to X₁₃ is N, and at least one selected from among X₂₁ to X₂₃ is N,
L₁₁, L₁₂, L₂₁, and L₂₂ are each independently a single bond, a C₃-C₆₀ carbocyclic group unsubstituted or substituted with at least one R₁₀ₐ, a C₁-C₆₀ heterocyclic group unsubstituted or substituted with at least one R₁₀ₐ, *—C(R₁ₐ)(R_{1b})—**, *—C(R₁ₐ)═**, *═C(R₁ₐ)—**, *—C(R₁ₐ)═C(R_{1b})—**, *—C≡C—**, *—N(R₁ₐ)—**, or *—Si(R₁ₐ)(R_{1b})—**,
L₃ is a single bond, a C₃-C₆₀ carbocyclic group unsubstituted or substituted with at least one R₁₀ₐ, a C₁-C₆₀ heterocyclic group unsubstituted or substituted with at least one R₁₀ₐ,
R₃ is absent when L₃ is a single bond,
n11, n12, n21, n22, and n3 are each independently an integer from 1 to 20,
R₁₁, R₁₂, R₂₁, R₂₂, R₃, R₁ₐ, R_{1b}, Y₁₁ to Y₁₃, and Y₂₁ to Y₂₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a11, a12, a21, a22, and a3 are each independently an integer from 1 to 20,
at least one selected from among R₁₁, R₁₂, R₂₁, R₂₂ and R₃ is a bridged C₅-C₁₂ polycyclic group unsubstituted or substituted with at least one R₁₀ₐ, wherein R₁₁, R₁₂, R₂₁, R₂₂, and R₅ are each not an adamantyl group,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₅-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The light-emitting device of claim 1, wherein
the first electrode is an anode,
the second electrode is a cathode,
the interlayer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or any combination thereof, and
the electron transport region comprises a hole blocking layer, an electron transport layer, an electron injection layer, or any combination thereof.

3. The light-emitting device of claim 2, wherein the heterocyclic compound is in the electron transport layer.

4. The light-emitting device of claim 2, wherein the electron transport region further comprises an electron transport auxiliary layer, and
the heterocyclic compound is in the electron transport auxiliary layer.

5. The light-emitting device of claim 1, wherein the emission layer is to emit light having a maximum emission wavelength in a range of about 430 nm to about 480 nm.

6. The light-emitting device of claim 1, wherein the light-emitting device has a driving voltage of 4.0 eV or less and current efficiency of 7.5 cd/A or more.

7. An electronic apparatus comprising the light-emitting device of claim 1.

8. The electronic apparatus of claim 7, further comprising a thin-film transistor, wherein
the thin-film transistor comprises a source electrode and a drain electrode, and
the first electrode of the light-emitting device is electrically connected to the source electrode or the drain electrode of the thin-film transistor.

9. An electronic equipment comprising the light-emitting device of claim 1.

10. The electronic equipment of claim 9, wherein the electronic equipment is at least one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, or a signboard.

11. A heterocyclic compound represented by Formula 1: 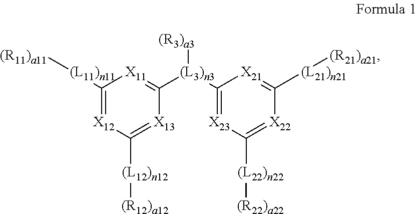
wherein, in Formula 1,
X₁₁ is C(Y₁₁) or N, X₁₂ is C(Y₁₂) or N, X₁₃ is C(Y₁₃) or N, X₂₁ is C(Y₂₁) or N, X₂₂ is C(Y₂₂) or N, and X₂₃ is C(Y₂₃) or N,
at least one selected from among X₁₁ to X₁₃ is N, and at least one selected from among X₂₁ to X₂₃ is N,
L₁₁, L₁₂, L₂₁, and L₂₂ are each independently a single bond, a C₃-C₆₀ carbocyclic group unsubstituted or substituted with at least one R₁₀ₐ, a C₁-C₆₀ heterocyclic group unsubstituted or substituted with at least one R₁₀ₐ, *—C(R₁ₐ)(R_{1b})—**, *—C(R₁ₐ)═**, *═C(R₁ₐ)—**, *—C(R₁ₐ)═C(R_{1b})—**, *—C≡C—**, *—N(R₁ₐ)—**, or *—Si(R₁ₐ)(R_{1b})—*″,
L₃ is a single bond, a C₃-C₆₀ carbocyclic group unsubstituted or substituted with at least one R₁₀ₐ, a C₁-C₆₀ heterocyclic group unsubstituted or substituted with at least one R₁₀ₐ,
R₃ is absent when L₃ is a single bond,
n11, n12, n21, n22, and n3 are each independently an integer from 1 to 20,
R₁₁, R₁₂, R₂₁, R₂₂, R₃, R₁ₐ, R_{1b}, Y₁₁ to Y₁₃, and Y₂₁ to Y₂₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a11, a12, a21, a22, and a3 are each independently an integer from 1 to 20,
at least one selected from among R₁₁, R₁₂, R₂₁, R₂₂ and R₃ is a bridged C₅-C₁₂ polycyclic group unsubstituted or substituted with at least one R₁₀ₐ, wherein R₁₁, R₁₂, R₂₁, R₂₂, and R₅ are each not an adamantyl group,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₅-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

12. The heterocyclic compound of claim 11, wherein at least two selected from among X₁₁ to X₁₃ are each N, and at least two selected from among X₂₁ to X₂₃ are each N.

13. The heterocyclic compound of claim 11, wherein L₁₁, L₁₂, L₂₁, L₂₂, and L₃ are each independently:
a single bond; or
a benzene group, a pentalene group, an indene group, a naphthalene group, an azulene group, a heptalene group, an indacene group, an acenaphthylene group, a fluorene group, a spiro-bifluorene group, a benzofluorene group, a phenalene group, a phenanthrene group, an anthracene group, a fluoranthene group, a triphenylene group, a pyrene group, a chrysene group, a naphthacene group, a picene group, a perylene group, a pentaphenylene group, a hexacene group, a pyrrole group, an imidazole group, a pyrazole group, a pyridine group, a pyrazine group, a pyrimidine group, a pyridazine group, an isoindole group, an indole group, an indazole group, a purine group, a quinoline group, an isoquinoline group, a benzoquinoline group, a phthalazine group, a naphthyridine group, a quinoxaline group, a quinazoline group, a cinnoline group, a carbazole group, a dibenzosilole group, a phenanthridine group, an acridine group, a phenanthroline group, a phenazine group, a benzoxazole group, a benzimidazole group, a furan group, a benzofuran group, a thiophene group, a benzothiophene group, a thiazole group, an isothiazole group, a benzothiazole group, an isoxazole group, an oxazole group, a triazole group, a tetrazole group, an oxadiazole group, a triazine group, a benzoxazole group, a dibenzofuran group, a dibenzothiophene group, a benzocarbazole group, or a dibenzocarbazole group, each unsubstituted or substituted with at least one of deuterium, —F, —Cl, —Br, —I, a hydroxyl group, a cyano group, a nitro group, a C₁-C₂₀ alkyl group, a C₁-C₂₀ alkoxy group, a C₆-C₂₀ aryl group, a C₁-C₂₀ heteroaryl group, —C(Q₃₁) (Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), or a combination thereof, and
wherein Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₅-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

14. The heterocyclic compound of claim 11, wherein L₁₁, L₁₂, L₂₁, L₂₂, and L₃ are each independently:
a single bond, a benzene group, a naphthalene group, an azulene group, a heptalene group, a phenalene group, a phenanthrene group, an anthracene group, a pyrrole group, a furan group, a thiophene group, a cyclopentadiene group, a silole group, a pyridine group, a pyrimidine group, a pyrazine group, a triazine group, a carbazole group, a dibenzofuran group, a dibenzothiophene group, a fluorene group, or a dibenzosilole group.

15. The heterocyclic compound of claim 11, wherein
L₁₁ is not a single bond when R₁₁ is a bridged C₅-C₁₂ polycyclic group unsubstituted or substituted with at least one R₁₀ₐ,
L₁₂ is not a single bond when R₁₂ is a bridged C₅-C₁₂ polycyclic group unsubstituted or substituted with at least one R₁₀ₐ,
L₂₁ is not a single bond when R₂₁ is a bridged C₅-C₁₂ polycyclic group unsubstituted or substituted with at least one R₁₀ₐ,
L₂₂ is not a single bond when R₂₂ is a bridged C₅-C₁₂ polycyclic group unsubstituted or substituted with at least one R₁₀ₐ, and
L₃ is not a single bond when R₃ is a bridged C₅-C₁₂ polycyclic group unsubstituted or substituted with at least one R₁₀ₐ.

16. The heterocyclic compound of claim 11, wherein R₁₁, R₁₂, R₂₁, R₂₂, and R₃ are each independently:
a cyclopentyl group, a cyclohexyl group, a cycloheptyl group, a cyclooctyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a pyrrolyl group, an imidazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a triazolyl group, a triazinyl group, a benzocarbazolyl group, a dibenzocarbazolyl group, an azacarbazolyl group, an azadibenzofuranyl group, a bicyclo[1.1.1]pentanyl group, a bicyclo[2.1.1]hexanyl group, a bicyclo[2.2.1]heptanyl group, a bicyclo[2.2.2]octanyl group, a bicyclo[3.2.1]octanyl group, a bicyclo[3.2.2]nonanyl group, a bicyclo[3.3.1]nonanyl group, a tricyclo[3.1.1.0(3,6)]heptanyl group, a tricyclo[4.1.1.0(3,7)]octanyl group, a bicyclo[1.1.1]cyclopentenyl group, a bicyclo[2.1.1]cyclohexenyl group, a bicyclo[2.2.1]cycloheptenyl group, a bicyclo[2.2.2]octenyl group, a bicyclo[3.2.1]octenyl group, a bicyclo[3.2.2]nonenyl group, a bicyclo[3.3.1]nonenyl group, a tricyclo[3.1.1.0(3,6)]cycloheptenyl group, or a tricyclo[4.1.1.0(3,7)]octenyl group, each unsubstituted or substituted with a deuterium, a cyano group, a C₁-C₂₀ alkyl group, a cyclopentyl group, a cyclohexyl group, a cycloheptyl group, a cyclooctyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a benzimidazolyl group, a triazolyl group, a triazinyl group, a benzocarbazolyl group, a dibenzocarbazolyl group, —N(Q₃₁)(Q₃₂), or any combination thereof, and
wherein Q₃₁ and Q₃₂ are each independently: hydrogen; deuterium; a cyano group; a C₁-C₆₀ alkyl group; a C₂-C₆₀ alkenyl group; a C₂-C₆₀ alkynyl group; or a C₃-C₆₀ carbocyclic group or a C₁-C₆₀ heterocyclic group, each unsubstituted or substituted with deuterium, a cyano group, a C₁-C₆₀ alkyl group, a phenyl group, a biphenyl group, or any combination thereof.

17. The heterocyclic compound of claim 11, wherein at least one selected from among R₁₁, R₁₂, R₂₁, R₂₂, and R₃ is:
a bicyclo[1.1.1]pentanyl group, a bicyclo[2.1.1]hexanyl group, a bicyclo[2.2.1]heptanyl group, a bicyclo[2.2.2]octanyl group, a bicyclo[3.2.1]octanyl group, a bicyclo[3.2.2]nonanyl group, a bicyclo[3.3.1]nonanyl group, a tricyclo[3.1.1.0(3,6)]heptanyl group, a tricyclo[4.1.1.0(3,7)]octanyl group, a bicyclo[1.1.1]cyclopentenyl group, a bicyclo[2.1.1]cyclohexenyl group, a bicyclo[2.2.1]cycloheptenyl group, a bicyclo[2.2.2]octenyl group, a bicyclo[3.2.1]octenyl group, a bicyclo[3.2.2]nonenyl group, a bicyclo[3.3.1]nonenyl group, a tricyclo[3.1.1.0(3,6)]cycloheptenyl group, or a tricyclo[4.1.1.0(3,7)]octenyl group.

18. The heterocyclic compound of claim 11, wherein L₁₁, L₁₂, L₂₁, and L₂₂ are each independently selected from groups represented by Formulae 1-1 to 1-37: 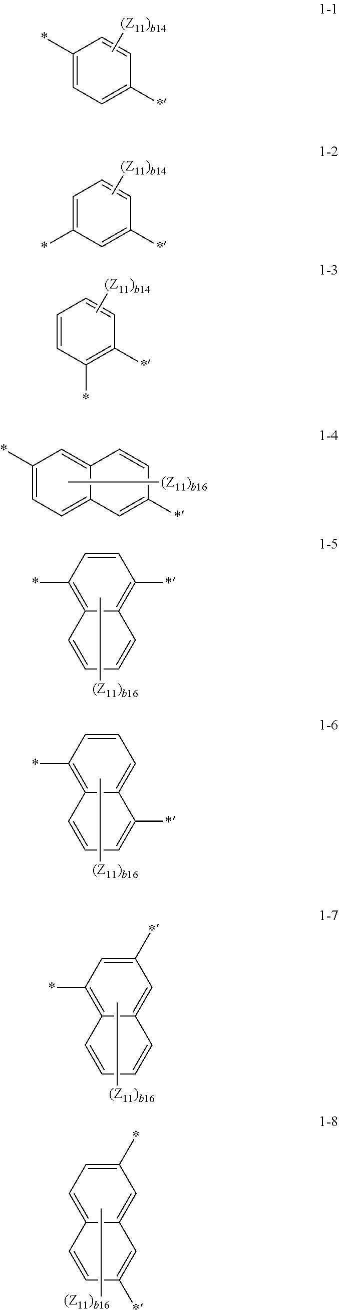 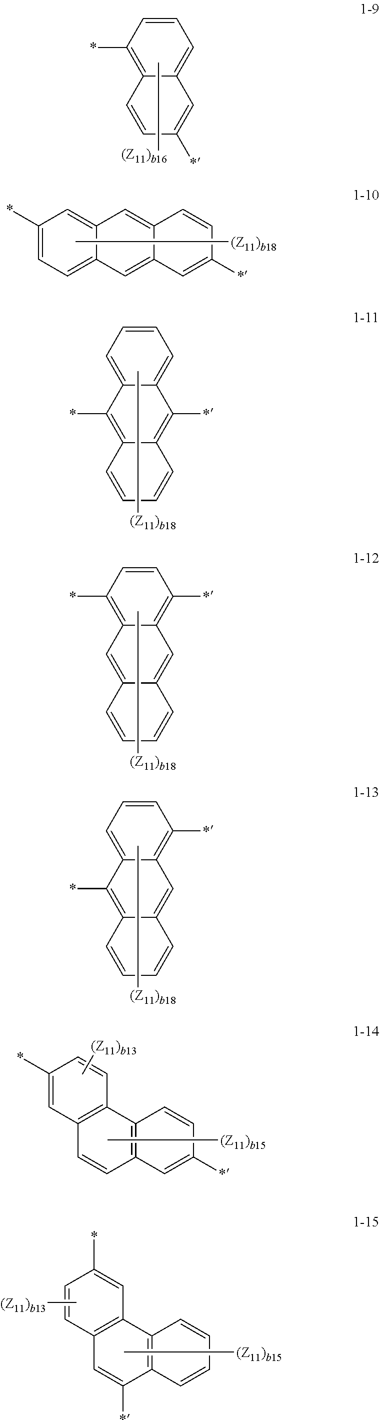 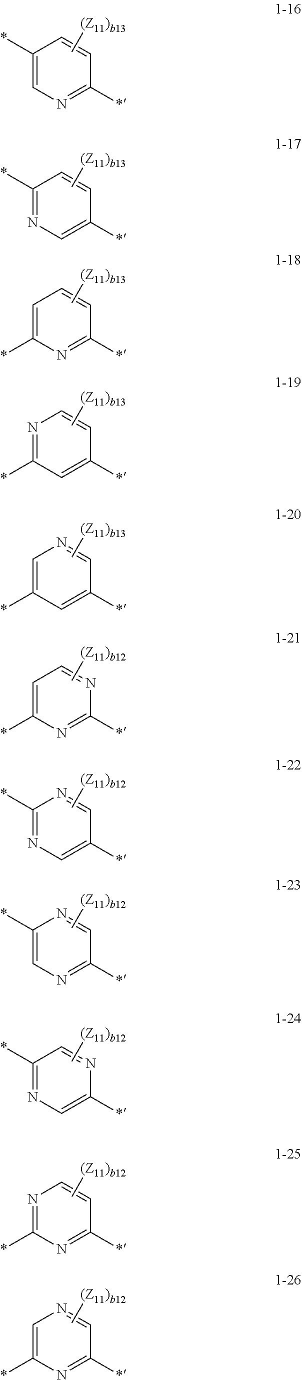 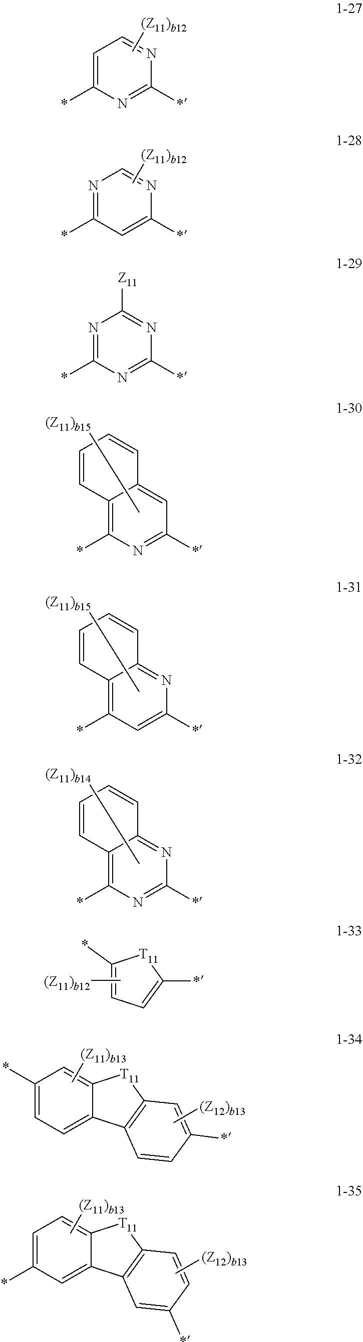 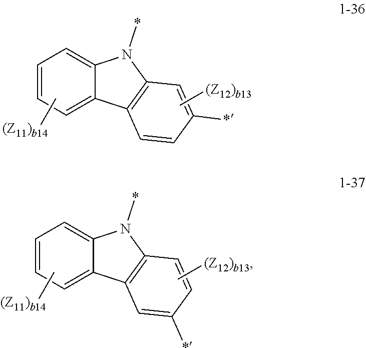
and
wherein, in Formulae 1-1 to 1-37,
T₁₁ is O, S, C(Z₁₃)(Z₁₄), N(Z₁₅), or Si(Z₁₆)(Z₁₇),
Z₁₁ to Z₁₇ are each independently selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, —Si(Q₃₁)(Q₃₂)(Q₃₃), —N(Q₃₁)(Q₃₂), and —B(Q₃₁)(Q₃₂),
b12 is 1 or 2,
b13 is an integer from 1 to 3,
b14 is an integer from 1 to 4,
b15 is an integer from 1 to 5,
b16 is an integer from 1 to 6,
b18 is an integer from 1 to 8,
Q₃₁ to Q₃₃ are each the same as defined in Formula 1, and
* and ** each indicate a binding site to a neighboring atom.

19. The heterocyclic compound of claim 11, wherein L₃ is selected from groups represented by Formulae 2-1 to 2-22: 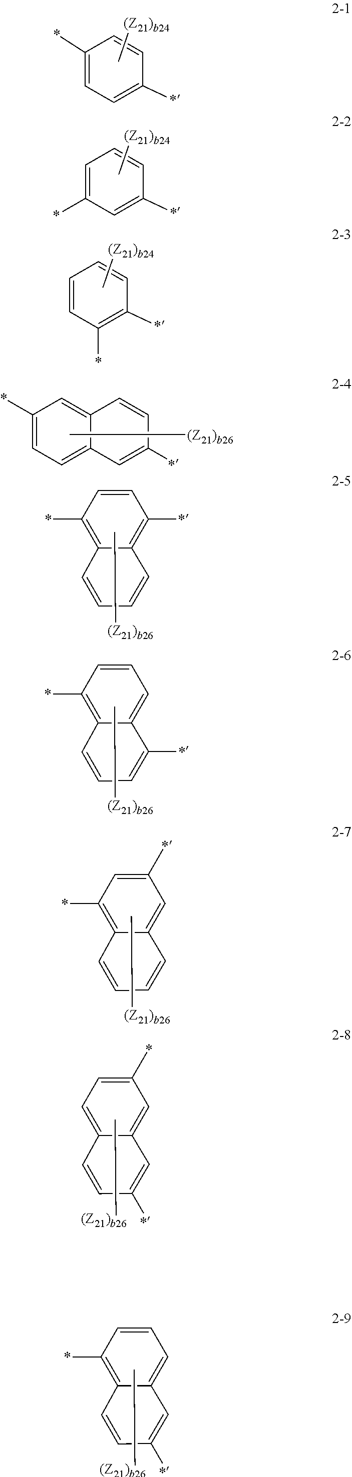 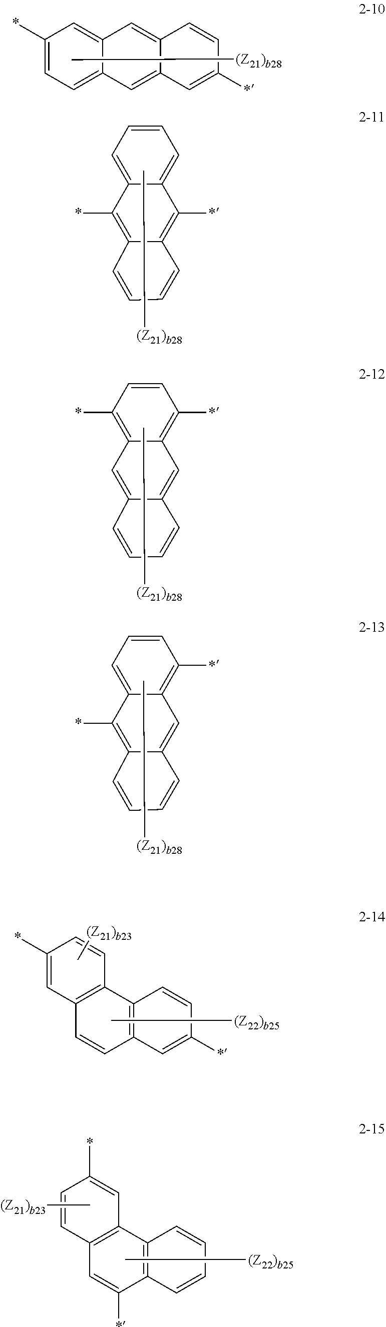 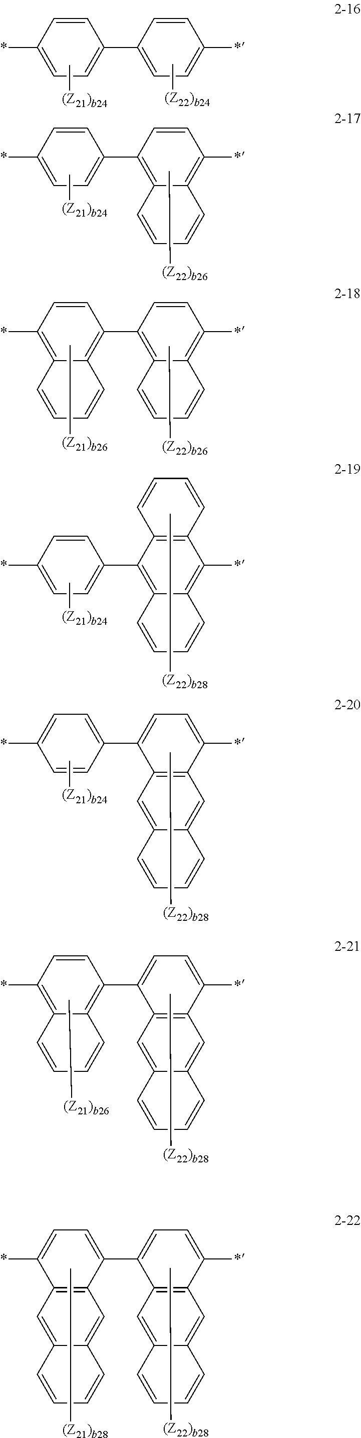
and
wherein, in Formulae 2-1 to 2-22,
Z₂₁ and Z₂₂ are each independently selected from *″, hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, —Si(Q₃₁)(Q₃₂)(Q₃₃), —N(Q₃₁)(Q₃₂), and —B(Q₃₁)(Q₃₂),
b23 is an integer from 1 to 3,
b24 is an integer from 1 to 4,
b25 is an integer from 1 to 5,
b26 is an integer from 1 to 6,
b28 is an integer from 1 to 8,
Q₃₁ to Q₃₃ are each the same as defined in Formula 1, and
*, *′, and *″ each indicate a binding site to a neighboring atom.

20. The heterocyclic compound of claim 11, wherein at least one selected from among R₁₁, R₁₂, R₂₁, R₂₂, and R₃ is selected from groups represented by Formulae 3-1 to 3-24: 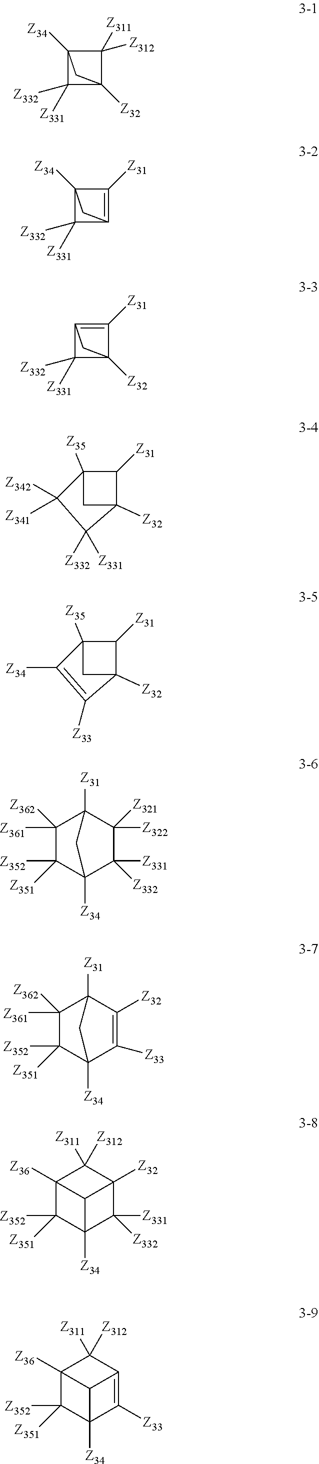 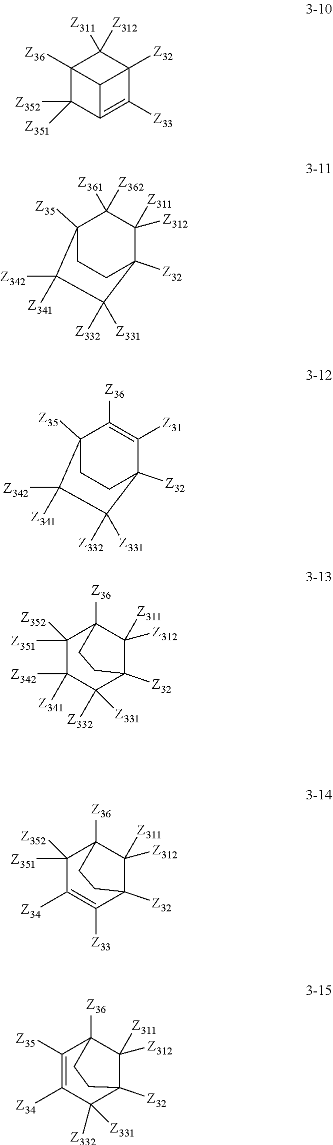 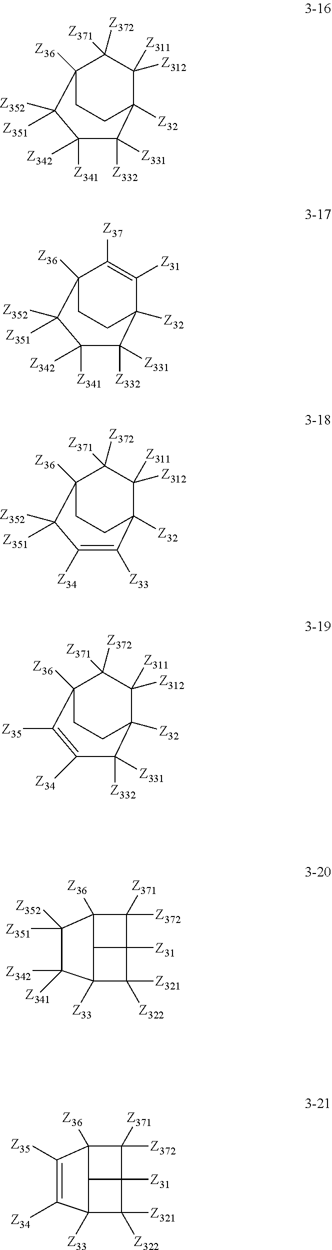 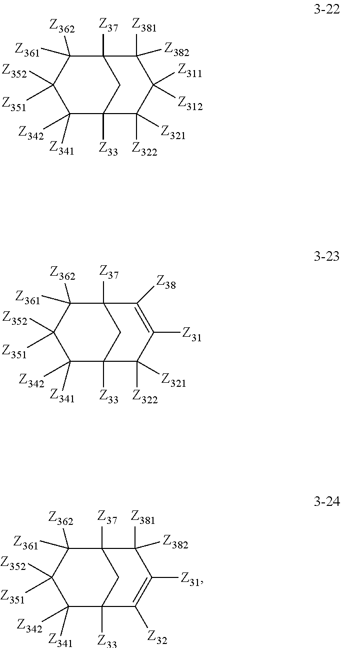
wherein, in Formulae 3-1 to 3-24,
Z₃₁ to Z₃₈, Z₃₁₁, Z₃₁₂, Z₃₂₁, Z₃₂₂, Z₃₃₁, Z₃₃₂, Z₃₄₁, Z₃₄₂, Z₃₅₁, Z₃₅₂, Z₃₆₁, Z₃₆₂, Z₃₇₁, Z₃₇₂, Z₃₈₁, and Z₃₈₂ are each independently:
hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, —Si(Q₃₁)(Q₃₂)(Q₃₃), —N(Q₃₁)(Q₃₂), or —B(Q₃₁)(Q₃₂); or
*, and
wherein Q₃₁ to Q₃₃ are each the same as defined in Formula 1, and
* indicates a binding site to a neighboring atom.
